(19) United States
(12) Patent Application Publication
(10) Pub. No.: US 2024/0392299 A1
(43) Pub. Date: Nov. 28, 2024

(54) TREATMENT OF MST1R RELATED DISEASES AND DISORDERS

(71) Applicant: Empirico Inc., San Diego, CA (US)

(72) Inventors: Omri GOTTESMAN, San Diego, CA (US); Shannon BRUSE, San Diego, CA (US); Paul BUSKE, Madison, WI (US); Brian CAJES, San Diego, CA (US); David JAKUBOSKY, San Diego, CA (US); Sarah KLEINSTEIN, San Diego, CA (US); David LEWIS, Madison, WI (US); David ROZEMA, Cross Plains, WI (US); John VEKICH, San Diego, CA (US)

(21) Appl. No.: 18/567,669

(22) PCT Filed: Jun. 14, 2022
(86) PCT No.: PCT/US2022/033350

§ 371 Date: Dec. 6, 2023

Related U.S. Application Data
(60) Provisional application No. 63/211,370, filed on Jun. 16, 2021.

(51) Int. Cl. C12N 15/113

(52) U.S. Cl. CPC C12N 15/1137; C12N 2310/11; C12N 2310/14; C12N 2310/321; C12N 2310/322; C12N 2310/351; C12Y 207/10001

(57) ABSTRACT

Disclosed herein are compositions comprising an oligonucleotide that targets MST1R. The oligonucleotide may include a small interfering RNA (siRNA) or an antisense oligonucleotide (ASO). Also provided herein are methods of treating conditions associated with MST1R mutations that include providing an oligonucleotide that targets MST1R to a subject.

### CROSS-REFERENCE

This application is a U.S. national stage entry of International Application No. PCT/US2022/033350, filed Jun. 14, 2022, which claims the benefit of U.S. Provisional Application No. 63/211,370, filed Jun. 16, 2021, which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 2, 2022, is named 54462-731_831_SL.txt and is 2,357,478 bytes in size.

### BACKGROUND

Lung disorders are a common problem, and may affect a wide variety of persons. Improved therapeutics are needed for treating these disorders.

### SUMMARY

Described herein are compositions comprising an oligonucleotide that targets MST1R. Described herein are compositions comprising an oligonucleotide that targets MST1R and when administered to a subject in an effective amount reduces a MST1R mRNA level or MST1R protein level. Described herein are compositions comprising an oligonucleotide that targets MST1R and when administered to a subject in an effective amount increases a lung function measurement. In some embodiments, the lung function measurement comprises a forced expiratory volume in 1 second (FEV1) measurement, a forced expiratory volume in 1 second percent predicted (FEV1pp) measurement, a forced vital capacity (FVC) measurement, a FEV1/FVC ratio measurement, a forced expiratory volume, or a peak expiratory flow measurement. In some embodiments, the lung function measurement is increased by about 10% or more, as compared to prior to administration. Described herein are compositions comprising an oligonucleotide that targets MST1R and when administered to a subject in an effective amount decreases a leukocyte measurement. In some embodiments, the leukocyte measurement comprises a lung leukocyte measurement. In some embodiments, the leukocyte measurement comprises a circulating leukocyte measurement. In some embodiments, the leukocyte measurement comprises a neutrophil measurement, eosinophil measurement, basophil measurement, monocyte measurement, or lymphocyte measurement, or a combination thereof. In some embodiments, the leukocyte measurement is decreased by about 10% or more, as compared to prior to administration. Described herein are compositions comprising an oligonucleotide that targets MST1R and when administered to a subject in an effective amount decreases a chronic obstructive pulmonary disease (COPD) or asthma exacerbation measurement. In some embodiments, the COPD or asthma exacerbation measurement is decreased by about 10% or more, as compared to prior to administration. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises 1, 2, 3, 4, 5, 6, 7, 8, 9, 10, 11, 12, 13, 14, 15, 16, 17, 18, 19, 20, or 21 modified nucleosides. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the oligonucleotide comprises a sugar moiety attached at a 3′ or 5′ terminus of the oligonucleotide. In some embodiments, the oligonucleotide comprises an integrin targeting ligand attached at a 3′ or 5′ terminus of the oligonucleotide. In some embodiments, the integrin comprises integrin alpha-v-beta-6. In some embodiments, the integrin targeting ligand comprises an arginine-glycine-aspartic acid (RGD) peptide. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. Described herein are compositions comprising an oligonucleotide that inhibits the expression of MST1R, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 9818. In some embodiments, any one of the following is true with regard to the sense strand: all purines comprise 2′ fluoro modified purines, and all pyrimidines comprise a mixture of 2′ fluoro and 2′ methyl modified pyrimidines; all purines comprise 2′ methyl modified purines, and all pyrimidines comprise a mixture of 2′ fluoro and 2′ methyl modified pyrimidines; all purines comprise 2′ fluoro modified purines, and all pyrimidines comprise 2′ methyl modified pyrimidines; all pyrimidines comprise 2′ fluoro modified pyrimidines, and all purines comprise a mixture of 2′ fluoro and 2′ methyl modified purines; all pyrimidines comprise 2′ methyl modified pyrimidines, and all purines comprise a mixture of 2′ fluoro and 2′ methyl modified purines; or all pyrimidines comprise 2′ fluoro modified pyrimidines, and all purines comprise 2′ methyl modified purines. In some embodiments, the sense strand comprises any one of modification patterns 1S, 2S, 3S, 4S, 5S, 6S, 7S, 8S, or 9S. In some embodiments, any one of the following is true with regard to the antisense strand: all purines comprise 2′ fluoro modified purines, and all pyrimidines comprise a mixture of 2′ fluoro and 2′ methyl modified pyrimidines; all purines comprise 2′ methyl modified purines, and all pyrimidines comprise a mixture of 2′ fluoro and 2′ methyl modified pyrimidines; all purines comprise 2′ methyl modified purines, and all pyrimidines comprise 2′ fluoro modified pyrimidines; all pyrimidines comprise 2′ fluoro modified pyrimidines, and all purines comprise a mixture of 2′ fluoro and 2′ methyl modified purines; all pyrimidines comprise 2′ methyl modified pyrimidines, and all purines comprise a mixture of 2′ fluoro and 2′ methyl modified purines; or all pyrimidines comprise 2′ methyl modified pyrimidines, and all purines comprise 2′ fluoro modified purines. In some embodiments, the antisense strand comprises any one of modification patterns 1AS, 2AS, 3AS, 4AS, 5AS, 6AS, 7AS, or 8AS. In some embodiments, the sense strand comprises the nucleic acid sequence of any one of SEQ ID NOs: 1-4754, and the antisense strand comprises the nucleic acid sequence of any one of SEQ ID NOs: 4755-9508. In some embodiments, the oligonucleotide comprises an antisense oligonucleotide (ASO). In some embodiments, the ASO is 12-30 nucleosides in length. Described herein are compositions comprising an oligonucleotide that inhibits the expression of MST1R, wherein the oligonucleotide comprises an ASO about 12-30 nucleosides in length and a nucleoside sequence complementary to about 12-30 contiguous nucleosides of SEQ ID NO: 9818. Some embodiments include a pharmaceutically acceptable carrier. Described herein are methods of treating a subject having a lung disorder, comprising administering an effective amount of the composition to the subject. In some embodiments, the lung disorder COPD, acute exacerbation of COPD, emphysema, chronic bronchitis, asthma, status asthmaticus, asthma-COPD overlap syndrome (ACOS), cough, lung cancer, interstitial lung disease, or pulmonary fibrosis.

### DETAILED DESCRIPTION

Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, lung) and a relevant indication.

The MST1R (macrophage-stimulating 1 receptor) gene is located on chromosome 3, and encodes macrophage-stimulating 1 receptor (MST1R), also known as macrophage-stimulating protein receptor or Recepteur d'Origine Nantais (RON) kinase. The MST1R gene may encode various transcripts or splice variants. MST1R may include 1400 amino acids and have a mass of about 152 kDa. MST1R may be cleaved into an alpha and beta chain. MST1R may comprise a receptor tyrosine kinase that transduces signals from the extracellular matrix into the cytoplasm by binding macrophage-stimulating protein (MSP) encoded by MST1 (macrophage-stimulating 1). MSP may also be referred to as MST1 protein. MST1R may be intracellular. MST1R may be cell membrane-bound. MST1R may be expressed in lungs. MST1R may bind or interact with MSP and stimulate lung ciliary motility. The MSP may be secreted by the liver and enter the bloodstream prior to interacting with MST1R at the lung. An example of an MST1R amino acid sequence, and further description of MST1R is included at uniprot.org under accession no. Q04912 (last modified Mar. 28, 2018).

Here, it is shown that a genetic variant that may result in loss of function of the MST1R gene in humans are associated with decreased risk of chronic obstructive pulmonary disease (COPD), family history of COPD, asthma, and use of inhaled beta agonist medication. Also shown is that genetic variants that may result in loss of function of the gene encoding MST1R's binding partner, MST1, are also associated with decreased risk of COPD, family history of COPD, asthma, and use of inhaled beta agonist medication. Therefore, inhibition of MST1R may serve as a therapeutic strategy for treatment of a lung disorder such as COPD, acute exacerbation of COPD, emphysema, chronic bronchitis, asthma, status asthmaticus, asthma-COPD overlap syndrome (ACOS), cough, lung cancer, interstitial lung disease, or pulmonary fibrosis.

Disclosed herein, are methods or compositions that inhibit or target MST1R. Where inhibition or targeting of MST1R is disclosed, it is contemplated that some embodiments may include inhibiting or targeting a MST1R protein or MST1R RNA. For example, by inhibiting or targeting an RNA (e.g. mRNA) encoded by the MST1R gene using an oligonucleotide described herein, the MST1R protein may be inhibited or targeted as a result of there being less production of the MST1R protein by translation of the MST1R RNA; or a MST1R protein may be targeted or inhibited by an oligonucleotide that binds or interacts with a MST1R RNA and reduces production of the MST1R protein from the MST1R RNA. Thus, targeting MST1R may refer to binding a MST1R RNA and reducing MST1R RNA or MST1R protein levels. The oligonucleotide may include a small interfering RNA (siRNA) or an antisense oligonucleotide (ASO). Also provided herein are methods of treating a lung disorder by providing an oligonucleotide that targets MST1R to a subject in need thereof.

### I. COMPOSITIONS

Disclosed herein, in some embodiments, are compositions comprising an oligonucleotide. In some embodiments, the composition comprises an oligonucleotide that targets MST1R. In some embodiments, the composition consists of an oligonucleotide that targets MST1R. In some embodiments, the oligonucleotide reduces MST1R mRNA expression in the subject. In some embodiments, the oligonucleotide reduces MST1R protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder as described herein.

Some embodiments include a composition comprising an oligonucleotide that targets MST1R and when administered to a subject in an effective amount decreases MST1R mRNA or MST1R protein levels in a cell, fluid or tissue. In some embodiments, the composition comprises an oligonucleotide that targets MST1R and when administered to a subject in an effective amount decreases MST1R mRNA levels in a cell or tissue. In some embodiments, the cell is a lung cell, lung epithelial cell, type I or II alveolar cell, macrophage, alveolar macrophage, goblet cell, club cell, or fibroblast. In some embodiments, the tissue is lung tissue. In some embodiments, the MST1R mRNA levels are decreased by about 2.5% or more, about 5% or more, or about 7.5% or more, as compared to prior to administration. In some embodiments, the MST1R mRNA levels are decreased by about 10% or more, as compared to prior to administration. In some embodiments, the MST1R mRNA levels are decreased by about 20% or more, about 30% or more, about 40% or more, about 50% or more, about 60% or more, about 70% or more, about 80% or more, about 90% or more, or about 100%, as compared to prior to administration. In some embodiments, the MST1R mRNA levels are decreased by no more than about 2.5%, no more than about 5%, or no more than about 7.5%, as compared to prior to administration. In some embodiments, the MST1R mRNA levels are decreased by no more than about 10%, as compared to prior to administration. In some embodiments, the MST1R mRNA levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the MST1R mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MST1R and when administered to a subject in an effective amount decreases MST1R protein levels in a cell or tissue. In some embodiments, the cell is a lung cell, lung epithelial cell, type I or II alveolar cell, macrophage, alveolar macrophage, goblet cell, club cell, or fibroblast. In some embodiments, the tissue is lung tissue. In some embodiments, the MST1R protein levels are decreased by about 2.5% or more, about 5% or more, or about 7.5% or more, as compared to prior to administration. In some embodiments, the MST1R protein levels are decreased by about 10% or more, as compared to prior to administration. In some embodiments, the MST1R protein levels are decreased by about 20% or more, about 30% or more, about 40% or more, about 50% or more, about 60% or more, about 70% or more, about 80% or more, about 90% or more, or about 100%, as compared to prior to administration. In some embodiments, the MST1R protein levels are decreased by no more than about 2.5%, no more than about 5%, or no more than about 7.5%, as compared to prior to administration. In some embodiments, the MST1R protein levels are decreased by no more than about 10%, as compared to prior to administration. In some embodiments, the MST1R protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the MST1R protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MST1R and when administered to a subject in an effective amount diminishes an adverse phenotype of lung disorder in the subject. The lung disorder may include chronic obstructive pulmonary disease (COPD), acute exacerbation of COPD, emphysema, chronic bronchitis, asthma, status asthmaticus, asthma-COPD overlap syndrome (ACOS), cough, lung cancer, interstitial lung disease, or pulmonary fibrosis. In some embodiments, the adverse phenotype is decreased by about 2.5% or more, about 5% or more, or about 7.5% or more, as compared to prior to administration. In some embodiments, the adverse phenotype is decreased by about 10% or more, as compared to prior to administration. In some embodiments, the adverse phenotype is decreased by about 20% or more, about 30% or more, about 40% or more, about 50% or more, about 60% or more, about 70% or more, about 80% or more, about 90% or more, or about 100%, as compared to prior to administration. In some embodiments, the adverse phenotype is decreased by no more than about 2.5%, no more than about 5%, or no more than about 7.5%, as compared to prior to administration. In some embodiments, the adverse phenotype is decreased by no more than about 10%, as compared to prior to administration. In some embodiments, the adverse phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the adverse phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MST1R and when administered to a subject in an effective amount enhances a protective phenotype of a lung disorder. The lung disorder may include chronic obstructive pulmonary disease (COPD), acute exacerbation of COPD, emphysema, chronic bronchitis, asthma, status asthmaticus, asthma-COPD overlap syndrome (ACOS), cough, lung cancer, interstitial lung disease, or pulmonary fibrosis. In some embodiments, the protective phenotype is increased by about 2.5% or more, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets MST1R and when administered to a subject in an effective amount improves (e.g. increases) a lung function measurement. The lung function measurement may include a measurement of forced expiratory volume in 1 second (FEV1), forced expiratory volume in 1 second percent predicted (FEV1pp), forced vital capacity (FVC), FEV1/FVC ratio, forced expiratory volume, or peak expiratory flow. In some embodiments, the lung function measurement is improved by about 2.5% or more, about 5% or more, or about 7.5% or more, as compared to prior to administration. In some embodiments, the lung function measurement is improved by about 10% or more, as compared to prior to administration. In some embodiments, the lung function measurement is improved by about 20% or more, about 30% or more, about 40% or more, about 50% or more, about 60% or more, about 70% or more, about 80% or more, about 90% or more, or about 100% or more, as compared to prior to administration. In some embodiments, the lung function measurement is improved by about 200% or more, about 300% or more, about 400% or more, about 500% or more, about 600% or more, about 700% or more, about 800% or more, about 900% or more, or about 1000% or more, as compared to prior to administration. In some embodiments, the lung function measurement is improved by no more than about 2.5%, no more than about 5%, or no more than about 7.5%, as compared to prior to administration. In some embodiments, the lung function measurement is improved by no more than about 10%, as compared to prior to administration. In some embodiments, the lung function measurement is improved by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the lung function measurement is improved by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the lung function measurement is improved by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

A leukocyte measurement may be affected by a lung disorder. For example, some inflammatory lung disorders that may include chronic obstructive pulmonary disease (COPD) or asthma may lead to increased inflammation and circulating white blood cell counts that may be treated using a composition comprising an oligonucleotide; or lung inflammation concomitant with a lung disorder may include an increase in leukocytes in a lung tissue or lung fluid (e.g. bronchoalveolar fluid). In some embodiments, the composition comprises an oligonucleotide that targets MST1R and when administered to a subject in an effective amount changes a leukocyte measurement in a cell, fluid or tissue of the subject. In some embodiments, the cell is a lung cell, lung epithelial cell, type I or II alveolar cell, macrophage, alveolar macrophage, goblet cell, club cell, or fibroblast. In some embodiments, the tissue is lung tissue. In some embodiments, the fluid is a blood, serum, or plasma sample. In some embodiments, the fluid is a lung fluid such as a bronchoalveolar fluid. The change may be a decrease (for example, when circulating levels of leukocytes, or levels of leukocytes in lungs are increased due to an inflammatory lung disorder). The change may be an increase in some embodiments. In some embodiments, the leukocyte measurement is changed by about 2.5% or more, about 5% or more, or about 7.5% or more, as compared to prior to administration. In some embodiments, the leukocyte measurement is changed by about 10% or more, as compared to prior to administration. In some embodiments, the leukocyte measurement is changed by about 20% or more, about 30% or more, about 40% or more, about 50% or more, about 60% or more, about 70% or more, or about 80% or more, as compared to prior to administration. In some embodiments, the leukocyte measurement is changed by no more than about 2.5%, no more than about 5%, or no more than about 7.5%, as compared to prior to administration. In some embodiments, the leukocyte measurement is changed by no more than about 10%, as compared to prior to administration. In some embodiments, the leukocyte measurement is changed by no more than about 20%, no more than about 30%, no more than about 40%, no more than about 50%, no more than about 60%, no more than about 70%, no more than about 80%, or no more than about 90%, as compared to prior to administration. In some embodiments, the leukocyte measurement is changed by 2.5%, 5%, 7.5%, 10%, 15%, 20%, 25%, 30%, 35%, 40%, 45%, 50%, 55%, 60%, 65%, 70%, 75%, 80%, 85%, or 90%, or by a range defined by any of the two aforementioned percentages.

In some embodiments, the composition comprises an oligonucleotide that targets MST1R and when administered to a subject in an effective amount decreases chronic obstructive pulmonary disease (COPD) exacerbations in the subject. In some embodiments, the COPD exacerbations are decreased by about 2.5% or more, about 5% or more, or about 7.5% or more, as compared to prior to administration. In some embodiments, the COPD exacerbations are decreased by about 10% or more, as compared to prior to administration. In some embodiments, the COPD exacerbations are decreased by about 20% or more, about 30% or more, about 40% or more, about 50% or more, about 60% or more, about 70% or more, about 80% or more, about 90% or more, or about 100%, as compared to prior to administration. In some embodiments, the COPD exacerbations are decreased by no more than about 2.5%, no more than about 5%, or no more than about 7.5%, as compared to prior to administration. In some embodiments, the COPD exacerbations are decreased by no more than about 10%, as compared to prior to administration. In some embodiments, the COPD exacerbations are decreased by no more than about 20%, no more than about 30%, no more than about 40%, no more than about 50%, no more than about 60%, no more than about 70%, no more than about 80%, or no more than about 90%, as compared to prior to administration. In some embodiments, the COPD exacerbations are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets MST1R and when administered to a subject in an effective amount decreases asthma exacerbations in the subject. In some embodiments, the asthma exacerbations are decreased by about 2.5% or more, about 5% or more, or about 7.5% or more, as compared to prior to administration. In some embodiments, the asthma exacerbations are decreased by about 10% or more, as compared to prior to administration. In some embodiments, the asthma exacerbations are decreased by about 20% or more, about 30% or more, about 40% or more, about 50% or more, about 60% or more, about 70% or more, about 80% or more, about 90% or more, or about 100%, as compared to prior to administration. In some embodiments, the asthma exacerbations are decreased by no more than about 2.5%, no more than about 5%, or no more than about 7.5%, as compared to prior to administration. In some embodiments, the asthma exacerbations are decreased by no more than about 10%, as compared to prior to administration. In some embodiments, the asthma exacerbations are decreased by no more than about 20%, no more than about 30%, no more than about 40%, no more than about 50%, no more than about 60%, no more than about 70%, no more than about 80%, or no more than about 90%, as compared to prior to administration. In some embodiments, the asthma exacerbations are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

A. siRNAs

In some embodiments, the composition comprises an oligonucleotide that targets MST1R, wherein the oligonucleotide comprises a small interfering RNA (siRNA). In some embodiments, the composition comprises an oligonucleotide that targets MST1R, wherein the oligonucleotide comprises a small interfering RNA (siRNA) comprising a sense strand and an antisense strand.

In some embodiments, the composition comprises an oligonucleotide that inhibits the expression of MST1R, wherein the oligonucleotide comprises an siRNA comprising a sense strand and an antisense strand, wherein the sense strand is 12-30 nucleosides in length. In some embodiments, the composition comprises a sense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The sense strand may be 14-30 nucleosides in length. In some embodiments, the composition comprises an antisense strand is 12-30 nucleosides in length. In some embodiments, the composition comprises an antisense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The antisense strand may be 14-30 nucleosides in length.

In some embodiments, the composition comprises an oligonucleotide that inhibits the expression of MST1R, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human MST1R mRNA sequence such as SEQ ID NO: 9818. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 9818.

In some embodiments, the composition comprises an oligonucleotide that inhibits the expression of MST1R, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of MST1R, wherein the oligonucleotide comprises an siRNA comprising a sense strand and an antisense strand, wherein the siRNA binds with a 19mer in a human MST1R mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human MST1R mRNA.

In some embodiments, the composition comprises an oligonucleotide that inhibits the expression of MST1R, wherein the oligonucleotide comprises an siRNA comprising a sense strand and an antisense strand, wherein the siRNA binds with a 17mer in a non-human primate MST1R mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate MST1R mRNA.

In some embodiments, the composition comprises an oligonucleotide that inhibits the expression of MST1R, wherein the oligonucleotide comprises an siRNA comprising a sense strand and an antisense strand, wherein the siRNA binds with a human MST1R mRNA and less than or equal to 20 human off-targets, with no more than 2 mismatches in the antisense strand. In some embodiments, the siRNA binds with a human MST1R mRNA and less than or equal to 10 human off-targets, with no more than 2 mismatches in the antisense strand. In some embodiments, the siRNA binds with a human MST1R mRNA and less than or equal to 30 human off-targets, with no more than 2 mismatches in the antisense strand. In some embodiments, the siRNA binds with a human MST1R mRNA and less than or equal to 40 human off-targets, with no more than 2 mismatches in the antisense strand. In some embodiments, the siRNA binds with a human MST1R mRNA and less than or equal to 50 human off-targets, with no more than 2 mismatches in the antisense strand. In some embodiments, the siRNA binds with a human MST1R mRNA and less than or equal to 10 human off-targets, with no more than 3 mismatches in the antisense strand. In some embodiments, the siRNA binds with a human MST1R mRNA and less than or equal to 20 human off-targets, with no more than 3 mismatches in the antisense strand. In some embodiments, the siRNA binds with a human MST1R mRNA and less than or equal to 30 human off-targets, with no more than 3 mismatches in the antisense strand. In some embodiments, the siRNA binds with a human MST1R mRNA and less than or equal to 40 human off-targets, with no more than 3 mismatches in the antisense strand. In some embodiments, the siRNA binds with a human MST1R mRNA and less than or equal to 50 human off-targets, with no more than 3 mismatches in the antisense strand.

In some embodiments, the composition comprises an oligonucleotide that inhibits the expression of MST1R, wherein the oligonucleotide comprises an siRNA comprising a sense strand and an antisense strand, siRNA binds with a human MST1R mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the composition comprises an oligonucleotide that inhibits the expression of MST1R, wherein the oligonucleotide comprises an siRNA comprising a sense strand and an antisense strand, wherein the sense strand comprises a nucleoside sequence comprising or consisting of the sequence of any one of SEQ ID NOs: 1-4754, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 1-4754, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 1-4754, or a nucleic acid sequence thereof having 1 or 2 nucleoside additions at the 3′ end. In some embodiments, the composition comprises an oligonucleotide that inhibits the expression of MST1R, wherein the oligonucleotide comprises an siRNA comprising a sense strand and an antisense strand, wherein the sense strand comprises a nucleoside sequence comprising or consisting of the sequence of any one of SEQ ID NOs: 1-4754.

In some embodiments, the composition comprises an oligonucleotide that inhibits the expression of MST1R, wherein the oligonucleotide comprises an siRNA comprising a sense strand and an antisense strand, wherein the antisense strand comprises a nucleoside sequence comprising or consisting of the sequence of any one of SEQ ID NOs: 4755-9508, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 4755-9508, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 4755-9508, or a nucleic acid sequence thereof having 1 or 2 nucleoside additions at the 3′ end. In some embodiments, the composition comprises an oligonucleotide that inhibits the expression of MST1R, wherein the oligonucleotide comprises an siRNA comprising a sense strand and an antisense strand, wherein the antisense strand comprises a nucleoside sequence comprising or consisting of the sequence of any one of SEQ ID NOs: 4755-9508.

In some embodiments, the siRNA comprises the sense strand and/or the antisense strand sequence of an siRNA in any one of Tables 3-8, or a nucleic acid sequence thereof having 3 or 4 nucleoside substitutions, additions, or deletions. In some embodiments, the siRNA comprises the sense strand and/or the antisense strand sequence of an siRNA in any one of Tables 3-8, or a nucleic acid sequence thereof having 1 or 2 nucleoside substitutions, additions, or deletions. In some embodiments, the siRNA comprises the sense strand and/or the antisense strand sequence of an siRNA in any one of Tables 3-8. In some embodiments, the siRNA is cross-reactive with a non-human primate (NHP) MST1R mRNA. The siRNA may include one or more internucleoside linkages and/or one or more nucleoside modifications.

In some embodiments, the siRNA comprises the sense strand and/or the antisense strand sequence of an siRNA of subset A, or a nucleic acid sequence thereof having 3 or 4 nucleoside substitutions, additions, or deletions. In some embodiments, the siRNA comprises the sense strand and/or the antisense strand sequence of an siRNA of subset A, or a nucleic acid sequence thereof having 1 or 2 nucleoside substitutions, additions, or deletions. In some embodiments, the siRNA comprises the sense strand and/or the antisense strand sequence of an siRNA of subset A. In some embodiments, the siRNA is cross-reactive with a non-human primate (NHP) MST1R mRNA. The siRNA may include one or more internucleoside linkages and/or one or more nucleoside modifications.

In some embodiments, the siRNA comprises the sense strand and/or the antisense strand sequence of an siRNA of subset B, or a nucleic acid sequence thereof having 3 or 4 nucleoside substitutions, additions, or deletions. In some embodiments, the siRNA comprises the sense strand and/or the antisense strand sequence of an siRNA of subset B, or a nucleic acid sequence thereof having 1 or 2 nucleoside substitutions, additions, or deletions. In some embodiments, the siRNA comprises the sense strand and/or the antisense strand sequence of an siRNA of subset B. In some embodiments, the siRNA is cross-reactive with a non-human primate (NHP) MST1R mRNA. The siRNA may include one or more internucleoside linkages and/or one or more nucleoside modifications.

In some embodiments, the siRNA comprises the sense strand and/or the antisense strand sequence of an siRNA of subset C, or a nucleic acid sequence thereof having 3 or 4 nucleoside substitutions, additions, or deletions. In some embodiments, the siRNA comprises the sense strand and/or the antisense strand sequence of an siRNA of subset C, or a nucleic acid sequence thereof having 1 or 2 nucleoside substitutions, additions, or deletions. In some embodiments, the siRNA comprises the sense strand and/or the antisense strand sequence of an siRNA of subset C. In some embodiments, the siRNA is cross-reactive with a non-human primate (NHP) MST1R mRNA. The siRNA may include one or more internucleoside linkages and/or one or more nucleoside modifications.

In some embodiments, the siRNA comprises the sense strand and/or the antisense strand sequence of an siRNA of subset D, or a nucleic acid sequence thereof having 3 or 4 nucleoside substitutions, additions, or deletions. In some embodiments, the siRNA comprises the sense strand and/or the antisense strand sequence of an siRNA of subset D, or a nucleic acid sequence thereof having 1 or 2 nucleoside substitutions, additions, or deletions. In some embodiments, the siRNA comprises the sense strand and/or the antisense strand sequence of an siRNA of subset D. In some embodiments, the siRNA is cross-reactive with a non-human primate (NHP) MST1R mRNA. The siRNA may include one or more internucleoside linkages and/or one or more nucleoside modifications.

In some embodiments, the siRNA comprises the sense strand and/or the antisense strand sequence of an siRNA of subset E, or a nucleic acid sequence thereof having 3 or 4 nucleoside substitutions, additions, or deletions. In some embodiments, the siRNA comprises the sense strand and/or the antisense strand sequence of an siRNA of subset E, or a nucleic acid sequence thereof having 1 or 2 nucleoside substitutions, additions, or deletions. In some embodiments, the siRNA comprises the sense strand and/or the antisense strand sequence of an siRNA of subset E. In some embodiments, the siRNA is cross-reactive with a non-human primate (NHP) MST1R mRNA. The siRNA may include one or more internucleoside linkages and/or one or more nucleoside modifications.

In some embodiments, the siRNA comprises the sense strand and/or the antisense strand sequence of an siRNA of subset F, or a nucleic acid sequence thereof having 3 or 4 nucleoside substitutions, additions, or deletions. In some embodiments, the siRNA comprises the sense strand and/or the antisense strand sequence of an siRNA of subset F, or a nucleic acid sequence thereof having 1 or 2 nucleoside substitutions, additions, or deletions. In some embodiments, the siRNA comprises the sense strand and/or the antisense strand sequence of an siRNA of subset F. In some embodiments, the siRNA is cross-reactive with a non-human primate (NHP) MST1R mRNA. The siRNA may include one or more internucleoside linkages and/or one or more nucleoside modifications.

### B. ASOs

In some embodiments, the composition comprises an oligonucleotide that inhibits the expression of MST1R, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of MST1R, wherein the oligonucleotide comprises an ASO about 12-30 nucleosides in length and comprising a nucleoside sequence complementary to about 12-30 contiguous nucleosides of a full-length human MST1R mRNA sequence such as SEQ ID NO: 9818; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 9818.

### C. Modification Patterns

In some embodiments, the composition comprises an oligonucleotide that inhibits the expression of MST1R, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. Benefits of the modified internucleoside linkage may include decreased toxicity or improved pharmacokinetics.

In some embodiments, the composition comprises an oligonucleotide that inhibits the expression of MST1R, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of MST1R, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

The oligonucleotide may be chemically conjugated to a targeting group, lipid (including, but not limited to cholesterol, cholesteryl derivatives, and fatty acids), nanoparticle, polymer, liposome, micelle, or other delivery system. A targeting group may be linked to a 3′ or 5′ end of the oligonucleotide (e.g. to a 3′ or 5′ end of a sense strand or an antisense strand). In some embodiments, a targeting group is linked internally to a nucleotide on a sense strand or an antisense strand of the oligonucleotide. In some embodiments, a targeting group is linked to the oligonucleotide via a linker.

In some embodiments, the composition comprises an oligonucleotide that inhibits the expression of MST1R, wherein the oligonucleotide comprises a moiety attached at a 3′ or 5′ terminus of the oligonucleotide. Examples of moieties include an integrin targeting ligand, a hydrophobic moiety, a sugar moiety, or a combination thereof. In some embodiments, the oligonucleotide is an siRNA having a sense strand, and the moiety is attached to a 5′ end of the sense strand. In some embodiments, the oligonucleotide is an siRNA having a sense strand, and the moiety is attached to a 3′ end of the sense strand. In some embodiments, the oligonucleotide is an siRNA having an antisense strand, and the moiety is attached to a 5′ end of the antisense strand. In some embodiments, the oligonucleotide is an siRNA having an antisense strand, and the moiety is attached to a 3′ end of the antisense strand. In some embodiments, the oligonucleotide is an ASO, and the moiety is attached to a 5′ end of the ASO. In some embodiments, the oligonucleotide is an ASO, and the moiety is attached to a 3′ end of the ASO.

In some embodiments, the oligonucleotide is delivered to a cell or tissue by linking the oligonucleotide to a targeting group. In some embodiments, the targeting group includes a cell receptor ligand, such as an integrin targeting ligand. Integrins may include a family of transmembrane receptors that facilitate cell-extracellular matrix (ECM) adhesion. In some embodiments, the moiety includes an epithelial-specific integrin. Integrin alpha-v-beta-6 (αvβ6) bay be an example of an epithelial-specific integrin αvβ6 may be a receptor for an ECM protein or TGF-beta latency-associated peptide (LAP). Integrin αvβ6 may be expressed in a cell or tissue. Integrin αvβ6 may be expressed or upregulated in injured pulmonary epithelium.

In some embodiments, the oligonucleotide is linked to an integrin targeting ligand that has affinity for integrin αvβ6. An integrin targeting ligand may include a compound that has affinity for integrin αvβ6 or integrin alpha-v-beta-3 (αvβ3), may be useful as a ligand to facilitate targeting or delivery of the oligonucleotide to which it is attached to a particular cell type or tissue (e.g., to cells expressing integrin αvβ3 or αvβ6). In some embodiments, multiple integrin targeting ligands are linked to the oligonucleotide. In some embodiments, the oligonucleotide-integrin targeting ligand conjugates are selectively internalized by lung epithelial cells, either through receptor-mediated endocytosis or by other means.

Examples of targeting groups useful for delivering the oligonucleotide that include integrin targeting ligands may be based upon peptides or peptide mimics containing an arginine-glycine-aspartic acid (RGD) peptide. In some embodiments, the composition comprises an oligonucleotide that inhibits the expression of MST1R, wherein the oligonucleotide comprises an RGD peptide. In some embodiments, the composition comprises an RGD peptide. In some embodiments, the composition comprises an RGD peptide derivative. In some embodiments, the RGD peptide is attached at a 3′ terminus of the oligonucleotide. In some embodiments, the RGD peptide is attached at a 5′ terminus of the oligonucleotide. In some embodiments, the composition comprises a sense strand, and the RGD peptide is attached to the sense strand (e.g. attached to a 5′ end of the sense strand, or attached to a 3′ end of the sense strand). In some embodiments, the composition comprises an antisense strand, and the RGD peptide is attached to the antisense strand (e.g. attached to a 5′ end of the antisense strand, or attached to a 3′ end of the antisense strand). In some embodiments, the composition comprises an RGD peptide attached at a 3′ or 5′ terminus of the oligonucleotide. In some embodiments, the oligonucleotide comprises an RGD peptide and a lipid attached at a 3′ or 5′ terminus of the oligonucleotide. The RGD peptide may be linear. The RGD peptide may be cyclic. An RGD peptide may include a D-amino acid. In some embodiments, the RGD peptide comprises Cyclo(-Arg-Gly-Asp-D-Phe-Cys) (SEQ ID NO: 9837). In some embodiments, the RGD peptide comprises Cyclo(-Arg-Gly-Asp-D-Phe-Lys) (SEQ ID NO: 9838). In some embodiments, the RGD peptide comprises Cyclo(-Arg-Gly-Asp-D-Phe-azido) (SEQ ID NO: 9839). In some embodiments, the RGD peptide comprises an amino benzoic acid derived RGD. In some embodiments, the RGD peptide comprises Cyclo(-Arg-Gly-Asp-D-Phe-Cys) (SEQ ID NO: 9837), Cyclo(-Arg-Gly-Asp-D-Phe-Lys) (SEQ ID NO: 9838), Cyclo(-Arg-Gly-Asp-D-Phe-azido) (SEQ ID NO: 9839), an amino benzoic acid derived RGD, or a combination thereof. In some embodiments, the RGD peptide comprises multiple of such RGD peptides. For example, the RGD peptide may include 2, 3, or 4 RGD peptides. Some embodiments include an arginine-glycine-glutamic acid peptide.

In some embodiments, the composition comprises an oligonucleotide that inhibits the expression of MST1R, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of MST1R, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′ fluoro modified purines. In some embodiments, purines of the oligonucleotide comprise 2′-O-methyl modified purines. In some embodiments, purines of the oligonucleotide comprise a mixture of 2′ fluoro and 2′-O-methyl modified purines. In some embodiments, all purines of the oligonucleotide comprise 2′ fluoro modified purines. In some embodiments, all purines of the oligonucleotide comprise 2′-O-methyl modified purines. In some embodiments, all purines of the oligonucleotide comprise a mixture of 2′ fluoro and 2′-O-methyl modified purines. 2′-O-methyl may include 2′ O-methyl. Where a 2′ O-methyl modification is included, it is contemplated that a 2′ methyl modification may be included, and vice versa.

In some embodiments, pyrimidines of the oligonucleotide comprise 2′ fluoro modified pyrimidines. In some embodiments, pyrimidines of the oligonucleotide comprise 2′-O-methyl modified pyrimidines. In some embodiments, pyrimidines of the oligonucleotide comprise a mixture of 2′ fluoro and 2′-O-methyl modified pyrimidines. In some embodiments, all pyrimidines of the oligonucleotide comprise 2′ fluoro modified pyrimidines. In some embodiments, all pyrimidines of the oligonucleotide comprise 2′-O-methyl modified pyrimidines. In some embodiments, all pyrimidines of the oligonucleotide comprise a mixture of 2′ fluoro and 2′-O-methyl modified pyrimidines.

In some embodiments, purines of the oligonucleotide comprise 2′ fluoro modified purines, and pyrimidines of the oligonucleotide comprise a mixture of 2′ fluoro and 2′-O-methyl modified pyrimidines. In some embodiments, purines of the oligonucleotide comprise 2′-O-methyl modified purines, and pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise 2′-O-methyl modified pyrimidines. In some embodiments, purines of the oligonucleotide comprise 2′-O-methyl modified purines, and pyrimidines of the oligonucleotide comprise 2′ fluoro modified pyrimidines. In some embodiments, pyrimidines of the oligonucleotide comprise 2′ fluoro modified pyrimidines, and purines of the oligonucleotide comprise a mixture of 2′ fluoro and 2′-O-methyl modified purines. In some embodiments, pyrimidines of the oligonucleotide comprise 2′-O-methyl modified pyrimidines, and purines of the oligonucleotide comprise a mixture of 2′ fluoro and 2′-O-methyl modified purines. In some embodiments, pyrimidines of the oligonucleotide comprise 2′ fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′ fluoro modified purines.

In some embodiments, all purines of the oligonucleotide comprise 2′ fluoro modified purines, and all pyrimidines of the oligonucleotide comprise a mixture of 2′ fluoro and 2′-O-methyl modified pyrimidines. In some embodiments, all purines of the oligonucleotide comprise 2′-O-methyl modified purines, and all pyrimidines of the oligonucleotide comprise a mixture of 2′ fluoro and 2′-O-methyl modified pyrimidines. In some embodiments, all purines of the oligonucleotide comprise 2′ fluoro modified purines, and all pyrimidines of the oligonucleotide comprise 2′-O-methyl modified pyrimidines. In some embodiments, all purines of the oligonucleotide comprise 2′-O-methyl modified purines, and all pyrimidines of the oligonucleotide comprise 2′ fluoro modified pyrimidines. In some embodiments, all pyrimidines of the oligonucleotide comprise 2′ fluoro modified pyrimidines, and all purines of the oligonucleotide comprise a mixture of 2′ fluoro and 2′-O-methyl modified purines. In some embodiments, all pyrimidines of the oligonucleotide comprise 2′-O-methyl modified pyrimidines, and all purines of the oligonucleotide comprise a mixture of 2′ fluoro and 2′-O-methyl modified purines. In some embodiments, all pyrimidines of the oligonucleotide comprise 2′ fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′ fluoro modified purines.

In some cases, the oligonucleotide comprises a particular modification pattern. In some embodiments, position 9 counting from the 5′ end of the of a strand of the oligonucleotide may have a 2′F modification. In some embodiments, when position 9 of a strand of the oligonucleotide is a pyrimidine, then all purines in a strand of the oligonucleotide have a 2′OMe modification. In some embodiments, when position 9 is the only pyrimidine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are pyrimidines, then both of these pyrimidines are the only two positions with a 2′F modification in a strand of the oligonucleotide. In some embodiments, when position 9 and only two other bases between positions 5 and 11 of a strand of the oligonucleotide are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of a strand of the oligonucleotide, then all combinations of pyrimidines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some embodiments, when position 9 of a strand of the oligonucleotide is a purine, then all purines in a strand of the oligonucleotide have a 2′OMe modification. In some embodiments, when position 9 is the only purine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are purines, then both of these purines are the only two positions with a 2′F modification in a strand of the oligonucleotide. In some embodiments, when position 9 and only two other bases between positions 5 and 11 of a strand of the oligonucleotide are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of a strand of the oligonucleotide, then all combinations of purines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some cases, position 9 of a strand of the oligonucleotide can be a 2′deoxy. In these cases, 2′F and 2′OMe modifications may occur at the other positions of a strand of the oligonucleotide. In some cases, a strand of the oligonucleotide of any of the siRNAs comprises a modification pattern which conforms to these a strand of the oligonucleotide rules.

In some embodiments, position nine of the sense strand comprises a 2′ fluoro-modified pyrimidine. In some embodiments, all purines of the sense strand comprise 2′-O-methyl modified purines. In some embodiments, 1, 2, 3, 4, or 5 pyrimidines between positions 5 and 11 comprise a 2′fluoro-modified pyrimidine, provided there are not three 2′ fluoro-modified pyrimidines in a row. In some embodiments, the odd-numbered positions of the antisense strand comprise 2′-O-methyl modified nucleotides. In some embodiments, the even-numbered positions of the antisense strand comprise 2′fluoro-modified nucleotides and unmodified deoxyribonucleotide. In some embodiments, the even-numbered positions of the antisense strand comprise 2′fluoro-modified nucleotides, 2′-O-methyl modified nucleotides and unmodified deoxyribonucleotide. In some embodiments, position nine of the sense strand comprises a 2′ fluoro-modified pyrimidine; all purines of the sense strand comprises 2′-O-methyl modified purines; 1, 2, 3, 4, or 5 pyrimidines between positions 5 and 11 comprise a 2′fluoro-modified pyrimidine, provided there are not three 2′ fluoro-modified pyrimidines in a row; the odd-numbered positions of the antisense strand comprise 2′-O-methyl modified nucleotides; and the even-numbered positions of the antisense strand comprise 2′fluoro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′fluoro-modified purine, provided there are not three 2′ fluoro-modified purine in a row. In some embodiments, the odd-numbered positions of the antisense strand comprise 2′-O-methyl modified nucleotides. In some embodiments, the even-numbered positions of the antisense strand comprise 2′fluoro-modified nucleotides and unmodified deoxyribonucleotide. In some embodiments, the even-numbered positions of the antisense strand comprise 2′fluoro-modified nucleotides, 2′-O-methyl modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines; 1, 2, 3, 4, or 5 purines between positions 5 and 11 comprise a 2′fluoro-modified purines, provided there are not three 2′ fluoro-modified purines in a row; the odd-numbered positions of the antisense strand comprise 2′-O-methyl modified nucleotides; and the even-numbered positions of the antisense strand comprise 2′fluoro-modified nucleotides and unmodified deoxyribonucleotides. In some embodiments, there are not three 2′ fluoro-modified purines in a row. In some embodiments, there are not three 2′ fluoro-modified pyrimidines in a row.

In some embodiments, position nine of the sense strand comprises an unmodified deoxyribonucleotide. In some embodiments, positions 5, 7, and 8 of the sense strand comprise 2′fluoro-modified nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′fluoro-modified purines. In some embodiments, the odd-numbered positions of the antisense strand comprise 2′-O-methyl modified nucleotides. In some embodiments, the even-numbered positions of the antisense strand comprise 2′fluoro-modified nucleotides and unmodified deoxyribonucleotides. In some embodiments, the even-numbered positions of the antisense strand comprise 2′fluoro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all pyrimidines in positions 10 to 21 of the sense strand comprise 2′-O-methyl modified pyrimidines and all purines in positions 10 to 21 of the comprise 2′-O-methyl modified purines or 2′fluoro-modified purines; the odd-numbered positions of the antisense strand comprise 2′-O-methyl modified nucleotides; and the even-numbered positions of the antisense strand comprise 2′fluoro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modified nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines. In some embodiments, the odd-numbered positions of the antisense strand comprise 2′-O-methyl modified nucleotides. In some embodiments, the even-numbered positions of the antisense strand comprise 2′fluoro-modified nucleotides and unmodified deoxyribonucleotides. In some embodiments, the even-numbered positions of the antisense strand comprise 2′fluoro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines; the odd-numbered positions of the antisense strand comprise 2′-O-methyl modified nucleotides; and the even-numbered positions of the antisense strand comprise 2′fluoro-modified nucleotides and unmodified deoxyribonucleotide.

In some embodiments, the moiety includes a negatively charged group attached at a 5′ end of the oligonucleotide. This may be referred to as a 5′-end group. In some embodiments, the negatively charged group is attached at a 5′ end of an antisense strand of an siRNA disclosed herein. The 5′-end group may be or include a 5′-end phosphorothioate, 5′-end phosphorodithioate, 5′-end vinylphosphonate (5′-VP), 5′-end methylphosphonate, 5′-end cyclopropyl phosphonate, or a 5′-deoxy-5′-C-malonyl. The 5′-end group may comprise 5′-VP. In some embodiments, the 5′-VP comprises a trans-vinylphosphate or cis-vinylphosphate. The 5′-end group may include an extra 5′ phosphate. A combination of 5′-end groups may be used.

In some embodiments, the oligonucleotide includes a negatively charged group. The negatively charged group may aid in cell or tissue penetration. The negatively charged group may be attached at a 5′ or 3′ end (e.g. a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphate. In some embodiments, the vinyl phosphonate comprises a cis-vinylphosphate. An example of a nucleotide that includes a vinyl phosphonate is shown below.

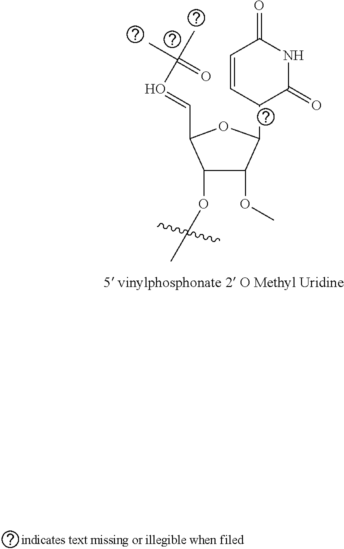

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### 1. Hydrophobic Moieties

In some embodiments, the composition comprises an oligonucleotide that inhibits the expression of MST1R, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of MST1R, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof.

In some embodiments, the composition comprises an oligonucleotide that inhibits the expression of MST1R, wherein the oligonucleotide comprises a hydrophobic ligand or moiety. In some embodiments, the hydrophobic ligand or moiety comprises cholesterol. In some embodiments, the hydrophobic ligand or moiety comprises a cholesterol derivative. In some embodiments, the hydrophobic ligand or moiety is attached at a 3′ terminus of the oligonucleotide. In some embodiments, the hydrophobic ligand or moiety s attached at a 5′ terminus of the oligonucleotide. In some embodiments, the composition comprises a sense strand, and the hydrophobic ligand or moiety is attached to the sense strand (e.g. attached to a 5′ end of the sense strand, or attached to a 3′ end of the sense strand). In some embodiments, the composition comprises an antisense strand, and the hydrophobic ligand or moiety is attached to the antisense strand (e.g. attached to a 5′ end of the antisense strand, or attached to a 3′ end of the antisense strand). In some embodiments, the composition comprises a hydrophobic ligand or moiety attached at a 3′ or 5′ terminus of the oligonucleotide.

In some embodiments, a hydrophobic moiety is attached to the oligonucleotide (e.g. a sense strand and/or an antisense strand of a siRNA). In some embodiments, a hydrophobic moiety is attached at a 3′ terminus of the oligonucleotide. In some embodiments, a hydrophobic moiety is attached at a 5′ terminus of the oligonucleotide. In some embodiments, the hydrophobic moiety comprises cholesterol. In some embodiments, the hydrophobic moiety includes a cyclohexanyl.

In some embodiments, the composition comprises an oligonucleotide that inhibits the expression of MST1R, wherein the oligonucleotide comprises a lipid attached at a 3′ or 5′ terminus of the oligonucleotide. In some embodiments, a lipid is attached at a 3′ terminus of the oligonucleotide. In some embodiments, a lipid is attached at a 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof. In some embodiments, the lipid comprises stearyl, lithocholyl, docosanyl, docosahexaenyl, or myristyl. In some embodiments, the lipid comprises cholesterol. In some embodiments, the lipid includes a sterol such as cholesterol. In some embodiments, the lipid comprises stearyl, t-butylphenol, n-butylphenol, octylphenol, dodecylphenol, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

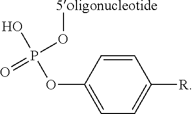

In some embodiments, the oligonucleotide comprises any aspect of the following structure:

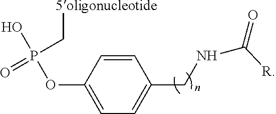

In some embodiments, the oligonucleotide comprises any aspect of the following structure:

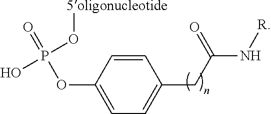

In some embodiments, the oligonucleotide comprises any aspect of the following structure: The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1. The example lipid moieties in Table 1 are shown attached at a 5′ end of an oligonucleotide, in which the 5′ terminal phosphate of the oligonucleotide is shown with the lipid moiety. In some embodiments, a lipid moiety in Table 1 may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1
Hydrophobic moiety examples

| Hydrophobic | Hydrophobic | |
|---|---|---|
| Moiety Description | Moiety Name | Example Conjugation |
| stearyl | ETL3 | 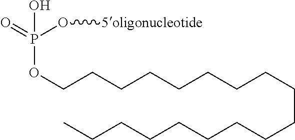 |
| t-butylphenyl | ETL7 | 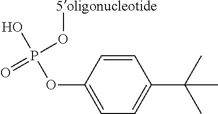 |
| n-butylphenyl | ETL8 | 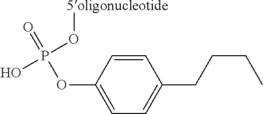 |
| octylphenyl | ETL9 | 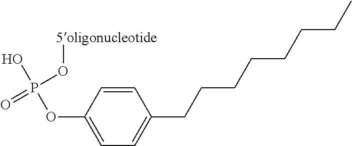 |
| dodecylphenyl | ETL10 | 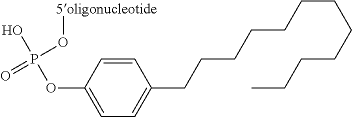 |
| phenyl n-dodecyl | ETL 12 | 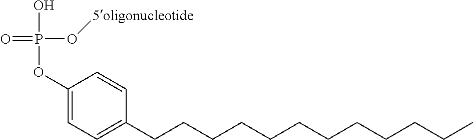 |
| octadecylbenzamide | ETL13 | 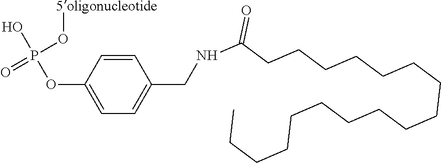 |
| hexadecylbenzamide | ETL15 | 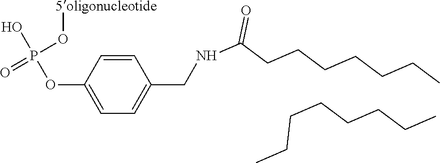 |
| octadecylcyclohexyl | ETL16 | 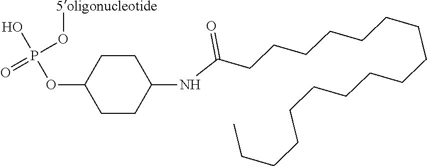 |

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g. 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g. the para, meta, or ortho phenyl configuration). In some embodiments, the lipid or hydrocarbon, and the end of the sense are connected to the phenyl or cyclohexyl linker in the 1,4 substitution pattern (e.g. the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure:

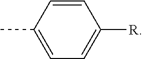

In some embodiments, the lipid moiety comprises or consists of the following structure:

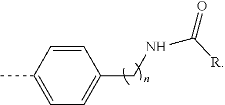

In some embodiments, the lipid moiety comprises the following structure:

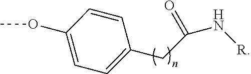

In some embodiments, the lipid moiety comprises or consist of the following structure:

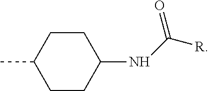

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g. a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

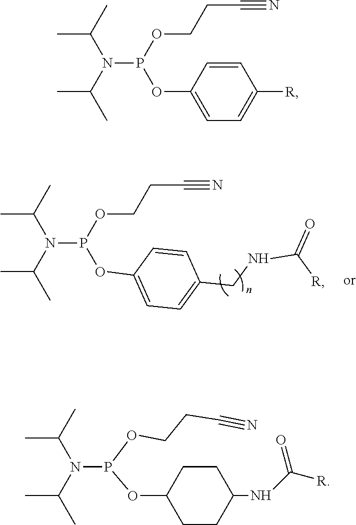

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

The lipid may be attached to the oligonucleotide by a linker. The linker may include a polyethyleneglycol (e.g. tetraethyleneglycol).

The modifications described herein may be useful for delivery to a cell or tissue, for example, extrahepatic delivery or targeting of an oligonucleotide composition. The modifications described herein may be useful for targeting an oligonucleotide composition to a cell or tissue.

### 2. Sugar Moieties

In some embodiments, the composition comprises an oligonucleotide that inhibits the expression of MST1R, wherein the oligonucleotide comprises a sugar moiety. The sugar moiety may include an N-acetyl galactose moiety (e.g. an N-acetylgalactosamine (GalNAc) moiety), an N-acetyl glucose moiety (e.g. an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages when they target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of MST1R, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker. The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of MST1R, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

Disclosed herein, in some embodiments, are compositions comprising an oligonucleotide that inhibits the expression of MST1R, wherein the oligonucleotide comprises a GalNAc moiety. The GalNAc moiety may be included in any formula, structure, or GalNAc moiety shown below. In some embodiments, described herein is a compound (e.g. oligonucleotide) represented by Formula (I) or (II):

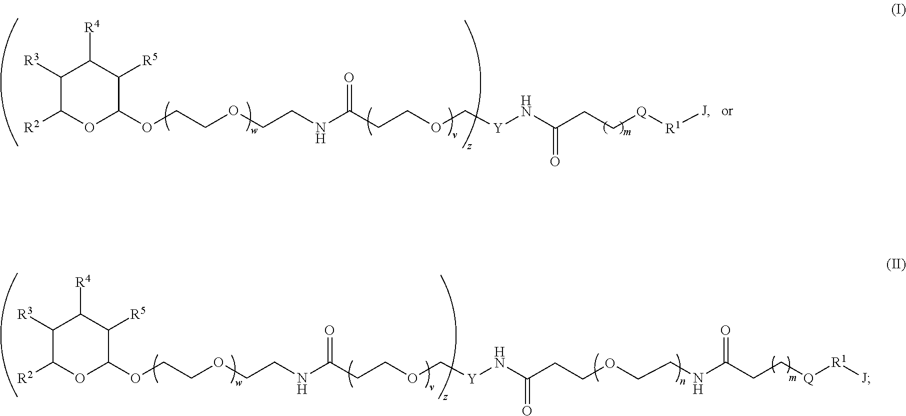

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
   —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
   C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
   —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
   —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
   hydrogen;
   halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
   C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, =O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, =O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆alkynyl, C₃₋₁₀carbocycle, 3- to 10-membered heterocycle, and C₁₋₆haloalkyl.

In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₋₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₋₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂—NR⁷. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, and —OP(OR⁷)O—. In some embodiments, R¹ is selected from —OP(O)(OR⁷)O—, —OP(S)(OR⁷)O—, —OP(O)(O⁻)O—, —OP(S)(O⁻)O—, —OP(O)(S⁻)O—, and —OP(OR⁷)O—. In some embodiments, R¹ is selected from —OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R² is selected from C₁₋₃ alkyl substituted with one or more substituents independently selected from —OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷—SR⁷, —OC(O)R⁷, and —N(R⁷)₂. In some embodiments, R³ is selected from —OR²— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷—SR⁷, —OC(O)R⁷, and —N(R⁷)₂. In some embodiments, R⁴ is selected from —OR⁷— and —OC(O)R⁷. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, =O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, =O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from —OP(O)(OR⁷)O—, —OP(S)(OR⁷)O—, —OP(O)(O—)O—, —OP(S)(O—)O—, —OP(O)(S—)O—, and —OP(OR⁷)O—; R² is C₁ alkyl substituted with —OH or —OC(O)CH₃;
R³ is —OH or —OC(O)CH₃; R⁴ is —OH or —OC(O)CH₃; and R⁵ is —NH(O)CH₃. In some embodiments, the compound comprises:

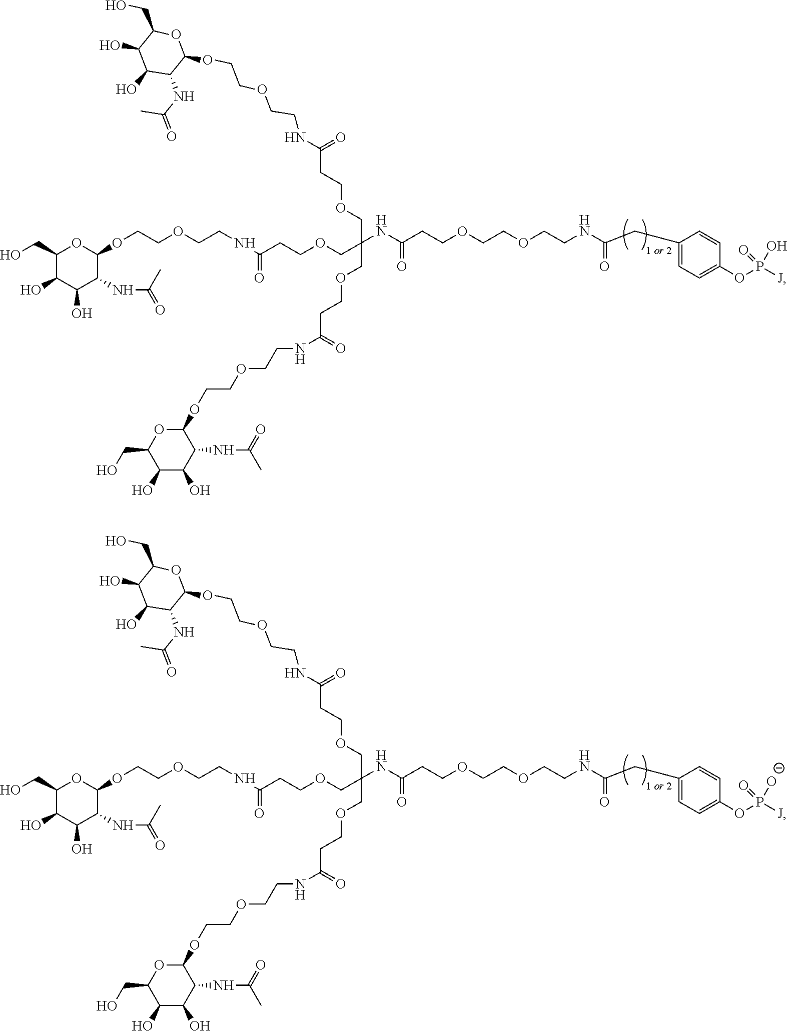

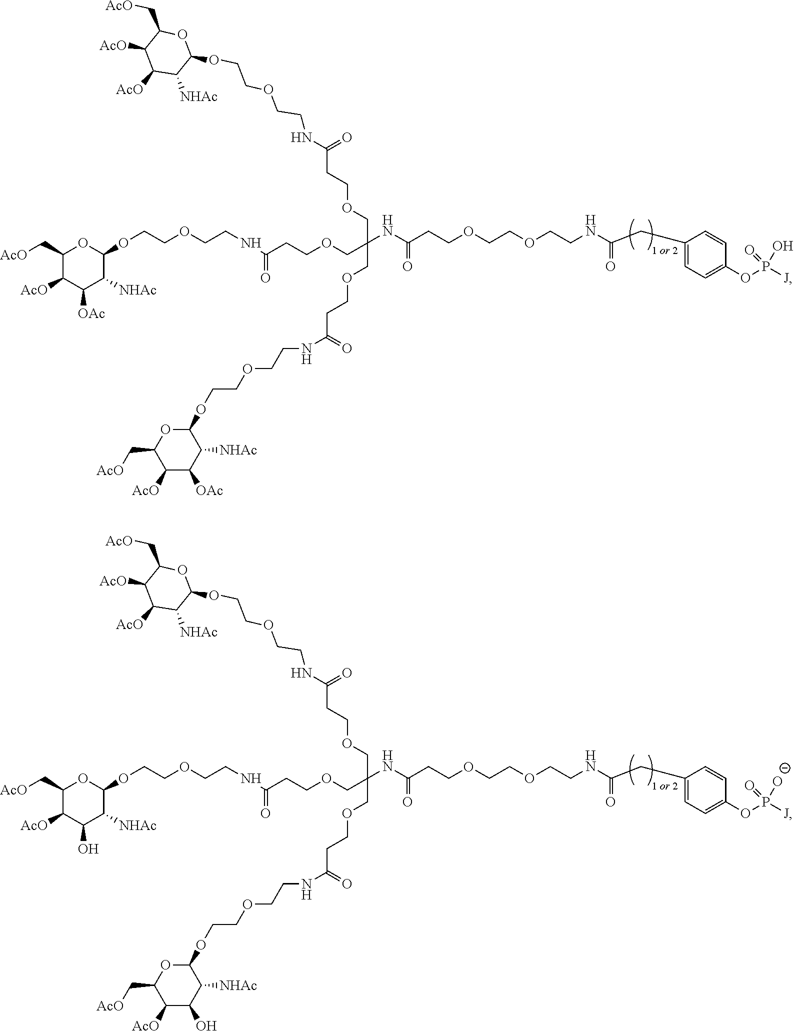

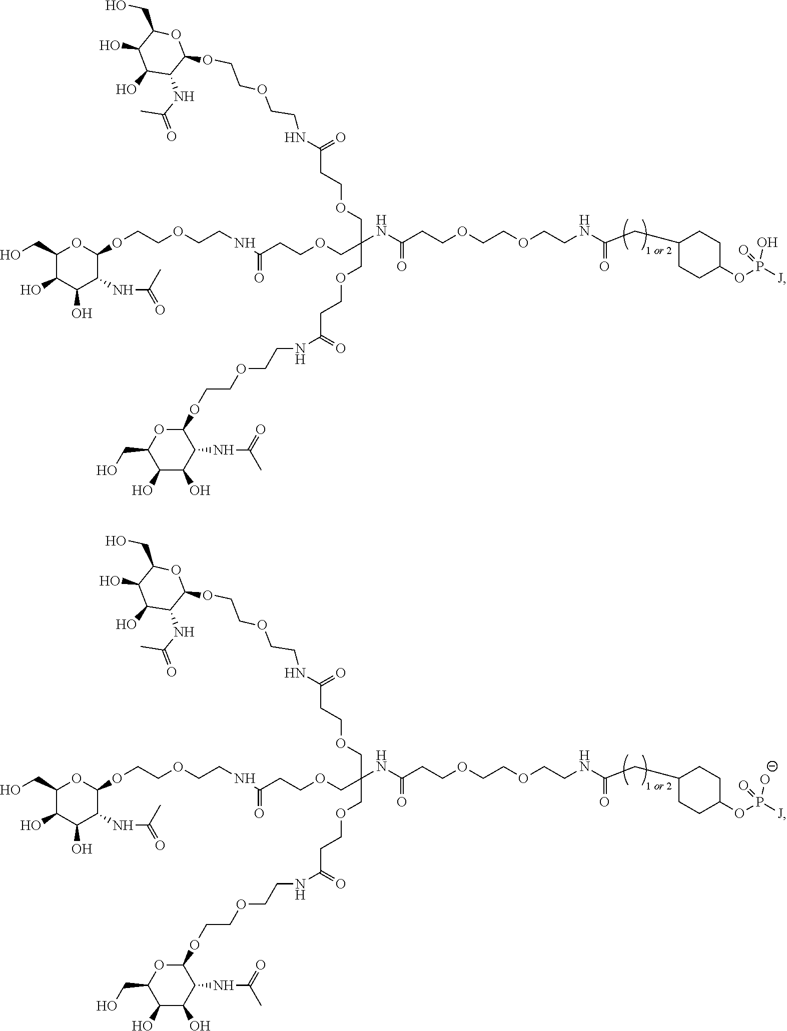

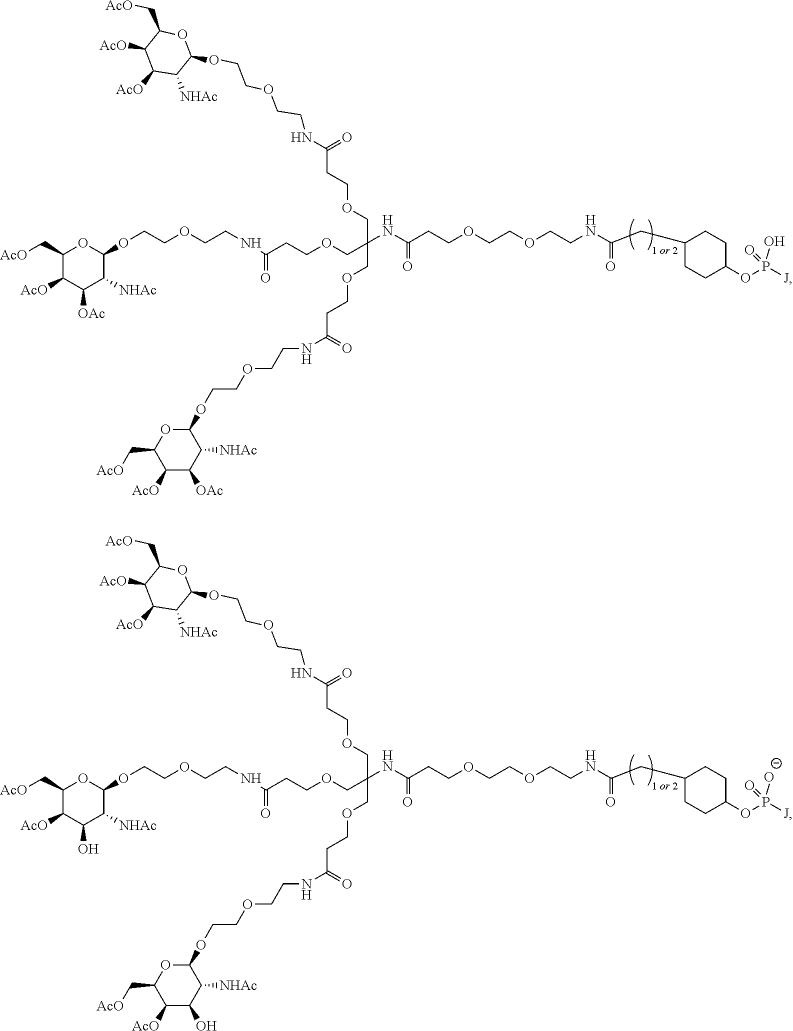

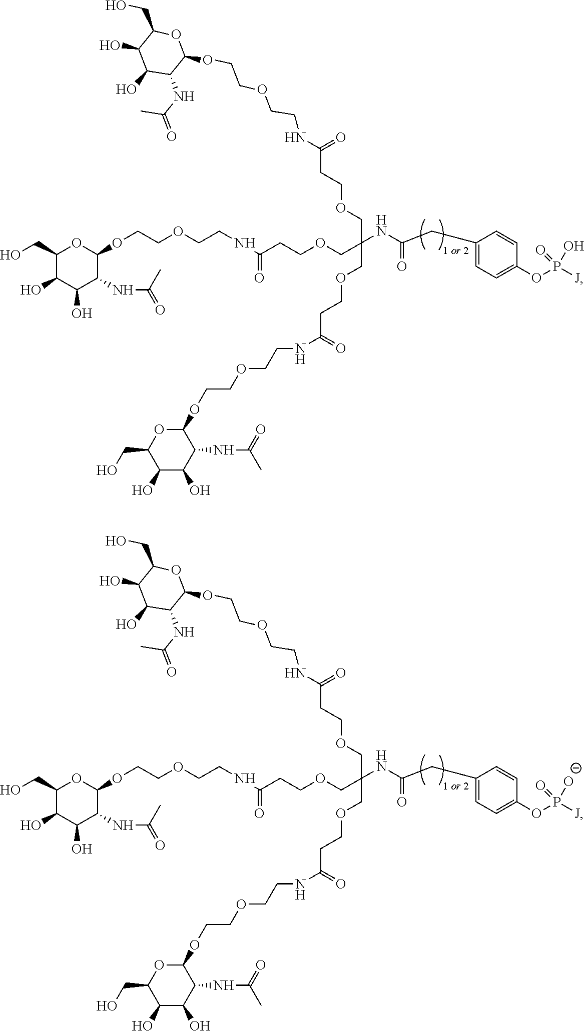

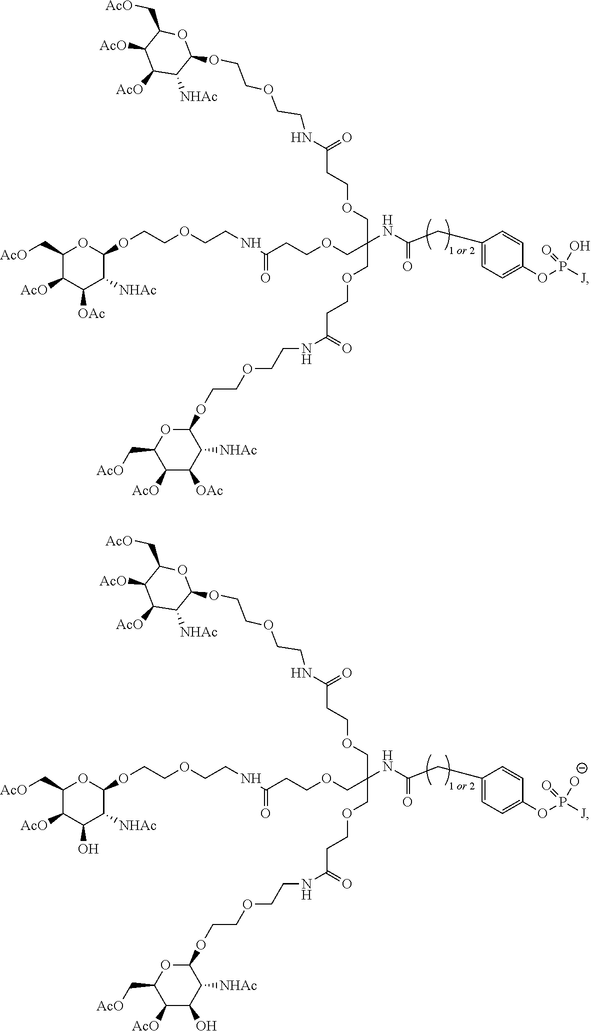

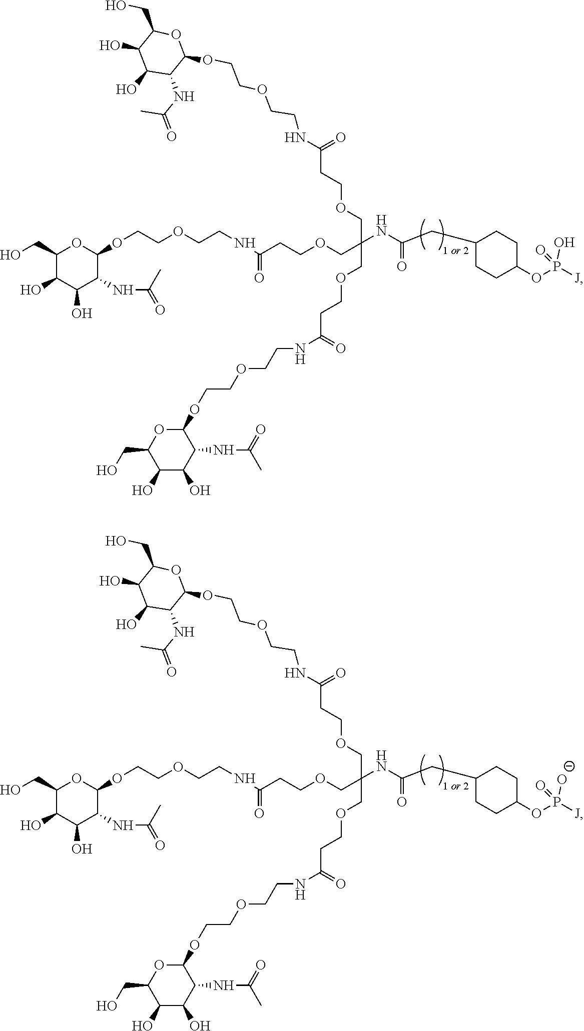

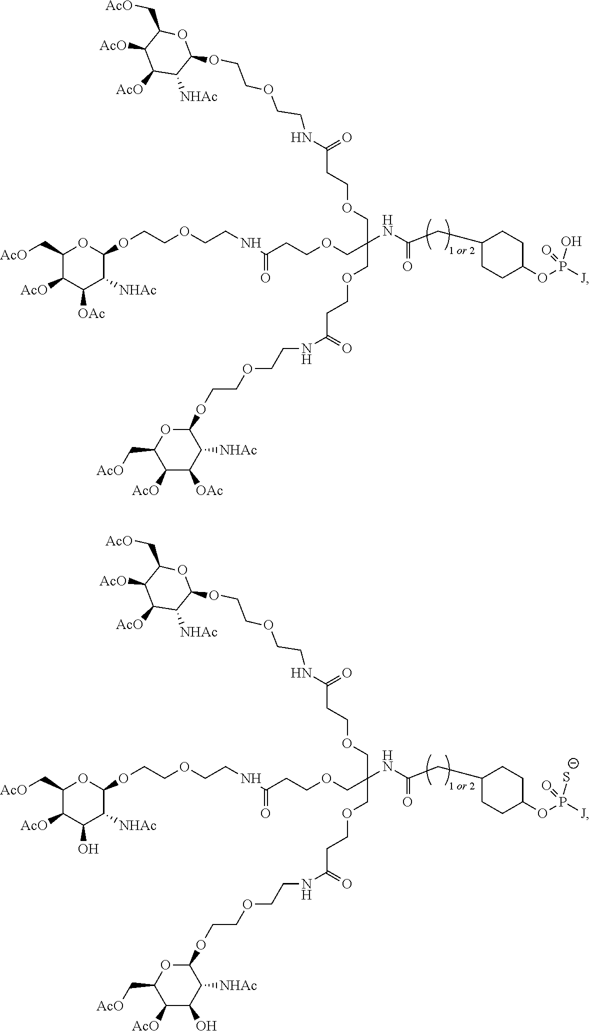

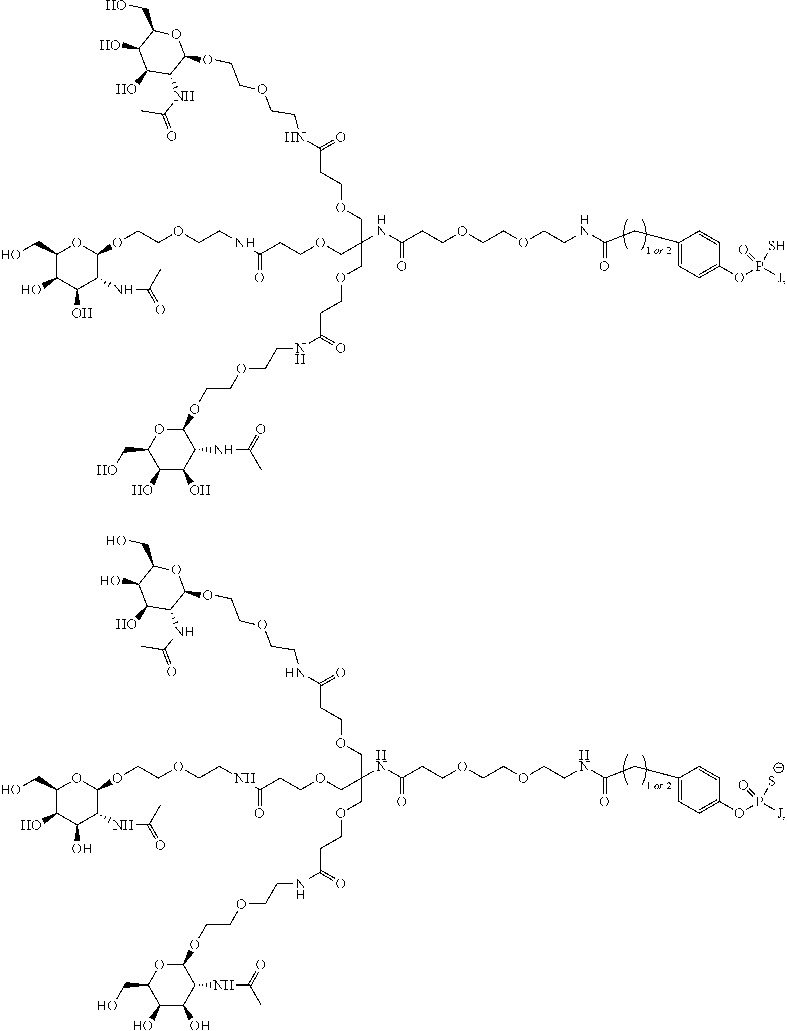

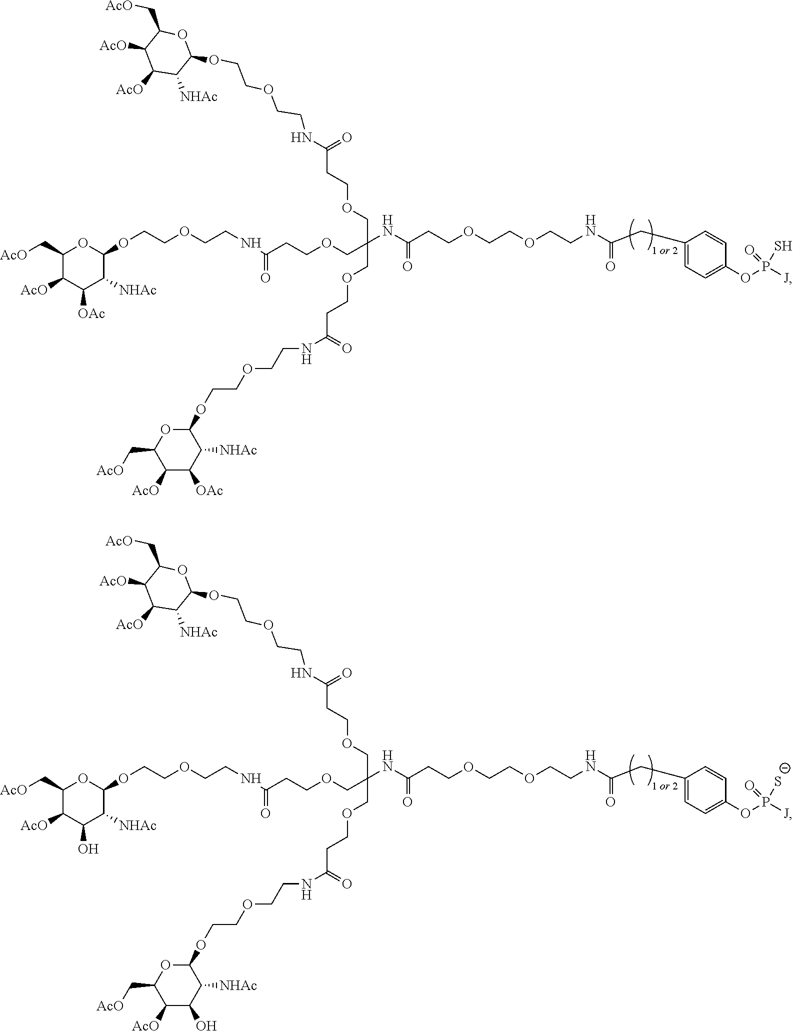

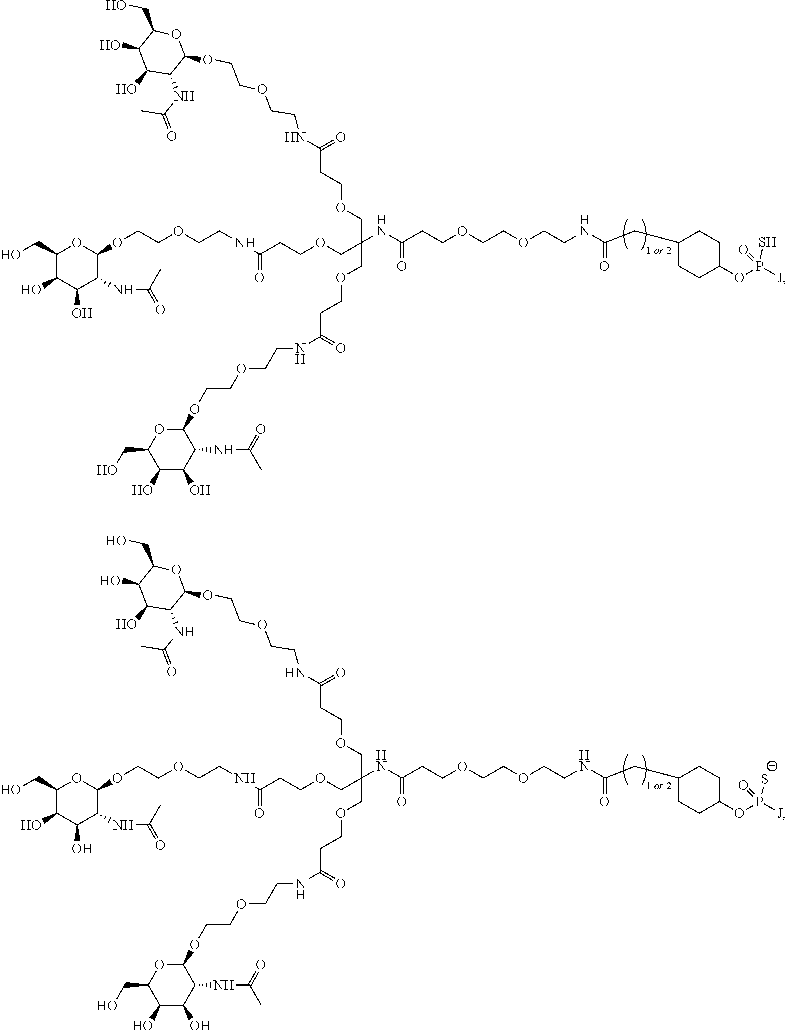

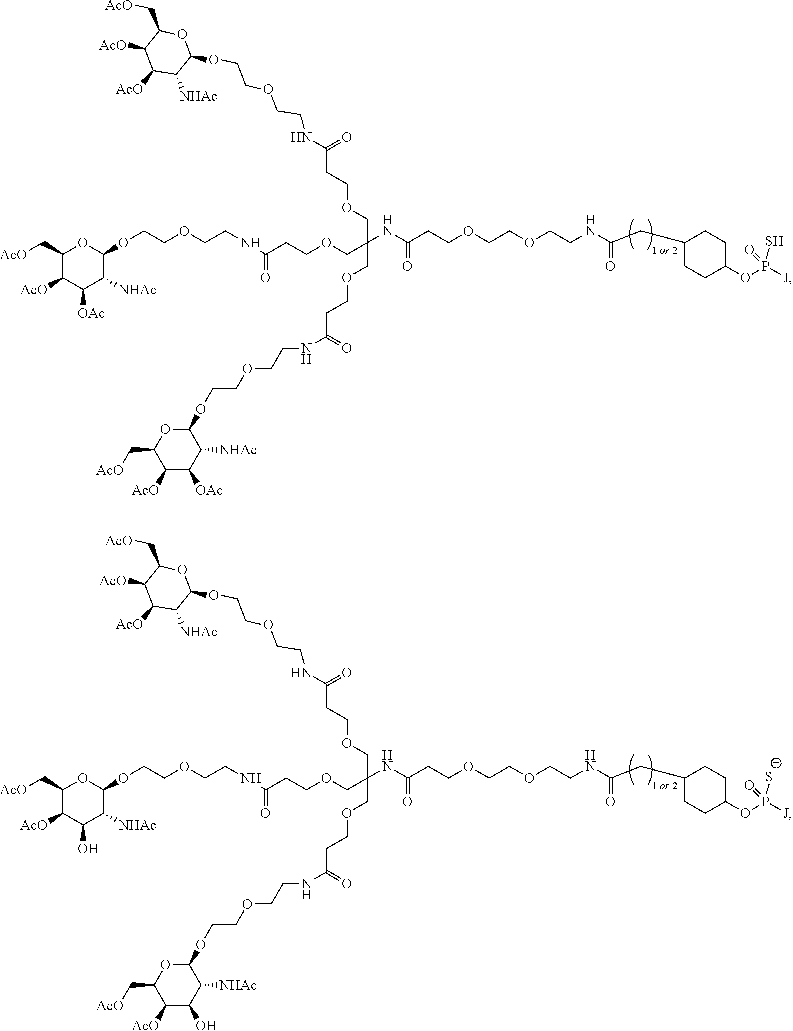

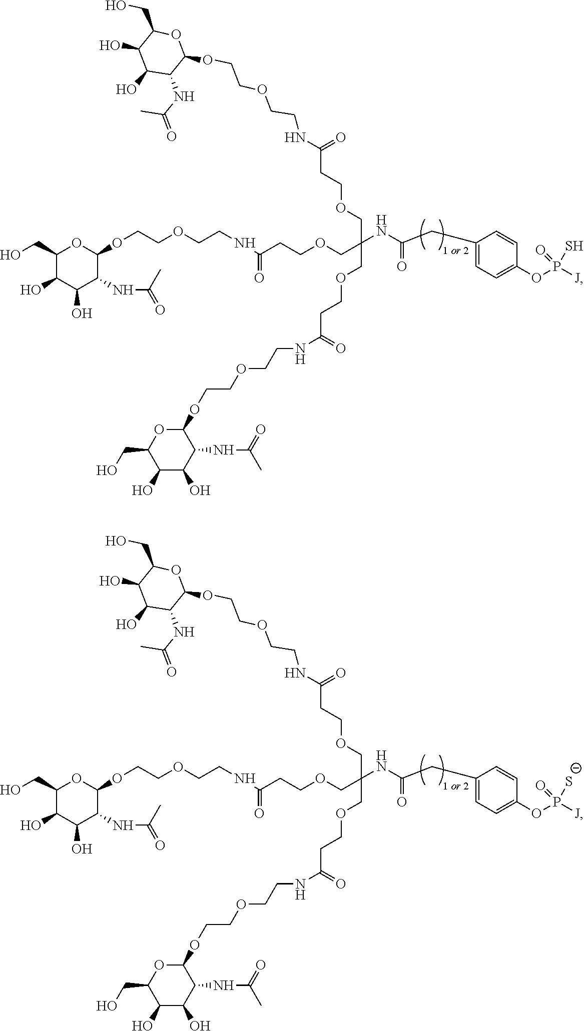

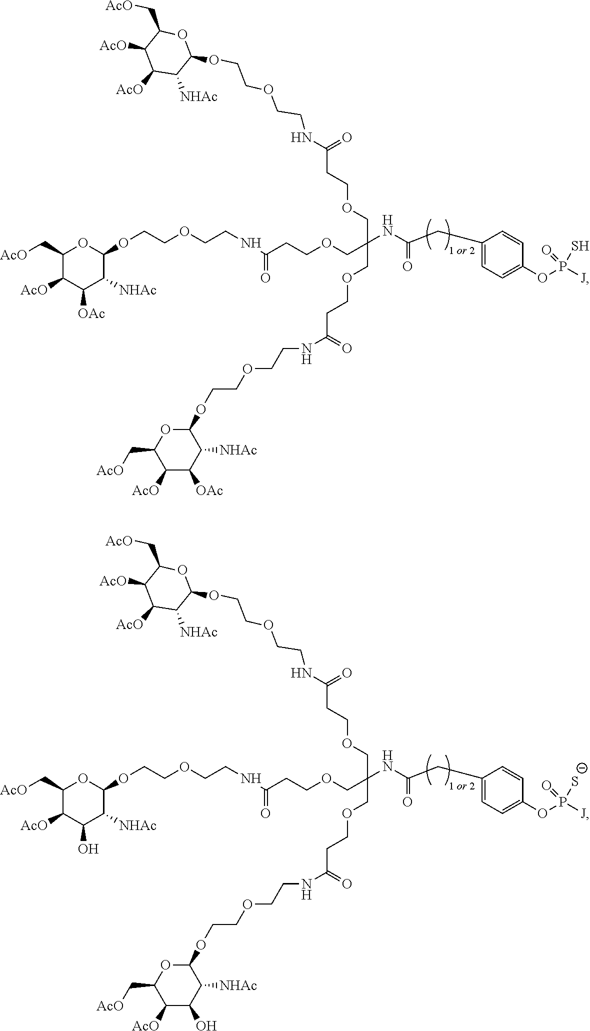

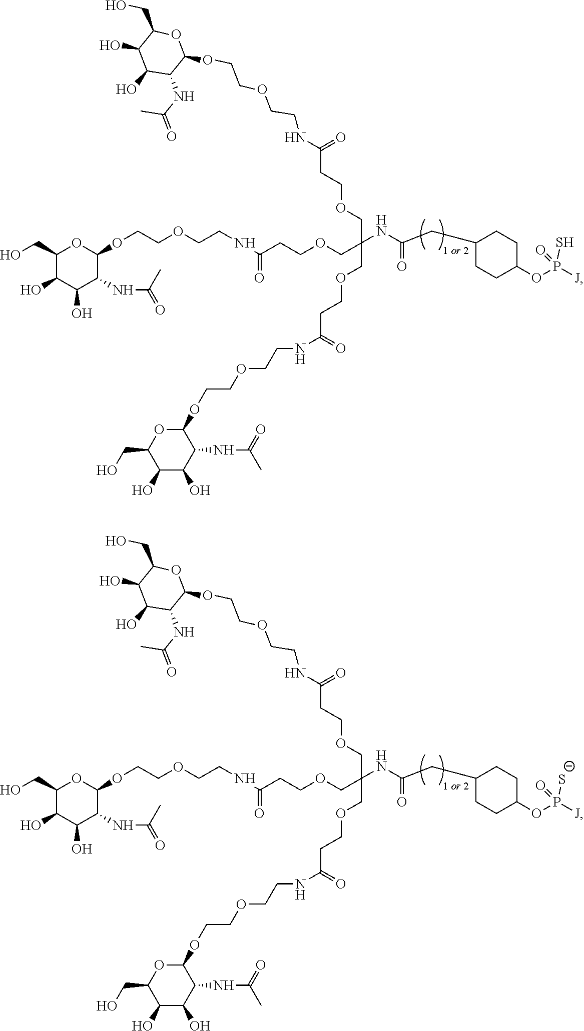

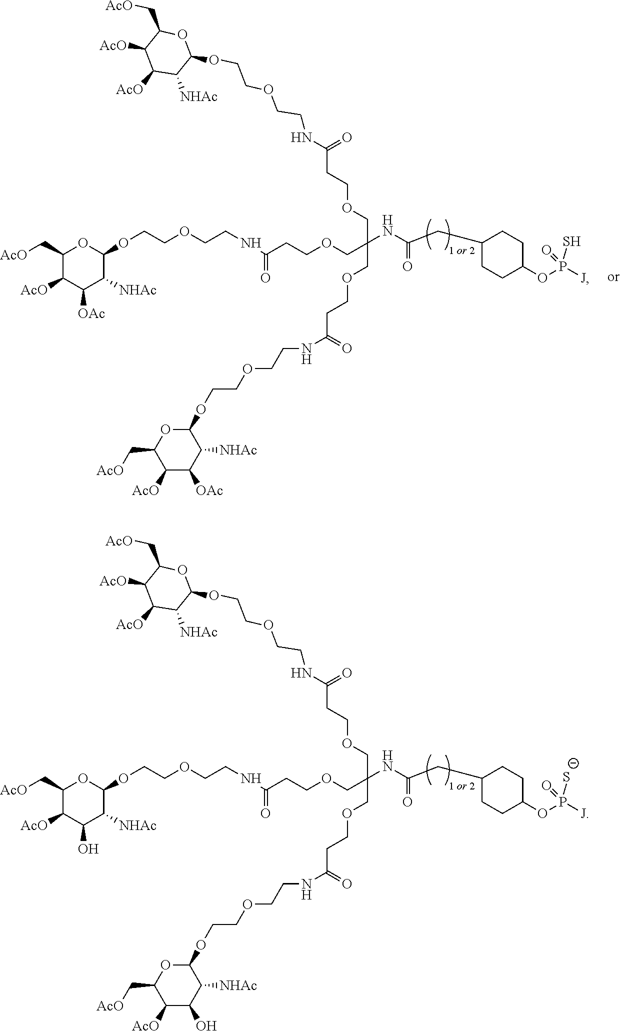

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

Some embodiments include the following, where J is the oligonucleotide:

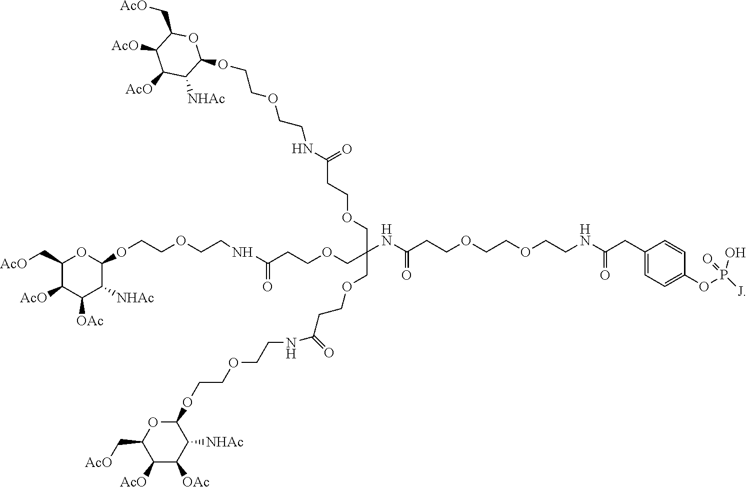

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

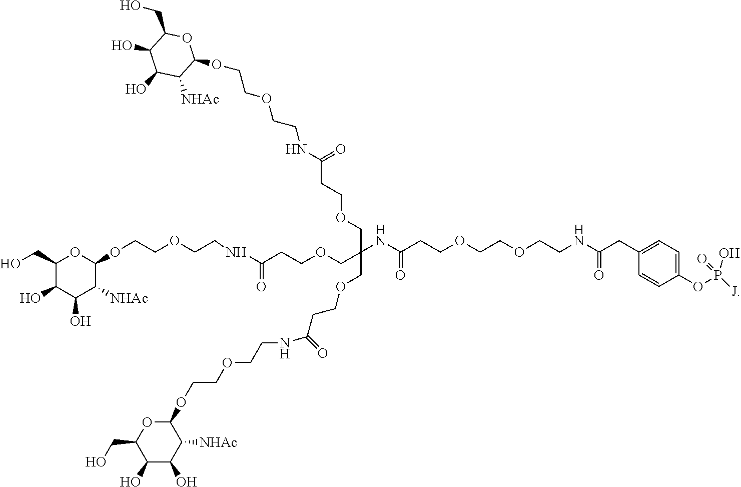

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

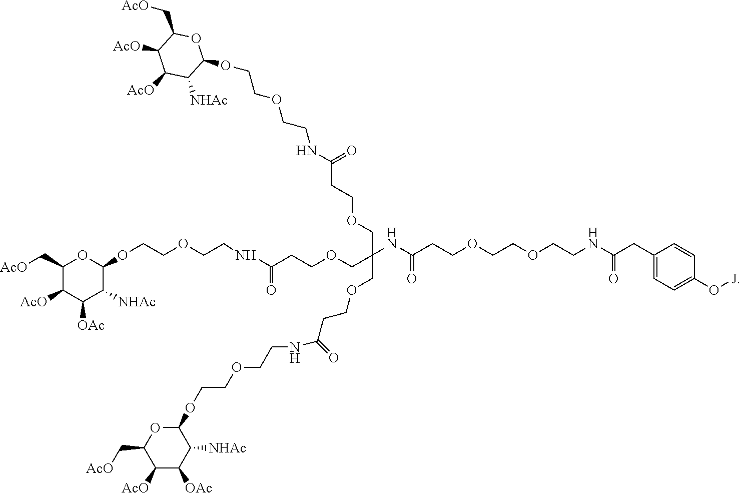

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

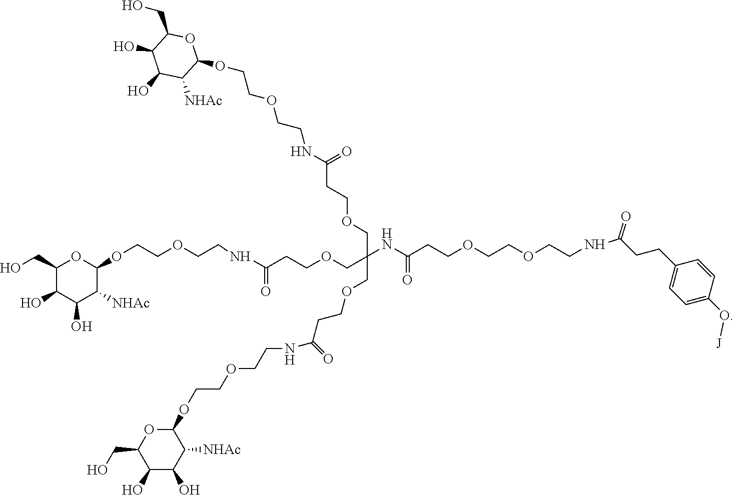

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where the phosphate or “5′” indicates a connection to the oligonucleotide:

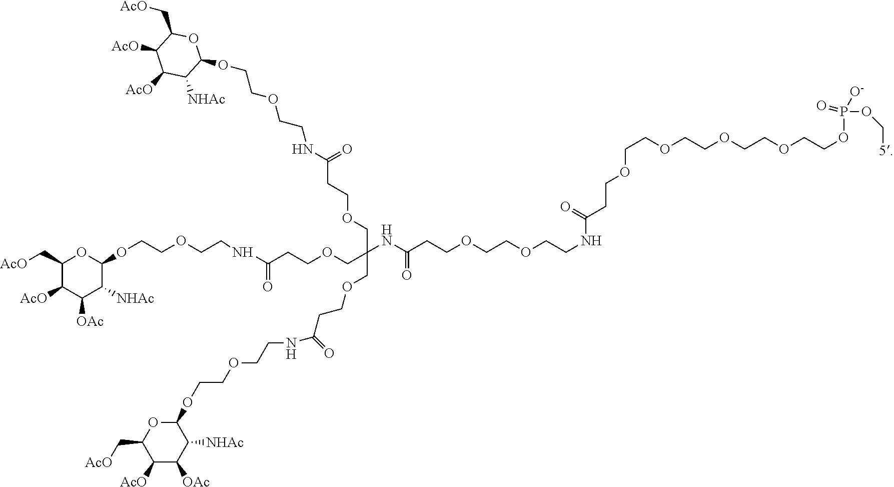

Some embodiments include the following, where the phosphate or “5′” indicates a connection to the oligonucleotide:

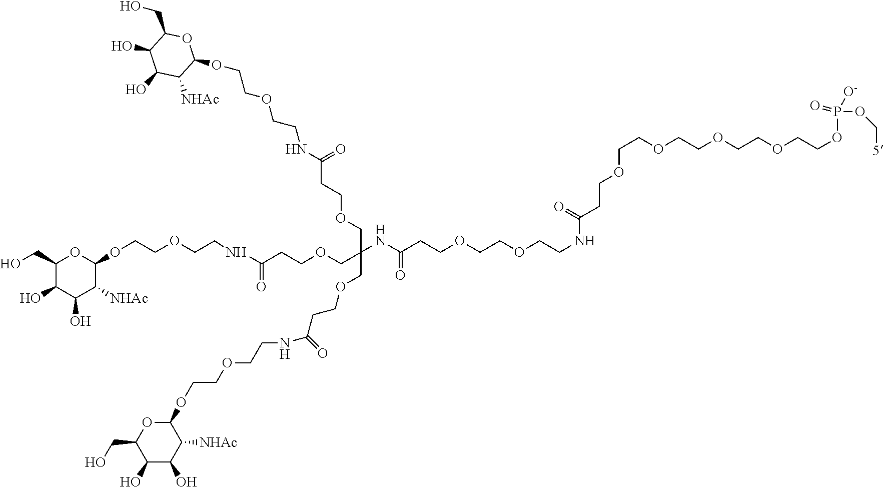

Some embodiments include the following, where J is the oligonucleotide:

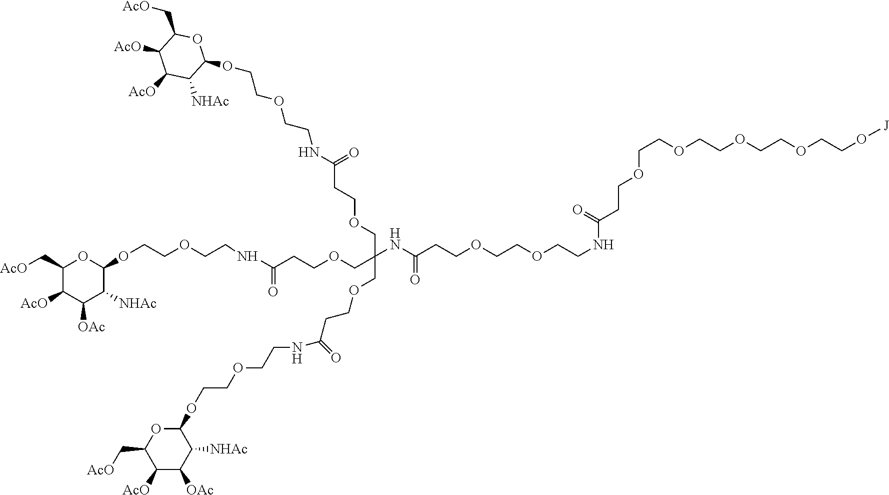

include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

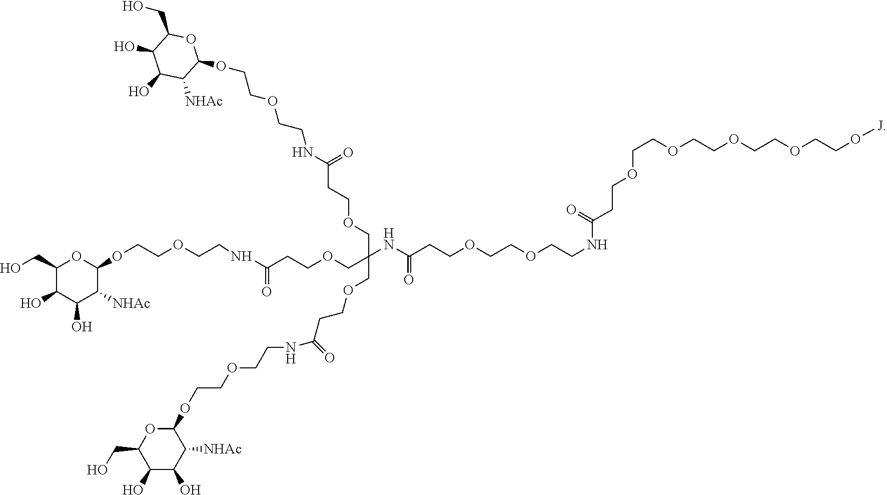

The structure in this compound attached to the oligonucleotide (J) may be referred to as “ETL1,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

3. siRNA Modification Patterns

In some embodiments, the composition comprises an oligonucleotide that inhibits the expression of MST1R, wherein the oligonucleotide comprises an siRNA comprising a sense strand and an antisense strand, wherein the sense strand comprises modification pattern 1S: 5′-NfsnsNfnNfnNfNfNfnNfnNfnNfnNfnNfsnsn-3′ (SEQ ID NO: 9819), wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′ (SEQ ID NO: 9820), wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′ (SEQ ID NO: 9821), wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-moiety-3′ (SEQ ID NO: 9822), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-moiety-3′ (SEQ ID NO: 9823), wherein “Nf” is a 2′ fluoro-modified nucleoside, “n” is a 2′ O-methyl modified nucleoside, “s” is a phosphorothioate linkage, and N comprises one or more nucleosides. In some embodiments, the moiety in modification pattern 4S or 5S includes an integrin targeting ligand. In some embodiments, the moiety in modification pattern 4S or 5S is a sugar moiety. In some embodiments, the sense strand comprises modification pattern 6S: 5′-NfsnsNfnNfnNfnNfnNfnNfnNfnNfnNfsnsn-3′ (SEQ ID NO: 9824), wherein “Nf” is a 2′ fluoro-modified nucleoside, “n” is a 2′ O-methyl modified nucleoside, and “s” is a phosphorothioate linkage. In some embodiments, the sense strand comprises modification pattern 7S: 5′-nsnsnnNfNfNfNfNfnnnnnnnnnnsnsn-3′ (SEQ ID NO: 9825), wherein “Nf” is a 2′ fluoro-modified nucleoside, “n” is a 2′ O-methyl modified nucleoside, and “s” is a phosphorothioate linkage. In some embodiments, the sense strand comprises modification pattern 8S: 5′-nsnsnnnNfNfNfNfnnnnnnnnnnsnsn-3′ (SEQ ID NO: 9826), wherein “Nf” is a 2′ fluoro-modified nucleoside, “n” is a 2′ O-methyl modified nucleoside, and “s” is a phosphorothioate linkage. In some embodiments, the sense strand comprises modification pattern 9S: 5′-nsnsnnnnNfNfNfNfnnnnnnnnnsnsn-3′ (SEQ ID NO: 9827), wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of MST1R, wherein the oligonucleotide comprises an siRNA comprising a sense strand and an antisense strand, wherein the antisense strand comprises modification pattern 1AS: 5′-nsNfsnNfnNfnNfnNfnnnNfnNfnNfnsnsn-3′ (SEQ ID NO: 9828), wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNfNfnnnnNfnNfnnnsnsn-3′ (SEQ ID NO: 9829), wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnNfnnnsnsn-3′ (SEQ ID NO: 9830), wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfnNfnnnsnsn-3′ (SEQ ID NO: 9831), wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnnnnnnnnnnnNfnNfnnnsnsn-3′ (SEQ ID NO: 9832), wherein “Nf” is a 2′ fluoro-modified nucleoside, “n” is a 2′ O-methyl modified nucleoside, and “s” is a phosphorothioate linkage. In some embodiments, the antisense strand comprises modification pattern 6AS: 5′-nsNfsnnnNfnnNfnnnnNfnNfnnnsnsn-3′ (SEQ ID NO: 9833), wherein “Nf” is a 2′ fluoro-modified nucleoside, “n” is a 2′ O-methyl modified nucleoside, and “s” is a phosphorothioate linkage. In some embodiments, the antisense strand comprises modification pattern 7AS: 5′-nsNfsnNfnNfnNfnNfnNfnNfnNfnNfnsnsn-3′ (SEQ ID NO: 9834), wherein “Nf” is a 2′ fluoro-modified nucleoside, “n” is a 2′ O-methyl modified nucleoside, and “s” is a phosphorothioate linkage. In some embodiments, the antisense strand comprises modification pattern 8AS: 5′-nsNfsnnnnnnnnnnnNfnnnnnsnsn-3′ (SEQ ID NO: 9835), wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of MST1R, wherein the oligonucleotide comprises an siRNA comprising a sense strand and an antisense strand, wherein the sense strand comprises pattern 1S and the antisense strand comprises pattern 1AS, 2AS, 3AS, 4AS, 5AS, 6AS, 7AS, or 8AS. In some embodiments, the sense strand comprises pattern 2S and the antisense strand comprises pattern 1AS, 2AS, 3AS, 4AS, 5AS, 6AS, 7AS, or 8AS. In some embodiments, the sense strand comprises pattern 3S and the antisense strand comprises pattern 1AS, 2AS, 3AS, 4AS, 5AS, 6AS, 7AS, or 8AS. In some embodiments, the sense strand comprises pattern 4S and the antisense strand comprises pattern 1AS, 2AS, 3AS, 4AS, 5AS, 6AS, 7AS, or 8AS. In some embodiments, the sense strand comprises pattern 5S and the antisense strand comprises pattern 1AS, 2AS, 3AS, 4AS, 5AS, 6AS, 7AS, or 8AS. In some embodiments, the sense strand comprises pattern 6S and the antisense strand comprises pattern 1AS, 2AS, 3AS, 4AS, 5AS, 6AS, 7AS, or 8AS. In some embodiments, the sense strand comprises pattern 7S and the antisense strand comprises pattern 1AS, 2AS, 3AS, 4AS, 5AS, 6AS, 7AS, or 8AS. In some embodiments, the sense strand comprises pattern 8S and the antisense strand comprises pattern 1AS, 2AS, 3AS, 4AS, 5AS, 6AS, 7AS, or 8AS. In some embodiments, the sense strand comprises pattern 9S and the antisense strand comprises pattern 1AS, 2AS, 3AS, 4AS, 5AS, 6AS, 7AS, or 8AS.

In some embodiments, the sense strand comprises pattern 1S, 2S, 3S, 4S, 5S, 6S, 7S, 8S, or 9S and the antisense strand comprises pattern 1AS. In some embodiments, the sense strand comprises pattern 1S, 2S, 3S, 4S, 5S, 6S, 7S, 8S, or 9S and the antisense strand comprises pattern 2AS. In some embodiments, the sense strand comprises pattern 1S, 2S, 3S, 4S, 5S, 6S, 7S, 8S, or 9S and the antisense strand comprises pattern 3AS. In some embodiments, the sense strand comprises pattern 1S, 2S, 3S, 4S, 5S, 6S, 7S, 8S, or 9S and the antisense strand comprises pattern 4AS. In some embodiments, the sense strand comprises pattern 1S, 2S, 3S, 4S, 5S, 6S, 7S, 8S, or 9S and the antisense strand comprises pattern 5AS. In some embodiments, the sense strand comprises pattern 1S, 2S, 3S, 4S, 5S, 6S, 7S, 8S, or 9S and the antisense strand comprises pattern 6AS. In some embodiments, the sense strand comprises pattern 1S, 2S, 3S, 4S, 5S, 6S, 7S, 8S, or 9S and the antisense strand comprises pattern 7AS. In some embodiments, the sense strand comprises pattern 1S, 2S, 3S, 4S, 5S, 6S, 7S, 8S, or 9S and the antisense strand comprises pattern 8AS.

In some embodiments, the sense strand comprises modification pattern 1AS, 2AS, 3AS, 4AS, 5AS, 6AS, 7AS, or 8AS. In some embodiments, the antisense strand comprises modification pattern 1S, 2S, 3S, 4S, 5S, 6S, 7S, 8S, or 9S. In some embodiments, the sense strand or the antisense strand comprises modification pattern ASO1.

In some embodiments, purines of the sense strand comprise 2′ fluoro modified purines. In some embodiments, purines of the sense strand comprise 2′-O-methyl modified purines. In some embodiments, purines of the sense strand comprise a mixture of 2′ fluoro and 2′-O-methyl modified purines. In some embodiments, all purines of the sense strand comprise 2′ fluoro modified purines. In some embodiments, all purines of the sense strand comprise 2′-O-methyl modified purines. In some embodiments, all purines of the sense strand comprise a mixture of 2′ fluoro and 2′-O-methyl modified purines.

In some embodiments, pyrimidines of the sense strand comprise 2′ fluoro modified pyrimidines. In some embodiments, pyrimidines of the sense strand comprise 2′-O-methyl modified pyrimidines. In some embodiments, pyrimidines of the sense strand comprise a mixture of 2′ fluoro and 2′-O-methyl modified pyrimidines. In some embodiments, all pyrimidines of the sense strand comprise 2′ fluoro modified pyrimidines. In some embodiments, all pyrimidines of the sense strand comprise 2′-O-methyl modified pyrimidines. In some embodiments, all pyrimidines of the sense strand comprise a mixture of 2′ fluoro and 2′-O-methyl modified pyrimidines.

In some embodiments, purines of the sense strand comprise 2′ fluoro modified purines, and pyrimidines of the sense strand comprise a mixture of 2′ fluoro and 2′-O-methyl modified pyrimidines. In some embodiments, purines of the sense strand comprise 2′-O-methyl modified purines, and pyrimidines of the sense strand comprise a mixture of 2′ fluoro and 2′-O-methyl modified pyrimidines. In some embodiments, purines of the sense strand comprise 2′ fluoro modified purines, and pyrimidines of the sense strand comprise 2′-O-methyl modified pyrimidines. In some embodiments, purines of the sense strand comprise 2′-O-methyl modified purines, and pyrimidines of the sense strand comprise 2′ fluoro modified pyrimidines. In some embodiments, pyrimidines of the sense strand comprise 2′ fluoro modified pyrimidines, and purines of the sense strand comprise a mixture of 2′ fluoro and 2′-O-methyl modified purines. In some embodiments, pyrimidines of the sense strand comprise 2′-O-methyl modified pyrimidines, and purines of the sense strand comprise a mixture of 2′ fluoro and 2′-O-methyl modified purines. In some embodiments, pyrimidines of the sense strand comprise 2′ fluoro modified pyrimidines, and purines of the sense strand comprise 2′-O-methyl modified purines. In some embodiments, pyrimidines of the sense strand comprise 2′-O-methyl modified pyrimidines, and purines of the sense strand comprise 2′ fluoro modified purines.

In some embodiments, all purines of the sense strand comprise 2′ fluoro modified purines, and all pyrimidines of the sense strand comprise a mixture of 2′ fluoro and 2′-O-methyl modified pyrimidines. In some embodiments, all purines of the sense strand comprise 2′-O-methyl modified purines, and all pyrimidines of the sense strand comprise a mixture of 2′ fluoro and 2′-O-methyl modified pyrimidines. In some embodiments, all purines of the sense strand comprise 2′ fluoro modified purines, and all pyrimidines of the sense strand comprise 2′-O-methyl modified pyrimidines. In some embodiments, all purines of the sense strand comprise 2′-O-methyl modified purines, and all pyrimidines of the sense strand comprise 2′ fluoro modified pyrimidines. In some embodiments, all pyrimidines of the sense strand comprise 2′ fluoro modified pyrimidines, and all purines of the sense strand comprise a mixture of 2′ fluoro and 2′-O-methyl modified purines. In some embodiments, all pyrimidines of the sense strand comprise 2′-O-methyl modified pyrimidines, and all purines of the sense strand comprise a mixture of 2′ fluoro and 2′-O-methyl modified purines. In some embodiments, all pyrimidines of the sense strand comprise 2′ fluoro modified pyrimidines, and all purines of the sense strand comprise 2′-O-methyl modified purines. In some embodiments, all pyrimidines of the sense strand comprise 2′-O-methyl modified pyrimidines, and all purines of the sense strand comprise 2′ fluoro modified purines.

In some embodiments, purines of the antisense strand comprise 2′ fluoro modified purines. In some embodiments, purines of the antisense strand comprise 2′-O-methyl modified purines. In some embodiments, purines of the antisense strand comprise a mixture of 2′ fluoro and 2′-O-methyl modified purines. In some embodiments, all purines of the antisense strand comprise 2′ fluoro modified purines. In some embodiments, all purines of the antisense strand comprise 2′-O-methyl modified purines. In some embodiments, all purines of the antisense strand comprise a mixture of 2′ fluoro and 2′-O-methyl modified purines.

In some embodiments, pyrimidines of the antisense strand comprise 2′ fluoro modified pyrimidines. In some embodiments, pyrimidines of the antisense strand comprise 2′-O-methyl modified pyrimidines. In some embodiments, pyrimidines of the antisense strand comprise a mixture of 2′ fluoro and 2′-O-methyl modified pyrimidines. In some embodiments, all pyrimidines of the antisense strand comprise 2′ fluoro modified pyrimidines. In some embodiments, all pyrimidines of the antisense strand comprise 2′-O-methyl modified pyrimidines. In some embodiments, all pyrimidines of the antisense strand comprise a mixture of 2′ fluoro and 2′-O-methyl modified pyrimidines.

In some embodiments, purines of the antisense strand comprise 2′ fluoro modified purines, and pyrimidines of the antisense strand comprise a mixture of 2′ fluoro and 2′-O-methyl modified pyrimidines. In some embodiments, purines of the antisense strand comprise 2′-O-methyl modified purines, and pyrimidines of the antisense strand comprise a mixture of 2′ fluoro and 2′-O-methyl modified pyrimidines. In some embodiments, purines of the antisense strand comprise 2′ fluoro modified purines, and pyrimidines of the antisense strand comprise 2′-O-methyl modified pyrimidines. In some embodiments, purines of the antisense strand comprise 2′-O-methyl modified purines, and pyrimidines of the antisense strand comprise 2′ fluoro modified pyrimidines. In some embodiments, pyrimidines of the antisense strand comprise 2′ fluoro modified pyrimidines, and purines of the antisense strand comprise a mixture of 2′ fluoro and 2′-O-methyl modified purines. In some embodiments, pyrimidines of the antisense strand comprise 2′-O-methyl modified pyrimidines, and purines of the antisense strand comprise a mixture of 2′ fluoro and 2′-O-methyl modified purines. In some embodiments, pyrimidines of the antisense strand comprise 2′ fluoro modified pyrimidines, and purines of the antisense strand comprise 2′-O-methyl modified purines. In some embodiments, pyrimidines of the antisense strand comprise 2′-O-methyl modified pyrimidines, and purines of the antisense strand comprise 2′ fluoro modified purines.

In some embodiments, all purines of the antisense strand comprise 2′ fluoro modified purines, and all pyrimidines of the antisense strand comprise a mixture of 2′ fluoro and 2′-O-methyl modified pyrimidines. In some embodiments, all purines of the antisense strand comprise 2′-O-methyl modified purines, and all pyrimidines of the antisense strand comprise a mixture of 2′ fluoro and 2′-O-methyl modified pyrimidines. In some embodiments, all purines of the antisense strand comprise 2′ fluoro modified purines, and all pyrimidines of the antisense strand comprise 2′-O-methyl modified pyrimidines. In some embodiments, all purines of the antisense strand comprise 2′-O-methyl modified purines, and all pyrimidines of the antisense strand comprise 2′ fluoro modified pyrimidines. In some embodiments, all pyrimidines of the antisense strand comprise 2′ fluoro modified pyrimidines, and all purines of the antisense strand comprise a mixture of 2′ fluoro and 2′-O-methyl modified purines. In some embodiments, all pyrimidines of the antisense strand comprise 2′-O-methyl modified pyrimidines, and all purines of the antisense strand comprise a mixture of 2′ fluoro and 2′-O-methyl modified purines. In some embodiments, all pyrimidines of the antisense strand comprise 2′ fluoro modified pyrimidines, and all purines of the antisense strand comprise 2′-O-methyl modified purines. In some embodiments, all pyrimidines of the antisense strand comprise 2′-O-methyl modified pyrimidines, and all purines of the antisense strand comprise 2′ fluoro modified purines.

Disclosed herein, in some embodiments, are modified oligonucleotides. The modified oligonucleotide may be an siRNA that includes modifications to the ribose rings, and phosphate linkages. The modifications may be in particular patterns that maximize cell delivery, stability, and efficiency. The siRNA may also include a vinyl phosphonate and a hydrophobic group. These modifications may aid in delivery to a cell or tissue within a subject. The modified oligonucleotide may be used in a method such as a treatment method or a method of reducing gene expression.

In some embodiments, the oligonucleotide comprises a duplex consisting of 21 nucleotide single strands with base pairing between 19 of the base pairs. In some embodiments, the duplex comprises single-stranded 2 nucleotide overhangs are at the 3′ ends of each strand. One strand (antisense strand) is complementary to a MST1R mRNA. Each end of the antisense strand has one to two phosphorothioate bonds. The 5′ end has an optional phosphate mimic such as a vinyl phosphonate. In some embodiments, the oligonucleotide is used to knock down a MST1R mRNA or a target protein. In some embodiments, the sense strand has the same sequence as the MST1R mRNA. In some embodiments, there are 1-2 phosphorothioates at the 3′ end. In some embodiments, there are 1 or no phosphorothioates at the 5′ end. In some embodiments, there is a hydrophobic conjugate of 12 to 25 carbons attached at the 5′ end via a phosphodiester bond.

In some cases, the sense strand of any of the siRNAs comprises siRNA with a particular modification pattern. In some embodiments of the modification pattern, position 9 counting from the 5′ end of the sense strand may have a 2′F modification. In some embodiments, when position 9 of the sense strand is a pyrimidine, then all purines in the sense strand have a 2′OMe modification. In some embodiments, when position 9 is the only pyrimidine between positions 5 and 11 of the sense stand, then position 9 is the only position with a 2′F modification in the sense strand. In some embodiments, when position 9 and only one other base between positions 5 and 11 of the sense strand are pyrimidines, then both of these pyrimidines are the only two positions with a 2′F modification in the sense strand. In some embodiments, when position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of the sense strand, then all combinations of pyrimidines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, when position 9 of the sense strand is a purine, then all purines in the sense strand have a 2′OMe modification. In some embodiments, when position 9 is the only purine between positions 5 and 11 of the sense stand, then position 9 is the only position with a 2′F modification in the sense strand. In some embodiments, when position 9 and only one other base between positions 5 and 11 of the sense strand are purines, then both of these purines are the only two positions with a 2′F modification in the sense strand. In some embodiments, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some cases, position 9 of the sense strand can be a 2′deoxy. In these cases, 2′F and 2′OMe modifications may occur at the other positions of the sense strand. In some cases, the sense strand of any of the siRNAs comprises a modification pattern which conforms to these sense strand rules.

In some cases, the sense strand of any of the siRNAs comprises a modification pattern which conforms to these sense strand rules.

In some embodiments, the siRNA comprises the sense strand and/or the antisense strand sequence of an siRNA in Table 9, or a nucleic acid sequence thereof having 3 or 4 nucleoside substitutions, additions, or deletions. In some embodiments, the siRNA comprises the sense strand and/or the antisense strand sequence of an siRNA in Table 9, or a nucleic acid sequence thereof having 1 or 2 nucleoside substitutions, additions, or deletions. In some embodiments, the siRNA comprises the sense strand and/or the antisense strand sequence of an siRNA in Table 9. The siRNA may include the same internucleoside linkage modifications or nucleoside modifications as those in Table 9. The siRNA may include any different internucleoside linkage modifications or nucleoside modifications different from those in Table 9. The siRNA may include some unmodified internucleoside linkages or nucleosides.

In some embodiments, the siRNA comprises the sense strand and/or the antisense strand sequence of an siRNA in Table 10, or a nucleic acid sequence thereof having 3 or 4 nucleoside substitutions, additions, or deletions. In some embodiments, the siRNA comprises the sense strand and/or the antisense strand sequence of an siRNA in Table 10, or a nucleic acid sequence thereof having 1 or 2 nucleoside substitutions, additions, or deletions. In some embodiments, the siRNA comprises the sense strand and/or the antisense strand sequence of an siRNA in Table 10. The siRNA may include the same internucleoside linkage modifications or nucleoside modifications as those in Table 10. The siRNA may include any different internucleoside linkage modifications or nucleoside modifications different from those in Table 10. The siRNA may include some unmodified internucleoside linkages or nucleosides.

### 4. ASO Modification Patterns

In some embodiments, the composition comprises an oligonucleotide that inhibits the expression of MST1R, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′ (SEQ ID NO: 9836), wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S, 2S, 3S, 4S, 5S, 6S, 7S, 8S, 9S, 1AS, 2AS, 3AS, 4AS, 5AS, 6AS, 7AS, or 8AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof. In some embodiments, the oligonucleotide is combined with lipids, nanoparticles, polymers, liposomes, micelles, or another delivery system.

In some embodiments, the composition is formulated for delivery to a subject's lungs. In some embodiments, the composition is formulated for inhalation. In some embodiments, the composition is formulated for aerosolization. In some embodiments, the composition is formulated for administration by a nebulizer.

### II. METHODS AND USES

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection. In some embodiments, the administration is to a subject's lungs. In some embodiments, the administration is by inhalation. In some embodiments, the administration is performed using a nebulizer.

### A. Disorders

Some embodiments of the methods described herein include treating a disorder in a subject in need thereof. In some embodiments, the disorder is a lung disorder. Non-limiting examples of lung disorders include chronic obstructive pulmonary disease (COPD), acute exacerbation of COPD, emphysema, chronic bronchitis, asthma, status asthmaticus, asthma-COPD overlap syndrome (ACOS), cough, lung cancer, interstitial lung disease, or pulmonary fibrosis. The lung disorder may include an obstructive airway disorder such as COPD or asthma. In some embodiments, the lung disorder includes COPD. In some embodiments, the lung disorder includes acute exacerbation of COPD. In some embodiments, the lung disorder includes emphysema. In some embodiments, the lung disorder includes chronic bronchitis. In some embodiments, the lung disorder includes asthma. In some embodiments, the lung disorder includes status asthmaticus. In some embodiments, the lung disorder includes ACOS. In some embodiments, the lung disorder includes cough. In some embodiments, the lung disorder includes lung cancer. In some embodiments, the lung disorder includes interstitial lung disease. In some embodiments, the lung disorder includes pulmonary fibrosis.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female. In some embodiments, the subject is an adult (e.g. at least 18 years old).

### C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline lung function measurement, a baseline leukocyte measurement, a baseline chronic obstructive pulmonary disease (COPD) exacerbation measurement, a baseline asthma exacerbation measurement, a baseline MST1R protein measurement, or a baseline MST1R mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline lung function measurement. In some embodiments, the baseline measurement is a baseline spirometry measurement. The baseline spirometry measurement may be obtained using a spirometer. The spirometer may generate a spirogram comprising a volume-time curve or a flow-volume loop. In some embodiments, the baseline spirometry measurement is obtained by having the subject breathe into a spirometer sensor. Examples of baseline spirometry measurements may include a baseline forced expiratory volume in 1 second (FEV1) measurement, a baseline forced expiratory volume in 1 second percent predicted (FEV1pp) measurement, a baseline forced vital capacity (FVC) measurement, a baseline FEV1/FVC ratio, a baseline forced expiratory volume, or a baseline peak expiratory flow measurement. In some embodiments, the baseline measurement includes a baseline forced expiratory volume in 1 second (FEV1) measurement. In some embodiments, the baseline measurement includes a baseline forced expiratory volume in 1 second percent predicted (FEV1pp) measurement. In some embodiments, the baseline measurement includes a baseline forced vital capacity (FVC) measurement. In some embodiments, the baseline measurement includes a baseline FEV1/FVC ratio. The baseline FEV1/FVC ratio may be below 70% or below 80%, in some cases. In some embodiments, the baseline measurement includes a baseline forced expiratory volume. In some embodiments, the baseline measurement includes a baseline peak expiratory flow measurement.

In some embodiments, the baseline measurement includes a baseline leukocyte measurement. In some embodiments, the baseline leukocyte measurement includes a baseline circulating leukocyte measurement. In some embodiments, the baseline leukocyte measurement includes a baseline lung tissue leukocyte measurement. In some embodiments, the baseline leukocyte measurement includes a baseline lung fluid (e.g. bronchoalveolar fluid) leukocyte measurement. In some embodiments, the baseline leukocyte measurement includes a baseline leukocyte count. In some embodiments, the baseline leukocyte measurement includes a baseline leukocyte concentration. In some embodiments, the baseline leukocyte measurement includes a baseline leukocyte percentage. The percentage may be in relation to other cells. Examples of leukocytes that may be included in the baseline leukocyte measurement include neutrophils, eosinophils, basophils, monocytes, or lymphocytes. The leukocytes may include neutrophils. The leukocytes may include eosinophils. The leukocytes may include basophils. The leukocytes may include monocytes. The leukocytes may include lymphocytes. In some embodiments, the baseline leukocyte measurement is obtained by an assay such as an immunoassay, a colorimetric assay, or a fluorescence assay. In some embodiments, the baseline leukocyte measurement is high, relative to a control leukocyte measurement. For example, a subject who has not been treated with a composition described herein and who has an inflammatory lung disorder may have a high leukocyte count in the subject's blood or lungs. In some embodiments, the baseline leukocyte measurement is determined in lung tissue or a lung fluid such as bronchoalveolar fluid, and may include a baseline measurement of neutrophils and macrophages.

In some embodiments, the baseline measurement includes a baseline chronic obstructive pulmonary disease (COPD) exacerbation measurement. A COPD exacerbation may include a COPD flare-up such as an acute increase in severity of a respiratory symptom such as difficulty breathing. The baseline COPD exacerbation measurement may include a baseline number of COPD flare-ups, and may be included in a given time frame such as flare-ups per day, week, month, or year. The baseline COPD exacerbation measurement may include a baseline frequency of COPD exacerbations. The baseline COPD exacerbation measurement may include a baseline measurement of worsening of a respiratory symptom, such as increased dyspnea, cough, sputum volume, or sputum purulence. The baseline COPD exacerbation measurement may include a baseline measurement of an event such as when a the subject's conditions change enough to require a change in treatment. The baseline COPD exacerbation measurement may include a baseline peak flow test, a baseline breath nitric oxide measurement, or a baseline blood oxygen level test.

In some embodiments, the baseline measurement includes a baseline asthma exacerbation measurement. An asthma exacerbation may include an asthma attack, for example narrowing of a bronchial tube that causes difficulty breathing. The baseline asthma exacerbation measurement may include a baseline number of number of asthma attacks, and may be included in a given time frame such as flare-ups per day, week, month, or year. The baseline asthma exacerbation measurement may include a baseline frequency of asthma exacerbations. The baseline asthma exacerbation measurement may include a baseline bronchial tube measurement such as a bronchial tube diameter, a bronchial tube circumference, or a bronchial tube area measurement. The baseline asthma exacerbation measurement may include a baseline amount of bronchial tube narrowing, such as a percent constriction. The baseline asthma exacerbation measurement may include a baseline wheezing measurement, a baseline coughing measurement, a baseline chest tightening measurement, a baseline shortness of breath measurement, a baseline agitation measurement, a baseline hyperventilation measurement, a baseline heart rate measurement, a baseline lung function measurement, or a baseline measurement of difficulty speaking or breathing. The baseline asthma exacerbation measurement may include a baseline peak flow test, a baseline breath nitric oxide measurement, or a baseline blood oxygen level test.

In some embodiments, the baseline measurement is a baseline MST1R protein measurement. In some embodiments, the baseline MST1R protein measurement comprises a baseline MST1R protein level. In some embodiments, the baseline MST1R protein level is indicated as a mass or percentage of MST1R protein per sample weight. In some embodiments, the baseline MST1R protein level is indicated as a mass or percentage of MST1R protein per sample volume. In some embodiments, the baseline MST1R protein level is indicated as a mass or percentage of MST1R protein per total protein within the sample. In some embodiments, the baseline MST1R protein measurement is a baseline lung MST1R protein measurement. In some embodiments, the baseline MST1R protein measurement is obtained by an assay such as an immunoassay, a colorimetric assay, or a fluorescence assay.

In some embodiments, the baseline measurement is a baseline MST1R mRNA measurement. In some embodiments, the baseline MST1R mRNA measurement comprises a baseline MST1R mRNA level. In some embodiments, the baseline MST1R mRNA level is indicated as an amount or percentage of MST1R mRNA per sample weight. In some embodiments, the baseline MST1R mRNA level is indicated as an amount or percentage of MST1R mRNA per sample volume. In some embodiments, the baseline MST1R mRNA level is indicated as an amount or percentage of MST1R mRNA per total mRNA within the sample. In some embodiments, the baseline MST1R mRNA level is indicated as an amount or percentage of MST1R mRNA per total nucleic acids within the sample. In some embodiments, the baseline MST1R mRNA level is indicated relative to another mRNA level, such as an mRNA level of a housekeeping gene, within the sample. In some embodiments, the baseline MST1R mRNA measurement is obtained by an assay such as a polymerase chain reaction (PCR) assay. In some embodiments, the PCR comprises quantitative PCR (qPCR). In some embodiments, the PCR comprises reverse transcription of the MST1R mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject.

In some embodiments, the sample comprises a fluid. In some embodiments, the sample is a fluid sample. For example, a fluid sample may be used in obtaining a leukocyte measurement or baseline measurement. In some embodiments, the sample is a blood, plasma, or serum sample. In some embodiments, the baseline MST1R mRNA measurement is obtained in a fluid sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample. In some embodiments, the fluid sample includes a lung fluid sample. In some embodiments, the lung fluid sample includes alveolar fluid. In some embodiments, the lung fluid sample includes bronchial fluid. In some embodiments, the lung fluid sample includes bronchoalveolar fluid. The lung fluid may be obtained via a lavage method such as a bronchoalveolar lavage method. The lavage method may include the use of a bronchoscope.

In some embodiments, the sample comprises a tissue. In some embodiments, the sample is a tissue sample. In some embodiments, the tissue comprises lung, or vascular tissue. For example, the baseline MST1R mRNA measurement, or the baseline MST1R protein measurement, may be obtained in a lung sample obtained from the patient. In some embodiments, the tissue comprises lung tissue. The lung may include lung epithelial cells, type I alveolar cells, type II alveolar cells, macrophages, alveolar macrophages, goblet cells, club cells, or fibroblasts. In some embodiments, the tissue comprises vascular tissue. The vascular tissue may include vascular endothelial cells. For example, the lung tissue may include vascular endothelial cells.

In some embodiments, the sample includes cells. In some embodiments, the sample comprises a cell. In some embodiments, the cell is a lung cell. In some embodiments, the lung cell is a lung epithelial cell. In some embodiments, the lung cell is a type I alveolar cell. In some embodiments, the lung cell is a type II alveolar cell. In some embodiments, the lung cell is a macrophage. In some embodiments, the lung cell is a alveolar macrophage. In some embodiments, the lung cell is a goblet cell. In some embodiments, the lung cell is a club cell. In some embodiments, the lung cell is a fibroblast. In some embodiments, the cell is a vasculature cell. In some embodiments, the vasculature cell is an endothelial cell.

### D. Effects

In some embodiments, the composition or administration of the composition affects a measurement such as a lung function measurement, a leukocyte measurement, a chronic obstructive pulmonary disease (COPD) exacerbation measurement, an asthma exacerbation measurement, a MST1R protein measurement (for example, lung MST1R protein levels), or a MST1R mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g. HPLC) assay, or a PCR assay. In some embodiments, the measurement is obtained by an assay such as an immunoassay, a colorimetric assay, a fluorescence assay, or a chromatography (e.g. HPLC) assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of a lung disorder may be reduced upon administration of the composition. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective lung phenotype may be increased upon administration of the composition. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a lung function measurement. In some embodiments, the measurement is a spirometry measurement. The spirometry measurement may be obtained using a spirometer. The spirometer may generate a spirogram comprising a volume-time curve or a flow-volume loop. In some embodiments, the spirometry measurement is obtained by having the subject breathe into a spirometer sensor. Examples of spirometry measurements may include a forced expiratory volume in 1 second (FEV1) measurement, a forced expiratory volume in 1 second percent predicted (FEV1pp) measurement, a forced vital capacity (FVC) measurement, a FEV1/FVC ratio, a forced expiratory volume, or a peak expiratory flow measurement. In some embodiments, the measurement includes a forced expiratory volume in 1 second (FEV1) measurement. In some embodiments, the measurement includes a forced expiratory volume in 1 second percent predicted (FEV1pp) measurement. In some embodiments, the measurement includes a forced vital capacity (FVC) measurement. In some embodiments, the measurement includes a FEV1/FVC ratio. The FEV1/FVC ratio may be below 70% or below 80%, in some cases. In some embodiments, the measurement includes a forced expiratory volume. In some embodiments, the measurement includes a peak expiratory flow measurement.

In some embodiments, the composition increases the lung function measurement relative to the baseline lung function measurement. In some embodiments, the increase is measured directly in the subject after administering the composition to the subject. In some embodiments, the lung function measurement is increased by about 2.5% or more, about 5% or more, or about 7.5% or more, relative to the baseline lung function measurement. In some embodiments, the lung function measurement is increased by about 10% or more, relative to the baseline lung function measurement. In some embodiments, the lung function measurement is increased by about 20% or more, about 30% or more, about 40% or more, about 50% or more, about 60% or more, about 70% or more, about 80% or more, about 90% or more, relative to the baseline lung function measurement. In some embodiments, the lung function measurement is increased by about 100% or more, increased by about 250% or more, increased by about 500% or more, increased by about 750% or more, or increased by about 1000% or more, relative to the baseline lung function measurement. In some embodiments, the lung function measurement is increased by no more than about 2.5%, no more than about 5%, or no more than about 7.5%, relative to the baseline lung function measurement. In some embodiments, the lung function measurement is increased by no more than about 10%, relative to the baseline lung function measurement. In some embodiments, the lung function measurement is increased by no more than about 20%, no more than about 30%, no more than about 40%, no more than about 50%, no more than about 60%, no more than about 70%, no more than about 80%, no more than about 90%, or no more than about 100% relative to the baseline lung function measurement. In some embodiments, the lung function measurement is increased by no more than about 100%, increased by no more than about 250%, increased by no more than about 500%, increased by no more than about 750%, or increased by no more than about 1000%, relative to the baseline lung function measurement. In some embodiments, the lung function measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement includes a leukocyte measurement. In some embodiments, the leukocyte measurement includes a circulating leukocyte measurement. In some embodiments, the leukocyte measurement includes a lung tissue leukocyte measurement. In some embodiments, the leukocyte measurement includes a lung fluid (e.g. bronchoalveolar fluid) leukocyte measurement. In some embodiments, the leukocyte measurement includes a leukocyte count. In some embodiments, the leukocyte measurement includes a leukocyte concentration. In some embodiments, the leukocyte measurement includes a leukocyte percentage. The percentage may be in relation to other cells. Examples of leukocytes that may be included in the leukocyte measurement include neutrophils, eosinophils, basophils, monocytes, or lymphocytes. The leukocytes may include neutrophils. The leukocytes may include eosinophils. The leukocytes may include basophils. The leukocytes may include monocytes. The leukocytes may include lymphocytes. In some embodiments, the leukocyte measurement is obtained by an assay such as an immunoassay, a colorimetric assay, or a fluorescence assay. In some embodiments, the leukocyte measurement is normal, relative to a control leukocyte measurement. For example, a subject who has been treated with a composition described herein and who has an inflammatory lung disorder may have had a high leukocyte count that is now low or normal. In some embodiments, the leukocyte measurement is determined in lung tissue or a lung fluid such as bronchoalveolar fluid, and may include a measurement of neutrophils and macrophages.

In some embodiments, the composition reduces the leukocyte measurement relative to the baseline leukocyte measurement. In some embodiments, the reduction is measured in a second sample obtained from the subject after administering the composition to the subject. In some embodiments, the leukocyte measurement is decreased by about 2.5% or more, about 5% or more, or about 7.5% or more, relative to the baseline leukocyte measurement. In some embodiments, the leukocyte measurement is decreased by about 10% or more, relative to the baseline leukocyte measurement. In some embodiments, the leukocyte measurement is decreased by about 20% or more, about 30% or more, about 40% or more, about 50% or more, about 60% or more, about 70% or more, or about 80% or more, relative to the baseline leukocyte measurement. In some embodiments, the leukocyte measurement is decreased by no more than about 2.5%, no more than about 5%, or no more than about 7.5%, relative to the baseline leukocyte measurement. In some embodiments, the leukocyte measurement is decreased by no more than about 10%, relative to the baseline leukocyte measurement. In some embodiments, the leukocyte measurement is decreased by no more than about 20%, no more than about 30%, no more than about 40%, no more than about 50%, no more than about 60%, no more than about 70%, or no more than about 80%, relative to the baseline leukocyte measurement. In some embodiments, the leukocyte measurement is decreased by 2.5%, 5%, 7.5%, 10%, 20%, 30%, 40%, 50%, 60%, 70%, 80%, 90%, or 100%, or by a range defined by any of the two aforementioned percentages. In some embodiments, the leukocyte measurement is increased by any of the aforementioned percentages or ranges of percentages, relative to the baseline leukocyte measurement.

In some embodiments, the measurement includes a chronic obstructive pulmonary disease (COPD) exacerbation measurement. A COPD exacerbation may include a COPD flare-up such as an acute increase in severity of a respiratory symptom such as difficulty breathing. The COPD exacerbation measurement may include a number of COPD flare-ups, and may be included in a given time frame such as flare-ups per day, week, month, or year. The COPD exacerbation measurement may include a frequency of COPD exacerbations. The COPD exacerbation measurement may include a measurement of worsening of a respiratory symptom, such as increased dyspnea, cough, sputum volume, or sputum purulence. The COPD exacerbation measurement may include a measurement of an event such as when a the subject's conditions change enough to require a change in treatment. The COPD exacerbation measurement may include a peak flow test, a breath nitric oxide measurement, or a blood oxygen level test.

In some embodiments, the composition reduces the COPD exacerbation measurement relative to the baseline COPD exacerbation measurement. In some embodiments, the reduction is measured in a second sample obtained from the subject after administering the composition to the subject. In some embodiments, the reduction is measured directly in the subject after administering the composition to the subject. In some embodiments, the COPD exacerbation measurement is decreased by about 2.5% or more, about 5% or more, or about 7.5% or more, relative to the baseline COPD exacerbation measurement. In some embodiments, the COPD exacerbation measurement is decreased by about 10% or more, relative to the baseline COPD exacerbation measurement. In some embodiments, the COPD exacerbation measurement is decreased by about 20% or more, about 30% or more, about 40% or more, about 50% or more, about 60% or more, about 70% or more, about 80% or more, about 90% or more, relative to the baseline COPD exacerbation measurement. In some embodiments, the COPD exacerbation measurement is decreased by no more than about 2.5%, no more than about 5%, or no more than about 7.5%, relative to the baseline COPD exacerbation measurement. In some embodiments, the COPD exacerbation measurement is decreased by no more than about 10%, relative to the baseline COPD exacerbation measurement. In some embodiments, the COPD exacerbation measurement is decreased by no more than about 20%, no more than about 30%, no more than about 40%, no more than about 50%, no more than about 60%, no more than about 70%, no more than about 80%, no more than about 90%, or no more than about 100% relative to the baseline COPD exacerbation measurement. In some embodiments, the COPD exacerbation measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement includes an asthma exacerbation measurement. An asthma exacerbation may include an asthma attack, for example narrowing of a bronchial tube that causes difficulty breathing. The asthma exacerbation measurement may include a number of number of asthma attacks, and may be included in a given time frame such as flare-ups per day, week, month, or year. The asthma exacerbation measurement may include a frequency of asthma exacerbations. The asthma exacerbation measurement may include a bronchial tube measurement such as a bronchial tube diameter, a bronchial tube circumference, or a bronchial tube area measurement. The asthma exacerbation measurement may include an amount of bronchial tube narrowing, such as a percent constriction. The asthma exacerbation measurement may include a wheezing measurement, a coughing measurement, a chest tightening measurement, a shortness of breath measurement, a agitation measurement, a hyperventilation measurement, a heart rate measurement, a lung function measurement, or a measurement of difficulty speaking or breathing. The asthma exacerbation measurement may include a peak flow test, a breath nitric oxide measurement, or a blood oxygen level test.

In some embodiments, the composition reduces the asthma exacerbation measurement relative to the baseline asthma exacerbation measurement. In some embodiments, the reduction is measured in a second sample obtained from the subject after administering the composition to the subject. In some embodiments, the reduction is measured directly in the subject after administering the composition to the subject. In some embodiments, the asthma exacerbation measurement is decreased by about 2.5% or more, about 5% or more, or about 7.5% or more, relative to the baseline asthma exacerbation measurement. In some embodiments, the asthma exacerbation measurement is decreased by about 10% or more, relative to the baseline asthma exacerbation measurement. In some embodiments, the asthma exacerbation measurement is decreased by about 20% or more, about 30% or more, about 40% or more, about 50% or more, about 60% or more, about 70% or more, about 80% or more, about 90% or more, relative to the baseline asthma exacerbation measurement. In some embodiments, the asthma exacerbation measurement is decreased by no more than about 2.5%, no more than about 5%, or no more than about 7.5%, relative to the baseline asthma exacerbation measurement. In some embodiments, the asthma exacerbation measurement is decreased by no more than about 10%, relative to the baseline asthma exacerbation measurement. In some embodiments, the asthma exacerbation measurement is decreased by no more than about 20%, no more than about 30%, no more than about 40%, no more than about 50%, no more than about 60%, no more than about 70%, no more than about 80%, no more than about 90%, or no more than about 100% relative to the baseline asthma exacerbation measurement. In some embodiments, the asthma exacerbation measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an MST1R protein measurement. In some embodiments, the MST1R protein measurement comprises an MST1R protein level. In some embodiments, the MST1R protein level is indicated as a mass or percentage of MST1R protein per sample weight. In some embodiments, the MST1R protein level is indicated as a mass or percentage of MST1R protein per sample volume. In some embodiments, the MST1R protein level is indicated as a mass or percentage of MST1R protein per total protein within the sample. In some embodiments, the MST1R protein measurement is a lung MST1R protein measurement. In some embodiments, the MST1R protein measurement is obtained by an assay such as an immunoassay, a colorimetric assay, or a fluorescence assay.

In some embodiments, the composition reduces the MST1R protein measurement relative to the baseline MST1R protein measurement. In some embodiments, the composition reduces tissue MST1R protein levels relative to the baseline MST1R protein measurement. In some embodiments, the composition reduces lung MST1R protein levels relative to the baseline MST1R protein measurement. In some embodiments, the reduced MST1R protein levels are measured in a second sample obtained from the subject after administering the composition to the subject. In some embodiments, the second sample is a lung sample. In some embodiments, the MST1R protein measurement is decreased by about 2.5% or more, about 5% or more, or about 7.5% or more, relative to the baseline MST1R protein measurement. In some embodiments, the MST1R protein measurement is decreased by about 10% or more, relative to the baseline MST1R protein measurement. In some embodiments, the MST1R protein measurement is decreased by about 20% or more, about 30% or more, about 40% or more, about 50% or more, about 60% or more, about 70% or more, about 80% or more, about 90% or more, or about 100%, relative to the baseline MST1R protein measurement. In some embodiments, the MST1R protein measurement is decreased by no more than about 2.5%, no more than about 5%, or no more than about 7.5%, relative to the baseline MST1R protein measurement. In some embodiments, the MST1R protein measurement is decreased by no more than about 10%, relative to the baseline MST1R protein measurement. In some embodiments, the MST1R protein measurement is decreased by no more than about 20%, no more than about 30%, no more than about 40%, no more than about 50%, no more than about 60%, no more than about 70%, no more than about 80%, no more than about 90%, or no more than about 100% relative to the baseline MST1R protein measurement. In some embodiments, the MST1R protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is an MST1R mRNA measurement. In some embodiments, the MST1R mRNA measurement comprises an MST1R mRNA level. In some embodiments, the MST1R mRNA level is indicated as an amount or percentage of MST1R mRNA per sample weight. In some embodiments, the MST1R mRNA level is indicated as an amount or percentage of MST1R mRNA per sample volume. In some embodiments, the MST1R mRNA level is indicated as an amount or percentage of MST1R mRNA per total mRNA within the sample. In some embodiments, the MST1R mRNA level is indicated as an amount or percentage of MST1R mRNA per total nucleic acids within the sample. In some embodiments, the MST1R mRNA level is indicated relative to another mRNA level, such as an mRNA level of a housekeeping gene, within the sample. In some embodiments, the MST1R mRNA measurement is obtained by an assay such as a PCR assay. In some embodiments, the PCR comprises qPCR. In some embodiments, the PCR comprises reverse transcription of the MST1R mRNA.

In some embodiments, the composition reduces the MST1R mRNA measurement relative to the baseline MST1R mRNA measurement. In some embodiments, the MST1R mRNA measurement is obtained in a second sample obtained from the subject after administering the composition to the subject. In some embodiments, the composition reduces MST1R mRNA levels relative to the baseline MST1R mRNA levels. In some embodiments, the reduced MST1R mRNA levels are measured in a second sample obtained from the subject after administering the composition to the subject. In some embodiments, the second sample is a lung sample. In some embodiments, the MST1R mRNA measurement is reduced by about 2.5% or more, about 5% or more, or about 7.5% or more, relative to the baseline MST1R mRNA measurement. In some embodiments, the MST1R mRNA measurement is decreased by about 10% or more, relative to the baseline MST1R mRNA measurement. In some embodiments, the MST1R mRNA measurement is decreased by about 20% or more, about 30% or more, about 40% or more, about 50% or more, about 60% or more, about 70% or more, about 80% or more, about 90% or more, or about 100%, relative to the baseline MST1R mRNA measurement. In some embodiments, the MST1R mRNA measurement is decreased by no more than about 2.5%, no more than about 5%, or no more than about 7.5%, relative to the baseline MST1R mRNA measurement. In some embodiments, the MST1R mRNA measurement is decreased by no more than about 10%, relative to the baseline MST1R mRNA measurement. In some embodiments, the MST1R mRNA measurement is decreased by no more than about 20%, no more than about 30%, no more than about 40%, no more than about 50%, no more than about 60%, no more than about 70%, no more than about 80%, no more than about 90%, or no more than about 100%, relative to the baseline MST1R mRNA measurement. In some embodiments, the MST1R mRNA measurement is decreased by 2.5%, 5%, 7.5%, 10%, 20%, 30%, 40%, 50%, 60%, 70%, 80%, 90%, or 100% or by a range defined by any of the two aforementioned percentages.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “Cx-y” or “Cx-Cy” when used in conjunction with a chemical moiety, such as alkyl, alkenyl, or alkynyl is meant to include groups that contain from x to y carbons in the chain. For example, the term “C1-6alkyl” refers to substituted or unsubstituted saturated hydrocarbon groups, including straight-chain alkyl and branched-chain alkyl groups that contain from 1 to 6 carbons.

The terms “Cx-yalkenyl” and “Cx-yalkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2 trifluoroethyl, 1 chloromethyl 2 fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3 benzodioxolyl, benzofuranyl, benzoxazolyl, benzo[d]thiazolyl, benzothiadiazolyl, benzo[b][1,4]dioxepinyl, benzo[b][1,4]oxazinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2 d]pyrimidinyl, benzotriazolyl, benzo[4,6]imidazo[1,2 a]pyridinyl, carbazolyl, cinnolinyl, cyclopenta[d]pyrimidinyl, 6,7 dihydro 5H cyclopenta[4,5]thieno[2,3 d]pyrimidinyl, 5,6 dihydrobenzo[h]quinazolinyl, 5,6 dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2 c]pyridinyl, 5,6,7,8,9,10 hexahydrocycloocta[d]pyrimidinyl, 5,6,7,8,9,10 hexahydrocycloocta[d]pyridazinyl, 5,6,7,8,9,10 hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8 methano 5,6,7,8 tetrahydroquinazolinyl, naphthyridinyl, 1,6 naphthyridinonyl, oxadiazolyl, 2 oxoazepinyl, oxazolyl, oxiranyl, 5,6,6a,7,8,9,10,10a octahydrobenzo[h]quinazolinyl, 1 phenyl 1H pyrrolyl, phenazinyl, phenothiazinyl, phenoxazinyl, phthalazinyl, pteridinyl, purinyl, pyrrolyl, pyrazolyl, pyrazolo[3,4 d]pyrimidinyl, pyridinyl, pyrido[3,2 d]pyrimidinyl, pyrido[3,4 d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8 tetrahydroquinazolinyl, 5,6,7,8 tetrahydrobenzo[4,5]thieno[2,3 d]pyrimidinyl, 6,7,8,9 tetrahydro 5H cyclohepta[4,5]thieno[2,3 d]pyrimidinyl, 5,6,7,8 tetrahydropyrido[4,5 c]pyridazinyl, thiazolyl, thiadiazolyl, triazolyl, tetrazolyl, triazinyl, thieno[2,3 d]pyrimidinyl, thieno[3,2 d]pyrimidinyl, thieno[2,3 c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, 2-oxa-6-azaspiro[3.3]heptane, and 1,1 dioxo thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH2 of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (=S), cyano (—CN), nitro (—NO2), imino (=N—H), oximo (=N—OH), hydrazino (=N—NH2), —Rb ORa, —Rb OC(O) Ra, —Rb OC(O) ORa, —Rb OC(O) N(Ra)2, —Rb N(Ra)2, —Rb C(O)Ra, —Rb C(O)ORa, —Rb C(O)N(Ra)2, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2), and —Rb S(O)tN(Ra)2 (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (=S), cyano (—CN), nitro (—NO2), imino (=N—H), oximo (=N—OH), hydrazine (=N—NH2), —Rb ORa, —Rb OC(O) Ra, —Rb OC(O) ORa, —Rb OC(O) N(Ra)2, —Rb N(Ra)2, —Rb C(O)Ra, —Rb C(O)ORa, —Rb C(O)N(Ra)2, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)₂ (where t is 1 or 2); wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each Ra, valence permitting, may be optionally substituted with alkyl, alkenyl, alkynyl, halogen, haloalkyl, haloalkenyl, haloalkynyl, oxo (═O), thioxo (=S), cyano (—CN), nitro (—NO2), imino (=N—H), oximo (=N—OH), hydrazine (=N—NH2), —Rb ORa, —Rb OC(O) Ra, —Rb OC(O) ORa, —Rb OC(O) N(Ra)2, —Rb N(Ra)2, —Rb C(O)Ra, —Rb C(O)ORa, —Rb C(O)N(Ra)2, —Rb O Rc C(O)N(Ra)2, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)2 (where t is 1 or 2); and wherein each Rb is independently selected from a direct bond or a straight or branched alkylene, alkenylene, or alkynylene chain, and each Rc is a straight or branched alkylene, alkenylene or alkynylene chain.

Double bonds to oxygen atoms, such as oxo groups, are represented herein as both “=O” and “(O)”. Double bonds to nitrogen atoms are represented as both “=NR” and “(NR)”. Double bonds to sulfur atoms are represented as both “=S” and “(S)”.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments. In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may comprise or consist of DNA. For example, an ASO may include DNA.

The section headings used herein are for organizational purposes only and are not to be construed as limiting the subject matter described.

### VI. EXAMPLES

### Example 1: Variants in Genes Encoding the Receptor MST1R and its Ligand MST1 Demonstrate Protective Associations for Obstructive Lung Diseases and Related Traits

Variants in MST1R and MST1 were evaluated for associations with lung diseases and related pulmonary and leukocyte traits in approximately 382,000 individuals with genotype data from the UK Biobank cohort. Variants evaluated included: (1) rs3020779, a common (AAF=0.82) MST1 synonymous variant (Pro153Pro; P153P) which is a MST1R lung↓eQTL (2) rs142690032, a low-frequency (AAF=0.02) MST1 stop-gained variant (Arg651Ter; R651Ter) which prematurely terminates MSP at amino acid 651, and (3) rs3197999, a common (AAF=0.29) MST1 missense variant (Arg703Cys; R703C) which has been experimentally characterized as impairing MSP's ability to bind MST1R. The rs3020779 variant was considered a hypomorphic MST1R variant that may result in a decrease in the abundance and/or activity of the MST1R gene product. The rs142690032 and rs3197999 variants were also considered to result in a decrease in abundance or activity of the MST1 gene product, and thus result in a decrease in signaling through the MST1R gene product.

Analyses used a logistic or linear regression model with age, sex and the first ten principal components of genetic ancestry as covariates. The analyses resulted in identification of associations for the individual MST1R and MST1 variants (Table 1A, 1B, 2A, and 2B). For example, there were protective associations with multiple lung-disease-related traits. The evaluated variants were associated with protection from COPD, asthma and lower risk of inhaled beta agonist prescription (Table 1A and 1B). Additionally, the evaluated variants were associated with increased lung function (FEV1 and FVC) and decreased circulating neutrophil counts (Table 2A and Table 2B).

TABLE 1A
MST1R and MST1 lung disease associations

| | | | | COPD (n = 22,308) | |
|---|---|---|---|---|---|
| Variant | Gene | Function | AAF | P value | OR |
| rs3020779 | MST1R | MST1R eQTL | 0.82 | 4.33E−09 | ↓0.941 |
| rs142690032 | MST1 | Stop-Gained; | 0.02 | 4.07E−07 | ↓0.859 |
| | | R651Ter | | | |
| rs3197999 | MST1 | Missense; | 0.29 | 2.16E−05 | ↓0.962 |
| | | R703C; MST1 | | | |
| | | pQTL | | | |

TABLE 1B
MST1R and MST1 lung disease associations

| | | | Family History | | Inhaled Beta | |
|---|---|---|---|---|---|---|
| | Asthma | | of COPD | | Agonist Medication | |
| | (n = 58,257) | | (n = 60,301) | | (n = 31,028) | |
| Variant | P value | OR | P value | OR | P value | OR |
| rs3020779 | 4.57E−04 | ↓0.979 | 0.004 | ↓0.985 | 5.32E−05 | ↓0.969 |
| rs142690032 | 0.004 | ↓0.955 | 0.105 | ↓0.980 | 4.47E−04 | ↓0.931 |
| rs3197999 | 0.002 | ↓0.983 | 0.001 | ↓0.984 | 2.56E−06 | ↓0.968 |

TABLE 2A
MST1R and MST1 lung function and neutrophil associations

| | | | | FEV1 (n = 287,050) | |
|---|---|---|---|---|---|
| Variant | Gene | Function | AAF | P value | Beta |
| rs3020779 | MST1R | MST1R eQTL | 0.82 | 3.06E−10 | ↑0.002 |
| rs142690032 | MST1 | Stop-Gained; | 0.02 | 0.003 | ↑0.015 |
| | | R651Ter | | | |
| rs3197999 | MST1 | Missense; | 0.29 | 3.29E−15 | ↑0.002 |
| | | R703C; MST1 | | | |
| | | pQTL | | | |

TABLE 2B
MST1R and MST1 lung function and neutrophil associations

| | FVC | | FEV1/FVC Ratio | | Neutrophil Count | |
|---|---|---|---|---|---|---|
| | (n = 286,925) | | (n = 286,523) | | (n = 366,089) | |
| Variant | P value | Beta | P value | Beta | P value | Beta |
| rs3020779 | 2.41E−06 | ↑0.001 | 9.32E−09 | ↑0.019 | 0.01 | ↓−0.010 |
| rs142690032 | 0.002 | ↑0.003 | 0.789 | ↑−0.003 | 0.005 | ↓−0.034 |
| rs3197999 | 1.03E−11 | ↑0.002 | 0.004 | ↑0.008 | 8.2E−08 | ↓−0.018 |

These results indicate that reduced abundance or activity of MST1R, and decreased MST1R signaling via reduced MSP abundance or activity, may protect against COPD or asthma, improve lung function, or lower circulating neutrophils, which may be pro-inflammatory cells involved in obstructive airway disease. These results further indicate that therapeutic inhibition of MST1R may result in similar disease-protective effects.

### Example 2: Bioinformatic Selection of Sequences in Order to Identify Therapeutic siRNAs to Downmodulate Expression of the MST1R mRNA

Screening sets were defined based on bioinformatic analysis. Therapeutic siRNAs were designed to target human MST1R, and the MST1R sequence of at least one toxicology-relevant species, in this case, the non-human primates (NHP) rhesus and cynomolgus monkeys. Drivers for the design of the screening set were predicted specificity of the siRNAs against the transcriptome of the relevant species as well as cross-reactivity between species. Predicted specificity in human, rhesus monkey, cynomolgus monkey, mouse and rat was determined for sense (S) and antisense (AS) strands. These were assigned a “specificity score” which considers the likelihood of unintended downregulation of any other transcript by full or partial complementarity of an siRNA strand (up to 4 mismatches within positions 2-18) as well as the number and positions of mismatches. Thus, off-target(s) for antisense and sense strands of each siRNA were identified. In addition, the number of potential off-targets was used as an additional specificity factor in the specificity score. As identified, siRNAs with high specificity and a low number of predicted off-targets provide a benefit of increased targeting specificity.

In addition to selecting siRNA sequences with high sequence specificity to MST1R mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions were avoided. Seed regions identified in miRNAs from human, mouse, rat, rhesus monkey, dog, rabbit and pig are referred to as “conserved”. Combining the “specificity score” with miRNA seed analysis yielded a “specificity category”. This is divided into categories 1-4, with 1 having the highest specificity and 4 having the lowest specificity. Each strand of the siRNA is assigned to a specificity category.

Specificity and species cross-reactivity was assessed for human, cynomolgus monkey, rhesus monkey, mouse and rat MST1R. The analysis was based on a canonical siRNA design using 19 bases and 17 bases (without considering positions 1 and 19) for cross-reactivity. Full match as well as single mismatch analyses were included.

Analysis of the human Single Nucleotide Polymorphism (SNP) database (NCBI-DB-SNP) to identify siRNAs targeting regions with known SNPs was also carried out to identify siRNAs that may be non-functional in individuals containing the SNP. Information regarding the positions of SNPs within the target sequence as well as minor allele frequency (MAF) in case data was obtained in this analysis.

Initial analysis of the relevant MST1R mRNA sequence revealed few sequences that fulfil the specificity parameters and at the same time target MST1R mRNA in all of the analyzed relevant species. Therefore, it was decided to design independent screening subsets for the therapeutic siRNAs.

The siRNAs in these subsets recognize the human, cynomolgus monkey, rhesus monkey MST1R sequences. Therefore, the siRNAs in these subsets can be used to target human MST1R in a therapeutic setting.

The number of siRNA sequences that can be derived from human MST1R mRNA (ENST00000296474.8, SEQ ID NO: 9818) without consideration of specificity or species cross-reactivity was 4754 (sense and antisense strand sequences included in SEQ ID NOS: 1-9508).

Prioritizing sequences for target specificity, species cross-reactivity, miRNA seed region sequences and SNPs as described above yields subset A. Subset A contains 336 siRNAs whose base sequences are shown in Table 3.

TABLE 3
Sequences in siRNA subset A

| SEQ ID | sense strand sequence | SEQ ID | antisense strand sequence |
|---|---|---|---|
| NO: | (5′-3′) | NO: | (5′-3′) |
| 469 | GCUGUGUUUGUAGCCAUAC | 5223 | GUAUGGCUACAAACACAGC |
| 471 | UGUGUUUGUAGCCAUACGC | 5225 | GCGUAUGGCUACAAACACA |
| 472 | GUGUUUGUAGCCAUACGCA | 5226 | UGCGUAUGGCUACAAACAC |
| 473 | UGUUUGUAGCCAUACGCAA | 5227 | UUGCGUAUGGCUACAAACA |
| 474 | GUUUGUAGCCAUACGCAAU | 5228 | AUUGCGUAUGGCUACAAAC |
| 475 | UUUGUAGCCAUACGCAAUC | 5229 | GAUUGCGUAUGGCUACAAA |
| 476 | UUGUAGCCAUACGCAAUCG | 5230 | CGAUUGCGUAUGGCUACAA |
| 477 | UGUAGCCAUACGCAAUCGC | 5231 | GCGAUUGCGUAUGGCUACA |
| 478 | GUAGCCAUACGCAAUCGCC | 5232 | GGCGAUUGCGUAUGGCUAC |
| 479 | UAGCCAUACGCAAUCGCCU | 5233 | AGGCGAUUGCGUAUGGCUA |
| 480 | AGCCAUACGCAAUCGCCUG | 5234 | CAGGCGAUUGCGUAUGGCU |
| 481 | GCCAUACGCAAUCGCCUGC | 5235 | GCAGGCGAUUGCGUAUGGC |
| 482 | CCAUACGCAAUCGCCUGCA | 5236 | UGCAGGCGAUUGCGUAUGG |
| 483 | CAUACGCAAUCGCCUGCAU | 5237 | AUGCAGGCGAUUGCGUAUG |
| 557 | ACCCUGGCUGCCAGACGUG | 5311 | CACGUCUGGCAGCCAGGGU |
| 562 | GGCUGCCAGACGUGUGCAG | 5316 | CUGCACACGUCUGGCAGCC |
| 564 | CUGCCAGACGUGUGCAGCC | 5318 | GGCUGCACACGUCUGGCAG |
| 568 | CAGACGUGUGCAGCCUGUG | 5322 | CACAGGCUGCACACGUCUG |
| 691 | UGCUUCCUGCAUGACCUAG | 5445 | CUAGGUCAUGCAGGAAGCA |
| 692 | GCUUCCUGCAUGACCUAGA | 5446 | UCUAGGUCAUGCAGGAAGC |
| 693 | CUUCCUGCAUGACCUAGAG | 5447 | CUCUAGGUCAUGCAGGAAG |
| 717 | AGGGACAGCCGUGCAUCUG | 5471 | CAGAUGCACGGCUGUCCCU |
| 718 | GGGACAGCCGUGCAUCUGG | 5472 | CCAGAUGCACGGCUGUCCC |
| 721 | ACAGCCGUGCAUCUGGCAG | 5475 | CUGCCAGAUGCACGGCUGU |
| 722 | CAGCCGUGCAUCUGGCAGC | 5476 | GCUGCCAGAUGCACGGCUG |
| 723 | AGCCGUGCAUCUGGCAGCG | 5477 | CGCUGCCAGAUGCACGGCU |
| 724 | GCCGUGCAUCUGGCAGCGC | 5478 | GCGCUGCCAGAUGCACGGC |
| 725 | CCGUGCAUCUGGCAGCGCC | 5479 | GGCGCUGCCAGAUGCACGG |
| 726 | CGUGCAUCUGGCAGCGCCA | 5480 | UGGCGCUGCCAGAUGCACG |
| 753 | CUUCUCAGCCCACCAUAAC | 5507 | GUUAUGGUGGGCUGAGAAG |
| 759 | AGCCCACCAUAACCGGCCC | 5513 | GGGCCGGUUAUGGUGGGCU |
| 761 | CCCACCAUAACCGGCCCGA | 5515 | UCGGGCCGGUUAUGGUGGG |
| 762 | CCACCAUAACCGGCCCGAU | 5516 | AUCGGGCCGGUUAUGGUGG |
| 763 | CACCAUAACCGGCCCGAUG | 5517 | CAUCGGGCCGGUUAUGGUG |
| 764 | ACCAUAACCGGCCCGAUGA | 5518 | UCAUCGGGCCGGUUAUGGU |
| 765 | CCAUAACCGGCCCGAUGAC | 5519 | GUCAUCGGGCCGGUUAUGG |
| 766 | CAUAACCGGCCCGAUGACU | 5520 | AGUCAUCGGGCCGGUUAUG |
| 767 | AUAACCGGCCCGAUGACUG | 5521 | CAGUCAUCGGGCCGGUUAU |
| 775 | CCCGAUGACUGCCCCGACU | 5529 | AGUCGGGGCAGUCAUCGGG |
| 776 | CCGAUGACUGCCCCGACUG | 5530 | CAGUCGGGGCAGUCAUCGG |
| 777 | CGAUGACUGCCCCGACUGU | 5531 | ACAGUCGGGGCAGUCAUCG |
| 801 | CAGCCCAUUGGGCACCCGU | 5555 | ACGGGUGCCCAAUGGGCUG |
| 804 | CCCAUUGGGCACCCGUGUA | 5558 | UACACGGGUGCCCAAUGGG |
| 824 | CUGUGGUUGAGCAAGGCCA | 5578 | UGGCCUUGCUCAACCACAG |
| 826 | GUGGUUGAGCAAGGCCAGG | 5580 | CCUGGCCUUGCUCAACCAC |
| 837 | AGGCCAGGCCUCCUAUUUC | 5591 | GAAAUAGGAGGCCUGGCCU |
| 842 | AGGCCUCCUAUUUCUACGU | 5596 | ACGUAGAAAUAGGAGGCCU |
| 845 | CCUCCUAUUUCUACGUGGC | 5599 | GCCACGUAGAAAUAGGAGG |
| 854 | UCUACGUGGCAUCCUCACU | 5608 | AGUGAGGAUGCCACGUAGA |
| 855 | CUACGUGGCAUCCUCACUG | 5609 | CAGUGAGGAUGCCACGUAG |
| 863 | CAUCCUCACUGGACGCAGC | 5617 | GCUGCGUCCAGUGAGGAUG |
| 864 | AUCCUCACUGGACGCAGCC | 5618 | GGCUGCGUCCAGUGAGGAU |
| 866 | CCUCACUGGACGCAGCCGU | 5620 | ACGGCUGCGUCCAGUGAGG |
| 888 | UGCCAGCUUCAGCCCACGC | 5642 | GCGUGGGCUGAAGCUGGCA |
| 895 | UUCAGCCCACGCUCAGUGU | 5649 | ACACUGAGCGUGGGCUGAA |
| 897 | CAGCCCACGCUCAGUGUCU | 5651 | AGACACUGAGCGUGGGCUG |
| 898 | AGCCCACGCUCAGUGUCUA | 5652 | UAGACACUGAGCGUGGGCU |
| 899 | GCCCACGCUCAGUGUCUAU | 5653 | AUAGACACUGAGCGUGGGC |
| 900 | CCCACGCUCAGUGUCUAUC | 5654 | GAUAGACACUGAGCGUGGG |
| 901 | CCACGCUCAGUGUCUAUCA | 5655 | UGAUAGACACUGAGCGUGG |
| 903 | ACGCUCAGUGUCUAUCAGG | 5657 | CCUGAUAGACACUGAGCGU |
| 904 | CGCUCAGUGUCUAUCAGGC | 5658 | GCCUGAUAGACACUGAGCG |
| 907 | UCAGUGUCUAUCAGGCGUC | 5661 | GACGCCUGAUAGACACUGA |
| 908 | CAGUGUCUAUCAGGCGUCU | 5662 | AGACGCCUGAUAGACACUG |
| 909 | AGUGUCUAUCAGGCGUCUC | 5663 | GAGACGCCUGAUAGACACU |
| 910 | GUGUCUAUCAGGCGUCUCA | 5664 | UGAGACGCCUGAUAGACAC |
| 911 | UGUCUAUCAGGCGUCUCAA | 5665 | UUGAGACGCCUGAUAGACA |
| 912 | GUCUAUCAGGCGUCUCAAG | 5666 | CUUGAGACGCCUGAUAGAC |
| 913 | UCUAUCAGGCGUCUCAAGG | 5667 | CCUUGAGACGCCUGAUAGA |
| 915 | UAUCAGGCGUCUCAAGGCU | 5669 | AGCCUUGAGACGCCUGAUA |
| 948 | CGCACCGGGCUUUGUGGCG | 5702 | CGCCACAAAGCCCGGUGCG |
| 966 | GUUGUCAGUGCUGCCCAAG | 5720 | CUUGGGCAGCACUGACAAC |
| 976 | CUGCCCAAGCAUCUUGUCU | 5730 | AGACAAGAUGCUUGGGCAG |
| 977 | UGCCCAAGCAUCUUGUCUC | 5731 | GAGACAAGAUGCUUGGGCA |
| 985 | CAUCUUGUCUCCUACAGUA | 5739 | UACUGUAGGAGACAAGAUG |
| 986 | AUCUUGUCUCCUACAGUAU | 5740 | AUACUGUAGGAGACAAGAU |
| 989 | UUGUCUCCUACAGUAUUGA | 5743 | UCAAUACUGUAGGAGACAA |
| 990 | UGUCUCCUACAGUAUUGAA | 5744 | UUCAAUACUGUAGGAGACA |
| 992 | UCUCCUACAGUAUUGAAUA | 5746 | UAUUCAAUACUGUAGGAGA |
| 1001 | GUAUUGAAUACGUGCACAG | 5755 | CUGUGCACGUAUUCAAUAC |
| 1003 | AUUGAAUACGUGCACAGCU | 5757 | AGCUGUGCACGUAUUCAAU |
| 1004 | UUGAAUACGUGCACAGCUU | 5758 | AAGCUGUGCACGUAUUCAA |
| 1005 | UGAAUACGUGCACAGCUUC | 5759 | GAAGCUGUGCACGUAUUCA |
| 1006 | GAAUACGUGCACAGCUUCC | 5760 | GGAAGCUGUGCACGUAUUC |
| 1042 | UACUUCCUGACUGUACAGC | 5796 | GCUGUACAGUCAGGAAGUA |
| 1043 | ACUUCCUGACUGUACAGCC | 5797 | GGCUGUACAGUCAGGAAGU |
| 1046 | UCCUGACUGUACAGCCGGC | 5800 | GCCGGCUGUACAGUCAGGA |
| 1048 | CUGACUGUACAGCCGGCCA | 5802 | UGGCCGGCUGUACAGUCAG |
| 1049 | UGACUGUACAGCCGGCCAG | 5803 | CUGGCCGGCUGUACAGUCA |
| 1050 | GACUGUACAGCCGGCCAGC | 5804 | GCUGGCCGGCUGUACAGUC |
| 1051 | ACUGUACAGCCGGCCAGCG | 5805 | CGCUGGCCGGCUGUACAGU |
| 1053 | UGUACAGCCGGCCAGCGUG | 5807 | CACGCUGGCCGGCUGUACA |
| 1054 | GUACAGCCGGCCAGCGUGA | 5808 | UCACGCUGGCCGGCUGUAC |
| 1055 | UACAGCCGGCCAGCGUGAC | 5809 | GUCACGCUGGCCGGCUGUA |
| 1111 | CUUAGCGCCACUGAGCCAG | 5865 | CUGGCUCAGUGGCGCUAAG |
| 1121 | CUGAGCCAGAGUUGGGUGA | 5875 | UCACCCAACUCUGGCUCAG |
| 1124 | AGCCAGAGUUGGGUGACUA | 5878 | UAGUCACCCAACUCUGGCU |
| 1129 | GAGUUGGGUGACUAUCGGG | 5883 | CCCGAUAGUCACCCAACUC |
| 1130 | AGUUGGGUGACUAUCGGGA | 5884 | UCCCGAUAGUCACCCAACU |
| 1131 | GUUGGGUGACUAUCGGGAG | 5885 | CUCCCGAUAGUCACCCAAC |
| 1132 | UUGGGUGACUAUCGGGAGC | 5886 | GCUCCCGAUAGUCACCCAA |
| 1134 | GGGUGACUAUCGGGAGCUG | 5888 | CAGCUCCCGAUAGUCACCC |
| 1137 | UGACUAUCGGGAGCUGGUC | 5891 | GACCAGCUCCCGAUAGUCA |
| 1138 | GACUAUCGGGAGCUGGUCC | 5892 | GGACCAGCUCCCGAUAGUC |
| 1139 | ACUAUCGGGAGCUGGUCCU | 5893 | AGGACCAGCUCCCGAUAGU |
| 1145 | GGGAGCUGGUCCUCGACUG | 5899 | CAGUCGAGGACCAGCUCCC |
| 1146 | GGAGCUGGUCCUCGACUGC | 5900 | GCAGUCGAGGACCAGCUCC |
| 1147 | GAGCUGGUCCUCGACUGCA | 5901 | UGCAGUCGAGGACCAGCUC |
| 1152 | GGUCCUCGACUGCAGAUUU | 5906 | AAAUCUGCAGUCGAGGACC |
| 1153 | GUCCUCGACUGCAGAUUUG | 5907 | CAAAUCUGCAGUCGAGGAC |
| 1154 | UCCUCGACUGCAGAUUUGC | 5908 | GCAAAUCUGCAGUCGAGGA |
| 1155 | CCUCGACUGCAGAUUUGCU | 5909 | AGCAAAUCUGCAGUCGAGG |
| 1156 | CUCGACUGCAGAUUUGCUC | 5910 | GAGCAAAUCUGCAGUCGAG |
| 1157 | UCGACUGCAGAUUUGCUCC | 5911 | GGAGCAAAUCUGCAGUCGA |
| 1158 | CGACUGCAGAUUUGCUCCA | 5912 | UGGAGCAAAUCUGCAGUCG |
| 1161 | CUGCAGAUUUGCUCCAAAA | 5915 | UUUUGGAGCAAAUCUGCAG |
| 1162 | UGCAGAUUUGCUCCAAAAC | 5916 | GUUUUGGAGCAAAUCUGCA |
| 1164 | CAGAUUUGCUCCAAAACGC | 5918 | GCGUUUUGGAGCAAAUCUG |
| 1165 | AGAUUUGCUCCAAAACGCA | 5919 | UGCGUUUUGGAGCAAAUCU |
| 1166 | GAUUUGCUCCAAAACGCAG | 5920 | CUGCGUUUUGGAGCAAAUC |
| 1170 | UGCUCCAAAACGCAGGCGC | 5924 | GCGCCUGCGUUUUGGAGCA |
| 1171 | GCUCCAAAACGCAGGCGCC | 5925 | GGCGCCUGCGUUUUGGAGC |
| 1172 | CUCCAAAACGCAGGCGCCG | 5926 | CGGCGCCUGCGUUUUGGAG |
| 1173 | UCCAAAACGCAGGCGCCGG | 5927 | CCGGCGCCUGCGUUUUGGA |
| 1204 | GGCGGACAGCCCUACCCUG | 5958 | CAGGGUAGGGCUGUCCGCC |
| 1350 | CAACUCUGUCGUCUGUGCC | 6104 | GGCACAGACGACAGAGUUG |
| 1356 | UGUCGUCUGUGCCUUCCCC | 6110 | GGGGAAGGCACAGACGACA |
| 1405 | GGUGUGGAGCGCUGUUGUG | 6159 | CACAACAGCGCUCCACACC |
| 1406 | GUGUGGAGCGCUGUUGUGA | 6160 | UCACAACAGCGCUCCACAC |
| 1408 | GUGGAGCGCUGUUGUGAAU | 6162 | AUUCACAACAGCGCUCCAC |
| 1409 | UGGAGCGCUGUUGUGAAUC | 6163 | GAUUCACAACAGCGCUCCA |
| 1411 | GAGCGCUGUUGUGAAUCCC | 6165 | GGGAUUCACAACAGCGCUC |
| 1421 | GUGAAUCCCCAGUCCAUCC | 6175 | GGAUGGACUGGGGAUUCAC |
| 1443 | CCUCCGGCGAGGCCUCGAC | 6197 | GUCGAGGCCUCGCCGGAGG |
| 1444 | CUCCGGCGAGGCCUCGACU | 6198 | AGUCGAGGCCUCGCCGGAG |
| 1445 | UCCGGCGAGGCCUCGACUU | 6199 | AAGUCGAGGCCUCGCCGGA |
| 1446 | CCGGCGAGGCCUCGACUUC | 6200 | GAAGUCGAGGCCUCGCCGG |
| 1447 | CGGCGAGGCCUCGACUUCU | 6201 | AGAAGUCGAGGCCUCGCCG |
| 1476 | CAGUUUUUGCCCCAACCCG | 6230 | CGGGUUGGGGCAAAAACUG |
| 1857 | GACCUGUGGGCGUUGCCUA | 6611 | UAGGCAACGCCCACAGGUC |
| 1858 | ACCUGUGGGCGUUGCCUAA | 6612 | UUAGGCAACGCCCACAGGU |
| 1862 | GUGGGCGUUGCCUAAGGGC | 6616 | GCCCUUAGGCAACGCCCAC |
| 1863 | UGGGCGUUGCCUAAGGGCA | 6617 | UGCCCUUAGGCAACGCCCA |
| 1969 | CCUAAGCUUACUGAGUUCC | 6723 | GGAACUCAGUAAGCUUAGG |
| 2124 | CAAGGACAGCUCAAAACUC | 6878 | GAGUUUUGAGCUGUCCUUG |
| 2125 | AAGGACAGCUCAAAACUCA | 6879 | UGAGUUUUGAGCUGUCCUU |
| 2142 | CAGACCAGUGCCCCGGAAA | 6896 | UUUCCGGGGCACUGGUCUG |
| 2143 | AGACCAGUGCCCCGGAAAG | 6897 | CUUUCCGGGGCACUGGUCU |
| 2144 | GACCAGUGCCCCGGAAAGA | 6898 | UCUUUCCGGGGCACUGGUC |
| 2148 | AGUGCCCCGGAAAGACUUU | 6902 | AAAGUCUUUCCGGGGCACU |
| 2149 | GUGCCCCGGAAAGACUUUG | 6903 | CAAAGUCUUUCCGGGGCAC |
| 2150 | UGCCCCGGAAAGACUUUGU | 6904 | ACAAAGUCUUUCCGGGGCA |
| 2151 | GCCCCGGAAAGACUUUGUA | 6905 | UACAAAGUCUUUCCGGGGC |
| 2152 | CCCCGGAAAGACUUUGUAG | 6906 | CUACAAAGUCUUUCCGGGG |
| 2153 | CCCGGAAAGACUUUGUAGA | 6907 | UCUACAAAGUCUUUCCGGG |
| 2154 | CCGGAAAGACUUUGUAGAG | 6908 | CUCUACAAAGUCUUUCCGG |
| 2165 | UUGUAGAGGAGUUUGAGUG | 6919 | CACUCAAACUCCUCUACAA |
| 2180 | AGUGUGAACUGGAGCCCUU | 6934 | AAGGGCUCCAGUUCACACU |
| 2208 | GGCAGUGGGGCCUACCAAC | 6962 | GUUGGUAGGCCCCACUGCC |
| 2226 | CGUCAGCCUCACCGUGACU | 6980 | AGUCACGGUGAGGCUGACG |
| 2228 | UCAGCCUCACCGUGACUAA | 6982 | UUAGUCACGGUGAGGCUGA |
| 2229 | CAGCCUCACCGUGACUAAC | 6983 | GUUAGUCACGGUGAGGCUG |
| 2230 | AGCCUCACCGUGACUAACA | 6984 | UGUUAGUCACGGUGAGGCU |
| 2232 | CCUCACCGUGACUAACAUG | 6986 | CAUGUUAGUCACGGUGAGG |
| 2233 | CUCACCGUGACUAACAUGC | 6987 | GCAUGUUAGUCACGGUGAG |
| 2234 | UCACCGUGACUAACAUGCC | 6988 | GGCAUGUUAGUCACGGUGA |
| 2235 | CACCGUGACUAACAUGCCA | 6989 | UGGCAUGUUAGUCACGGUG |
| 2237 | CCGUGACUAACAUGCCACC | 6991 | GGUGGCAUGUUAGUCACGG |
| 2240 | UGACUAACAUGCCACCGGG | 6994 | CCCGGUGGCAUGUUAGUCA |
| 2241 | GACUAACAUGCCACCGGGC | 6995 | GCCCGGUGGCAUGUUAGUC |
| 2242 | ACUAACAUGCCACCGGGCA | 6996 | UGCCCGGUGGCAUGUUAGU |
| 2243 | CUAACAUGCCACCGGGCAA | 6997 | UUGCCCGGUGGCAUGUUAG |
| 2244 | UAACAUGCCACCGGGCAAG | 6998 | CUUGCCCGGUGGCAUGUUA |
| 2245 | AACAUGCCACCGGGCAAGC | 6999 | GCUUGCCCGGUGGCAUGUU |
| 2246 | ACAUGCCACCGGGCAAGCA | 7000 | UGCUUGCCCGGUGGCAUGU |
| 2252 | CACCGGGCAAGCACUUCCG | 7006 | CGGAAGUGCUUGCCCGGUG |
| 2253 | ACCGGGCAAGCACUUCCGG | 7007 | CCGGAAGUGCUUGCCCGGU |
| 2257 | GGCAAGCACUUCCGGGUAG | 7011 | CUACCCGGAAGUGCUUGCC |
| 2258 | GCAAGCACUUCCGGGUAGA | 7012 | UCUACCCGGAAGUGCUUGC |
| 2259 | CAAGCACUUCCGGGUAGAC | 7013 | GUCUACCCGGAAGUGCUUG |
| 2263 | CACUUCCGGGUAGACGGCA | 7017 | UGCCGUCUACCCGGAAGUG |
| 2264 | ACUUCCGGGUAGACGGCAC | 7018 | GUGCCGUCUACCCGGAAGU |
| 2265 | CUUCCGGGUAGACGGCACC | 7019 | GGUGCCGUCUACCCGGAAG |
| 2266 | UUCCGGGUAGACGGCACCU | 7020 | AGGUGCCGUCUACCCGGAA |
| 2267 | UCCGGGUAGACGGCACCUC | 7021 | GAGGUGCCGUCUACCCGGA |
| 2269 | CGGGUAGACGGCACCUCCG | 7023 | CGGAGGUGCCGUCUACCCG |
| 2318 | UGCUGAUAGCAGUGCAACC | 7072 | GGUUGCACUGCUAUCAGCA |
| 2319 | GCUGAUAGCAGUGCAACCC | 7073 | GGGUUGCACUGCUAUCAGC |
| 2320 | CUGAUAGCAGUGCAACCCC | 7074 | GGGGUUGCACUGCUAUCAG |
| 2322 | GAUAGCAGUGCAACCCCUC | 7076 | GAGGGGUUGCACUGCUAUC |
| 2332 | CAACCCCUCUUUGGCCCAC | 7086 | GUGGGCCAAAGAGGGGUUG |
| 2366 | GUCUCACUCUUGAAGGCCA | 7120 | UGGCCUUCAAGAGUGAGAC |
| 2369 | UCACUCUUGAAGGCCAGAG | 7123 | CUCUGGCCUUCAAGAGUGA |
| 2371 | ACUCUUGAAGGCCAGAGUC | 7125 | GACUCUGGCCUUCAAGAGU |
| 2386 | AGUCUGUCUGUAGGCACCA | 7140 | UGGUGCCUACAGACAGACU |
| 2412 | UGUGCUGGUCAAUGGGACU | 7166 | AGUCCCAUUGACCAGCACA |
| 2413 | GUGCUGGUCAAUGGGACUG | 7167 | CAGUCCCAUUGACCAGCAC |
| 2418 | GGUCAAUGGGACUGAGUGU | 7172 | ACACUCAGUCCCAUUGACC |
| 2420 | UCAAUGGGACUGAGUGUCU | 7174 | AGACACUCAGUCCCAUUGA |
| 2421 | CAAUGGGACUGAGUGUCUG | 7175 | CAGACACUCAGUCCCAUUG |
| 2422 | AAUGGGACUGAGUGUCUGC | 7176 | GCAGACACUCAGUCCCAUU |
| 2425 | GGGACUGAGUGUCUGCUAG | 7179 | CUAGCAGACACUCAGUCCC |
| 2427 | GACUGAGUGUCUGCUAGCA | 7181 | UGCUAGCAGACACUCAGUC |
| 2430 | UGAGUGUCUGCUAGCACGG | 7184 | CCGUGCUAGCAGACACUCA |
| 2432 | AGUGUCUGCUAGCACGGGU | 7186 | ACCCGUGCUAGCAGACACU |
| 2435 | GUCUGCUAGCACGGGUCAG | 7189 | CUGACCCGUGCUAGCAGAC |
| 2437 | CUGCUAGCACGGGUCAGUG | 7191 | CACUGACCCGUGCUAGCAG |
| 2438 | UGCUAGCACGGGUCAGUGA | 7192 | UCACUGACCCGUGCUAGCA |
| 2440 | CUAGCACGGGUCAGUGAGG | 7194 | CCUCACUGACCCGUGCUAG |
| 2443 | GCACGGGUCAGUGAGGGGC | 7197 | GCCCCUCACUGACCCGUGC |
| 2445 | ACGGGUCAGUGAGGGGCAG | 7199 | CUGCCCCUCACUGACCCGU |
| 2455 | GAGGGGCAGCUUUUAUGUG | 7209 | CACAUAAAAGCUGCCCCUC |
| 2460 | GCAGCUUUUAUGUGCCACA | 7214 | UGUGGCACAUAAAAGCUGC |
| 2462 | AGCUUUUAUGUGCCACACC | 7216 | GGUGUGGCACAUAAAAGCU |
| 2464 | CUUUUAUGUGCCACACCCC | 7218 | GGGGUGUGGCACAUAAAAG |
| 2566 | GAAGACCCUGUCGUGCUAA | 7320 | UUAGCACGACAGGGUCUUC |
| 2567 | AAGACCCUGUCGUGCUAAG | 7321 | CUUAGCACGACAGGGUCUU |
| 2571 | CCCUGUCGUGCUAAGCAUC | 7325 | GAUGCUUAGCACGACAGGG |
| 2572 | CCUGUCGUGCUAAGCAUCA | 7326 | UGAUGCUUAGCACGACAGG |
| 2575 | GUCGUGCUAAGCAUCAGCC | 7329 | GGCUGAUGCUUAGCACGAC |
| 2577 | CGUGCUAAGCAUCAGCCCC | 7331 | GGGGCUGAUGCUUAGCACG |
| 2581 | CUAAGCAUCAGCCCCAACU | 7335 | AGUUGGGGCUGAUGCUUAG |
| 2582 | UAAGCAUCAGCCCCAACUG | 7336 | CAGUUGGGGCUGAUGCUUA |
| 2584 | AGCAUCAGCCCCAACUGUG | 7338 | CACAGUUGGGGCUGAUGCU |
| 2628 | CUGUGGCCAGCAUCUAACU | 7382 | AGUUAGAUGCUGGCCACAG |
| 2629 | UGUGGCCAGCAUCUAACUU | 7383 | AAGUUAGAUGCUGGCCACA |
| 2644 | ACUUCAGCAUGGCACUUAG | 7398 | CUAAGUGCCAUGCUGAAGU |
| 2646 | UUCAGCAUGGCACUUAGUG | 7400 | CACUAAGUGCCAUGCUGAA |
| 2648 | CAGCAUGGCACUUAGUGCU | 7402 | AGCACUAAGUGCCAUGCUG |
| 2652 | AUGGCACUUAGUGCUGUCA | 7406 | UGACAGCACUAAGUGCCAU |
| 2655 | GCACUUAGUGCUGUCAUUC | 7409 | GAAUGACAGCACUAAGUGC |
| 2656 | CACUUAGUGCUGUCAUUCC | 7410 | GGAAUGACAGCACUAAGUG |
| 2661 | AGUGCUGUCAUUCCAUGAC | 7415 | GUCAUGGAAUGACAGCACU |
| 2663 | UGCUGUCAUUCCAUGACGG | 7417 | CCGUCAUGGAAUGACAGCA |
| 2664 | GCUGUCAUUCCAUGACGGG | 7418 | CCCGUCAUGGAAUGACAGC |
| 2666 | UGUCAUUCCAUGACGGGCU | 7420 | AGCCCGUCAUGGAAUGACA |
| 2668 | UCAUUCCAUGACGGGCUUA | 7422 | UAAGCCCGUCAUGGAAUGA |
| 2669 | CAUUCCAUGACGGGCUUAG | 7423 | CUAAGCCCGUCAUGGAAUG |
| 2670 | AUUCCAUGACGGGCUUAGG | 7424 | CCUAAGCCCGUCAUGGAAU |
| 2671 | UUCCAUGACGGGCUUAGGG | 7425 | CCCUAAGCCCGUCAUGGAA |
| 2673 | CCAUGACGGGCUUAGGGCA | 7427 | UGCCCUAAGCCCGUCAUGG |
| 2676 | UGACGGGCUUAGGGCAGUG | 7430 | CACUGCCCUAAGCCCGUCA |
| 2678 | ACGGGCUUAGGGCAGUGGA | 7432 | UCCACUGCCCUAAGCCCGU |
| 2808 | AGCUGCUGGCUUUACACUG | 7562 | CAGUGUAAAGCCAGCAGCU |
| 2816 | GCUUUACACUGCCUGGCUU | 7570 | AAGCCAGGCAGUGUAAAGC |
| 2887 | CCUGAGGAGCAUGCCAUUA | 7641 | UAAUGGCAUGCUCCUCAGG |
| 2889 | UGAGGAGCAUGCCAUUAAG | 7643 | CUUAAUGGCAUGCUCCUCA |
| 2891 | AGGAGCAUGCCAUUAAGUU | 7645 | AACUUAAUGGCAUGCUCCU |
| 2895 | GCAUGCCAUUAAGUUUGAG | 7649 | CUCAAACUUAAUGGCAUGC |
| 3084 | UAUCCUGGGUAGAGUGGUG | 7838 | CACCACUCUACCCAGGAUA |
| 3186 | UGCACUGGUCUUCAGCUAC | 7940 | GUAGCUGAAGACCAGUGCA |
| 3191 | UGGUCUUCAGCUACUGGUG | 7945 | CACCAGUAGCUGAAGACCA |
| 3192 | GGUCUUCAGCUACUGGUGG | 7946 | CCACCAGUAGCUGAAGACC |
| 3223 | CUAGUUCUUCCUCCCAACC | 7977 | GGUUGGGAGGAAGAACUAG |
| 3279 | CACACCCCUGCCUAUUCUG | 8033 | CAGAAUAGGCAGGGGUGUG |
| 3297 | GUACUCGGGCUCUGACUAC | 8051 | GUAGUCAGAGCCCGAGUAC |
| 3298 | UACUCGGGCUCUGACUACA | 8052 | UGUAGUCAGAGCCCGAGUA |
| 3299 | ACUCGGGCUCUGACUACAG | 8053 | CUGUAGUCAGAGCCCGAGU |
| 3300 | CUCGGGCUCUGACUACAGA | 8054 | UCUGUAGUCAGAGCCCGAG |
| 3301 | UCGGGCUCUGACUACAGAA | 8055 | UUCUGUAGUCAGAGCCCGA |
| 3537 | CCACUUUGGAGUUGUCUAC | 8291 | GUAGACAACUCCAAAGUGG |
| 3538 | CACUUUGGAGUUGUCUACC | 8292 | GGUAGACAACUCCAAAGUG |
| 3560 | GAGAAUACAUAGACCAGGC | 8314 | GCCUGGUCUAUGUAUUCUC |
| 3563 | AAUACAUAGACCAGGCCCA | 8317 | UGGGCCUGGUCUAUGUAUU |
| 3564 | AUACAUAGACCAGGCCCAG | 8318 | CUGGGCCUGGUCUAUGUAU |
| 3568 | AUAGACCAGGCCCAGAAUC | 8322 | GAUUCUGGGCCUGGUCUAU |
| 3570 | AGACCAGGCCCAGAAUCGA | 8324 | UCGAUUCUGGGCCUGGUCU |
| 3574 | CAGGCCCAGAAUCGAAUCC | 8328 | GGAUUCGAUUCUGGGCCUG |
| 3576 | GGCCCAGAAUCGAAUCCAA | 8330 | UUGGAUUCGAUUCUGGGCC |
| 3577 | GCCCAGAAUCGAAUCCAAU | 8331 | AUUGGAUUCGAUUCUGGGC |
| 3578 | CCCAGAAUCGAAUCCAAUG | 8332 | CAUUGGAUUCGAUUCUGGG |
| 3580 | CAGAAUCGAAUCCAAUGUG | 8334 | CACAUUGGAUUCGAUUCUG |
| 3581 | AGAAUCGAAUCCAAUGUGC | 8335 | GCACAUUGGAUUCGAUUCU |
| 3582 | GAAUCGAAUCCAAUGUGCC | 8336 | GGCACAUUGGAUUCGAUUC |
| 3583 | AAUCGAAUCCAAUGUGCCA | 8337 | UGGCACAUUGGAUUCGAUU |
| 3584 | AUCGAAUCCAAUGUGCCAU | 8338 | AUGGCACAUUGGAUUCGAU |
| 3586 | CGAAUCCAAUGUGCCAUCA | 8340 | UGAUGGCACAUUGGAUUCG |
| 3587 | GAAUCCAAUGUGCCAUCAA | 8341 | UUGAUGGCACAUUGGAUUC |
| 3588 | AAUCCAAUGUGCCAUCAAG | 8342 | CUUGAUGGCACAUUGGAUU |
| 3592 | CAAUGUGCCAUCAAGUCAC | 8346 | GUGACUUGAUGGCACAUUG |
| 3594 | AUGUGCCAUCAAGUCACUA | 8348 | UAGUGACUUGAUGGCACAU |
| 3595 | UGUGCCAUCAAGUCACUAA | 8349 | UUAGUGACUUGAUGGCACA |
| 3596 | GUGCCAUCAAGUCACUAAG | 8350 | CUUAGUGACUUGAUGGCAC |
| 3597 | UGCCAUCAAGUCACUAAGU | 8351 | ACUUAGUGACUUGAUGGCA |
| 3600 | CAUCAAGUCACUAAGUCGC | 8354 | GCGACUUAGUGACUUGAUG |
| 3655 | GAGGGGCUGCUCAUGCGUG | 8409 | CACGCAUGAGCAGCCCCUC |
| 3656 | AGGGGCUGCUCAUGCGUGG | 8410 | CCACGCAUGAGCAGCCCCU |
| 3657 | GGGGCUGCUCAUGCGUGGC | 8411 | GCCACGCAUGAGCAGCCCC |
| 3659 | GGCUGCUCAUGCGUGGCCU | 8413 | AGGCCACGCAUGAGCAGCC |
| 3663 | GCUCAUGCGUGGCCUGAAC | 8417 | GUUCAGGCCACGCAUGAGC |
| 3666 | CAUGCGUGGCCUGAACCAC | 8420 | GUGGUUCAGGCCACGCAUG |
| 3670 | CGUGGCCUGAACCACCCGA | 8424 | UCGGGUGGUUCAGGCCACG |
| 3671 | GUGGCCUGAACCACCCGAA | 8425 | UUCGGGUGGUUCAGGCCAC |
| 3672 | UGGCCUGAACCACCCGAAU | 8426 | AUUCGGGUGGUUCAGGCCA |
| 3673 | GGCCUGAACCACCCGAAUG | 8427 | CAUUCGGGUGGUUCAGGCC |
| 3674 | GCCUGAACCACCCGAAUGU | 8428 | ACAUUCGGGUGGUUCAGGC |
| 3676 | CUGAACCACCCGAAUGUGC | 8430 | GCACAUUCGGGUGGUUCAG |
| 3685 | CCGAAUGUGCUGGCUCUCA | 8439 | UGAGAGCCAGCACAUUCGG |
| 3701 | UCAUUGGUAUCAUGUUGCC | 8455 | GGCAACAUGAUACCAAUGA |
| 3702 | CAUUGGUAUCAUGUUGCCA | 8456 | UGGCAACAUGAUACCAAUG |
| 3705 | UGGUAUCAUGUUGCCACCU | 8459 | AGGUGGCAACAUGAUACCA |
| 3742 | CUGCUGCCCUAUAUGUGCC | 8496 | GGCACAUAUAGGGCAGCAG |
| 3746 | UGCCCUAUAUGUGCCACGG | 8500 | CCGUGGCACAUAUAGGGCA |
| 3748 | CCCUAUAUGUGCCACGGUG | 8502 | CACCGUGGCACAUAUAGGG |
| 3749 | CCUAUAUGUGCCACGGUGA | 8503 | UCACCGUGGCACAUAUAGG |
| 3750 | CUAUAUGUGCCACGGUGAC | 8504 | GUCACCGUGGCACAUAUAG |
| 3751 | UAUAUGUGCCACGGUGACC | 8505 | GGUCACCGUGGCACAUAUA |
| 3752 | AUAUGUGCCACGGUGACCU | 8506 | AGGUCACCGUGGCACAUAU |
| 3755 | UGUGCCACGGUGACCUGCU | 8509 | AGCAGGUCACCGUGGCACA |
| 3756 | GUGCCACGGUGACCUGCUC | 8510 | GAGCAGGUCACCGUGGCAC |
| 3762 | CGGUGACCUGCUCCAGUUC | 8516 | GAACUGGAGCAGGUCACCG |
| 3770 | UGCUCCAGUUCAUCCGCUC | 8524 | GAGCGGAUGAACUGGAGCA |
| 3771 | GCUCCAGUUCAUCCGCUCA | 8525 | UGAGCGGAUGAACUGGAGC |
| 3773 | UCCAGUUCAUCCGCUCACC | 8527 | GGUGAGCGGAUGAACUGGA |
| 3775 | CAGUUCAUCCGCUCACCUC | 8529 | GAGGUGAGCGGAUGAACUG |
| 3777 | GUUCAUCCGCUCACCUCAG | 8531 | CUGAGGUGAGCGGAUGAAC |
| 3781 | AUCCGCUCACCUCAGCGGA | 8535 | UCCGCUGAGGUGAGCGGAU |
| 3783 | CCGCUCACCUCAGCGGAAC | 8537 | GUUCCGCUGAGGUGAGCGG |
| 3788 | CACCUCAGCGGAACCCCAC | 8542 | GUGGGGUUCCGCUGAGGUG |
| 3791 | CUCAGCGGAACCCCACCGU | 8545 | ACGGUGGGGUUCCGCUGAG |
| 3793 | CAGCGGAACCCCACCGUGA | 8547 | UCACGGUGGGGUUCCGCUG |
| 3794 | AGCGGAACCCCACCGUGAA | 8548 | UUCACGGUGGGGUUCCGCU |
| 3795 | GCGGAACCCCACCGUGAAG | 8549 | CUUCACGGUGGGGUUCCGC |
| 3796 | CGGAACCCCACCGUGAAGG | 8550 | CCUUCACGGUGGGGUUCCG |
| 3798 | GAACCCCACCGUGAAGGAC | 8552 | GUCCUUCACGGUGGGGUUC |
| 3799 | AACCCCACCGUGAAGGACC | 8553 | GGUCCUUCACGGUGGGGUU |
| 3800 | ACCCCACCGUGAAGGACCU | 8554 | AGGUCCUUCACGGUGGGGU |
| 3801 | CCCCACCGUGAAGGACCUC | 8555 | GAGGUCCUUCACGGUGGGG |
| 3871 | AAGUUUGUGCACAGGGACC | 8625 | GGUCCCUGUGCACAAACUU |
| 4284 | GGUGGAGCAGAUAGUGUCU | 9038 | AGACACUAUCUGCUCCACC |
| 4292 | AGAUAGUGUCUGCACUGCU | 9046 | AGCAGUGCAGACACUAUCU |
| 4308 | GCUUGGGGACCAUUAUGUG | 9062 | CACAUAAUGGUCCCCAAGC |
| 4311 | UGGGGACCAUUAUGUGCAG | 9065 | CUGCACAUAAUGGUCCCCA |
| 4313 | GGGACCAUUAUGUGCAGCU | 9067 | AGCUGCACAUAAUGGUCCC |
| 4333 | CCAGCAACCUACAUGAACU | 9087 | AGUUCAUGUAGGUUGCUGG |
| 4449 | GCCUCCUCGGCCCACUUGA | 9203 | UCAAGUGGGCCGAGGAGGC |

The siRNAs in subset A have the following characteristics:
Cross-reactivity: With 19mer in human MST1R mRNA, with 17mer/19mer in NHP MST1R
Specificity category: For human and NHP: AS2 or better, SS3 or better
miRNA seeds: AS+SS strand: seed region not conserved in human, mouse, and rat and not present in >4 species
Off-target frequency: ≤20 human off-targets matched with 2 mismatches in antisense strand
SNPs: siRNA target sites do not harbor SNPs with a MAF ≥1% (pos. 2-18)

The siRNA sequences in subset A were selected for more stringent specificity to yield subset B. Subset B includes 297 siRNAs whose base sequences are shown in Table 4.

TABLE 4
Sequences in siRNA subset B

| SEQ ID | sense strand sequence | SEQ ID | antisense strand sequence |
|---|---|---|---|
| NO: | (5′-3′) | NO: | (5′-3′) |
| 469 | GCUGUGUUUGUAGCCAUAC | 5223 | GUAUGGCUACAAACACAGC |
| 471 | UGUGUUUGUAGCCAUACGC | 5225 | GCGUAUGGCUACAAACACA |
| 472 | GUGUUUGUAGCCAUACGCA | 5226 | UGCGUAUGGCUACAAACAC |
| 473 | UGUUUGUAGCCAUACGCAA | 5227 | UUGCGUAUGGCUACAAACA |
| 474 | GUUUGUAGCCAUACGCAAU | 5228 | AUUGCGUAUGGCUACAAAC |
| 475 | UUUGUAGCCAUACGCAAUC | 5229 | GAUUGCGUAUGGCUACAAA |
| 476 | UUGUAGCCAUACGCAAUCG | 5230 | CGAUUGCGUAUGGCUACAA |
| 477 | UGUAGCCAUACGCAAUCGC | 5231 | GCGAUUGCGUAUGGCUACA |
| 478 | GUAGCCAUACGCAAUCGCC | 5232 | GGCGAUUGCGUAUGGCUAC |
| 479 | UAGCCAUACGCAAUCGCCU | 5233 | AGGCGAUUGCGUAUGGCUA |
| 480 | AGCCAUACGCAAUCGCCUG | 5234 | CAGGCGAUUGCGUAUGGCU |
| 481 | GCCAUACGCAAUCGCCUGC | 5235 | GCAGGCGAUUGCGUAUGGC |
| 482 | CCAUACGCAAUCGCCUGCA | 5236 | UGCAGGCGAUUGCGUAUGG |
| 483 | CAUACGCAAUCGCCUGCAU | 5237 | AUGCAGGCGAUUGCGUAUG |
| 562 | GGCUGCCAGACGUGUGCAG | 5316 | CUGCACACGUCUGGCAGCC |
| 564 | CUGCCAGACGUGUGCAGCC | 5318 | GGCUGCACACGUCUGGCAG |
| 568 | CAGACGUGUGCAGCCUGUG | 5322 | CACAGGCUGCACACGUCUG |
| 691 | UGCUUCCUGCAUGACCUAG | 5445 | CUAGGUCAUGCAGGAAGCA |
| 692 | GCUUCCUGCAUGACCUAGA | 5446 | UCUAGGUCAUGCAGGAAGC |
| 693 | CUUCCUGCAUGACCUAGAG | 5447 | CUCUAGGUCAUGCAGGAAG |
| 717 | AGGGACAGCCGUGCAUCUG | 5471 | CAGAUGCACGGCUGUCCCU |
| 718 | GGGACAGCCGUGCAUCUGG | 5472 | CCAGAUGCACGGCUGUCCC |
| 721 | ACAGCCGUGCAUCUGGCAG | 5475 | CUGCCAGAUGCACGGCUGU |
| 722 | CAGCCGUGCAUCUGGCAGC | 5476 | GCUGCCAGAUGCACGGCUG |
| 724 | GCCGUGCAUCUGGCAGCGC | 5478 | GCGCUGCCAGAUGCACGGC |
| 725 | CCGUGCAUCUGGCAGCGCC | 5479 | GGCGCUGCCAGAUGCACGG |
| 726 | CGUGCAUCUGGCAGCGCCA | 5480 | UGGCGCUGCCAGAUGCACG |
| 753 | CUUCUCAGCCCACCAUAAC | 5507 | GUUAUGGUGGGCUGAGAAG |
| 759 | AGCCCACCAUAACCGGCCC | 5513 | GGGCCGGUUAUGGUGGGCU |
| 761 | CCCACCAUAACCGGCCCGA | 5515 | UCGGGCCGGUUAUGGUGGG |
| 762 | CCACCAUAACCGGCCCGAU | 5516 | AUCGGGCCGGUUAUGGUGG |
| 763 | CACCAUAACCGGCCCGAUG | 5517 | CAUCGGGCCGGUUAUGGUG |
| 764 | ACCAUAACCGGCCCGAUGA | 5518 | UCAUCGGGCCGGUUAUGGU |
| 765 | CCAUAACCGGCCCGAUGAC | 5519 | GUCAUCGGGCCGGUUAUGG |
| 766 | CAUAACCGGCCCGAUGACU | 5520 | AGUCAUCGGGCCGGUUAUG |
| 767 | AUAACCGGCCCGAUGACUG | 5521 | CAGUCAUCGGGCCGGUUAU |
| 775 | CCCGAUGACUGCCCCGACU | 5529 | AGUCGGGGCAGUCAUCGGG |
| 776 | CCGAUGACUGCCCCGACUG | 5530 | CAGUCGGGGCAGUCAUCGG |
| 777 | CGAUGACUGCCCCGACUGU | 5531 | ACAGUCGGGGCAGUCAUCG |
| 801 | CAGCCCAUUGGGCACCCGU | 5555 | ACGGGUGCCCAAUGGGCUG |
| 804 | CCCAUUGGGCACCCGUGUA | 5558 | UACACGGGUGCCCAAUGGG |
| 826 | GUGGUUGAGCAAGGCCAGG | 5580 | CCUGGCCUUGCUCAACCAC |
| 842 | AGGCCUCCUAUUUCUACGU | 5596 | ACGUAGAAAUAGGAGGCCU |
| 845 | CCUCCUAUUUCUACGUGGC | 5599 | GCCACGUAGAAAUAGGAGG |
| 854 | UCUACGUGGCAUCCUCACU | 5608 | AGUGAGGAUGCCACGUAGA |
| 855 | CUACGUGGCAUCCUCACUG | 5609 | CAGUGAGGAUGCCACGUAG |
| 863 | CAUCCUCACUGGACGCAGC | 5617 | GCUGCGUCCAGUGAGGAUG |
| 866 | CCUCACUGGACGCAGCCGU | 5620 | ACGGCUGCGUCCAGUGAGG |
| 895 | UUCAGCCCACGCUCAGUGU | 5649 | ACACUGAGCGUGGGCUGAA |
| 897 | CAGCCCACGCUCAGUGUCU | 5651 | AGACACUGAGCGUGGGCUG |
| 898 | AGCCCACGCUCAGUGUCUA | 5652 | UAGACACUGAGCGUGGGCU |
| 899 | GCCCACGCUCAGUGUCUAU | 5653 | AUAGACACUGAGCGUGGGC |
| 900 | CCCACGCUCAGUGUCUAUC | 5654 | GAUAGACACUGAGCGUGGG |
| 901 | CCACGCUCAGUGUCUAUCA | 5655 | UGAUAGACACUGAGCGUGG |
| 903 | ACGCUCAGUGUCUAUCAGG | 5657 | CCUGAUAGACACUGAGCGU |
| 904 | CGCUCAGUGUCUAUCAGGC | 5658 | GCCUGAUAGACACUGAGCG |
| 907 | UCAGUGUCUAUCAGGCGUC | 5661 | GACGCCUGAUAGACACUGA |
| 908 | CAGUGUCUAUCAGGCGUCU | 5662 | AGACGCCUGAUAGACACUG |
| 909 | AGUGUCUAUCAGGCGUCUC | 5663 | GAGACGCCUGAUAGACACU |
| 910 | GUGUCUAUCAGGCGUCUCA | 5664 | UGAGACGCCUGAUAGACAC |
| 911 | UGUCUAUCAGGCGUCUCAA | 5665 | UUGAGACGCCUGAUAGACA |
| 912 | GUCUAUCAGGCGUCUCAAG | 5666 | CUUGAGACGCCUGAUAGAC |
| 913 | UCUAUCAGGCGUCUCAAGG | 5667 | CCUUGAGACGCCUGAUAGA |
| 915 | UAUCAGGCGUCUCAAGGCU | 5669 | AGCCUUGAGACGCCUGAUA |
| 966 | GUUGUCAGUGCUGCCCAAG | 5720 | CUUGGGCAGCACUGACAAC |
| 976 | CUGCCCAAGCAUCUUGUCU | 5730 | AGACAAGAUGCUUGGGCAG |
| 977 | UGCCCAAGCAUCUUGUCUC | 5731 | GAGACAAGAUGCUUGGGCA |
| 985 | CAUCUUGUCUCCUACAGUA | 5739 | UACUGUAGGAGACAAGAUG |
| 986 | AUCUUGUCUCCUACAGUAU | 5740 | AUACUGUAGGAGACAAGAU |
| 992 | UCUCCUACAGUAUUGAAUA | 5746 | UAUUCAAUACUGUAGGAGA |
| 1001 | GUAUUGAAUACGUGCACAG | 5755 | CUGUGCACGUAUUCAAUAC |
| 1003 | AUUGAAUACGUGCACAGCU | 5757 | AGCUGUGCACGUAUUCAAU |
| 1004 | UUGAAUACGUGCACAGCUU | 5758 | AAGCUGUGCACGUAUUCAA |
| 1005 | UGAAUACGUGCACAGCUUC | 5759 | GAAGCUGUGCACGUAUUCA |
| 1046 | UCCUGACUGUACAGCCGGC | 5800 | GCCGGCUGUACAGUCAGGA |
| 1048 | CUGACUGUACAGCCGGCCA | 5802 | UGGCCGGCUGUACAGUCAG |
| 1049 | UGACUGUACAGCCGGCCAG | 5803 | CUGGCCGGCUGUACAGUCA |
| 1050 | GACUGUACAGCCGGCCAGC | 5804 | GCUGGCCGGCUGUACAGUC |
| 1051 | ACUGUACAGCCGGCCAGCG | 5805 | CGCUGGCCGGCUGUACAGU |
| 1053 | UGUACAGCCGGCCAGCGUG | 5807 | CACGCUGGCCGGCUGUACA |
| 1054 | GUACAGCCGGCCAGCGUGA | 5808 | UCACGCUGGCCGGCUGUAC |
| 1055 | UACAGCCGGCCAGCGUGAC | 5809 | GUCACGCUGGCCGGCUGUA |
| 1111 | CUUAGCGCCACUGAGCCAG | 5865 | CUGGCUCAGUGGCGCUAAG |
| 1129 | GAGUUGGGUGACUAUCGGG | 5883 | CCCGAUAGUCACCCAACUC |
| 1130 | AGUUGGGUGACUAUCGGGA | 5884 | UCCCGAUAGUCACCCAACU |
| 1131 | GUUGGGUGACUAUCGGGAG | 5885 | CUCCCGAUAGUCACCCAAC |
| 1132 | UUGGGUGACUAUCGGGAGC | 5886 | GCUCCCGAUAGUCACCCAA |
| 1134 | GGGUGACUAUCGGGAGCUG | 5888 | CAGCUCCCGAUAGUCACCC |
| 1137 | UGACUAUCGGGAGCUGGUC | 5891 | GACCAGCUCCCGAUAGUCA |
| 1138 | GACUAUCGGGAGCUGGUCC | 5892 | GGACCAGCUCCCGAUAGUC |
| 1139 | ACUAUCGGGAGCUGGUCCU | 5893 | AGGACCAGCUCCCGAUAGU |
| 1146 | GGAGCUGGUCCUCGACUGC | 5900 | GCAGUCGAGGACCAGCUCC |
| 1147 | GAGCUGGUCCUCGACUGCA | 5901 | UGCAGUCGAGGACCAGCUC |
| 1152 | GGUCCUCGACUGCAGAUUU | 5906 | AAAUCUGCAGUCGAGGACC |
| 1153 | GUCCUCGACUGCAGAUUUG | 5907 | CAAAUCUGCAGUCGAGGAC |
| 1154 | UCCUCGACUGCAGAUUUGC | 5908 | GCAAAUCUGCAGUCGAGGA |
| 1155 | CCUCGACUGCAGAUUUGCU | 5909 | AGCAAAUCUGCAGUCGAGG |
| 1156 | CUCGACUGCAGAUUUGCUC | 5910 | GAGCAAAUCUGCAGUCGAG |
| 1157 | UCGACUGCAGAUUUGCUCC | 5911 | GGAGCAAAUCUGCAGUCGA |
| 1158 | CGACUGCAGAUUUGCUCCA | 5912 | UGGAGCAAAUCUGCAGUCG |
| 1161 | CUGCAGAUUUGCUCCAAAA | 5915 | UUUUGGAGCAAAUCUGCAG |
| 1162 | UGCAGAUUUGCUCCAAAAC | 5916 | GUUUUGGAGCAAAUCUGCA |
| 1164 | CAGAUUUGCUCCAAAACGC | 5918 | GCGUUUUGGAGCAAAUCUG |
| 1165 | AGAUUUGCUCCAAAACGCA | 5919 | UGCGUUUUGGAGCAAAUCU |
| 1166 | GAUUUGCUCCAAAACGCAG | 5920 | CUGCGUUUUGGAGCAAAUC |
| 1170 | UGCUCCAAAACGCAGGCGC | 5924 | GCGCCUGCGUUUUGGAGCA |
| 1171 | GCUCCAAAACGCAGGCGCC | 5925 | GGCGCCUGCGUUUUGGAGC |
| 1172 | CUCCAAAACGCAGGCGCCG | 5926 | CGGCGCCUGCGUUUUGGAG |
| 1173 | UCCAAAACGCAGGCGCCGG | 5927 | CCGGCGCCUGCGUUUUGGA |
| 1204 | GGCGGACAGCCCUACCCUG | 5958 | CAGGGUAGGGCUGUCCGCC |
| 1350 | CAACUCUGUCGUCUGUGCC | 6104 | GGCACAGACGACAGAGUUG |
| 1405 | GGUGUGGAGCGCUGUUGUG | 6159 | CACAACAGCGCUCCACACC |
| 1406 | GUGUGGAGCGCUGUUGUGA | 6160 | UCACAACAGCGCUCCACAC |
| 1408 | GUGGAGCGCUGUUGUGAAU | 6162 | AUUCACAACAGCGCUCCAC |
| 1409 | UGGAGCGCUGUUGUGAAUC | 6163 | GAUUCACAACAGCGCUCCA |
| 1411 | GAGCGCUGUUGUGAAUCCC | 6165 | GGGAUUCACAACAGCGCUC |
| 1443 | CCUCCGGCGAGGCCUCGAC | 6197 | GUCGAGGCCUCGCCGGAGG |
| 1444 | CUCCGGCGAGGCCUCGACU | 6198 | AGUCGAGGCCUCGCCGGAG |
| 1445 | UCCGGCGAGGCCUCGACUU | 6199 | AAGUCGAGGCCUCGCCGGA |
| 1446 | CCGGCGAGGCCUCGACUUC | 6200 | GAAGUCGAGGCCUCGCCGG |
| 1447 | CGGCGAGGCCUCGACUUCU | 6201 | AGAAGUCGAGGCCUCGCCG |
| 1476 | CAGUUUUUGCCCCAACCCG | 6230 | CGGGUUGGGGCAAAAACUG |
| 1857 | GACCUGUGGGCGUUGCCUA | 6611 | UAGGCAACGCCCACAGGUC |
| 1858 | ACCUGUGGGCGUUGCCUAA | 6612 | UUAGGCAACGCCCACAGGU |
| 1862 | GUGGGCGUUGCCUAAGGGC | 6616 | GCCCUUAGGCAACGCCCAC |
| 1863 | UGGGCGUUGCCUAAGGGCA | 6617 | UGCCCUUAGGCAACGCCCA |
| 1969 | CCUAAGCUUACUGAGUUCC | 6723 | GGAACUCAGUAAGCUUAGG |
| 2125 | AAGGACAGCUCAAAACUCA | 6879 | UGAGUUUUGAGCUGUCCUU |
| 2142 | CAGACCAGUGCCCCGGAAA | 6896 | UUUCCGGGGCACUGGUCUG |
| 2143 | AGACCAGUGCCCCGGAAAG | 6897 | CUUUCCGGGGCACUGGUCU |
| 2144 | GACCAGUGCCCCGGAAAGA | 6898 | UCUUUCCGGGGCACUGGUC |
| 2148 | AGUGCCCCGGAAAGACUUU | 6902 | AAAGUCUUUCCGGGGCACU |
| 2149 | GUGCCCCGGAAAGACUUUG | 6903 | CAAAGUCUUUCCGGGGCAC |
| 2150 | UGCCCCGGAAAGACUUUGU | 6904 | ACAAAGUCUUUCCGGGGCA |
| 2151 | GCCCCGGAAAGACUUUGUA | 6905 | UACAAAGUCUUUCCGGGGC |
| 2152 | CCCCGGAAAGACUUUGUAG | 6906 | CUACAAAGUCUUUCCGGGG |
| 2153 | CCCGGAAAGACUUUGUAGA | 6907 | UCUACAAAGUCUUUCCGGG |
| 2154 | CCGGAAAGACUUUGUAGAG | 6908 | CUCUACAAAGUCUUUCCGG |
| 2208 | GGCAGUGGGGCCUACCAAC | 6962 | GUUGGUAGGCCCCACUGCC |
| 2226 | CGUCAGCCUCACCGUGACU | 6980 | AGUCACGGUGAGGCUGACG |
| 2228 | UCAGCCUCACCGUGACUAA | 6982 | UUAGUCACGGUGAGGCUGA |
| 2229 | CAGCCUCACCGUGACUAAC | 6983 | GUUAGUCACGGUGAGGCUG |
| 2230 | AGCCUCACCGUGACUAACA | 6984 | UGUUAGUCACGGUGAGGCU |
| 2232 | CCUCACCGUGACUAACAUG | 6986 | CAUGUUAGUCACGGUGAGG |
| 2233 | CUCACCGUGACUAACAUGC | 6987 | GCAUGUUAGUCACGGUGAG |
| 2234 | UCACCGUGACUAACAUGCC | 6988 | GGCAUGUUAGUCACGGUGA |
| 2235 | CACCGUGACUAACAUGCCA | 6989 | UGGCAUGUUAGUCACGGUG |
| 2237 | CCGUGACUAACAUGCCACC | 6991 | GGUGGCAUGUUAGUCACGG |
| 2240 | UGACUAACAUGCCACCGGG | 6994 | CCCGGUGGCAUGUUAGUCA |
| 2241 | GACUAACAUGCCACCGGGC | 6995 | GCCCGGUGGCAUGUUAGUC |
| 2242 | ACUAACAUGCCACCGGGCA | 6996 | UGCCCGGUGGCAUGUUAGU |
| 2243 | CUAACAUGCCACCGGGCAA | 6997 | UUGCCCGGUGGCAUGUUAG |
| 2244 | UAACAUGCCACCGGGCAAG | 6998 | CUUGCCCGGUGGCAUGUUA |
| 2245 | AACAUGCCACCGGGCAAGC | 6999 | GCUUGCCCGGUGGCAUGUU |
| 2246 | ACAUGCCACCGGGCAAGCA | 7000 | UGCUUGCCCGGUGGCAUGU |
| 2252 | CACCGGGCAAGCACUUCCG | 7006 | CGGAAGUGCUUGCCCGGUG |
| 2253 | ACCGGGCAAGCACUUCCGG | 7007 | CCGGAAGUGCUUGCCCGGU |
| 2257 | GGCAAGCACUUCCGGGUAG | 7011 | CUACCCGGAAGUGCUUGCC |
| 2258 | GCAAGCACUUCCGGGUAGA | 7012 | UCUACCCGGAAGUGCUUGC |
| 2259 | CAAGCACUUCCGGGUAGAC | 7013 | GUCUACCCGGAAGUGCUUG |
| 2263 | CACUUCCGGGUAGACGGCA | 7017 | UGCCGUCUACCCGGAAGUG |
| 2264 | ACUUCCGGGUAGACGGCAC | 7018 | GUGCCGUCUACCCGGAAGU |
| 2265 | CUUCCGGGUAGACGGCACC | 7019 | GGUGCCGUCUACCCGGAAG |
| 2266 | UUCCGGGUAGACGGCACCU | 7020 | AGGUGCCGUCUACCCGGAA |
| 2267 | UCCGGGUAGACGGCACCUC | 7021 | GAGGUGCCGUCUACCCGGA |
| 2269 | CGGGUAGACGGCACCUCCG | 7023 | CGGAGGUGCCGUCUACCCG |
| 2318 | UGCUGAUAGCAGUGCAACC | 7072 | GGUUGCACUGCUAUCAGCA |
| 2319 | GCUGAUAGCAGUGCAACCC | 7073 | GGGUUGCACUGCUAUCAGC |
| 2320 | CUGAUAGCAGUGCAACCCC | 7074 | GGGGUUGCACUGCUAUCAG |
| 2322 | GAUAGCAGUGCAACCCCUC | 7076 | GAGGGGUUGCACUGCUAUC |
| 2332 | CAACCCCUCUUUGGCCCAC | 7086 | GUGGGCCAAAGAGGGGUUG |
| 2366 | GUCUCACUCUUGAAGGCCA | 7120 | UGGCCUUCAAGAGUGAGAC |
| 2369 | UCACUCUUGAAGGCCAGAG | 7123 | CUCUGGCCUUCAAGAGUGA |
| 2386 | AGUCUGUCUGUAGGCACCA | 7140 | UGGUGCCUACAGACAGACU |
| 2413 | GUGCUGGUCAAUGGGACUG | 7167 | CAGUCCCAUUGACCAGCAC |
| 2418 | GGUCAAUGGGACUGAGUGU | 7172 | ACACUCAGUCCCAUUGACC |
| 2420 | UCAAUGGGACUGAGUGUCU | 7174 | AGACACUCAGUCCCAUUGA |
| 2421 | CAAUGGGACUGAGUGUCUG | 7175 | CAGACACUCAGUCCCAUUG |
| 2422 | AAUGGGACUGAGUGUCUGC | 7176 | GCAGACACUCAGUCCCAUU |
| 2425 | GGGACUGAGUGUCUGCUAG | 7179 | CUAGCAGACACUCAGUCCC |
| 2427 | GACUGAGUGUCUGCUAGCA | 7181 | UGCUAGCAGACACUCAGUC |
| 2430 | UGAGUGUCUGCUAGCACGG | 7184 | CCGUGCUAGCAGACACUCA |
| 2432 | AGUGUCUGCUAGCACGGGU | 7186 | ACCCGUGCUAGCAGACACU |
| 2435 | GUCUGCUAGCACGGGUCAG | 7189 | CUGACCCGUGCUAGCAGAC |
| 2437 | CUGCUAGCACGGGUCAGUG | 7191 | CACUGACCCGUGCUAGCAG |
| 2438 | UGCUAGCACGGGUCAGUGA | 7192 | UCACUGACCCGUGCUAGCA |
| 2440 | CUAGCACGGGUCAGUGAGG | 7194 | CCUCACUGACCCGUGCUAG |
| 2443 | GCACGGGUCAGUGAGGGGC | 7197 | GCCCCUCACUGACCCGUGC |
| 2445 | ACGGGUCAGUGAGGGGCAG | 7199 | CUGCCCCUCACUGACCCGU |
| 2455 | GAGGGGCAGCUUUUAUGUG | 7209 | CACAUAAAAGCUGCCCCUC |
| 2460 | GCAGCUUUUAUGUGCCACA | 7214 | UGUGGCACAUAAAAGCUGC |
| 2462 | AGCUUUUAUGUGCCACACC | 7216 | GGUGUGGCACAUAAAAGCU |
| 2566 | GAAGACCCUGUCGUGCUAA | 7320 | UUAGCACGACAGGGUCUUC |
| 2567 | AAGACCCUGUCGUGCUAAG | 7321 | CUUAGCACGACAGGGUCUU |
| 2571 | CCCUGUCGUGCUAAGCAUC | 7325 | GAUGCUUAGCACGACAGGG |
| 2572 | CCUGUCGUGCUAAGCAUCA | 7326 | UGAUGCUUAGCACGACAGG |
| 2575 | GUCGUGCUAAGCAUCAGCC | 7329 | GGCUGAUGCUUAGCACGAC |
| 2577 | CGUGCUAAGCAUCAGCCCC | 7331 | GGGGCUGAUGCUUAGCACG |
| 2581 | CUAAGCAUCAGCCCCAACU | 7335 | AGUUGGGGCUGAUGCUUAG |
| 2582 | UAAGCAUCAGCCCCAACUG | 7336 | CAGUUGGGGCUGAUGCUUA |
| 2628 | CUGUGGCCAGCAUCUAACU | 7382 | AGUUAGAUGCUGGCCACAG |
| 2629 | UGUGGCCAGCAUCUAACUU | 7383 | AAGUUAGAUGCUGGCCACA |
| 2644 | ACUUCAGCAUGGCACUUAG | 7398 | CUAAGUGCCAUGCUGAAGU |
| 2646 | UUCAGCAUGGCACUUAGUG | 7400 | CACUAAGUGCCAUGCUGAA |
| 2648 | CAGCAUGGCACUUAGUGCU | 7402 | AGCACUAAGUGCCAUGCUG |
| 2652 | AUGGCACUUAGUGCUGUCA | 7406 | UGACAGCACUAAGUGCCAU |
| 2655 | GCACUUAGUGCUGUCAUUC | 7409 | GAAUGACAGCACUAAGUGC |
| 2656 | CACUUAGUGCUGUCAUUCC | 7410 | GGAAUGACAGCACUAAGUG |
| 2661 | AGUGCUGUCAUUCCAUGAC | 7415 | GUCAUGGAAUGACAGCACU |
| 2663 | UGCUGUCAUUCCAUGACGG | 7417 | CCGUCAUGGAAUGACAGCA |
| 2664 | GCUGUCAUUCCAUGACGGG | 7418 | CCCGUCAUGGAAUGACAGC |
| 2666 | UGUCAUUCCAUGACGGGCU | 7420 | AGCCCGUCAUGGAAUGACA |
| 2668 | UCAUUCCAUGACGGGCUUA | 7422 | UAAGCCCGUCAUGGAAUGA |
| 2669 | CAUUCCAUGACGGGCUUAG | 7423 | CUAAGCCCGUCAUGGAAUG |
| 2670 | AUUCCAUGACGGGCUUAGG | 7424 | CCUAAGCCCGUCAUGGAAU |
| 2671 | UUCCAUGACGGGCUUAGGG | 7425 | CCCUAAGCCCGUCAUGGAA |
| 2673 | CCAUGACGGGCUUAGGGCA | 7427 | UGCCCUAAGCCCGUCAUGG |
| 2676 | UGACGGGCUUAGGGCAGUG | 7430 | CACUGCCCUAAGCCCGUCA |
| 2678 | ACGGGCUUAGGGCAGUGGA | 7432 | UCCACUGCCCUAAGCCCGU |
| 2891 | AGGAGCAUGCCAUUAAGUU | 7645 | AACUUAAUGGCAUGCUCCU |
| 3084 | UAUCCUGGGUAGAGUGGUG | 7838 | CACCACUCUACCCAGGAUA |
| 3186 | UGCACUGGUCUUCAGCUAC | 7940 | GUAGCUGAAGACCAGUGCA |
| 3191 | UGGUCUUCAGCUACUGGUG | 7945 | CACCAGUAGCUGAAGACCA |
| 3192 | GGUCUUCAGCUACUGGUGG | 7946 | CCACCAGUAGCUGAAGACC |
| 3223 | CUAGUUCUUCCUCCCAACC | 7977 | GGUUGGGAGGAAGAACUAG |
| 3279 | CACACCCCUGCCUAUUCUG | 8033 | CAGAAUAGGCAGGGGUGUG |
| 3297 | GUACUCGGGCUCUGACUAC | 8051 | GUAGUCAGAGCCCGAGUAC |
| 3298 | UACUCGGGCUCUGACUACA | 8052 | UGUAGUCAGAGCCCGAGUA |
| 3299 | ACUCGGGCUCUGACUACAG | 8053 | CUGUAGUCAGAGCCCGAGU |
| 3300 | CUCGGGCUCUGACUACAGA | 8054 | UCUGUAGUCAGAGCCCGAG |
| 3301 | UCGGGCUCUGACUACAGAA | 8055 | UUCUGUAGUCAGAGCCCGA |
| 3560 | GAGAAUACAUAGACCAGGC | 8314 | GCCUGGUCUAUGUAUUCUC |
| 3563 | AAUACAUAGACCAGGCCCA | 8317 | UGGGCCUGGUCUAUGUAUU |
| 3564 | AUACAUAGACCAGGCCCAG | 8318 | CUGGGCCUGGUCUAUGUAU |
| 3568 | AUAGACCAGGCCCAGAAUC | 8322 | GAUUCUGGGCCUGGUCUAU |
| 3570 | AGACCAGGCCCAGAAUCGA | 8324 | UCGAUUCUGGGCCUGGUCU |
| 3574 | CAGGCCCAGAAUCGAAUCC | 8328 | GGAUUCGAUUCUGGGCCUG |
| 3576 | GGCCCAGAAUCGAAUCCAA | 8330 | UUGGAUUCGAUUCUGGGCC |
| 3577 | GCCCAGAAUCGAAUCCAAU | 8331 | AUUGGAUUCGAUUCUGGGC |
| 3578 | CCCAGAAUCGAAUCCAAUG | 8332 | CAUUGGAUUCGAUUCUGGG |
| 3580 | CAGAAUCGAAUCCAAUGUG | 8334 | CACAUUGGAUUCGAUUCUG |
| 3581 | AGAAUCGAAUCCAAUGUGC | 8335 | GCACAUUGGAUUCGAUUCU |
| 3582 | GAAUCGAAUCCAAUGUGCC | 8336 | GGCACAUUGGAUUCGAUUC |
| 3583 | AAUCGAAUCCAAUGUGCCA | 8337 | UGGCACAUUGGAUUCGAUU |
| 3584 | AUCGAAUCCAAUGUGCCAU | 8338 | AUGGCACAUUGGAUUCGAU |
| 3586 | CGAAUCCAAUGUGCCAUCA | 8340 | UGAUGGCACAUUGGAUUCG |
| 3587 | GAAUCCAAUGUGCCAUCAA | 8341 | UUGAUGGCACAUUGGAUUC |
| 3588 | AAUCCAAUGUGCCAUCAAG | 8342 | CUUGAUGGCACAUUGGAUU |
| 3592 | CAAUGUGCCAUCAAGUCAC | 8346 | GUGACUUGAUGGCACAUUG |
| 3594 | AUGUGCCAUCAAGUCACUA | 8348 | UAGUGACUUGAUGGCACAU |
| 3595 | UGUGCCAUCAAGUCACUAA | 8349 | UUAGUGACUUGAUGGCACA |
| 3596 | GUGCCAUCAAGUCACUAAG | 8350 | CUUAGUGACUUGAUGGCAC |
| 3597 | UGCCAUCAAGUCACUAAGU | 8351 | ACUUAGUGACUUGAUGGCA |
| 3600 | CAUCAAGUCACUAAGUCGC | 8354 | GCGACUUAGUGACUUGAUG |
| 3655 | GAGGGGCUGCUCAUGCGUG | 8409 | CACGCAUGAGCAGCCCCUC |
| 3656 | AGGGGCUGCUCAUGCGUGG | 8410 | CCACGCAUGAGCAGCCCCU |
| 3657 | GGGGCUGCUCAUGCGUGGC | 8411 | GCCACGCAUGAGCAGCCCC |
| 3659 | GGCUGCUCAUGCGUGGCCU | 8413 | AGGCCACGCAUGAGCAGCC |
| 3666 | CAUGCGUGGCCUGAACCAC | 8420 | GUGGUUCAGGCCACGCAUG |
| 3671 | GUGGCCUGAACCACCCGAA | 8425 | UUCGGGUGGUUCAGGCCAC |
| 3672 | UGGCCUGAACCACCCGAAU | 8426 | AUUCGGGUGGUUCAGGCCA |
| 3673 | GGCCUGAACCACCCGAAUG | 8427 | CAUUCGGGUGGUUCAGGCC |
| 3674 | GCCUGAACCACCCGAAUGU | 8428 | ACAUUCGGGUGGUUCAGGC |
| 3676 | CUGAACCACCCGAAUGUGC | 8430 | GCACAUUCGGGUGGUUCAG |
| 3685 | CCGAAUGUGCUGGCUCUCA | 8439 | UGAGAGCCAGCACAUUCGG |
| 3701 | UCAUUGGUAUCAUGUUGCC | 8455 | GGCAACAUGAUACCAAUGA |
| 3702 | CAUUGGUAUCAUGUUGCCA | 8456 | UGGCAACAUGAUACCAAUG |
| 3705 | UGGUAUCAUGUUGCCACCU | 8459 | AGGUGGCAACAUGAUACCA |
| 3746 | UGCCCUAUAUGUGCCACGG | 8500 | CCGUGGCACAUAUAGGGCA |
| 3748 | CCCUAUAUGUGCCACGGUG | 8502 | CACCGUGGCACAUAUAGGG |
| 3749 | CCUAUAUGUGCCACGGUGA | 8503 | UCACCGUGGCACAUAUAGG |
| 3750 | CUAUAUGUGCCACGGUGAC | 8504 | GUCACCGUGGCACAUAUAG |
| 3751 | UAUAUGUGCCACGGUGACC | 8505 | GGUCACCGUGGCACAUAUA |
| 3752 | AUAUGUGCCACGGUGACCU | 8506 | AGGUCACCGUGGCACAUAU |
| 3755 | UGUGCCACGGUGACCUGCU | 8509 | AGCAGGUCACCGUGGCACA |
| 3762 | CGGUGACCUGCUCCAGUUC | 8516 | GAACUGGAGCAGGUCACCG |
| 3770 | UGCUCCAGUUCAUCCGCUC | 8524 | GAGCGGAUGAACUGGAGCA |
| 3771 | GCUCCAGUUCAUCCGCUCA | 8525 | UGAGCGGAUGAACUGGAGC |
| 3773 | UCCAGUUCAUCCGCUCACC | 8527 | GGUGAGCGGAUGAACUGGA |
| 3775 | CAGUUCAUCCGCUCACCUC | 8529 | GAGGUGAGCGGAUGAACUG |
| 3777 | GUUCAUCCGCUCACCUCAG | 8531 | CUGAGGUGAGCGGAUGAAC |
| 3783 | CCGCUCACCUCAGCGGAAC | 8537 | GUUCCGCUGAGGUGAGCGG |
| 3788 | CACCUCAGCGGAACCCCAC | 8542 | GUGGGGUUCCGCUGAGGUG |
| 3791 | CUCAGCGGAACCCCACCGU | 8545 | ACGGUGGGGUUCCGCUGAG |
| 3793 | CAGCGGAACCCCACCGUGA | 8547 | UCACGGUGGGGUUCCGCUG |
| 3794 | AGCGGAACCCCACCGUGAA | 8548 | UUCACGGUGGGGUUCCGCU |
| 3795 | GCGGAACCCCACCGUGAAG | 8549 | CUUCACGGUGGGGUUCCGC |
| 3798 | GAACCCCACCGUGAAGGAC | 8552 | GUCCUUCACGGUGGGGUUC |
| 3799 | AACCCCACCGUGAAGGACC | 8553 | GGUCCUUCACGGUGGGGUU |
| 3800 | ACCCCACCGUGAAGGACCU | 8554 | AGGUCCUUCACGGUGGGGU |
| 3871 | AAGUUUGUGCACAGGGACC | 8625 | GGUCCCUGUGCACAAACUU |
| 4284 | GGUGGAGCAGAUAGUGUCU | 9038 | AGACACUAUCUGCUCCACC |
| 4292 | AGAUAGUGUCUGCACUGCU | 9046 | AGCAGUGCAGACACUAUCU |
| 4308 | GCUUGGGGACCAUUAUGUG | 9062 | CACAUAAUGGUCCCCAAGC |
| 4311 | UGGGGACCAUUAUGUGCAG | 9065 | CUGCACAUAAUGGUCCCCA |
| 4313 | GGGACCAUUAUGUGCAGCU | 9067 | AGCUGCACAUAAUGGUCCC |
| 4333 | CCAGCAACCUACAUGAACU | 9087 | AGUUCAUGUAGGUUGCUGG |

The siRNAs in subset B have the following characteristics:
Cross-reactivity: With 19mer in human MST1R mRNA, with 17mer/19mer in NHP MST1R
Specificity category: For human and NHP: AS2 or better, SS3 or better
miRNA seeds: AS+SS strand: seed region not conserved in human, mouse, and rat and not present in >4 species
Off-target frequency: ≤15 human off-targets matched with 2 mismatches in antisense strand
SNPs: siRNA target sites do not harbor SNPs with a MAF ≥1% (pos. 2-18)

The siRNA sequences in subset B were further selected for absence of seed regions in the AS strand that are identical to a seed region of known human miRNA to yield subset C. Subset C includes 184 siRNAs whose base sequences are shown in Table 5.

TABLE 5
Sequences in siRNA subset C

| SEQ ID | sense strand sequence | SEQ ID | antisense strand sequence |
|---|---|---|---|
| NO: | (5′-3′) | NO: | (5′-3′) |
| 471 | UGUGUUUGUAGCCAUACGC | 5225 | GCGUAUGGCUACAAACACA |
| 472 | GUGUUUGUAGCCAUACGCA | 5226 | UGCGUAUGGCUACAAACAC |
| 473 | UGUUUGUAGCCAUACGCAA | 5227 | UUGCGUAUGGCUACAAACA |
| 474 | GUUUGUAGCCAUACGCAAU | 5228 | AUUGCGUAUGGCUACAAAC |
| 475 | UUUGUAGCCAUACGCAAUC | 5229 | GAUUGCGUAUGGCUACAAA |
| 476 | UUGUAGCCAUACGCAAUCG | 5230 | CGAUUGCGUAUGGCUACAA |
| 477 | UGUAGCCAUACGCAAUCGC | 5231 | GCGAUUGCGUAUGGCUACA |
| 478 | GUAGCCAUACGCAAUCGCC | 5232 | GGCGAUUGCGUAUGGCUAC |
| 480 | AGCCAUACGCAAUCGCCUG | 5234 | CAGGCGAUUGCGUAUGGCU |
| 482 | CCAUACGCAAUCGCCUGCA | 5236 | UGCAGGCGAUUGCGUAUGG |
| 562 | GGCUGCCAGACGUGUGCAG | 5316 | CUGCACACGUCUGGCAGCC |
| 564 | CUGCCAGACGUGUGCAGCC | 5318 | GGCUGCACACGUCUGGCAG |
| 691 | UGCUUCCUGCAUGACCUAG | 5445 | CUAGGUCAUGCAGGAAGCA |
| 692 | GCUUCCUGCAUGACCUAGA | 5446 | UCUAGGUCAUGCAGGAAGC |
| 693 | CUUCCUGCAUGACCUAGAG | 5447 | CUCUAGGUCAUGCAGGAAG |
| 717 | AGGGACAGCCGUGCAUCUG | 5471 | CAGAUGCACGGCUGUCCCU |
| 724 | GCCGUGCAUCUGGCAGCGC | 5478 | GCGCUGCCAGAUGCACGGC |
| 725 | CCGUGCAUCUGGCAGCGCC | 5479 | GGCGCUGCCAGAUGCACGG |
| 726 | CGUGCAUCUGGCAGCGCCA | 5480 | UGGCGCUGCCAGAUGCACG |
| 753 | CUUCUCAGCCCACCAUAAC | 5507 | GUUAUGGUGGGCUGAGAAG |
| 763 | CACCAUAACCGGCCCGAUG | 5517 | CAUCGGGCCGGUUAUGGUG |
| 764 | ACCAUAACCGGCCCGAUGA | 5518 | UCAUCGGGCCGGUUAUGGU |
| 765 | CCAUAACCGGCCCGAUGAC | 5519 | GUCAUCGGGCCGGUUAUGG |
| 766 | CAUAACCGGCCCGAUGACU | 5520 | AGUCAUCGGGCCGGUUAUG |
| 767 | AUAACCGGCCCGAUGACUG | 5521 | CAGUCAUCGGGCCGGUUAU |
| 775 | CCCGAUGACUGCCCCGACU | 5529 | AGUCGGGGCAGUCAUCGGG |
| 776 | CCGAUGACUGCCCCGACUG | 5530 | CAGUCGGGGCAGUCAUCGG |
| 777 | CGAUGACUGCCCCGACUGU | 5531 | ACAGUCGGGGCAGUCAUCG |
| 801 | CAGCCCAUUGGGCACCCGU | 5555 | ACGGGUGCCCAAUGGGCUG |
| 842 | AGGCCUCCUAUUUCUACGU | 5596 | ACGUAGAAAUAGGAGGCCU |
| 845 | CCUCCUAUUUCUACGUGGC | 5599 | GCCACGUAGAAAUAGGAGG |
| 895 | UUCAGCCCACGCUCAGUGU | 5649 | ACACUGAGCGUGGGCUGAA |
| 898 | AGCCCACGCUCAGUGUCUA | 5652 | UAGACACUGAGCGUGGGCU |
| 899 | GCCCACGCUCAGUGUCUAU | 5653 | AUAGACACUGAGCGUGGGC |
| 900 | CCCACGCUCAGUGUCUAUC | 5654 | GAUAGACACUGAGCGUGGG |
| 901 | CCACGCUCAGUGUCUAUCA | 5655 | UGAUAGACACUGAGCGUGG |
| 903 | ACGCUCAGUGUCUAUCAGG | 5657 | CCUGAUAGACACUGAGCGU |
| 904 | CGCUCAGUGUCUAUCAGGC | 5658 | GCCUGAUAGACACUGAGCG |
| 907 | UCAGUGUCUAUCAGGCGUC | 5661 | GACGCCUGAUAGACACUGA |
| 909 | AGUGUCUAUCAGGCGUCUC | 5663 | GAGACGCCUGAUAGACACU |
| 910 | GUGUCUAUCAGGCGUCUCA | 5664 | UGAGACGCCUGAUAGACAC |
| 912 | GUCUAUCAGGCGUCUCAAG | 5666 | CUUGAGACGCCUGAUAGAC |
| 913 | UCUAUCAGGCGUCUCAAGG | 5667 | CCUUGAGACGCCUGAUAGA |
| 966 | GUUGUCAGUGCUGCCCAAG | 5720 | CUUGGGCAGCACUGACAAC |
| 976 | CUGCCCAAGCAUCUUGUCU | 5730 | AGACAAGAUGCUUGGGCAG |
| 977 | UGCCCAAGCAUCUUGUCUC | 5731 | GAGACAAGAUGCUUGGGCA |
| 1001 | GUAUUGAAUACGUGCACAG | 5755 | CUGUGCACGUAUUCAAUAC |
| 1003 | AUUGAAUACGUGCACAGCU | 5757 | AGCUGUGCACGUAUUCAAU |
| 1004 | UUGAAUACGUGCACAGCUU | 5758 | AAGCUGUGCACGUAUUCAA |
| 1046 | UCCUGACUGUACAGCCGGC | 5800 | GCCGGCUGUACAGUCAGGA |
| 1049 | UGACUGUACAGCCGGCCAG | 5803 | CUGGCCGGCUGUACAGUCA |
| 1053 | UGUACAGCCGGCCAGCGUG | 5807 | CACGCUGGCCGGCUGUACA |
| 1054 | GUACAGCCGGCCAGCGUGA | 5808 | UCACGCUGGCCGGCUGUAC |
| 1055 | UACAGCCGGCCAGCGUGAC | 5809 | GUCACGCUGGCCGGCUGUA |
| 1129 | GAGUUGGGUGACUAUCGGG | 5883 | CCCGAUAGUCACCCAACUC |
| 1130 | AGUUGGGUGACUAUCGGGA | 5884 | UCCCGAUAGUCACCCAACU |
| 1131 | GUUGGGUGACUAUCGGGAG | 5885 | CUCCCGAUAGUCACCCAAC |
| 1132 | UUGGGUGACUAUCGGGAGC | 5886 | GCUCCCGAUAGUCACCCAA |
| 1134 | GGGUGACUAUCGGGAGCUG | 5888 | CAGCUCCCGAUAGUCACCC |
| 1138 | GACUAUCGGGAGCUGGUCC | 5892 | GGACCAGCUCCCGAUAGUC |
| 1146 | GGAGCUGGUCCUCGACUGC | 5900 | GCAGUCGAGGACCAGCUCC |
| 1147 | GAGCUGGUCCUCGACUGCA | 5901 | UGCAGUCGAGGACCAGCUC |
| 1152 | GGUCCUCGACUGCAGAUUU | 5906 | AAAUCUGCAGUCGAGGACC |
| 1153 | GUCCUCGACUGCAGAUUUG | 5907 | CAAAUCUGCAGUCGAGGAC |
| 1155 | CCUCGACUGCAGAUUUGCU | 5909 | AGCAAAUCUGCAGUCGAGG |
| 1157 | UCGACUGCAGAUUUGCUCC | 5911 | GGAGCAAAUCUGCAGUCGA |
| 1162 | UGCAGAUUUGCUCCAAAAC | 5916 | GUUUUGGAGCAAAUCUGCA |
| 1164 | CAGAUUUGCUCCAAAACGC | 5918 | GCGUUUUGGAGCAAAUCUG |
| 1166 | GAUUUGCUCCAAAACGCAG | 5920 | CUGCGUUUUGGAGCAAAUC |
| 1173 | UCCAAAACGCAGGCGCCGG | 5927 | CCGGCGCCUGCGUUUUGGA |
| 1350 | CAACUCUGUCGUCUGUGCC | 6104 | GGCACAGACGACAGAGUUG |
| 1408 | GUGGAGCGCUGUUGUGAAU | 6162 | AUUCACAACAGCGCUCCAC |
| 1409 | UGGAGCGCUGUUGUGAAUC | 6163 | GAUUCACAACAGCGCUCCA |
| 1443 | CCUCCGGCGAGGCCUCGAC | 6197 | GUCGAGGCCUCGCCGGAGG |
| 1444 | CUCCGGCGAGGCCUCGACU | 6198 | AGUCGAGGCCUCGCCGGAG |
| 1445 | UCCGGCGAGGCCUCGACUU | 6199 | AAGUCGAGGCCUCGCCGGA |
| 1446 | CCGGCGAGGCCUCGACUUC | 6200 | GAAGUCGAGGCCUCGCCGG |
| 1447 | CGGCGAGGCCUCGACUUCU | 6201 | AGAAGUCGAGGCCUCGCCG |
| 1857 | GACCUGUGGGCGUUGCCUA | 6611 | UAGGCAACGCCCACAGGUC |
| 1862 | GUGGGCGUUGCCUAAGGGC | 6616 | GCCCUUAGGCAACGCCCAC |
| 2125 | AAGGACAGCUCAAAACUCA | 6879 | UGAGUUUUGAGCUGUCCUU |
| 2143 | AGACCAGUGCCCCGGAAAG | 6897 | CUUUCCGGGGCACUGGUCU |
| 2151 | GCCCCGGAAAGACUUUGUA | 6905 | UACAAAGUCUUUCCGGGGC |
| 2154 | CCGGAAAGACUUUGUAGAG | 6908 | CUCUACAAAGUCUUUCCGG |
| 2208 | GGCAGUGGGGCCUACCAAC | 6962 | GUUGGUAGGCCCCACUGCC |
| 2226 | CGUCAGCCUCACCGUGACU | 6980 | AGUCACGGUGAGGCUGACG |
| 2228 | UCAGCCUCACCGUGACUAA | 6982 | UUAGUCACGGUGAGGCUGA |
| 2229 | CAGCCUCACCGUGACUAAC | 6983 | GUUAGUCACGGUGAGGCUG |
| 2230 | AGCCUCACCGUGACUAACA | 6984 | UGUUAGUCACGGUGAGGCU |
| 2232 | CCUCACCGUGACUAACAUG | 6986 | CAUGUUAGUCACGGUGAGG |
| 2233 | CUCACCGUGACUAACAUGC | 6987 | GCAUGUUAGUCACGGUGAG |
| 2234 | UCACCGUGACUAACAUGCC | 6988 | GGCAUGUUAGUCACGGUGA |
| 2240 | UGACUAACAUGCCACCGGG | 6994 | CCCGGUGGCAUGUUAGUCA |
| 2242 | ACUAACAUGCCACCGGGCA | 6996 | UGCCCGGUGGCAUGUUAGU |
| 2243 | CUAACAUGCCACCGGGCAA | 6997 | UUGCCCGGUGGCAUGUUAG |
| 2244 | UAACAUGCCACCGGGCAAG | 6998 | CUUGCCCGGUGGCAUGUUA |
| 2245 | AACAUGCCACCGGGCAAGC | 6999 | GCUUGCCCGGUGGCAUGUU |
| 2252 | CACCGGGCAAGCACUUCCG | 7006 | CGGAAGUGCUUGCCCGGUG |
| 2257 | GGCAAGCACUUCCGGGUAG | 7011 | CUACCCGGAAGUGCUUGCC |
| 2258 | GCAAGCACUUCCGGGUAGA | 7012 | UCUACCCGGAAGUGCUUGC |
| 2259 | CAAGCACUUCCGGGUAGAC | 7013 | GUCUACCCGGAAGUGCUUG |
| 2263 | CACUUCCGGGUAGACGGCA | 7017 | UGCCGUCUACCCGGAAGUG |
| 2264 | ACUUCCGGGUAGACGGCAC | 7018 | GUGCCGUCUACCCGGAAGU |
| 2265 | CUUCCGGGUAGACGGCACC | 7019 | GGUGCCGUCUACCCGGAAG |
| 2267 | UCCGGGUAGACGGCACCUC | 7021 | GAGGUGCCGUCUACCCGGA |
| 2366 | GUCUCACUCUUGAAGGCCA | 7120 | UGGCCUUCAAGAGUGAGAC |
| 2369 | UCACUCUUGAAGGCCAGAG | 7123 | CUCUGGCCUUCAAGAGUGA |
| 2418 | GGUCAAUGGGACUGAGUGU | 7172 | ACACUCAGUCCCAUUGACC |
| 2421 | CAAUGGGACUGAGUGUCUG | 7175 | CAGACACUCAGUCCCAUUG |
| 2430 | UGAGUGUCUGCUAGCACGG | 7184 | CCGUGCUAGCAGACACUCA |
| 2432 | AGUGUCUGCUAGCACGGGU | 7186 | ACCCGUGCUAGCAGACACU |
| 2435 | GUCUGCUAGCACGGGUCAG | 7189 | CUGACCCGUGCUAGCAGAC |
| 2438 | UGCUAGCACGGGUCAGUGA | 7192 | UCACUGACCCGUGCUAGCA |
| 2445 | ACGGGUCAGUGAGGGGCAG | 7199 | CUGCCCCUCACUGACCCGU |
| 2455 | GAGGGGCAGCUUUUAUGUG | 7209 | CACAUAAAAGCUGCCCCUC |
| 2571 | CCCUGUCGUGCUAAGCAUC | 7325 | GAUGCUUAGCACGACAGGG |
| 2572 | CCUGUCGUGCUAAGCAUCA | 7326 | UGAUGCUUAGCACGACAGG |
| 2628 | CUGUGGCCAGCAUCUAACU | 7382 | AGUUAGAUGCUGGCCACAG |
| 2629 | UGUGGCCAGCAUCUAACUU | 7383 | AAGUUAGAUGCUGGCCACA |
| 2644 | ACUUCAGCAUGGCACUUAG | 7398 | CUAAGUGCCAUGCUGAAGU |
| 2646 | UUCAGCAUGGCACUUAGUG | 7400 | CACUAAGUGCCAUGCUGAA |
| 2648 | CAGCAUGGCACUUAGUGCU | 7402 | AGCACUAAGUGCCAUGCUG |
| 2655 | GCACUUAGUGCUGUCAUUC | 7409 | GAAUGACAGCACUAAGUGC |
| 2656 | CACUUAGUGCUGUCAUUCC | 7410 | GGAAUGACAGCACUAAGUG |
| 2661 | AGUGCUGUCAUUCCAUGAC | 7415 | GUCAUGGAAUGACAGCACU |
| 2663 | UGCUGUCAUUCCAUGACGG | 7417 | CCGUCAUGGAAUGACAGCA |
| 2664 | GCUGUCAUUCCAUGACGGG | 7418 | CCCGUCAUGGAAUGACAGC |
| 2666 | UGUCAUUCCAUGACGGGCU | 7420 | AGCCCGUCAUGGAAUGACA |
| 2668 | UCAUUCCAUGACGGGCUUA | 7422 | UAAGCCCGUCAUGGAAUGA |
| 2669 | CAUUCCAUGACGGGCUUAG | 7423 | CUAAGCCCGUCAUGGAAUG |
| 2670 | AUUCCAUGACGGGCUUAGG | 7424 | CCUAAGCCCGUCAUGGAAU |
| 2671 | UUCCAUGACGGGCUUAGGG | 7425 | CCCUAAGCCCGUCAUGGAA |
| 2676 | UGACGGGCUUAGGGCAGUG | 7430 | CACUGCCCUAAGCCCGUCA |
| 2891 | AGGAGCAUGCCAUUAAGUU | 7645 | AACUUAAUGGCAUGCUCCU |
| 3186 | UGCACUGGUCUUCAGCUAC | 7940 | GUAGCUGAAGACCAGUGCA |
| 3191 | UGGUCUUCAGCUACUGGUG | 7945 | CACCAGUAGCUGAAGACCA |
| 3279 | CACACCCCUGCCUAUUCUG | 8033 | CAGAAUAGGCAGGGGUGUG |
| 3297 | GUACUCGGGCUCUGACUAC | 8051 | GUAGUCAGAGCCCGAGUAC |
| 3298 | UACUCGGGCUCUGACUACA | 8052 | UGUAGUCAGAGCCCGAGUA |
| 3299 | ACUCGGGCUCUGACUACAG | 8053 | CUGUAGUCAGAGCCCGAGU |
| 3560 | GAGAAUACAUAGACCAGGC | 8314 | GCCUGGUCUAUGUAUUCUC |
| 3570 | AGACCAGGCCCAGAAUCGA | 8324 | UCGAUUCUGGGCCUGGUCU |
| 3574 | CAGGCCCAGAAUCGAAUCC | 8328 | GGAUUCGAUUCUGGGCCUG |
| 3576 | GGCCCAGAAUCGAAUCCAA | 8330 | UUGGAUUCGAUUCUGGGCC |
| 3578 | CCCAGAAUCGAAUCCAAUG | 8332 | CAUUGGAUUCGAUUCUGGG |
| 3580 | CAGAAUCGAAUCCAAUGUG | 8334 | CACAUUGGAUUCGAUUCUG |
| 3581 | AGAAUCGAAUCCAAUGUGC | 8335 | GCACAUUGGAUUCGAUUCU |
| 3583 | AAUCGAAUCCAAUGUGCCA | 8337 | UGGCACAUUGGAUUCGAUU |
| 3584 | AUCGAAUCCAAUGUGCCAU | 8338 | AUGGCACAUUGGAUUCGAU |
| 3586 | CGAAUCCAAUGUGCCAUCA | 8340 | UGAUGGCACAUUGGAUUCG |
| 3588 | AAUCCAAUGUGCCAUCAAG | 8342 | CUUGAUGGCACAUUGGAUU |
| 3592 | CAAUGUGCCAUCAAGUCAC | 8346 | GUGACUUGAUGGCACAUUG |
| 3594 | AUGUGCCAUCAAGUCACUA | 8348 | UAGUGACUUGAUGGCACAU |
| 3597 | UGCCAUCAAGUCACUAAGU | 8351 | ACUUAGUGACUUGAUGGCA |
| 3600 | CAUCAAGUCACUAAGUCGC | 8354 | GCGACUUAGUGACUUGAUG |
| 3655 | GAGGGGCUGCUCAUGCGUG | 8409 | CACGCAUGAGCAGCCCCUC |
| 3656 | AGGGGCUGCUCAUGCGUGG | 8410 | CCACGCAUGAGCAGCCCCU |
| 3657 | GGGGCUGCUCAUGCGUGGC | 8411 | GCCACGCAUGAGCAGCCCC |
| 3659 | GGCUGCUCAUGCGUGGCCU | 8413 | AGGCCACGCAUGAGCAGCC |
| 3671 | GUGGCCUGAACCACCCGAA | 8425 | UUCGGGUGGUUCAGGCCAC |
| 3672 | UGGCCUGAACCACCCGAAU | 8426 | AUUCGGGUGGUUCAGGCCA |
| 3673 | GGCCUGAACCACCCGAAUG | 8427 | CAUUCGGGUGGUUCAGGCC |
| 3674 | GCCUGAACCACCCGAAUGU | 8428 | ACAUUCGGGUGGUUCAGGC |
| 3676 | CUGAACCACCCGAAUGUGC | 8430 | GCACAUUCGGGUGGUUCAG |
| 3685 | CCGAAUGUGCUGGCUCUCA | 8439 | UGAGAGCCAGCACAUUCGG |
| 3701 | UCAUUGGUAUCAUGUUGCC | 8455 | GGCAACAUGAUACCAAUGA |
| 3705 | UGGUAUCAUGUUGCCACCU | 8459 | AGGUGGCAACAUGAUACCA |
| 3748 | CCCUAUAUGUGCCACGGUG | 8502 | CACCGUGGCACAUAUAGGG |
| 3749 | CCUAUAUGUGCCACGGUGA | 8503 | UCACCGUGGCACAUAUAGG |
| 3750 | CUAUAUGUGCCACGGUGAC | 8504 | GUCACCGUGGCACAUAUAG |
| 3752 | AUAUGUGCCACGGUGACCU | 8506 | AGGUCACCGUGGCACAUAU |
| 3771 | GCUCCAGUUCAUCCGCUCA | 8525 | UGAGCGGAUGAACUGGAGC |
| 3773 | UCCAGUUCAUCCGCUCACC | 8527 | GGUGAGCGGAUGAACUGGA |
| 3775 | CAGUUCAUCCGCUCACCUC | 8529 | GAGGUGAGCGGAUGAACUG |
| 3777 | GUUCAUCCGCUCACCUCAG | 8531 | CUGAGGUGAGCGGAUGAAC |
| 3793 | CAGCGGAACCCCACCGUGA | 8547 | UCACGGUGGGGUUCCGCUG |
| 3794 | AGCGGAACCCCACCGUGAA | 8548 | UUCACGGUGGGGUUCCGCU |
| 3795 | GCGGAACCCCACCGUGAAG | 8549 | CUUCACGGUGGGGUUCCGC |
| 3799 | AACCCCACCGUGAAGGACC | 8553 | GGUCCUUCACGGUGGGGUU |
| 3800 | ACCCCACCGUGAAGGACCU | 8554 | AGGUCCUUCACGGUGGGGU |
| 3871 | AAGUUUGUGCACAGGGACC | 8625 | GGUCCCUGUGCACAAACUU |
| 4308 | GCUUGGGGACCAUUAUGUG | 9062 | CACAUAAUGGUCCCCAAGC |
| 4311 | UGGGGACCAUUAUGUGCAG | 9065 | CUGCACAUAAUGGUCCCCA |
| 4313 | GGGACCAUUAUGUGCAGCU | 9067 | AGCUGCACAUAAUGGUCCC |

The siRNAs in subset C have the following characteristics:
Cross-reactivity: With 19mer in human MST1R mRNA, with 17mer/19mer in NHP MST1R
Specificity category: For human and NHP: AS2 or better, SS3 or better
miRNA seeds: AS+SS strand: seed region not conserved in human, mouse, and rat and not present in >4 species. AS strand: seed region not identical to seed region of known human miRNA
Off-target frequency: ≤15 human off-targets matched with 2 mismatches by antisense strand
SNPs: siRNA target sites do not harbor SNPs with a MAF ≥1% (pos. 2-18)

The siRNA sequences in subset C were also selected for absence of seed regions in the AS or S strands that are identical to a seed region of known human miRNA to yield subset D. Subset D includes 121 siRNAs whose base sequences are shown in Table 6.

TABLE 6
Sequences in siRNA subset D

| SEQ ID | sense strand sequence | SEQ ID | antisense strand sequence |
|---|---|---|---|
| NO: | (5′-3′) | NO : | (5′-3′) |
| 471 | UGUGUUUGUAGCCAUACGC | 5225 | GCGUAUGGCUACAAACACA |
| 472 | GUGUUUGUAGCCAUACGCA | 5226 | UGCGUAUGGCUACAAACAC |
| 474 | GUUUGUAGCCAUACGCAAU | 5228 | AUUGCGUAUGGCUACAAAC |
| 476 | UUGUAGCCAUACGCAAUCG | 5230 | CGAUUGCGUAUGGCUACAA |
| 477 | UGUAGCCAUACGCAAUCGC | 5231 | GCGAUUGCGUAUGGCUACA |
| 480 | AGCCAUACGCAAUCGCCUG | 5234 | CAGGCGAUUGCGUAUGGCU |
| 482 | CCAUACGCAAUCGCCUGCA | 5236 | UGCAGGCGAUUGCGUAUGG |
| 692 | GCUUCCUGCAUGACCUAGA | 5446 | UCUAGGUCAUGCAGGAAGC |
| 717 | AGGGACAGCCGUGCAUCUG | 5471 | CAGAUGCACGGCUGUCCCU |
| 726 | CGUGCAUCUGGCAGCGCCA | 5480 | UGGCGCUGCCAGAUGCACG |
| 753 | CUUCUCAGCCCACCAUAAC | 5507 | GUUAUGGUGGGCUGAGAAG |
| 763 | CACCAUAACCGGCCCGAUG | 5517 | CAUCGGGCCGGUUAUGGUG |
| 764 | ACCAUAACCGGCCCGAUGA | 5518 | UCAUCGGGCCGGUUAUGGU |
| 765 | CCAUAACCGGCCCGAUGAC | 5519 | GUCAUCGGGCCGGUUAUGG |
| 766 | CAUAACCGGCCCGAUGACU | 5520 | AGUCAUCGGGCCGGUUAUG |
| 767 | AUAACCGGCCCGAUGACUG | 5521 | CAGUCAUCGGGCCGGUUAU |
| 775 | CCCGAUGACUGCCCCGACU | 5529 | AGUCGGGGCAGUCAUCGGG |
| 776 | CCGAUGACUGCCCCGACUG | 5530 | CAGUCGGGGCAGUCAUCGG |
| 777 | CGAUGACUGCCCCGACUGU | 5531 | ACAGUCGGGGCAGUCAUCG |
| 801 | CAGCCCAUUGGGCACCCGU | 5555 | ACGGGUGCCCAAUGGGCUG |
| 824 | CUGUGGUUGAGCAAGGCCA | 5578 | UGGCCUUGCUCAACCACAG |
| 845 | CCUCCUAUUUCUACGUGGC | 5599 | GCCACGUAGAAAUAGGAGG |
| 864 | AUCCUCACUGGACGCAGCC | 5618 | GGCUGCGUCCAGUGAGGAU |
| 898 | AGCCCACGCUCAGUGUCUA | 5652 | UAGACACUGAGCGUGGGCU |
| 900 | CCCACGCUCAGUGUCUAUC | 5654 | GAUAGACACUGAGCGUGGG |
| 901 | CCACGCUCAGUGUCUAUCA | 5655 | UGAUAGACACUGAGCGUGG |
| 903 | ACGCUCAGUGUCUAUCAGG | 5657 | CCUGAUAGACACUGAGCGU |
| 910 | GUGUCUAUCAGGCGUCUCA | 5664 | UGAGACGCCUGAUAGACAC |
| 912 | GUCUAUCAGGCGUCUCAAG | 5666 | CUUGAGACGCCUGAUAGAC |
| 913 | UCUAUCAGGCGUCUCAAGG | 5667 | CCUUGAGACGCCUGAUAGA |
| 948 | CGCACCGGGCUUUGUGGCG | 5702 | CGCCACAAAGCCCGGUGCG |
| 966 | GUUGUCAGUGCUGCCCAAG | 5720 | CUUGGGCAGCACUGACAAC |
| 976 | CUGCCCAAGCAUCUUGUCU | 5730 | AGACAAGAUGCUUGGGCAG |
| 977 | UGCCCAAGCAUCUUGUCUC | 5731 | GAGACAAGAUGCUUGGGCA |
| 990 | UGUCUCCUACAGUAUUGAA | 5744 | UUCAAUACUGUAGGAGACA |
| 1001 | GUAUUGAAUACGUGCACAG | 5755 | CUGUGCACGUAUUCAAUAC |
| 1003 | AUUGAAUACGUGCACAGCU | 5757 | AGCUGUGCACGUAUUCAAU |
| 1042 | UACUUCCUGACUGUACAGC | 5796 | GCUGUACAGUCAGGAAGUA |
| 1046 | UCCUGACUGUACAGCCGGC | 5800 | GCCGGCUGUACAGUCAGGA |
| 1049 | UGACUGUACAGCCGGCCAG | 5803 | CUGGCCGGCUGUACAGUCA |
| 1053 | UGUACAGCCGGCCAGCGUG | 5807 | CACGCUGGCCGGCUGUACA |
| 1054 | GUACAGCCGGCCAGCGUGA | 5808 | UCACGCUGGCCGGCUGUAC |
| 1124 | AGCCAGAGUUGGGUGACUA | 5878 | UAGUCACCCAACUCUGGCU |
| 1131 | GUUGGGUGACUAUCGGGAG | 5885 | CUCCCGAUAGUCACCCAAC |
| 1134 | GGGUGACUAUCGGGAGCUG | 5888 | CAGCUCCCGAUAGUCACCC |
| 1138 | GACUAUCGGGAGCUGGUCC | 5892 | GGACCAGCUCCCGAUAGUC |
| 1147 | GAGCUGGUCCUCGACUGCA | 5901 | UGCAGUCGAGGACCAGCUC |
| 1153 | GUCCUCGACUGCAGAUUUG | 5907 | CAAAUCUGCAGUCGAGGAC |
| 1155 | CCUCGACUGCAGAUUUGCU | 5909 | AGCAAAUCUGCAGUCGAGG |
| 1157 | UCGACUGCAGAUUUGCUCC | 5911 | GGAGCAAAUCUGCAGUCGA |
| 1162 | UGCAGAUUUGCUCCAAAAC | 5916 | GUUUUGGAGCAAAUCUGCA |
| 1164 | CAGAUUUGCUCCAAAACGC | 5918 | GCGUUUUGGAGCAAAUCUG |
| 1166 | GAUUUGCUCCAAAACGCAG | 5920 | CUGCGUUUUGGAGCAAAUC |
| 1173 | UCCAAAACGCAGGCGCCGG | 5927 | CCGGCGCCUGCGUUUUGGA |
| 1409 | UGGAGCGCUGUUGUGAAUC | 6163 | GAUUCACAACAGCGCUCCA |
| 1443 | CCUCCGGCGAGGCCUCGAC | 6197 | GUCGAGGCCUCGCCGGAGG |
| 1444 | CUCCGGCGAGGCCUCGACU | 6198 | AGUCGAGGCCUCGCCGGAG |
| 1445 | UCCGGCGAGGCCUCGACUU | 6199 | AAGUCGAGGCCUCGCCGGA |
| 1447 | CGGCGAGGCCUCGACUUCU | 6201 | AGAAGUCGAGGCCUCGCCG |
| 1857 | GACCUGUGGGCGUUGCCUA | 6611 | UAGGCAACGCCCACAGGUC |
| 2143 | AGACCAGUGCCCCGGAAAG | 6897 | CUUUCCGGGGCACUGGUCU |
| 2154 | CCGGAAAGACUUUGUAGAG | 6908 | CUCUACAAAGUCUUUCCGG |
| 2226 | CGUCAGCCUCACCGUGACU | 6980 | AGUCACGGUGAGGCUGACG |
| 2228 | UCAGCCUCACCGUGACUAA | 6982 | UUAGUCACGGUGAGGCUGA |
| 2230 | AGCCUCACCGUGACUAACA | 6984 | UGUUAGUCACGGUGAGGCU |
| 2233 | CUCACCGUGACUAACAUGC | 6987 | GCAUGUUAGUCACGGUGAG |
| 2234 | UCACCGUGACUAACAUGCC | 6988 | GGCAUGUUAGUCACGGUGA |
| 2242 | ACUAACAUGCCACCGGGCA | 6996 | UGCCCGGUGGCAUGUUAGU |
| 2244 | UAACAUGCCACCGGGCAAG | 6998 | CUUGCCCGGUGGCAUGUUA |
| 2245 | AACAUGCCACCGGGCAAGC | 6999 | GCUUGCCCGGUGGCAUGUU |
| 2257 | GGCAAGCACUUCCGGGUAG | 7011 | CUACCCGGAAGUGCUUGCC |
| 2258 | GCAAGCACUUCCGGGUAGA | 7012 | UCUACCCGGAAGUGCUUGC |
| 2263 | CACUUCCGGGUAGACGGCA | 7017 | UGCCGUCUACCCGGAAGUG |
| 2264 | ACUUCCGGGUAGACGGCAC | 7018 | GUGCCGUCUACCCGGAAGU |
| 2267 | UCCGGGUAGACGGCACCUC | 7021 | GAGGUGCCGUCUACCCGGA |
| 2418 | GGUCAAUGGGACUGAGUGU | 7172 | ACACUCAGUCCCAUUGACC |
| 2445 | ACGGGUCAGUGAGGGGCAG | 7199 | CUGCCCCUCACUGACCCGU |
| 2572 | CCUGUCGUGCUAAGCAUCA | 7326 | UGAUGCUUAGCACGACAGG |
| 2644 | ACUUCAGCAUGGCACUUAG | 7398 | CUAAGUGCCAUGCUGAAGU |
| 2648 | CAGCAUGGCACUUAGUGCU | 7402 | AGCACUAAGUGCCAUGCUG |
| 2655 | GCACUUAGUGCUGUCAUUC | 7409 | GAAUGACAGCACUAAGUGC |
| 2661 | AGUGCUGUCAUUCCAUGAC | 7415 | GUCAUGGAAUGACAGCACU |
| 2668 | UCAUUCCAUGACGGGCUUA | 7422 | UAAGCCCGUCAUGGAAUGA |
| 2669 | CAUUCCAUGACGGGCUUAG | 7423 | CUAAGCCCGUCAUGGAAUG |
| 2676 | UGACGGGCUUAGGGCAGUG | 7430 | CACUGCCCUAAGCCCGUCA |
| 3186 | UGCACUGGUCUUCAGCUAC | 7940 | GUAGCUGAAGACCAGUGCA |
| 3191 | UGGUCUUCAGCUACUGGUG | 7945 | CACCAGUAGCUGAAGACCA |
| 3279 | CACACCCCUGCCUAUUCUG | 8033 | CAGAAUAGGCAGGGGUGUG |
| 3297 | GUACUCGGGCUCUGACUAC | 8051 | GUAGUCAGAGCCCGAGUAC |
| 3298 | UACUCGGGCUCUGACUACA | 8052 | UGUAGUCAGAGCCCGAGUA |
| 3299 | ACUCGGGCUCUGACUACAG | 8053 | CUGUAGUCAGAGCCCGAGU |
| 3537 | CCACUUUGGAGUUGUCUAC | 8291 | GUAGACAACUCCAAAGUGG |
| 3538 | CACUUUGGAGUUGUCUACC | 8292 | GGUAGACAACUCCAAAGUG |
| 3560 | GAGAAUACAUAGACCAGGC | 8314 | GCCUGGUCUAUGUAUUCUC |
| 3570 | AGACCAGGCCCAGAAUCGA | 8324 | UCGAUUCUGGGCCUGGUCU |
| 3576 | GGCCCAGAAUCGAAUCCAA | 8330 | UUGGAUUCGAUUCUGGGCC |
| 3580 | CAGAAUCGAAUCCAAUGUG | 8334 | CACAUUGGAUUCGAUUCUG |
| 3581 | AGAAUCGAAUCCAAUGUGC | 8335 | GCACAUUGGAUUCGAUUCU |
| 3583 | AAUCGAAUCCAAUGUGCCA | 8337 | UGGCACAUUGGAUUCGAUU |
| 3584 | AUCGAAUCCAAUGUGCCAU | 8338 | AUGGCACAUUGGAUUCGAU |
| 3586 | CGAAUCCAAUGUGCCAUCA | 8340 | UGAUGGCACAUUGGAUUCG |
| 3588 | AAUCCAAUGUGCCAUCAAG | 8342 | CUUGAUGGCACAUUGGAUU |
| 3597 | UGCCAUCAAGUCACUAAGU | 8351 | ACUUAGUGACUUGAUGGCA |
| 3600 | CAUCAAGUCACUAAGUCGC | 8354 | GCGACUUAGUGACUUGAUG |
| 3659 | GGCUGCUCAUGCGUGGCCU | 8413 | AGGCCACGCAUGAGCAGCC |
| 3663 | GCUCAUGCGUGGCCUGAAC | 8417 | GUUCAGGCCACGCAUGAGC |
| 3673 | GGCCUGAACCACCCGAAUG | 8427 | CAUUCGGGUGGUUCAGGCC |
| 3676 | CUGAACCACCCGAAUGUGC | 8430 | GCACAUUCGGGUGGUUCAG |
| 3685 | CCGAAUGUGCUGGCUCUCA | 8439 | UGAGAGCCAGCACAUUCGG |
| 3701 | UCAUUGGUAUCAUGUUGCC | 8455 | GGCAACAUGAUACCAAUGA |
| 3705 | UGGUAUCAUGUUGCCACCU | 8459 | AGGUGGCAACAUGAUACCA |
| 3748 | CCCUAUAUGUGCCACGGUG | 8502 | CACCGUGGCACAUAUAGGG |
| 3749 | CCUAUAUGUGCCACGGUGA | 8503 | UCACCGUGGCACAUAUAGG |
| 3750 | CUAUAUGUGCCACGGUGAC | 8504 | GUCACCGUGGCACAUAUAG |
| 3752 | AUAUGUGCCACGGUGACCU | 8506 | AGGUCACCGUGGCACAUAU |
| 3773 | UCCAGUUCAUCCGCUCACC | 8527 | GGUGAGCGGAUGAACUGGA |
| 3777 | GUUCAUCCGCUCACCUCAG | 8531 | CUGAGGUGAGCGGAUGAAC |
| 3794 | AGCGGAACCCCACCGUGAA | 8548 | UUCACGGUGGGGUUCCGCU |
| 3795 | GCGGAACCCCACCGUGAAG | 8549 | CUUCACGGUGGGGUUCCGC |
| 3799 | AACCCCACCGUGAAGGACC | 8553 | GGUCCUUCACGGUGGGGUU |
| 3871 | AAGUUUGUGCACAGGGACC | 8625 | GGUCCCUGUGCACAAACUU |

The siRNAs in subset D have the following characteristics:
Cross-reactivity: With 19mer in human MST1R mRNA, with 17mer/19mer in NHP MST1R
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: ≤20 human off-targets matched with 2 mismatches by antisense strand
SNPs: siRNA target sites do not harbor SNPs with a MAF >1% (pos. 2-18)

The siRNA sequences in subset D were further selected for more stringent specificity to yield subset E. Subset E includes 112 siRNAs whose base sequences are shown in Table 7.

TABLE 7
Sequences in siRNA subset E

| SEQ ID | sense strand sequence | SEQ ID | antisense strand sequence |
|---|---|---|---|
| NO: | (5′-3′) | NO: | (5′-3′) |
| 471 | UGUGUUUGUAGCCAUACGC | 5225 | GCGUAUGGCUACAAACACA |
| 472 | GUGUUUGUAGCCAUACGCA | 5226 | UGCGUAUGGCUACAAACAC |
| 474 | GUUUGUAGCCAUACGCAAU | 5228 | AUUGCGUAUGGCUACAAAC |
| 476 | UUGUAGCCAUACGCAAUCG | 5230 | CGAUUGCGUAUGGCUACAA |
| 477 | UGUAGCCAUACGCAAUCGC | 5231 | GCGAUUGCGUAUGGCUACA |
| 480 | AGCCAUACGCAAUCGCCUG | 5234 | CAGGCGAUUGCGUAUGGCU |
| 482 | CCAUACGCAAUCGCCUGCA | 5236 | UGCAGGCGAUUGCGUAUGG |
| 692 | GCUUCCUGCAUGACCUAGA | 5446 | UCUAGGUCAUGCAGGAAGC |
| 717 | AGGGACAGCCGUGCAUCUG | 5471 | CAGAUGCACGGCUGUCCCU |
| 726 | CGUGCAUCUGGCAGCGCCA | 5480 | UGGCGCUGCCAGAUGCACG |
| 753 | CUUCUCAGCCCACCAUAAC | 5507 | GUUAUGGUGGGCUGAGAAG |
| 763 | CACCAUAACCGGCCCGAUG | 5517 | CAUCGGGCCGGUUAUGGUG |
| 764 | ACCAUAACCGGCCCGAUGA | 5518 | UCAUCGGGCCGGUUAUGGU |
| 765 | CCAUAACCGGCCCGAUGAC | 5519 | GUCAUCGGGCCGGUUAUGG |
| 766 | CAUAACCGGCCCGAUGACU | 5520 | AGUCAUCGGGCCGGUUAUG |
| 767 | AUAACCGGCCCGAUGACUG | 5521 | CAGUCAUCGGGCCGGUUAU |
| 775 | CCCGAUGACUGCCCCGACU | 5529 | AGUCGGGGCAGUCAUCGGG |
| 776 | CCGAUGACUGCCCCGACUG | 5530 | CAGUCGGGGCAGUCAUCGG |
| 777 | CGAUGACUGCCCCGACUGU | 5531 | ACAGUCGGGGCAGUCAUCG |
| 801 | CAGCCCAUUGGGCACCCGU | 5555 | ACGGGUGCCCAAUGGGCUG |
| 845 | CCUCCUAUUUCUACGUGGC | 5599 | GCCACGUAGAAAUAGGAGG |
| 898 | AGCCCACGCUCAGUGUCUA | 5652 | UAGACACUGAGCGUGGGCU |
| 900 | CCCACGCUCAGUGUCUAUC | 5654 | GAUAGACACUGAGCGUGGG |
| 901 | CCACGCUCAGUGUCUAUCA | 5655 | UGAUAGACACUGAGCGUGG |
| 903 | ACGCUCAGUGUCUAUCAGG | 5657 | CCUGAUAGACACUGAGCGU |
| 910 | GUGUCUAUCAGGCGUCUCA | 5664 | UGAGACGCCUGAUAGACAC |
| 912 | GUCUAUCAGGCGUCUCAAG | 5666 | CUUGAGACGCCUGAUAGAC |
| 913 | UCUAUCAGGCGUCUCAAGG | 5667 | CCUUGAGACGCCUGAUAGA |
| 966 | GUUGUCAGUGCUGCCCAAG | 5720 | CUUGGGCAGCACUGACAAC |
| 976 | CUGCCCAAGCAUCUUGUCU | 5730 | AGACAAGAUGCUUGGGCAG |
| 977 | UGCCCAAGCAUCUUGUCUC | 5731 | GAGACAAGAUGCUUGGGCA |
| 1001 | GUAUUGAAUACGUGCACAG | 5755 | CUGUGCACGUAUUCAAUAC |
| 1003 | AUUGAAUACGUGCACAGCU | 5757 | AGCUGUGCACGUAUUCAAU |
| 1046 | UCCUGACUGUACAGCCGGC | 5800 | GCCGGCUGUACAGUCAGGA |
| 1049 | UGACUGUACAGCCGGCCAG | 5803 | CUGGCCGGCUGUACAGUCA |
| 1053 | UGUACAGCCGGCCAGCGUG | 5807 | CACGCUGGCCGGCUGUACA |
| 1054 | GUACAGCCGGCCAGCGUGA | 5808 | UCACGCUGGCCGGCUGUAC |
| 1131 | GUUGGGUGACUAUCGGGAG | 5885 | CUCCCGAUAGUCACCCAAC |
| 1134 | GGGUGACUAUCGGGAGCUG | 5888 | CAGCUCCCGAUAGUCACCC |
| 1138 | GACUAUCGGGAGCUGGUCC | 5892 | GGACCAGCUCCCGAUAGUC |
| 1147 | GAGCUGGUCCUCGACUGCA | 5901 | UGCAGUCGAGGACCAGCUC |
| 1153 | GUCCUCGACUGCAGAUUUG | 5907 | CAAAUCUGCAGUCGAGGAC |
| 1155 | CCUCGACUGCAGAUUUGCU | 5909 | AGCAAAUCUGCAGUCGAGG |
| 1157 | UCGACUGCAGAUUUGCUCC | 5911 | GGAGCAAAUCUGCAGUCGA |
| 1162 | UGCAGAUUUGCUCCAAAAC | 5916 | GUUUUGGAGCAAAUCUGCA |
| 1164 | CAGAUUUGCUCCAAAACGC | 5918 | GCGUUUUGGAGCAAAUCUG |
| 1166 | GAUUUGCUCCAAAACGCAG | 5920 | CUGCGUUUUGGAGCAAAUC |
| 1173 | UCCAAAACGCAGGCGCCGG | 5927 | CCGGCGCCUGCGUUUUGGA |
| 1409 | UGGAGCGCUGUUGUGAAUC | 6163 | GAUUCACAACAGCGCUCCA |
| 1443 | CCUCCGGCGAGGCCUCGAC | 6197 | GUCGAGGCCUCGCCGGAGG |
| 1444 | CUCCGGCGAGGCCUCGACU | 6198 | AGUCGAGGCCUCGCCGGAG |
| 1445 | UCCGGCGAGGCCUCGACUU | 6199 | AAGUCGAGGCCUCGCCGGA |
| 1447 | CGGCGAGGCCUCGACUUCU | 6201 | AGAAGUCGAGGCCUCGCCG |
| 1857 | GACCUGUGGGCGUUGCCUA | 6611 | UAGGCAACGCCCACAGGUC |
| 2143 | AGACCAGUGCCCCGGAAAG | 6897 | CUUUCCGGGGCACUGGUCU |
| 2154 | CCGGAAAGACUUUGUAGAG | 6908 | CUCUACAAAGUCUUUCCGG |
| 2226 | CGUCAGCCUCACCGUGACU | 6980 | AGUCACGGUGAGGCUGACG |
| 2228 | UCAGCCUCACCGUGACUAA | 6982 | UUAGUCACGGUGAGGCUGA |
| 2230 | AGCCUCACCGUGACUAACA | 6984 | UGUUAGUCACGGUGAGGCU |
| 2233 | CUCACCGUGACUAACAUGC | 6987 | GCAUGUUAGUCACGGUGAG |
| 2234 | UCACCGUGACUAACAUGCC | 6988 | GGCAUGUUAGUCACGGUGA |
| 2242 | ACUAACAUGCCACCGGGCA | 6996 | UGCCCGGUGGCAUGUUAGU |
| 2244 | UAACAUGCCACCGGGCAAG | 6998 | CUUGCCCGGUGGCAUGUUA |
| 2245 | AACAUGCCACCGGGCAAGC | 6999 | GCUUGCCCGGUGGCAUGUU |
| 2257 | GGCAAGCACUUCCGGGUAG | 7011 | CUACCCGGAAGUGCUUGCC |
| 2258 | GCAAGCACUUCCGGGUAGA | 7012 | UCUACCCGGAAGUGCUUGC |
| 2263 | CACUUCCGGGUAGACGGCA | 7017 | UGCCGUCUACCCGGAAGUG |
| 2264 | ACUUCCGGGUAGACGGCAC | 7018 | GUGCCGUCUACCCGGAAGU |
| 2267 | UCCGGGUAGACGGCACCUC | 7021 | GAGGUGCCGUCUACCCGGA |
| 2418 | GGUCAAUGGGACUGAGUGU | 7172 | ACACUCAGUCCCAUUGACC |
| 2445 | ACGGGUCAGUGAGGGGCAG | 7199 | CUGCCCCUCACUGACCCGU |
| 2572 | CCUGUCGUGCUAAGCAUCA | 7326 | UGAUGCUUAGCACGACAGG |
| 2644 | ACUUCAGCAUGGCACUUAG | 7398 | CUAAGUGCCAUGCUGAAGU |
| 2648 | CAGCAUGGCACUUAGUGCU | 7402 | AGCACUAAGUGCCAUGCUG |
| 2655 | GCACUUAGUGCUGUCAUUC | 7409 | GAAUGACAGCACUAAGUGC |
| 2661 | AGUGCUGUCAUUCCAUGAC | 7415 | GUCAUGGAAUGACAGCACU |
| 2668 | UCAUUCCAUGACGGGCUUA | 7422 | UAAGCCCGUCAUGGAAUGA |
| 2669 | CAUUCCAUGACGGGCUUAG | 7423 | CUAAGCCCGUCAUGGAAUG |
| 2676 | UGACGGGCUUAGGGCAGUG | 7430 | CACUGCCCUAAGCCCGUCA |
| 3186 | UGCACUGGUCUUCAGCUAC | 7940 | GUAGCUGAAGACCAGUGCA |
| 3191 | UGGUCUUCAGCUACUGGUG | 7945 | CACCAGUAGCUGAAGACCA |
| 3279 | CACACCCCUGCCUAUUCUG | 8033 | CAGAAUAGGCAGGGGUGUG |
| 3297 | GUACUCGGGCUCUGACUAC | 8051 | GUAGUCAGAGCCCGAGUAC |
| 3298 | UACUCGGGCUCUGACUACA | 8052 | UGUAGUCAGAGCCCGAGUA |
| 3299 | ACUCGGGCUCUGACUACAG | 8053 | CUGUAGUCAGAGCCCGAGU |
| 3560 | GAGAAUACAUAGACCAGGC | 8314 | GCCUGGUCUAUGUAUUCUC |
| 3570 | AGACCAGGCCCAGAAUCGA | 8324 | UCGAUUCUGGGCCUGGUCU |
| 3576 | GGCCCAGAAUCGAAUCCAA | 8330 | UUGGAUUCGAUUCUGGGCC |
| 3580 | CAGAAUCGAAUCCAAUGUG | 8334 | CACAUUGGAUUCGAUUCUG |
| 3581 | AGAAUCGAAUCCAAUGUGC | 8335 | GCACAUUGGAUUCGAUUCU |
| 3583 | AAUCGAAUCCAAUGUGCCA | 8337 | UGGCACAUUGGAUUCGAUU |
| 3584 | AUCGAAUCCAAUGUGCCAU | 8338 | AUGGCACAUUGGAUUCGAU |
| 3586 | CGAAUCCAAUGUGCCAUCA | 8340 | UGAUGGCACAUUGGAUUCG |
| 3588 | AAUCCAAUGUGCCAUCAAG | 8342 | CUUGAUGGCACAUUGGAUU |
| 3597 | UGCCAUCAAGUCACUAAGU | 8351 | ACUUAGUGACUUGAUGGCA |
| 3600 | CAUCAAGUCACUAAGUCGC | 8354 | GCGACUUAGUGACUUGAUG |
| 3659 | GGCUGCUCAUGCGUGGCCU | 8413 | AGGCCACGCAUGAGCAGCC |
| 3673 | GGCCUGAACCACCCGAAUG | 8427 | CAUUCGGGUGGUUCAGGCC |
| 3676 | CUGAACCACCCGAAUGUGC | 8430 | GCACAUUCGGGUGGUUCAG |
| 3685 | CCGAAUGUGCUGGCUCUCA | 8439 | UGAGAGCCAGCACAUUCGG |
| 3701 | UCAUUGGUAUCAUGUUGCC | 8455 | GGCAACAUGAUACCAAUGA |
| 3705 | UGGUAUCAUGUUGCCACCU | 8459 | AGGUGGCAACAUGAUACCA |
| 3748 | CCCUAUAUGUGCCACGGUG | 8502 | CACCGUGGCACAUAUAGGG |
| 3749 | CCUAUAUGUGCCACGGUGA | 8503 | UCACCGUGGCACAUAUAGG |
| 3750 | CUAUAUGUGCCACGGUGAC | 8504 | GUCACCGUGGCACAUAUAG |
| 3752 | AUAUGUGCCACGGUGACCU | 8506 | AGGUCACCGUGGCACAUAU |
| 3773 | UCCAGUUCAUCCGCUCACC | 8527 | GGUGAGCGGAUGAACUGGA |
| 3777 | GUUCAUCCGCUCACCUCAG | 8531 | CUGAGGUGAGCGGAUGAAC |
| 3794 | AGCGGAACCCCACCGUGAA | 8548 | UUCACGGUGGGGUUCCGCU |
| 3795 | GCGGAACCCCACCGUGAAG | 8549 | CUUCACGGUGGGGUUCCGC |
| 3799 | AACCCCACCGUGAAGGACC | 8553 | GGUCCUUCACGGUGGGGUU |
| 3871 | AAGUUUGUGCACAGGGACC | 8625 | GGUCCCUGUGCACAAACUU |

The siRNAs in subset E have the following characteristics:
Cross-reactivity: With 19mer in human MST1R mRNA, with 17mer/19mer in NHP MST1R
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: ≤15 human off-targets matched with 2 mismatches by antisense strand
SNPs: siRNA target sites do not harbor SNPs with a MAF >1% (pos. 2-18)

Subset F includes 103 siRNAs. The siRNAs in subset F include siRNAs from subset A, and are included in Table 8. In some cases, the sense strand of any of the siRNAs of subset F comprises modification pattern 6S (Table 9). In some cases, the antisense strand of any of the siRNAs of subset F comprises modification pattern 7AS (Table 9). In some cases, the sense strand of any of the siRNAs of subset F contains an alternative modification pattern (Table 10). In some cases, the antisense strand of any of the siRNAs of subset F comprises modification pattern 7AS (Table 10). The siRNAs in subset F may comprise any other modification pattern(s). In Table 9 and Table 10, Nf (e.g. Af, Cf, Gf, Tf, or Uf) is a 2′ fluoro-modified nucleoside, n (e.g. a, c, g, t, or u) is a 2′ O-methyl modified nucleoside, and “s” is a phosphorothioate linkage.

TABLE 8
Sequences in siRNA subset F

| SEQ ID | sense strand sequence | SEQ ID | antisense strand sequence |
|---|---|---|---|
| NO: | (5′-3′) | NO : | (5′-3′) |
| 469 | GCUGUGUUUGUAGCCAUAC | 5223 | GUAUGGCUACAAACACAGC |
| 471 | UGUGUUUGUAGCCAUACGC | 5225 | GCGUAUGGCUACAAACACA |
| 475 | UUUGUAGCCAUACGCAAUC | 5229 | GAUUGCGUAUGGCUACAAA |
| 476 | UUGUAGCCAUACGCAAUCG | 5230 | CGAUUGCGUAUGGCUACAA |
| 477 | UGUAGCCAUACGCAAUCGC | 5231 | GCGAUUGCGUAUGGCUACA |
| 483 | CAUACGCAAUCGCCUGCAU | 5237 | AUGCAGGCGAUUGCGUAUG |
| 692 | GCUUCCUGCAUGACCUAGA | 5446 | UCUAGGUCAUGCAGGAAGC |
| 693 | CUUCCUGCAUGACCUAGAG | 5447 | CUCUAGGUCAUGCAGGAAG |
| 753 | CUUCUCAGCCCACCAUAAC | 5507 | GUUAUGGUGGGCUGAGAAG |
| 845 | CCUCCUAUUUCUACGUGGC | 5599 | GCCACGUAGAAAUAGGAGG |
| 901 | CCACGCUCAGUGUCUAUCA | 5655 | UGAUAGACACUGAGCGUGG |
| 908 | CAGUGUCUAUCAGGCGUCU | 5662 | AGACGCCUGAUAGACACUG |
| 910 | GUGUCUAUCAGGCGUCUCA | 5664 | UGAGACGCCUGAUAGACAC |
| 911 | UGUCUAUCAGGCGUCUCAA | 5665 | UUGAGACGCCUGAUAGACA |
| 912 | GUCUAUCAGGCGUCUCAAG | 5666 | CUUGAGACGCCUGAUAGAC |
| 915 | UAUCAGGCGUCUCAAGGCU | 5669 | AGCCUUGAGACGCCUGAUA |
| 977 | UGCCCAAGCAUCUUGUCUC | 5731 | GAGACAAGAUGCUUGGGCA |
| 985 | CAUCUUGUCUCCUACAGUA | 5739 | UACUGUAGGAGACAAGAUG |
| 989 | UUGUCUCCUACAGUAUUGA | 5743 | UCAAUACUGUAGGAGACAA |
| 990 | UGUCUCCUACAGUAUUGAA | 5744 | UUCAAUACUGUAGGAGACA |
| 992 | UCUCCUACAGUAUUGAAUA | 5746 | UAUUCAAUACUGUAGGAGA |
| 1001 | GUAUUGAAUACGUGCACAG | 5755 | CUGUGCACGUAUUCAAUAC |
| 1003 | AUUGAAUACGUGCACAGCU | 5757 | AGCUGUGCACGUAUUCAAU |
| 1004 | UUGAAUACGUGCACAGCUU | 5758 | AAGCUGUGCACGUAUUCAA |
| 1006 | GAAUACGUGCACAGCUUCC | 5760 | GGAAGCUGUGCACGUAUUC |
| 1043 | ACUUCCUGACUGUACAGCC | 5797 | GGCUGUACAGUCAGGAAGU |
| 1124 | AGCCAGAGUUGGGUGACUA | 5878 | UAGUCACCCAACUCUGGCU |
| 1152 | GGUCCUCGACUGCAGAUUU | 5906 | AAAUCUGCAGUCGAGGACC |
| 1153 | GUCCUCGACUGCAGAUUUG | 5907 | CAAAUCUGCAGUCGAGGAC |
| 1154 | UCCUCGACUGCAGAUUUGC | 5908 | GCAAAUCUGCAGUCGAGGA |
| 1155 | CCUCGACUGCAGAUUUGCU | 5909 | AGCAAAUCUGCAGUCGAGG |
| 1158 | CGACUGCAGAUUUGCUCCA | 5912 | UGGAGCAAAUCUGCAGUCG |
| 1161 | CUGCAGAUUUGCUCCAAAA | 5915 | UUUUGGAGCAAAUCUGCAG |
| 1162 | UGCAGAUUUGCUCCAAAAC | 5916 | GUUUUGGAGCAAAUCUGCA |
| 1164 | CAGAUUUGCUCCAAAACGC | 5918 | GCGUUUUGGAGCAAAUCUG |
| 1408 | GUGGAGCGCUGUUGUGAAU | 6162 | AUUCACAACAGCGCUCCAC |
| 1409 | UGGAGCGCUGUUGUGAAUC | 6163 | GAUUCACAACAGCGCUCCA |
| 2124 | CAAGGACAGCUCAAAACUC | 6878 | GAGUUUUGAGCUGUCCUUG |
| 2150 | UGCCCCGGAAAGACUUUGU | 6904 | ACAAAGUCUUUCCGGGGCA |
| 2151 | GCCCCGGAAAGACUUUGUA | 6905 | UACAAAGUCUUUCCGGGGC |
| 2152 | CCCCGGAAAGACUUUGUAG | 6906 | CUACAAAGUCUUUCCGGGG |
| 2153 | CCCGGAAAGACUUUGUAGA | 6907 | UCUACAAAGUCUUUCCGGG |
| 2165 | UUGUAGAGGAGUUUGAGUG | 6919 | CACUCAAACUCCUCUACAA |
| 2230 | AGCCUCACCGUGACUAACA | 6984 | UGUUAGUCACGGUGAGGCU |
| 2233 | CUCACCGUGACUAACAUGC | 6987 | GCAUGUUAGUCACGGUGAG |
| 2234 | UCACCGUGACUAACAUGCC | 6988 | GGCAUGUUAGUCACGGUGA |
| 2235 | CACCGUGACUAACAUGCCA | 6989 | UGGCAUGUUAGUCACGGUG |
| 2318 | UGCUGAUAGCAGUGCAACC | 7072 | GGUUGCACUGCUAUCAGCA |
| 2366 | GUCUCACUCUUGAAGGCCA | 7120 | UGGCCUUCAAGAGUGAGAC |
| 2369 | UCACUCUUGAAGGCCAGAG | 7123 | CUCUGGCCUUCAAGAGUGA |
| 2412 | UGUGCUGGUCAAUGGGACU | 7166 | AGUCCCAUUGACCAGCACA |
| 2418 | GGUCAAUGGGACUGAGUGU | 7172 | ACACUCAGUCCCAUUGACC |
| 2420 | UCAAUGGGACUGAGUGUCU | 7174 | AGACACUCAGUCCCAUUGA |
| 2421 | CAAUGGGACUGAGUGUCUG | 7175 | CAGACACUCAGUCCCAUUG |
| 2427 | GACUGAGUGUCUGCUAGCA | 7181 | UGCUAGCAGACACUCAGUC |
| 2455 | GAGGGGCAGCUUUUAUGUG | 7209 | CACAUAAAAGCUGCCCCUC |
| 2460 | GCAGCUUUUAUGUGCCACA | 7214 | UGUGGCACAUAAAAGCUGC |
| 2462 | AGCUUUUAUGUGCCACACC | 7216 | GGUGUGGCACAUAAAAGCU |
| 2566 | GAAGACCCUGUCGUGCUAA | 7320 | UUAGCACGACAGGGUCUUC |
| 2567 | AAGACCCUGUCGUGCUAAG | 7321 | CUUAGCACGACAGGGUCUU |
| 2581 | CUAAGCAUCAGCCCCAACU | 7335 | AGUUGGGGCUGAUGCUUAG |
| 2582 | UAAGCAUCAGCCCCAACUG | 7336 | CAGUUGGGGCUGAUGCUUA |
| 2628 | CUGUGGCCAGCAUCUAACU | 7382 | AGUUAGAUGCUGGCCACAG |
| 2644 | ACUUCAGCAUGGCACUUAG | 7398 | CUAAGUGCCAUGCUGAAGU |
| 2646 | UUCAGCAUGGCACUUAGUG | 7400 | CACUAAGUGCCAUGCUGAA |
| 2648 | CAGCAUGGCACUUAGUGCU | 7402 | AGCACUAAGUGCCAUGCUG |
| 2656 | CACUUAGUGCUGUCAUUCC | 7410 | GGAAUGACAGCACUAAGUG |
| 2663 | UGCUGUCAUUCCAUGACGG | 7417 | CCGUCAUGGAAUGACAGCA |
| 2666 | UGUCAUUCCAUGACGGGCU | 7420 | AGCCCGUCAUGGAAUGACA |
| 2669 | CAUUCCAUGACGGGCUUAG | 7423 | CUAAGCCCGUCAUGGAAUG |
| 2670 | AUUCCAUGACGGGCUUAGG | 7424 | CCUAAGCCCGUCAUGGAAU |
| 2889 | UGAGGAGCAUGCCAUUAAG | 7643 | CUUAAUGGCAUGCUCCUCA |
| 2895 | GCAUGCCAUUAAGUUUGAG | 7649 | CUCAAACUUAAUGGCAUGC |
| 3084 | UAUCCUGGGUAGAGUGGUG | 7838 | CACCACUCUACCCAGGAUA |
| 3191 | UGGUCUUCAGCUACUGGUG | 7945 | CACCAGUAGCUGAAGACCA |
| 3223 | CUAGUUCUUCCUCCCAACC | 7977 | GGUUGGGAGGAAGAACUAG |
| 3298 | UACUCGGGCUCUGACUACA | 8052 | UGUAGUCAGAGCCCGAGUA |
| 3538 | CACUUUGGAGUUGUCUACC | 8292 | GGUAGACAACUCCAAAGUG |
| 3560 | GAGAAUACAUAGACCAGGC | 8314 | GCCUGGUCUAUGUAUUCUC |
| 3568 | AUAGACCAGGCCCAGAAUC | 8322 | GAUUCUGGGCCUGGUCUAU |
| 3576 | GGCCCAGAAUCGAAUCCAA | 8330 | UUGGAUUCGAUUCUGGGCC |
| 3578 | CCCAGAAUCGAAUCCAAUG | 8332 | CAUUGGAUUCGAUUCUGGG |
| 3580 | CAGAAUCGAAUCCAAUGUG | 8334 | CACAUUGGAUUCGAUUCUG |
| 3581 | AGAAUCGAAUCCAAUGUGC | 8335 | GCACAUUGGAUUCGAUUCU |
| 3582 | GAAUCGAAUCCAAUGUGCC | 8336 | GGCACAUUGGAUUCGAUUC |
| 3586 | CGAAUCCAAUGUGCCAUCA | 8340 | UGAUGGCACAUUGGAUUCG |
| 3587 | GAAUCCAAUGUGCCAUCAA | 8341 | UUGAUGGCACAUUGGAUUC |
| 3592 | CAAUGUGCCAUCAAGUCAC | 8346 | GUGACUUGAUGGCACAUUG |
| 3595 | UGUGCCAUCAAGUCACUAA | 8349 | UUAGUGACUUGAUGGCACA |
| 3596 | GUGCCAUCAAGUCACUAAG | 8350 | CUUAGUGACUUGAUGGCAC |
| 3597 | UGCCAUCAAGUCACUAAGU | 8351 | ACUUAGUGACUUGAUGGCA |
| 3600 | CAUCAAGUCACUAAGUCGC | 8354 | GCGACUUAGUGACUUGAUG |
| 3701 | UCAUUGGUAUCAUGUUGCC | 8455 | GGCAACAUGAUACCAAUGA |
| 3749 | CCUAUAUGUGCCACGGUGA | 8503 | UCACCGUGGCACAUAUAGG |
| 3750 | CUAUAUGUGCCACGGUGAC | 8504 | GUCACCGUGGCACAUAUAG |
| 3751 | UAUAUGUGCCACGGUGACC | 8505 | GGUCACCGUGGCACAUAUA |
| 3752 | AUAUGUGCCACGGUGACCU | 8506 | AGGUCACCGUGGCACAUAU |
| 3871 | AAGUUUGUGCACAGGGACC | 8625 | GGUCCCUGUGCACAAACUU |
| 4284 | GGUGGAGCAGAUAGUGUCU | 9038 | AGACACUAUCUGCUCCACC |
| 4292 | AGAUAGUGUCUGCACUGCU | 9046 | AGCAGUGCAGACACUAUCU |
| 4311 | UGGGGACCAUUAUGUGCAG | 9065 | CUGCACAUAAUGGUCCCCA |
| 4313 | GGGACCAUUAUGUGCAGCU | 9067 | AGCUGCACAUAAUGGUCCC |
| 4333 | CCAGCAACCUACAUGAACU | 9087 | AGUUCAUGUAGGUUGCUGG |

TABLE 9
Modified siRNA subset F sequences (a.k.a. subset G)

| | SEQ | | SEQ | |
|---|---|---|---|---|
| siRNA | ID | | ID | |
| Name | NO: | Sense strand sequence (5′-3′) | NO: | Antisense strand sequence (5′-3′) |
| siRNA | 9509 | GfscsUfgUfgUfuUfgUfaGfcCfaUfaAfsusu | 9612 | usUfsaUfgGfcUfaCfaAfaCfaCfaGfcsusu |
| 9509 | | | | |
| siRNA | 9510 | UfsgsUfgUfuUfgUfaGfcCfaUfaCfgAfsusu | 9613 | usCfsgUfaUfgGfcUfaCfaAfaCfaCfasusu |
| 9510 | | | | |
| siRNA | 9511 | UfsusUfgUfaGfcCfaUfaCfgCfaAfuAfsusu | 9614 | usAfsuUfgCfgUfaUfgGfcUfaCfaAfasusu |
| 9511 | | | | |
| siRNA | 9512 | UfsusGfuAfgCfcAfuAfcGfcAfaUfcAfsusu | 9615 | usGfsaUfuGfcGfuAfuGfgCfuAfcAfasusu |
| 9512 | | | | |
| siRNA | 9513 | UfsgsUfaGfcCfaUfaCfgCfaAfuCfgAfsusu | 9616 | usCfsgAfuUfgCfgUfaUfgGfcUfaCfasusu |
| 9513 | | | | |
| siRNA | 9514 | CfsasUfaCfgCfaAfuCfgCfcUfgCfaAfsusu | 9617 | usUfsgCfaGfgCfgAfuUfgCfgUfaUfgsusu |
| 9514 | | | | |
| siRNA | 9515 | GfscsUfuCfcUfgCfaUfgAfcCfuAfgAfsusu | 9618 | usCfsuAfgGfuCfaUfgCfaGfgAfaGfcsusu |
| 9515 | | | | |
| siRNA | 9516 | CfsusUfcCfuGfcAfuGfaCfcUfaGfaAfsusu | 9619 | usUfscUfaGfgUfcAfuGfcAfgGfaAfgsusu |
| 9516 | | | | |
| siRNA | 9517 | CfsusUfcUfcAfgCfcCfaCfcAfuAfaAfsusu | 9620 | usUfsuAfuGfgUfgGfgCfuGfaGfaAfgsusu |
| 9517 | | | | |
| siRNA | 9518 | CfscsUfcCfuAfuUfuCfuAfcGfuGfgAfsusu | 9621 | usCfscAfcGfuAfgAfaAfuAfgGfaGfgsusu |
| 9518 | | | | |
| siRNA | 9519 | CfscsAfcGfcUfcAfgUfgUfcUfaUfcAfsusu | 9622 | usGfsaUfaGfaCfaCfuGfaGfcGfuGfgsusu |
| 9519 | | | | |
| siRNA | 9520 | CfsasGfuGfuCfuAfuCfaGfgCfgUfcAfsusu | 9623 | usGfsaCfgCfcUfgAfuAfgAfcAfcUfgsusu |
| 9520 | | | | |
| siRNA | 9521 | GfsusGfuCfuAfuCfaGfgCfgUfcUfcAfsusu | 9624 | usGfsaGfaCfgCfcUfgAfuAfgAfcAfcsusu |
| 9521 | | | | |
| siRNA | 9522 | UfsgsUfcUfaUfcAfgGfcGfuCfuCfaAfsusu | 9625 | usUfsgAfgAfcGfcCfuGfaUfaGfaCfasusu |
| 9522 | | | | |
| siRNA | 9523 | GfsusCfuAfuCfaGfgCfgUfcUfcAfaAfsusu | 9626 | usUfsuGfaGfaCfgCfcUfgAfuAfgAfcsusu |
| 9523 | | | | |
| siRNA | 9524 | UfsasUfcAfgGfcGfuCfuCfaAfgGfcAfsusu | 9627 | usGfscCfuUfgAfgAfcGfcCfuGfaUfasusu |
| 9524 | | | | |
| siRNA | 9525 | UfsgsCfcCfaAfgCfaUfcUfuGfuCfuAfsusu | 9628 | usAfsgAfcAfaGfaUfgCfuUfgGfgCfasusu |
| 9525 | | | | |
| siRNA | 9526 | CfsasUfcUfuGfuCfuCfcUfaCfaGfuAfsusu | 9629 | usAfscUfgUfaGfgAfgAfcAfaGfaUfgsusu |
| 9526 | | | | |
| siRNA | 9527 | UfsusGfuCfuCfcUfaCfaGfuAfuUfgAfsusu | 9630 | usCfsaAfuAfcUfgUfaGfgAfgAfcAfasusu |
| 9527 | | | | |
| siRNA | 9528 | UfsgsUfcUfcCfuAfcAfgUfaUfuGfaAfsusu | 9631 | usUfscAfaUfaCfuGfuAfgGfaGfaCfasusu |
| 9528 | | | | |
| siRNA | 9529 | UfscsUfcCfuAfcAfgUfaUfuGfaAfuAfsusu | 9632 | usAfsuUfcAfaUfaCfuGfuAfgGfaGfasusu |
| 9529 | | | | |
| siRNA | 9530 | GfsusAfuUfgAfaUfaCfgUfgCfaCfaAfsusu | 9633 | usUfsgUfgCfaCfgUfaUfuCfaAfuAfcsusu |
| 9530 | | | | |
| siRNA | 9531 | AfsusUfgAfaUfaCfgUfgCfaCfaGfcAfsusu | 9634 | usGfscUfgUfgCfaCfgUfaUfuCfaAfususu |
| 9531 | | | | |
| siRNA | 9532 | UfsusGfaAfuAfcGfuGfcAfcAfgCfuAfsusu | 9635 | usAfsgCfuGfuGfcAfcGfuAfuUfcAfasusu |
| 9532 | | | | |
| siRNA | 9533 | GfsasAfuAfcGfuGfcAfcAfgCfuUfcAfsusu | 9636 | usGfsaAfgCfuGfuGfcAfcGfuAfuUfcsusu |
| 9533 | | | | |
| siRNA | 9534 | AfscsUfuCfcUfgAfcUfgUfaCfaGfcAfsusu | 9637 | usGfscUfgUfaCfaGfuCfaGfgAfaGfususu |
| 9534 | | | | |
| siRNA | 9535 | AfsgsCfcAfgAfgUfuGfgGfuGfaCfuAfsusu | 9638 | usAfsgUfcAfcCfcAfaCfuCfuGfgCfususu |
| 9535 | | | | |
| siRNA | 9536 | GfsgsUfcCfuCfgAfcUfgCfaGfaUfuAfsusu | 9639 | usAfsaUfcUfgCfaGfuCfgAfgGfaCfcsusu |
| 9536 | | | | |
| siRNA | 9537 | GfsusCfcUfcGfaCfuGfcAfgAfuUfuAfsusu | 9640 | usAfsaAfuCfuGfcAfgUfcGfaGfgAfcsusu |
| 9537 | | | | |
| siRNA | 9538 | UfscsCfuCfgAfcUfgCfaGfaUfuUfgAfsusu | 9641 | usCfsaAfaUfcUfgCfaGfuCfgAfgGfasusu |
| 9538 | | | | |
| siRNA | 9539 | CfscsUfcGfaCfuGfcAfgAfuUfuGfcAfsusu | 9642 | usGfscAfaAfuCfuGfcAfgUfcGfaGfgsusu |
| 9539 | | | | |
| siRNA | 9540 | CfsgsAfcUfgCfaGfaUfuUfgCfuCfcAfsusu | 9643 | usGfsgAfgCfaAfaUfcUfgCfaGfuCfgsusu |
| 9540 | | | | |
| siRNA | 9541 | CfsusGfcAfgAfuUfuGfcUfcCfaAfaAfsusu | 9644 | usUfsuUfgGfaGfcAfaAfuCfuGfcAfgsusu |
| 9541 | | | | |
| siRNA | 9542 | UfsgsCfaGfaUfuUfgCfuCfcAfaAfaAfsusu | 9645 | usUfsuUfuGfgAfgCfaAfaUfcUfgCfasusu |
| 9542 | | | | |
| siRNA | 9543 | CfsasGfaUfuUfgCfuCfcAfaAfaCfgAfsusu | 9646 | usCfsgUfuUfuGfgAfgCfaAfaUfcUfgsusu |
| 9543 | | | | |
| siRNA | 9544 | GfsusGfgAfgCfgCfuGfuUfgUfgAfaAfsusu | 9647 | usUfsuCfaCfaAfcAfgCfgCfuCfcAfcsusu |
| 9544 | | | | |
| siRNA | 9545 | UfsgsGfaGfcGfcUfgUfuGfuGfaAfuAfsusu | 9648 | usAfsuUfcAfcAfaCfaGfcGfcUfcCfasusu |
| 9545 | | | | |
| siRNA | 9546 | CfsasAfgGfaCfaGfcUfcAfaAfaCfuAfsusu | 9649 | usAfsgUfuUfuGfaGfcUfgUfcCfuUfgsusu |
| 9546 | | | | |
| siRNA | 9547 | UfsgsCfcCfcGfgAfaAfgAfcUfuUfgAfsusu | 9650 | usCfsaAfaGfuCfuUfuCfcGfgGfgCfasusu |
| 9547 | | | | |
| siRNA | 9548 | GfscsCfcCfgGfaAfaGfaCfuUfuGfuAfsusu | 9651 | usAfscAfaAfgUfcUfuUfcCfgGfgGfcsusu |
| 9548 | | | | |
| siRNA | 9549 | CfscsCfcGfgAfaAfgAfcUfuUfgUfaAfsusu | 9652 | usUfsaCfaAfaGfuCfuUfuCfcGfgGfgsusu |
| 9549 | | | | |
| siRNA | 9550 | CfscsCfgGfaAfaGfaCfuUfuGfuAfgAfsusu | 9653 | usCfsuAfcAfaAfgUfcUfuUfcCfgGfgsusu |
| 9550 | | | | |
| siRNA | 9551 | UfsusGfuAfgAfgGfaGfuUfuGfaGfuAfsusu | 9654 | usAfscUfcAfaAfcUfcCfuCfuAfcAfasusu |
| 9551 | | | | |
| siRNA | 9552 | AfsgsCfcUfcAfcCfgUfgAfcUfaAfcAfsusu | 9655 | usGfsuUfaGfuCfaCfgGfuGfaGfgCfususu |
| 9552 | | | | |
| siRNA | 9553 | CfsusCfaCfcGfuGfaCfuAfaCfaUfgAfsusu | 9656 | usCfsaUfgUfuAfgUfcAfcGfgUfgAfgsusu |
| 9553 | | | | |
| siRNA | 9554 | UfscsAfcCfgUfgAfcUfaAfcAfuGfcAfsusu | 9657 | usGfscAfuGfuUfaGfuCfaCfgGfuGfasusu |
| 9554 | | | | |
| siRNA | 9555 | CfsasCfcGfuGfaCfuAfaCfaUfgCfcAfsusu | 9658 | usGfsgCfaUfgUfuAfgUfcAfcGfgUfgsusu |
| 9555 | | | | |
| siRNA | 9556 | UfsgsCfuGfaUfaGfcAfgUfgCfaAfcAfsusu | 9659 | usGfsuUfgCfaCfuGfcUfaUfcAfgCfasusu |
| 9556 | | | | |
| siRNA | 9557 | GfsusCfuCfaCfuCfuUfgAfaGfgCfcAfsusu | 9660 | usGfsgCfcUfuCfaAfgAfgUfgAfgAfcsusu |
| 9557 | | | | |
| siRNA | 9558 | UfscsAfcUfcUfuGfaAfgGfcCfaGfaAfsusu | 9661 | usUfscUfgGfcCfuUfcAfaGfaGfuGfasusu |
| 9558 | | | | |
| siRNA | 9559 | UfsgsUfgCfuGfgUfcAfaUfgGfgAfcAfsusu | 9662 | usGfsuCfcCfaUfuGfaCfcAfgCfaCfasusu |
| 9559 | | | | |
| siRNA | 9560 | GfsgsUfcAfaUfgGfgAfcUfgAfgUfgAfsusu | 9663 | usCfsaCfuCfaGfuCfcCfaUfuGfaCfcsusu |
| 9560 | | | | |
| siRNA | 9561 | UfscsAfaUfgGfgAfcUfgAfgUfgUfcAfsusu | 9664 | usGfsaCfaCfuCfaGfuCfcCfaUfuGfasusu |
| 9561 | | | | |
| siRNA | 9562 | CfsasAfuGfgGfaCfuGfaGfuGfuCfuAfsusu | 9665 | usAfsgAfcAfcUfcAfgUfcCfcAfuUfgsusu |
| 9562 | | | | |
| siRNA | 9563 | GfsasCfuGfaGfuGfuCfuGfcUfaGfcAfsusu | 9666 | usGfscUfaGfcAfgAfcAfcUfcAfgUfcsusu |
| 9563 | | | | |
| siRNA | 9564 | GfsasGfgGfgCfaGfcUfuUfuAfuGfuAfsusu | 9667 | usAfscAfuAfaAfaGfcUfgCfcCfcUfcsusu |
| 9564 | | | | |
| siRNA | 9565 | GfscsAfgCfuUfuUfaUfgUfgCfcAfcAfsusu | 9668 | usGfsuGfgCfaCfaUfaAfaAfgCfuGfcsusu |
| 9565 | | | | |
| siRNA | 9566 | AfsgsCfuUfuUfaUfgUfgCfcAfcAfcAfsusu | 9669 | usGfsuGfuGfgCfaCfaUfaAfaAfgCfususu |
| 9566 | | | | |
| siRNA | 9567 | GfsasAfgAfcCfcUfgUfcGfuGfcUfaAfsusu | 9670 | usUfsaGfcAfcGfaCfaGfgGfuCfuUfcsusu |
| 9567 | | | | |
| siRNA | 9568 | AfsasGfaCfcCfuGfuCfgUfgCfuAfaAfsusu | 9671 | usUfsuAfgCfaCfgAfcAfgGfgUfcUfususu |
| 9568 | | | | |
| siRNA | 9569 | CfsusAfaGfcAfuCfaGfcCfcCfaAfcAfsusu | 9672 | usGfsuUfgGfgGfcUfgAfuGfcUfuAfgsusu |
| 9569 | | | | |
| siRNA | 9570 | UfsasAfgCfaUfcAfgCfcCfcAfaCfuAfsusu | 9673 | usAfsgUfuGfgGfgCfuGfaUfgCfuUfasusu |
| 9570 | | | | |
| siRNA | 9571 | CfsusGfuGfgCfcAfgCfaUfcUfaAfcAfsusu | 9674 | usGfsuUfaGfaUfgCfuGfgCfcAfcAfgsusu |
| 9571 | | | | |
| siRNA | 9572 | AfscsUfuCfaGfcAfuGfgCfaCfuUfaAfsusu | 9675 | usUfsaAfgUfgCfcAfuGfcUfgAfaGfususu |
| 9572 | | | | |
| siRNA | 9573 | UfsusCfaGfcAfuGfgCfaCfuUfaGfuAfsusu | 9676 | usAfscUfaAfgUfgCfcAfuGfcUfgAfasusu |
| 9573 | | | | |
| siRNA | 9574 | CfsasGfcAfuGfgCfaCfuUfaGfuGfcAfsusu | 9677 | usGfscAfcUfaAfgUfgCfcAfuGfcUfgsusu |
| 9574 | | | | |
| siRNA | 9575 | CfsasCfuUfaGfuGfcUfgUfcAfuUfcAfsusu | 9678 | usGfsaAfuGfaCfaGfcAfcUfaAfgUfgsusu |
| 9575 | | | | |
| siRNA | 9576 | UfsgsCfuGfuCfaUfuCfcAfuGfaCfgAfsusu | 9679 | usCfsgUfcAfuGfgAfaUfgAfcAfgCfasusu |
| 9576 | | | | |
| siRNA | 9577 | UfsgsUfcAfuUfcCfaUfgAfcGfgGfcAfsusu | 9680 | usGfscCfcGfuCfaUfgGfaAfuGfaCfasusu |
| 9577 | | | | |
| siRNA | 9578 | CfsasUfuCfcAfuGfaCfgGfgCfuUfaAfsusu | 9681 | usUfsaAfgCfcCfgUfcAfuGfgAfaUfgsusu |
| 9578 | | | | |
| siRNA | 9579 | AfsusUfcCfaUfgAfcGfgGfcUfuAfgAfsusu | 9682 | usCfsuAfaGfcCfcGfuCfaUfgGfaAfususu |
| 9579 | | | | |
| siRNA | 9580 | UfsgsAfgGfaGfcAfuGfcCfaUfuAfaAfsusu | 9683 | usUfsuAfaUfgGfcAfuGfcUfcCfuCfasusu |
| 9580 | | | | |
| siRNA | 9581 | GfscsAfuGfcCfaUfuAfaGfuUfuGfaAfsusu | 9684 | usUfscAfaAfcUfuAfaUfgGfcAfuGfcsusu |
| 9581 | | | | |
| siRNA | 9582 | UfsasUfcCfuGfgGfuAfgAfgUfgGfuAfsusu | 9685 | usAfscCfaCfuCfuAfcCfcAfgGfaUfasusu |
| 9582 | | | | |
| siRNA | 9583 | UfsgsGfuCfuUfcAfgCfuAfcUfgGfuAfsusu | 9686 | usAfscCfaGfuAfgCfuGfaAfgAfcCfasusu |
| 9583 | | | | |
| siRNA | 9584 | CfsusAfgUfuCfuUfcCfuCfcCfaAfcAfsusu | 9687 | usGfsuUfgGfgAfgGfaAfgAfaCfuAfgsusu |
| 9584 | | | | |
| siRNA | 9585 | UfsasCfuCfgGfgCfuCfuGfaCfuAfcAfsusu | 9688 | usGfsuAfgUfcAfgAfgCfcCfgAfgUfasusu |
| 9585 | | | | |
| siRNA | 9586 | CfsasCfuUfuGfgAfgUfuGfuCfuAfcAfsusu | 9689 | usGfsuAfgAfcAfaCfuCfcAfaAfgUfgsusu |
| 9586 | | | | |
| siRNA | 9587 | GfsasGfaAfuAfcAfuAfgAfcCfaGfgAfsusu | 9690 | usCfscUfgGfuCfuAfuGfuAfuUfcUfcsusu |
| 9587 | | | | |
| siRNA | 9588 | AfsusAfgAfcCfaGfgCfcCfaGfaAfuAfsusu | 9691 | usAfsuUfcUfgGfgCfcUfgGfuCfuAfususu |
| 9588 | | | | |
| siRNA | 9589 | GfsgsCfcCfaGfaAfuCfgAfaUfcCfaAfsusu | 9692 | usUfsgGfaUfuCfgAfuUfcUfgGfgCfcsusu |
| 9589 | | | | |
| siRNA | 9590 | CfscsCfaGfaAfuCfgAfaUfcCfaAfuAfsusu | 9693 | usAfsuUfgGfaUfuCfgAfuUfcUfgGfgsusu |
| 9590 | | | | |
| siRNA | 9591 | CfsasGfaAfuCfgAfaUfcCfaAfuGfuAfsusu | 9694 | usAfscAfuUfgGfaUfuCfgAfuUfcUfgsusu |
| 9591 | | | | |
| siRNA | 9592 | AfsgsAfaUfcGfaAfuCfcAfaUfgUfgAfsusu | 9695 | usCfsaCfaUfuGfgAfuUfcGfaUfuCfususu |
| 9592 | | | | |
| siRNA | 9593 | GfsasAfuCfgAfaUfcCfaAfuGfuGfcAfsusu | 9696 | usGfscAfcAfuUfgGfaUfuCfgAfuUfcsusu |
| 9593 | | | | |
| siRNA | 9594 | CfsgsAfaUfcCfaAfuGfuGfcCfaUfcAfsusu | 9697 | usGfsaUfgGfcAfcAfuUfgGfaUfuCfgsusu |
| 9594 | | | | |
| siRNA | 9595 | GfsasAfuCfcAfaUfgUfgCfcAfuCfaAfsusu | 9698 | usUfsgAfuGfgCfaCfaUfuGfgAfuUfcsusu |
| 9595 | | | | |
| siRNA | 9596 | CfsasAfuGfuGfcCfaUfcAfaGfuCfaAfsusu | 9699 | usUfsgAfcUfuGfaUfgGfcAfcAfuUfgsusu |
| 9596 | | | | |
| siRNA | 9597 | UfsgsUfgCfcAfuCfaAfgUfcAfcUfaAfsusu | 9700 | usUfsaGfuGfaCfuUfgAfuGfgCfaCfasusu |
| 9597 | | | | |
| siRNA | 9598 | GfsusGfcCfaUfcAfaGfuCfaCfuAfaAfsusu | 9701 | usUfsuAfgUfgAfcUfuGfaUfgGfcAfcsusu |
| 9598 | | | | |
| siRNA | 9599 | UfsgsCfcAfuCfaAfgUfcAfcUfaAfgAfsusu | 9702 | usCfsuUfaGfuGfaCfuUfgAfuGfgCfasusu |
| 9599 | | | | |
| siRNA | 9600 | CfsasUfcAfaGfuCfaCfuAfaGfuCfgAfsusu | 9703 | usCfsgAfcUfuAfgUfgAfcUfuGfaUfgsusu |
| 9600 | | | | |
| siRNA | 9601 | UfscsAfuUfgGfuAfuCfaUfgUfuGfcAfsusu | 9704 | usGfscAfaCfaUfgAfuAfcCfaAfuGfasusu |
| 9601 | | | | |
| siRNA | 9602 | CfscsUfaUfaUfgUfgCfcAfcGfgUfgAfsusu | 9705 | usCfsaCfcGfuGfgCfaCfaUfaUfaGfgsusu |
| 9602 | | | | |
| siRNA | 9603 | CfsusAfuAfuGfuGfcCfaCfgGfuGfaAfsusu | 9706 | usUfscAfcCfgUfgGfcAfcAfuAfuAfgsusu |
| 9603 | | | | |
| siRNA | 9604 | UfsasUfaUfgUfgCfcAfcGfgUfgAfcAfsusu | 9707 | usGfsuCfaCfcGfuGfgCfaCfaUfaUfasusu |
| 9604 | | | | |
| siRNA | 9605 | AfsusAfuGfuGfcCfaCfgGfuGfaCfcAfsusu | 9708 | usGfsgUfcAfcCfgUfgGfcAfcAfuAfususu |
| 9605 | | | | |
| siRNA | 9606 | AfsasGfuUfuGfuGfcAfcAfgGfgAfcAfsusu | 9709 | usGfsuCfcCfuGfuGfcAfcAfaAfcUfususu |
| 9606 | | | | |
| siRNA | 9607 | GfsgsUfgGfaGfcAfgAfuAfgUfgUfcAfsusu | 9710 | usGfsaCfaCfuAfuCfuGfcUfcCfaCfcsusu |
| 9607 | | | | |
| siRNA | 9608 | AfsgsAfuAfgUfgUfcUfgCfaCfuGfcAfsusu | 9711 | usGfscAfgUfgCfaGfaCfaCfuAfuCfususu |
| 9608 | | | | |
| siRNA | 9609 | UfsgsGfgGfaCfcAfuUfaUfgUfgCfaAfsusu | 9712 | usUfsgCfaCfaUfaAfuGfgUfcCfcCfasusu |
| 9609 | | | | |
| siRNA | 9610 | GfsgsGfaCfcAfuUfaUfgUfgCfaGfcAfsusu | 9713 | usGfscUfgCfaCfaUfaAfuGfgUfcCfcsusu |
| 9610 | | | | |
| siRNA | 9611 | CfscsAfgCfaAfcCfuAfcAfuGfaAfcAfsusu | 9714 | usGfsuUfcAfuGfuAfgGfuUfgCfuGfgsusu |
| 9611 | | | | |

TABLE 10
Alternatively modified siRNA subset F sequences (a.k.a. subset H)

| siRNA | SEQ ID | sense strand sequence | SEQ ID | antisense strand |
|---|---|---|---|---|
| Name | NO: | (5′-3′) | NO: | (5′-3′) |
| ETD01363 | 9715 | gscsugUfgUfuUfgUfagcc | 9612 | usUfsaUfgGfcUfaCfaAfaCfa |
| | | auaasusu | | CfaGfcsusu |
| ETD01364 | 9716 | usgsugUfuUfgUfagccaua | 9613 | usCfsgUfaUfgGfcUfaCfaAfa |
| | | cgasusu | | CfaCfasusu |
| ETD01365 | 9717 | ususugUfagCfCfaUfacgc | 9614 | usAfsuUfgCfgUfaUfgGfcUfa |
| | | aauasusu | | CfaAfasusu |
| ETD01366 | 9718 | ususguAfgccAfuAfcgcaa | 9615 | usGfsaUfuGfcGfuAfuGfgCfu |
| | | ucasusu | | AfcAfasusu |
| ETD01367 | 9719 | usgsuagCfCfaUfaCfgcaa | 9616 | usCfsgAfuUfgCfgUfaUfgGfc |
| | | ucgasusu | | UfaCfasusu |
| ETD01368 | 9720 | csasuacGfcAfAfucgccug | 9617 | usUfsgCfaGfgCfgAfuUfgCfg |
| | | caasusu | | UfaUfgsusu |
| ETD01369 | 9721 | gscsuuCfCfugCfaugaccu | 9618 | usCfsuAfgGfuCfaUfgCfaGfg |
| | | agasusu | | AfaGfcsusu |
| ETD01370 | 9722 | csusuccuGfcAfuGfaccua | 9619 | usUfscUfaGfgUfcAfuGfcAfg |
| | | gaasusu | | GfaAfgsusu |
| ETD01371 | 9723 | csusucuCfagCfcCfaccau | 9620 | usUfsuAfuGfgUfgGfgCfuGfa |
| | | aaasusu | | GfaAfgsusu |
| ETD01372 | 9724 | cscsuccUfaUfUfUfCfuac | 9621 | usCfscAfcGfuAfgAfaAfuAfg |
| | | guggasusu | | GfaGfgsusu |
| ETD01373 | 9725 | cscsacGfcucAfGfugucua | 9622 | usGfsaUfaGfaCfaCfuGfaGfc |
| | | ucasusu | | GfuGfgsusu |
| ETD01374 | 9726 | csasguGfucuAfucaggcgu | 9623 | usGfsaCfgCfcUfgAfuAfgAfc |
| | | casusu | | AfcUfgsusu |
| ETD01375 | 9727 | gsusguCfUfaUfCfaggcgu | 9624 | usGfsaGfaCfgCfcUfgAfuAfg |
| | | cucasusu | | AfcAfcsusu |
| ETD01376 | 9728 | usgsucuAfucAfgGfcgucu | 9625 | usUfsgAfgAfcGfcCfuGfaUfa |
| | | caasusu | | GfaCfasusu |
| ETD01377 | 9729 | gsuscuAfucaGfGfcgucuc | 9626 | usUfsuGfaGfaCfgCfcUfgAfu |
| | | aaasusu | | AfgAfcsusu |
| ETD01378 | 9730 | usasucaGfGfcGfucucaag | 9627 | usGfscCfuUfgAfgAfcGfcCfu |
| | | gcasusu | | GfaUfasusu |
| ETD01379 | 9731 | usgsccCfaagCfaUfcuugu | 9628 | usAfsgAfcAfaGfaUfgCfuUfg |
| | | cuasusu | | GfgCfasusu |
| ETD01380 | 9732 | csasucUfugUfCfUfCfcua | 9629 | usAfscUfgUfaGfgAfgAfcAfa |
| | | caguasusu | | GfaUfgsusu |
| ETD01381 | 9733 | ususguCfUfCfCfUfacagu | 9630 | usCfsaAfuAfcUfgUfaGfgAfg |
| | | auugasusu | | AfcAfasusu |
| ETD01382 | 9734 | usgsucuccuAfcAfguauug | 9631 | usUfscAfaUfaCfuGfuAfgGfa |
| | | aasusu | | GfaCfasusu |
| ETD01383 | 9735 | uscsuccuAfcAfGfuauuga | 9632 | usAfsuUfcAfaUfaCfuGfuAfg |
| | | auasusu | | GfaGfasusu |
| ETD01384 | 9736 | gsusauUfgaaUfaCfgugca | 9633 | usUfsgUfgCfaCfgUfaUfuCfa |
| | | caasusu | | AfuAfcsusu |
| ETD01385 | 9737 | asusugaaUfaCfgUfgcaca | 9634 | usGfscUfgUfgCfaCfgUfaUfu |
| | | gcasusu | | CfaAfususu |
| ETD01386 | 9738 | ususgaAfuAfcGfuGfcaca | 9635 | usAfsgCfuGfuGfcAfcGfuAfu |
| | | gcuasusu | | UfcAfasusu |
| ETD01387 | 9739 | gsasauAfcGfuGfcAfcagc | 9636 | usGfsaAfgCfuGfuGfcAfcGfu |
| | | uucasusu | | AfuUfcsusu |
| ETD01388 | 9740 | ascsuuccuGfAfcuguacag | 9637 | usGfscUfgUfaCfaGfuCfaGfg |
| | | casusu | | AfaGfususu |
| ETD01389 | 9741 | asgsccagagUfUfgggugac | 9638 | usAfsgUfcAfcCfcAfaCfuCfu |
| | | uasusu | | GfgCfususu |
| ETD01390 | 9742 | gsgsuccucGfAfcugcagau | 9639 | usAfsaUfcUfgCfaGfuCfgAfg |
| | | uasusu | | GfaCfcsusu |
| ETD01391 | 9743 | gsusccUfCfgaCfUfgcaga | 9640 | usAfsaAfuCfuGfcAfgUfcGfa |
| | | uuuasusu | | GfgAfcsusu |
| ETD01392 | 9744 | uscscuCfgaCfUfgCfagau | 9641 | usCfsaAfaUfcUfgCfaGfuCfg |
| | | uugasusu | | AfgGfasusu |
| ETD01393 | 9745 | cscsucGfAfcuGfcAfgauu | 9642 | usGfscAfaAfuCfuGfcAfgUfc |
| | | ugcasusu | | GfaGfgsusu |
| ETD01394 | 9746 | csgsacuGfcAfGfauuugcu | 9643 | usGfsgAfgCfaAfaUfcUfgCfa |
| | | ccasusu | | GfuCfgsusu |
| ETD01395 | 9747 | csusgcagaUfUfugcuccaa | 9644 | usUfsuUfgGfaGfcAfaAfuCfu |
| | | aasusu | | GfcAfgsusu |
| ETD01396 | 9748 | usgscagaUfuUfgCfuccaa | 9645 | usUfsuUfuGfgAfgCfaAfaUfc |
| | | aaasusu | | UfgCfasusu |
| ETD01397 | 9749 | csasgaUfuUfgCfuCfcaaa | 9646 | usCfsgUfuUfuGfgAfgCfaAfa |
| | | acgasusu | | UfcUfgsusu |
| ETD01398 | 9750 | gsusggagCfgCfUfguugug | 9647 | usUfsuCfaCfaAfcAfgCfgCfu |
| | | aaasusu | | CfcAfcsusu |
| ETD01399 | 9751 | usgsgagCfgCfUfgUfugug | 9648 | usAfsuUfcAfcAfaCfaGfcGfc |
| | | aauasusu | | UfcCfasusu |
| ETD01400 | 9752 | csasagGfAfcAfGfcucaaa | 9649 | usAfsgUfuUfuGfaGfcUfgUfc |
| | | acuasusu | | CfuUfgsusu |
| ETD01401 | 9753 | usgsccccGfGfAfAfAfgac | 9650 | usCfsaAfaGfuCfuUfuCfcGfg |
| | | uuugasusu | | GfgCfasusu |
| ETD01402 | 9754 | gscscccgGfAfAfAfgacuu | 9651 | usAfscAfaAfgUfcUfuUfcCfg |
| | | uguasusu | | GfgGfcsusu |
| ETD01403 | 9755 | cscsccggAfAfAfGfAfcuu | 9652 | usUfsaCfaAfaGfuCfuUfuCfc |
| | | uguaasusu | | GfgGfgsusu |
| ETD01404 | 9756 | cscscgGfaAfAfGfAfcuuu | 9653 | usCfsuAfcAfaAfgUfcUfuUfc |
| | | guagasusu | | CfgGfgsusu |
| ETD01405 | 9757 | ususguaGfaGfGfaGfuuug | 9654 | usAfscUfcAfaAfcUfcCfuCfu |
| | | aguasusu | | AfcAfasusu |
| ETD01406 | 9758 | asgsccuCfaCfCfgugacua | 9655 | usGfsuUfaGfuCfaCfgGfuGfa |
| | | acasusu | | GfgCfususu |
| ETD01407 | 9759 | csuscaccGfuGfAfcuaaca | 9656 | usCfsaUfgUfuAfgUfcAfcGfg |
| | | ugasusu | | UfgAfgsusu |
| ETD01408 | 9760 | uscsaccGfuGfAfcuaacau | 9657 | usGfscAfuGfuUfaGfuCfaCfg |
| | | gcasusu | | GfuGfasusu |
| ETD01409 | 9761 | csasccgUfgaCfUfaacaug | 9658 | usGfsgCfaUfgUfuAfgUfcAfc |
| | | ccasusu | | GfgUfgsusu |
| ETD01410 | 9762 | usgscuGfAfuAfGfcAfgug | 9659 | usGfsuUfgCfaCfuGfcUfaUfc |
| | | caacasusu | | AfgCfasusu |
| ETD01411 | 9763 | gsuscuCfaCfuCfuugaagg | 9660 | usGfsgCfcUfuCfaAfgAfgUfg |
| | | ccasusu | | AfgAfcsusu |
| ETD01412 | 9764 | uscsacucuuGfaAfggccag | 9661 | usUfscUfgGfcCfuUfcAfaGfa |
| | | aasusu | | GfuGfasusu |
| ETD01413 | 9765 | usgsugCfUfggUfCfaaugg | 9662 | usGfsuCfcCfaUfuGfaCfcAfg |
| | | gacasusu | | CfaCfasusu |
| ETD01414 | 9766 | gsgsucAfauGfGfGfAfcug | 9663 | usCfsaCfuCfaGfuCfcCfaUfu |
| | | agugasusu | | GfaCfcsusu |
| ETD01415 | 9767 | uscsaauGfGfGfAfcugagu | 9664 | usGfsaCfaCfuCfaGfuCfcCfa |
| | | gucasusu | | UfuGfasusu |
| ETD01416 | 9768 | csasaugggaCfUfgaguguc | 9665 | usAfsgAfcAfcUfcAfgUfcCfc |
| | | uasusu | | AfuUfgsusu |
| ETD01417 | 9769 | gsascuGfaGfuGfucugcua | 9666 | usGfscUfaGfcAfgAfcAfcUfc |
| | | gcasusu | | AfgUfcsusu |
| ETD01418 | 9770 | gsasggGfGfcaGfcuuuuau | 9667 | usAfscAfuAfaAfaGfcUfgCfc |
| | | guasusu | | CfcUfcsusu |
| ETD01419 | 9771 | gscsagcUfUfUfUfaUfgug | 9668 | usGfsuGfgCfaCfaUfaAfaAfg |
| | | ccacasusu | | CfuGfcsusu |
| ETD01420 | 9772 | asgscuUfuUfaUfgUfgcca | 9669 | usGfsuGfuGfgCfaCfaUfaAfa |
| | | cacasusu | | AfgCfususu |
| ETD01421 | 9773 | gsasagaCfCfCfUfgUfcgu | 9670 | usUfsaGfcAfcGfaCfaGfgGfu |
| | | gcuaasusu | | CfuUfcsusu |
| ETD01422 | 9774 | asasgacccuGfucgugcuaa | 9671 | usUfsuAfgCfaCfgAfcAfgGfg |
| | | asusu | | UfcUfususu |
| ETD01423 | 9775 | csusaagCfaUfCfagcccca | 9672 | usGfsuUfgGfgGfcUfgAfuGfc |
| | | acasusu | | UfuAfgsusu |
| ETD01424 | 9776 | usasagcAfucAfGfccccaa | 9673 | usAfsgUfuGfgGfgCfuGfaUfg |
| | | cuasusu | | CfuUfasusu |
| ETD01425 | 9777 | csusguGfGfccAfGfcaucu | 9674 | usGfsuUfaGfaUfgCfuGfgCfc |
| | | aacasusu | | AfcAfgsusu |
| ETD01426 | 9778 | ascsuucAfGfcAfuGfgcac | 9675 | usUfsaAfgUfgCfcAfuGfcUfg |
| | | uuaasusu | | AfaGfususu |
| ETD01427 | 9779 | ususcaGfcauGfGfcacuua | 9676 | usAfscUfaAfgUfgCfcAfuGfc |
| | | guasusu | | UfgAfasusu |
| ETD01428 | 9780 | csasgcaUfggCfaCfuuagu | 9677 | usGfscAfcUfaAfgUfgCfcAfu |
| | | gcasusu | | GfcUfgsusu |
| ETD01429 | 9781 | csascuuAfGfuGfcugucau | 9678 | usGfsaAfuGfaCfaGfcAfcUfa |
| | | ucasusu | | AfgUfgsusu |
| ETD01430 | 9782 | usgscugUfcaUfUfccauga | 9679 | usCfsgUfcAfuGfgAfaUfgAfc |
| | | cgasusu | | AfgCfasusu |
| ETD01431 | 9783 | usgsucaUfUfCfCfaUfgac | 9680 | usGfscCfcGfuCfaUfgGfaAfu |
| | | gggcasusu | | GfaCfasusu |
| ETD01432 | 9784 | csasuuccAfuGfAfcgggcu | 9681 | usUfsaAfgCfcCfgUfcAfuGfg |
| | | uaasusu | | AfaUfgsusu |
| ETD01433 | 9785 | asusuccAfuGfAfcGfggcu | 9682 | usCfsuAfaGfcCfcGfuCfaUfg |
| | | uagasusu | | GfaAfususu |
| ETD01434 | 9786 | usgsagGfAfgcAfuGfccau | 9683 | usUfsuAfaUfgGfcAfuGfcUfc |
| | | uaaasusu | | CfuCfasusu |
| ETD01435 | 9787 | gscsaugCfCfaUfUfaaguu | 9684 | usUfscAfaAfcUfuAfaUfgGfc |
| | | ugaasusu | | AfuGfcsusu |
| ETD01436 | 9788 | usasuccuGfgGfuAfgagug | 9685 | usAfscCfaCfuCfuAfcCfcAfg |
| | | guasusu | | GfaUfasusu |
| ETD01437 | 9789 | usgsgucuucAfGfcuacugg | 9686 | usAfscCfaGfuAfgCfuGfaAfg |
| | | uasusu | | AfcCfasusu |
| ETD01438 | 9790 | csusagUfUfcUfUfccuccc | 9687 | usGfsuUfgGfgAfgGfaAfgAfa |
| | | aacasusu | | CfuAfgsusu |
| ETD01439 | 9791 | usascuCfgggCfuCfugacu | 9688 | usGfsuAfgUfcAfgAfgCfcCfg |
| | | acasusu | | AfgUfasusu |
| ETD01440 | 9792 | csascuuuGfGfAfGfuuguc | 9689 | usGfsuAfgAfcAfaCfuCfcAfa |
| | | uacasusu | | AfgUfgsusu |
| ETD01441 | 9793 | gsasgaAfuAfcAfuAfgacc | 9690 | usCfscUfgGfuCfuAfuGfuAfu |
| | | aggasusu | | UfcUfcsusu |
| ETD01442 | 9794 | asusagAfccAfGfGfcccag | 9691 | usAfsuUfcUfgGfgCfcUfgGfu |
| | | aauasusu | | CfuAfususu |
| ETD01443 | 9795 | gsgscccAfgAfAfucgaauc | 9692 | usUfsgGfaUfuCfgAfuUfcUfg |
| | | caasusu | | GfgCfcsusu |
| ETD01444 | 9796 | cscscagaaUfCfgaauccaa | 9693 | usAfsuUfgGfaUfuCfgAfuUfc |
| | | uasusu | | UfgGfgsusu |
| ETD01445 | 9797 | csasgaAfucgAfAfuccaau | 9694 | usAfscAfuUfgGfaUfuCfgAfu |
| | | guasusu | | UfcUfgsusu |
| ETD01446 | 9798 | asgsaaucgAfAfuccaaugu | 9695 | usCfsaCfaUfuGfgAfuUfcGfa |
| | | gasusu | | UfuCfususu |
| ETD01447 | 9799 | gsasauCfgaaUfCfcaaugu | 9696 | usGfscAfcAfuUfgGfaUfuCfg |
| | | gcasusu | | AfuUfcsusu |
| ETD01448 | 9800 | csgsaauccAfAfuGfugcca | 9697 | usGfsaUfgGfcAfcAfuUfgGfa |
| | | ucasusu | | UfuCfgsusu |
| ETD01449 | 9801 | gsasauCfCfaaUfgUfgcca | 9698 | usUfsgAfuGfgCfaCfaUfuGfg |
| | | ucaasusu | | AfuUfcsusu |
| ETD01450 | 9802 | csasaugUfgCfCfaUfcaag | 9699 | usUfsgAfcUfuGfaUfgGfcAfc |
| | | ucaasusu | | AfuUfgsusu |
| ETD01451 | 9803 | usgsugCfCfauCfaagucac | 9700 | usUfsaGfuGfaCfuUfgAfuGfg |
| | | uaasusu | | CfaCfasusu |
| ETD01452 | 9804 | gsusgccAfucAfAfgucacu | 9701 | usUfsuAfgUfgAfcUfuGfaUfg |
| | | aaasusu | | GfcAfcsusu |
| ETD01453 | 9805 | usgsccAfucAfAfgucacua | 9702 | usCfsuUfaGfuGfaCfuUfgAfu |
| | | agasusu | | GfgCfasusu |
| ETD01454 | 9806 | csasucaagUfCfaCfuaagu | 9703 | usCfsgAfcUfuAfgUfgAfcUfu |
| | | cgasusu | | GfaUfgsusu |
| ETD01455 | 9807 | uscsauuGfGfuAfucauguu | 9704 | usGfscAfaCfaUfgAfuAfcCfa |
| | | gcasusu | | AfuGfasusu |
| ETD01456 | 9808 | cscsuaUfaUfgUfgccacgg | 9705 | usCfsaCfcGfuGfgCfaCfaUfa |
| | | ugasusu | | UfaGfgsusu |
| ETD01457 | 9809 | csusauAfuGfuGfccacggu | 9706 | usUfscAfcCfgUfgGfcAfcAfu |
| | | gaasusu | | AfuAfgsusu |
| ETD01458 | 9810 | usasuaUfgUfgCfCfacggu | 9707 | usGfsuCfaCfcGfuGfgCfaCfa |
| | | gacasusu | | UfaUfasusu |
| ETD01459 | 9811 | asusaugugCfCfaCfgguga | 9708 | usGfsgUfcAfcCfgUfgGfcAfc |
| | | ccasusu | | AfuAfususu |
| ETD01460 | 9812 | asasguuuGfuGfcAfcaggg | 9709 | usGfsuCfcCfuGfuGfcAfcAfa |
| | | acasusu | | AfcUfususu |
| ETD01461 | 9813 | gsgsuggAfgcAfgAfuagug | 9710 | usGfsaCfaCfuAfuCfuGfcUfc |
| | | ucasusu | | CfaCfcsusu |
| ETD01462 | 9814 | asgsauagUfgUfcUfgcacu | 9711 | usGfscAfgUfgCfaGfaCfaCfu |
| | | gcasusu | | AfuCfususu |
| ETD01463 | 9815 | usgsggGfAfccAfuuaugug | 9712 | usUfsgCfaCfaUfaAfuGfgUfc |
| | | caasusu | | CfcCfasusu |
| ETD01464 | 9816 | gsgsgaccaUfUfaUfgugca | 9713 | usGfscUfgCfaCfaUfaAfuGfg |
| | | gcasusu | | UfcCfcsusu |
| ETD01465 | 9817 | cscsagCfaaCfCfuacauga | 9714 | usGfsuUfcAfuGfuAfgGfuUfg |
| | | acasusu | | CfuGfgsusu |

Any siRNA among any of subsets A-H may comprise any modification pattern described herein. If a sequence is a different number of nucleotides in length than a modification pattern, the modification pattern may still be used with the appropriate number of additional nucleotides added 5′ or 3′ to match the number of nucleotides in the modification pattern. For example, if a sense or antisense strand of the siRNA among any of subsets A-F comprises 19 nucleotides, and a modification pattern comprises 21 nucleotides, UU may be added onto the 5′ end of the sense or antisense strand.

### Example 3: Screening MST1R siRNAs for Activity in Human Cells in Culture

Chemically modified MST1R siRNAs cross reactive for human and non-human primate and derived from sequences in siRNA subset F (Table 8) will be assayed for MST1R mRNA knockdown activity in cells in culture. A-431 (ATCC® CRL-1555™) cells will be seeded in 96-well tissue culture plates at a cell density of 10,000 cells per well in DMEM (ATCC Catalog No. 30-2002) supplemented with 10% fetal bovine serum and incubated overnight in a water-jacketed, humidified incubator at 37° C. in an atmosphere composed of air plus 5% carbon dioxide. The MST1R siRNAs will be individually transfected into A-431 cells in duplicate wells at 10 nM final concentration using 0.15 μL Lipofectamine RNAiMax (Fisher) per well. Silencer Select Negative Control #1 (ThermoFisher, Catalog #4390843) will be transfected at 10 nM final concentration as a control. After incubation for 48 hours at 37° C., total RNA will be harvested from each well and cDNA prepared using TaqMan® Fast Advanced Cells-to-CT™ Kit (ThermoFisher, Catalog #A35374) according to the manufacturer's instructions. The level of MST1R mRNA from each well will be measured in triplicate by real-time qPCR on a QuantStudio™ 6 Pro Real-Time PCR System using TaqMan Gene Expression Assay for human MST1R (ThermoFisher, assay #Hs00899921_g1). The level of PPIA mRNA will be measured using TaqMan Gene Expression Assay (ThermoFisher, assay #Hs99999904_m1) and used to determine relative MST1R mRNA levels in each well using the delta-delta Ct method. All data will be normalized to relative MST1R mRNA levels in untreated A-431 cells.

### Example 4: Determining the IC50 of MST1R siRNAs

The IC50 values for knockdown of MST1R mRNA by select MST1R siRNAs will be determined in A-431 (ATCC® CRL-1555™) cells. The siRNAs will be assayed individually at 30 nM, 10 nM, 3 nM, 1 nM and 0.3 nM, or 3 nM, 1 nM, 0.3 nM, 0.1 nM and 0.03 nM, or 30 nM, 10 nM, 3 nM, 1 nM, 0.3 nM, 0.1 nM and 0.03 nM. The A-431 cells will be seeded in 96-well tissue culture plates at a cell density of 7,500 cells per well in DMEM (ATCC Catalog No. 30-2002) supplemented with 10% fetal bovine serum and incubated overnight in a water-jacketed, humidified incubator at 37° C. in an atmosphere composed of air plus 5% carbon dioxide. The MST1R siRNAs will be individually transfected into A-431 cells in triplicate wells using 0.15 μL Lipofectamine RNAiMax (Fisher) per well. After incubation for 48 hours at 37° C., total RNA will be harvested from each well and cDNA prepared using TaqMan® Fast Advanced Cells-to-CT™ Kit (ThermoFisher, Catalog #A35374) according to the manufacturer's instructions. The level of MST1R mRNA from each well will be measured in triplicate by real-time qPCR on a QuantStudio™ 6 Pro Real-Time PCR System using TaqMan Gene Expression Assay for human MST1R (ThermoFisher, assay #Hs00899921_g1). The level of PPIA mRNA will be measured using TaqMan Gene Expression Assay (ThermoFisher, assay #Hs99999904 ml) and used to determine relative MST1R mRNA levels in each well using the delta-delta Ct method. All data will be normalized to relative MST1R mRNA levels in untreated A-431 cells. Curve fit will be accomplish using the [inhibitor] vs. response (three parameters) function in GraphPad Prism software.

### Example 5: siRNA-Mediated Knockdown of MST1R in A549 Cell Line

siRNAs targeted to MST1R mRNA that downregulate levels of MST1R mRNA may lead to a decrease in AKT activation upon MST1 protein stimulation, when administered to the cultured human lung epithelial cell line, A549.

On Day 0, A549 cells are seeded at 150,000 cells/mL into a Falcon 24-well tissue culture plate (ThermoFisher Cat. No. 353047) at 0.5 mL per well.

On Day 1, MST1R siRNA and negative control siRNA master mixes are prepared. The MST1R siRNA master mix contains 350 μL of Opti-MEM (ThermoFisher Cat. No. 4427037—s1288 Lot No. AS02B02D) and 3.5 μL of a mixture of two MST1R siRNAs (10 μM stock). The negative control siRNA master mix contains 350 μL of Opti-MEM and 3.5 μL of negative control siRNA (ThermoFisher Cat. No. 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of A549 cells with a final siRNA concentration of 10 nM.

On Day 3, 48 hours post transfection, media containing MST1 protein is added and the cells are lysed 24 hrs after MST1 stimulation using the Cells-to-Ct kit according to the manufacturer's protocol (ThermoFisher Cat. No. 4399002). For the Cells-to-Ct, cells are washed with 50 μL using cold 1×PBS and lysed by adding 49.5 μL of Lysis Solution and 0.5 μL DNase I per well and pipetting up and down 5 times and incubating for 5 minutes at room temperature. The Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/MST1R using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. The primary immunoblotting antibodies used are anti-MST1R, anti-AKT and anti-AKT-P (Abcam, Cambridge, UK).

A decrease in MST1R mRNA expression in the A549 cells is expected after transfection with the MST1R siRNAs compared to MST1R mRNA levels in A549 cells transfected with the non-specific control siRNA 72 hours after transfection. There is also an expected decrease in the amount of activated AKT, measured by quantifying the total amount of AKT protein relative to phosphorylated AKT (AKT-P) in A549 cells transfected with the MST1R siRNAs relative to the amount of MST1R RNA containing A549 cells transfected with a non-specific control siRNA 24 hours after MST1 stimulation. These results will show that MST1R siRNAs may elicit knockdown of MST1R mRNA in A549 cells and that decreased MST1R expression may correspond with a decrease in activated AKT.

### Example 6: ASO-Mediated Knockdown of MST1R in A549 Cell Line

ASOs targeted to MST1R mRNA that downregulate levels of MST1R mRNA may lead to a decrease in AKT activation upon MST1 protein stimulation, when administered to the cultured human lung epithelial cell line, A549.

On Day 0, A549 cells are seeded at 150,000 cells/mL into a Falcon 24-well tissue culture plate (ThermoFisher Cat. No. 353047) at 0.5 mL per well.

On Day 1, MST1R ASO and negative control ASO master mixes are prepared. The MST1R ASO master mix contains 350 μL of Opti-MEM (ThermoFisher Cat. No. 4427037—s1288 Lot No. AS02B02D) and 3.5 μL of a mixture of the MST1R ASOs (10 μM stock). The negative control ASO master mix contains 350 μL of Opti-MEM and 3.5 μL of negative control ASO (ThermoFisher Cat. No. 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of A549 cells with a final ASO concentration of 10 nM.

On Day 3, 48 hours post transfection, media containing MST1 protein is added and the cells are lysed 24 hrs after MST1 stimulation using the Cells-to-Ct kit according to the manufacturer's protocol (ThermoFisher Cat. No. 4399002). For the Cells-to-Ct, cells are washed with 50 μL using cold 1×PBS and lysed by adding 49.5 μL of Lysis Solution and 0.5 μL DNase I per well and pipetting up and down 5 times and incubating for 5 minutes at room temperature. The Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/MST1R using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. The primary immunoblotting antibodies used are anti-MST1R, anti-AKT and anti-AKT-P (Abcam, Cambridge, UK).

A decrease in MST1R mRNA expression in the A549 cells is expected after transfection with the MST1R ASOs compared to MST1R mRNA levels in A549 cells transfected with the non-specific control ASO 72 hours after transfection. There is also an expected decrease in the amount of activated AKT, measured by quantifying the total amount of AKT protein relative to phosphorylated AKT (AKT-P) in A549 cells transfected with the MST1R ASOs relative to the amount of MST1R RNA containing A549 cells transfected with a non-specific control ASO 24 hours after MST1 stimulation. These results will show that the MST1R ASOs may elicit knockdown of MST1R mRNA in A549 cells and that decreased MST1R expression may correspond with a decrease in activated AKT.

### Example 7: Inhibition of MST1R in a Mouse Model of Lung Inflammation Via Acute Cigarette Smoke Exposure Using MST1R siRNAs or ASOs

In this experiment, a mouse model of lung inflammation induced by acute cigarette smoke exposure will be used to evaluate the effect of siRNA or ASO inhibition of MST1R. In this cigarette smoke induced model, mice are exposed to cigarette smoke for 3 hours which will result in a transient inflammatory response. Lung inflammation is assessed by measuring neutrophils and macrophages in bronchoalveolar lavage fluid and lung tissue.

Mice are divided into six groups: Group 1—a group treated with non-targeting control siRNA and cigarette smoke inhalation, Group 2—a group treated with non-targeting control ASO and cigarette smoke inhalation, Group 3—a group treated with MST1R siRNA1 and cigarette smoke inhalation, Group 4—a group treated with MST1R ASO1 and cigarette smoke inhalation, Group 5—a group treated with vehicle and cigarette smoke inhalation, Group 6—a group treated with vehicle and not receiving cigarette smoke stimulus. Each group contains eight mice (4 males, 4 females).

Administration of siRNA, ASO or vehicle is achieved with 10 μg/kg of siRNA or ASO suspended in 0.9% sodium chloride (Baxter Cat. No. JB1323) delivered via inhalation using a Lovelace nebulizer (model 01-100) at a flow rate of 1 liter/min. Restrained mice are treated for a total of 10 min. Group 1 mice receive non-targeting control siRNA, Group 2 mice receive non-targeting control ASO, Group 3 mice receive siRNA1 targeting mouse MST1R, Group 4 mice receive ASO1 targeting mouse MST1R, and Group 5 and 6 mice receive vehicle.

24 hours after the smoke inhalation treatment, bronchoalveolar lavage fluid is collected and the mice are sacrificed by cervical dislocation following an intraperitoneal injection of 0.3 ml Nembutal (5 mg/ml) (Sigma Cat. No. 1507002). Final blood samples are collected, and livers and lungs are removed, and a section placed in RNAlater for mRNA isolation.

mRNA is isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/MST1R using a BioRad CFX96 Cat. No. 1855195).

A decrease in MST1R mRNA expression in the lung tissue from mice dosed with the MST1R siRNA1 or ASO1 is expected compared to MST1R mRNA expression in lung tissue from mice dosed with the non-specific controls. There is an expected decrease in neutrophil and macrophage counts in the bronchoalveolar lavage fluid in cigarette smoke exposed mice that receive the MST1R siRNA or ASO compared to the neutrophil and macrophage counts in the bronchoalveolar lavage fluid in cigarette smoke exposed mice that receive the non-specific control. These results will show that the MST1R siRNA or ASO may elicit knockdown of MST1R mRNA in lung tissue and that the decrease in MST1R expression may correspond with a decrease in neutrophil and macrophage counts in the bronchoalveolar lavage fluid in mice exposed to cigarette smoke.

### Example 8: Inhibition of MST1R in a Mouse Model of COPD Using MST1R siRNAs or ASOs

In this experiment, a mouse model of cigarette smoke induced COPD will be used to evaluate the effect of siRNA or ASO inhibition of MST1R. In the cigarette smoke induced COPD model, mice are exposed to cigarette smoke for 6 months to mimic patients with a substantial history of cigarette smoking. Lung inflammation is assessed by measuring neutrophil and macrophage in bronchoalveolar lavage fluid and lung tissue. Lung function is also assessed by measuring tidal volume, resistance and dynamic compliance. Additionally, lung morphology and air space enlargement is assessed by fixing and staining the lungs and measuring structural parameters such as air space, septal wall thickness and mean linear intercept.

Mice are divided into six groups: Group 1—a group treated with non-targeting control siRNA and cigarette smoke inhalation, Group 2—a group treated with non-targeting control ASO and cigarette smoke inhalation, Group 3—a group treated with MST1R siRNA1 and cigarette smoke inhalation, Group 4—a group treated with MST1R ASO1 and cigarette smoke inhalation, Group 5—a group treated with vehicle and cigarette smoke inhalation, Group 6—a group treated with vehicle and not receiving cigarette smoke stimulus. Each group contains eight mice (4 males, 4 females).

Administration of siRNA, ASO or vehicle is achieved with 10 μg/kg of siRNA or ASO suspended in 0.9% sodium chloride (Baxter Cat. No. JB1323) delivered via inhalation using a Lovelace nebulizer (model 01-100) at a flow rate of 1 liter/min. Restrained mice are treated for a total of 10 min. Group 1 mice receive non-targeting control siRNA, Group 2 mice receive non-targeting control ASO, Group 3 mice receive siRNA1 targeting mouse MST1R, Group 4 mice receive ASO1 targeting mouse MST1R, and Group 5 and 6 mice receive vehicle. Every 7 days after the first administration animals from each group will be dosed for a total of 12 administrations.

24 hours after the final smoke inhalation treatment, bronchoalveolar lavage fluid is collected and the mice are sacrificed by cervical dislocation following an intraperitoneal injection of 0.3 ml Nembutal (5 mg/ml) (Sigma Cat. No. 1507002). Final blood samples are collected, and livers and lungs are removed, and a section placed in RNAlater for mRNA isolation or fixed with paraformaldehyde and then embedded in paraffin for tissue sectioning.

mRNA is isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/MST1R using a BioRad CFX96 Cat. No. 1855195).

A decrease in MST1R mRNA expression in the lung tissue from mice dosed with the MST1R siRNA1 or ASO1 is expected compared to MST1R mRNA expression in the lung tissue from mice dosed with the non-specific controls. There is an expected decrease in neutrophil and macrophage counts in the bronchoalveolar lavage fluid in cigarette smoke exposed mice that receive the MST1R siRNA or ASO compared to the neutrophil and macrophage counts in the bronchoalveolar lavage fluid in cigarette smoke exposed mice that receive the non-specific control. There is also an expected decrease in air space and mean linear intercept and an increase in septal wall thickness in cigarette smoke exposed mice that receive the MST1R siRNA or ASO compared to the air space, mean linear intercept and septal wall thickness in cigarette smoke exposed mice that receive the non-specific control. Additionally, there is also an expected decrease in compliance and tidal volume and an increase in resistance in cigarette smoke exposed mice that receive the MST1R siRNA or ASO compared to the compliance, tidal volume and resistance in cigarette smoke exposed mice that receive the non-specific control. These results will show that an MST1R siRNA or ASO may elicit knockdown of MST1R mRNA in lung and that the decrease in MST1R expression may correspond with a decrease in neutrophil and macrophage counts in the bronchoalveolar lavage fluid and increased lung function and decreased pathology in mice exposed to cigarette smoke.

### Example 9: Oligonucleotide Synthesis

Oligonucleotides such as siRNAs may be synthesized according to phosphoramidite technology on a solid phase. For example, a K&A oligonucleotide synthesizer may be used. Syntheses may be performed on a solid support made of controlled pore glass (CPG, 500 Å or 600 Å, obtained from AM Chemicals, Oceanside, CA, USA). All 2′-OMe and 2′-F phosphoramidites may be purchased from Hongene Biotech (Union City, CA, USA). All phosphoramidites may be dissolved in anhydrous acetonitrile (100 mM) and molecular sieves (3 Å) may be added. 5-Benzylthio-1H-tetrazole (BTT, 250 mM in acetonitrile) or 5-Ethylthio-1H-tetrazole (ETT, 250 mM in acetonitrile) may be used as activator solution. Coupling times may be 9-18 min (e.g. with a GalNAc such as ETL17), 6 min (e.g. with 2′OMe and 2′F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile may be employed.

After solid phase synthesis, the dried solid support may be treated with a 1:1 volume solution of 40 wt. % methylamine in water and 28% ammonium hydroxide solution (Aldrich) for two hours at 30° C. The solution may be evaporated and the solid residue may be reconstituted in water and purified by anionic exchange HPLC using a TKSgel SuperQ-5PW 13u column. Buffer A may be 20 mM Tris, 5 mM EDTA, pH 9.0 and contained 20% Acetonitrile and buffer B may be the same as buffer A with the addition of 1 M sodium chloride. UV traces at 260 nm may be recorded. Appropriate fractions may be pooled then desalted using Sephadex G-25 medium.

Equimolar amounts of sense and antisense strand may be combined to prepare a duplex. The duple solution may be prepared in 0.1×PBS (Phosphate-Buffered Saline, 1×, Gibco). The duplex solution may be annealed at 95° C. for 5 min, and cooled to room temperature slowly. Duplex concentration may be determined by measuring the solution absorbance on a UV-Vis spectrometer at 260 urn in 0.1×PBS, For some experiments, a conversion factor may be calculated from an experimentally determined extinction coefficient.

### Example 10: GalNAc Ligand for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. Reagents for GalNAc conjugation to oligonucleotides are shown in Table 11.

TABLE 11
GalNAc Conjugation Reagents

| Type of | |
|---|---|
| conjugation | Structure |
| Solid phase 3′ attachment where squiggly line is rest of oligonucleotide chain and right-most OH is where attachment to solid phase is. | 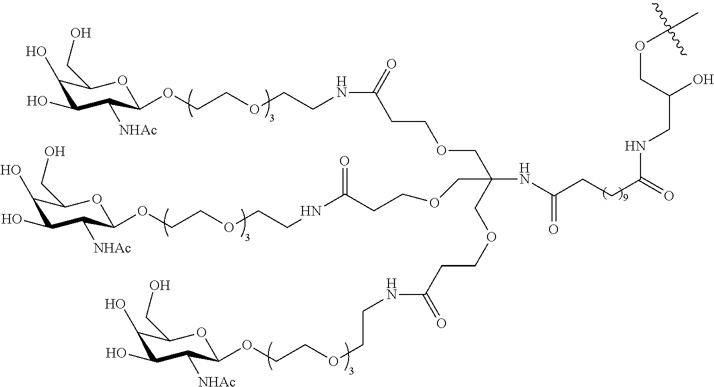 |
| | This GalNAc ligand may be referred to as “GalNAc23” or “GalNAc#23.” |
| Solid phase 5′ attachment phosphoramidite | 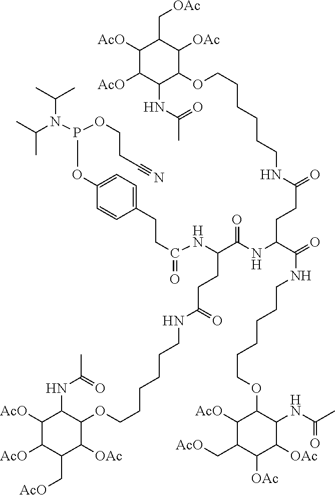 [SMILES: CC(=O)NC1C(OCCCCCCNC(=O)CCC(NC(=O)CCc2ccc(OP(OCCC#N)N(C(C)C)C(C)C)cc2)C(=O)NC(CCC(=O)NCCCCCCOC2C(NC(C)=O)C(OC(C)=O)C(OC(C)=O)C(COC(C)=O)C2OC(C)=O)C(=O)NCCCCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solid phase 5′ attachment Phosphoramidite | 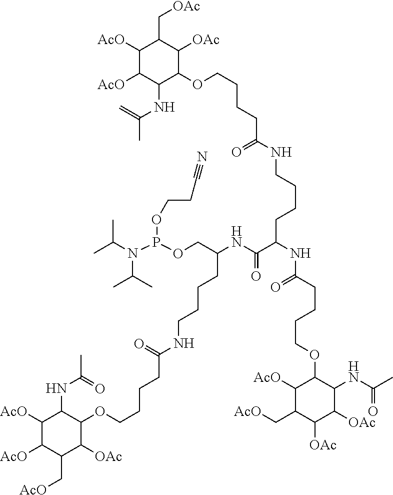 [SMILES: C=C(C)NC1C(OCCCCC(=O)NCCCCC(NC(=O)CCCCOC2C(NC(C)=O)C(OC(C)=O)C(OC(C)=O)C(COC(C)=O)C2OC(C)=O)C(=O)NC(CCCCNC(=O)CCCCOC2C(NC(C)=O)C(OC(C)=O)C(OC(C)=O)C(COC(C)=O)C2OC(C)=O)COP(OCCC#N)N(C(C)C)C(C)C)C(OC(C)=O)C(COC(C)=O)C(OC(C)=O)C1OC(C)=O] |
| Solution phase Carboxylic acid for amide coupling anywhere on oligonucleotide | 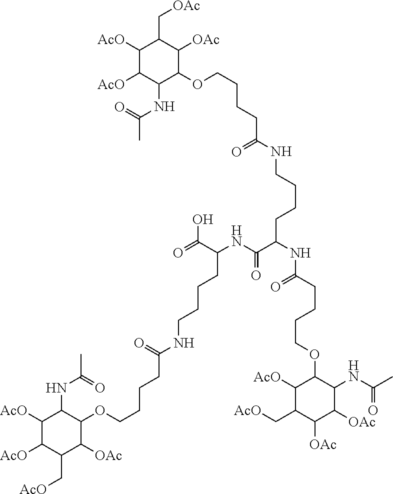 [SMILES: CC(=O)NC1C(OCCCCC(=O)NCCCCC(NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(=O)O)C(OC(C)=O)C(COC(C)=O)C(OC(C)=O)C1OC(C)=O] |
| | Where Ac is an acetyl group or other hydroxyl protecting group that can be removed |
| | under basic, acid or reducing conditions. |

In solution phase conjugation, the oligonucleotide sequence-including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide), EDC (N-(3-dimethylaminopropyl)-N′-ethylcarbodiimide) or EDC·HCl (N-(3-dimethylaminopropyl)-N′-ethylcarbodiimide hydrochloride and an additive like HOBt (1-hydroxybenztriazole), HOSu (N-hydroxysuccinimide), TBTU (N,N,N′,N′-Tetramethyl-O-(benzotriazol-1-yl)uronium tetrafluoroborate, HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between.

Non-limiting examples of reagents for oligonucleotide synthesis to incorporate an amino group include:
5′ attachment:
6-(4-Monomethoxytritylamino)hexyl-(2-cyanoethyl)-(N,N-diisopropyl)-phosphoramidite CAS Number: 114616-27-2
5′-Amino-Modifier TEG CE-Phosphoramidite
10-(O-trifluoroacetamido-N-ethyl)-triethyleneglycol-1-[(2-cyanoethyl)-(N,N-diisopropyl)]-phosphoramidite
3′ attachment:
3′-Amino-Modifier Serinol CPG
3-Dimethoxytrityloxy-2-(3-(fluorenylmethoxycarbonylamino)propanamido)propyl-1-O-succinyl-long chain alkylamino-CPG (where CPG stands for controlled-pore glass and is the solid support)
Amino-Modifier Serinol Phosphoramidite
3-Dimethoxytrityloxy-2-(3-(fluorenylmethoxycarbonylamino)propanamido)propyl-1-O-(2-cyanoethyl)-(N,N-diisopropyl)-phosphoramidite

### Internal (Base Modified):

Amino-Modifier C6 dT
5′-Dimethoxytrityl-5-[N-(trifluoroacetylaminohexyl)-3-acrylimido]-2′-deoxyUridine,3′-[(2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

### Example 11: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 12.

TABLE 12
GalNAc Conjugation Reagent

| Type of | |
|---|---|
| conjugation | Structure |
| Solid phase 5′ attachment phosphor- amidite | 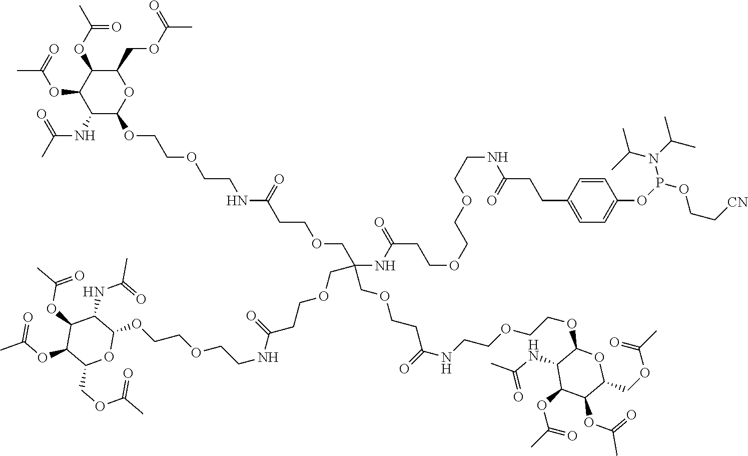 [SMILES: CC(=O)N[C@@H]1[C@H](OCCOCCNC(=O)CCOCC(COCCC(=O)NCCOCCO[C@H]2O[C@H](COC(C)=O)[C@@H](OC(C)=O)[C@@H](OC(C)=O)[C@@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)N(C(C)C)C(C)C)cc2)O[C@H](COC(C)=O)[C@@H](OC(C)=O)[C@H]1OC(C)=O] |

The following includes examples of synthesis reactions used to create a GalNAc moiety:

### Scheme for the Preparation of NAcegal-Linker-TMSOTf

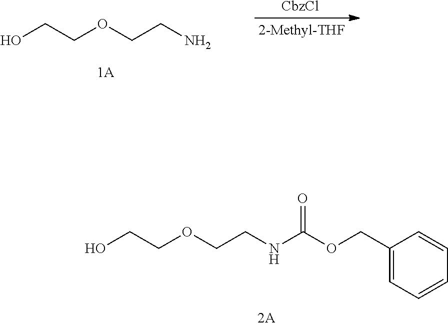 [SMILES: NCCOCCO ; "CbzCl" ; O=C(NCCOCCO)OCc1ccccc1]

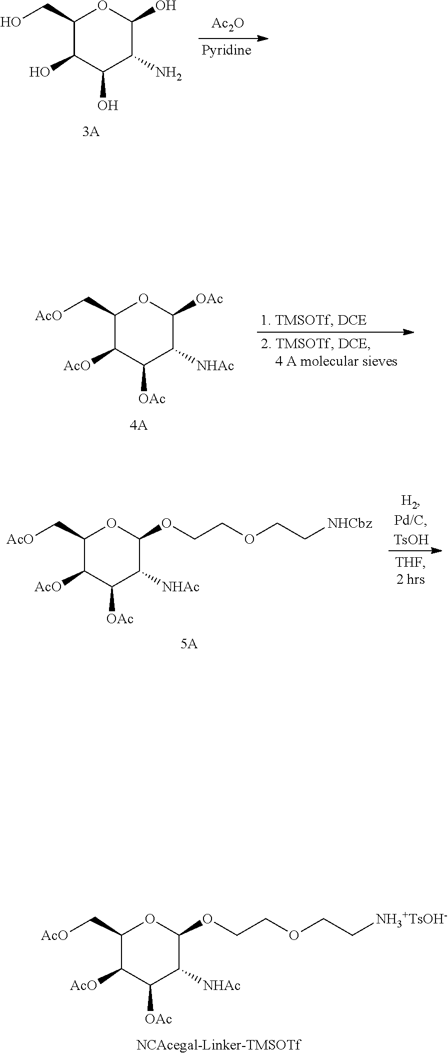

### General Procedure for Preparation of Compound 2A

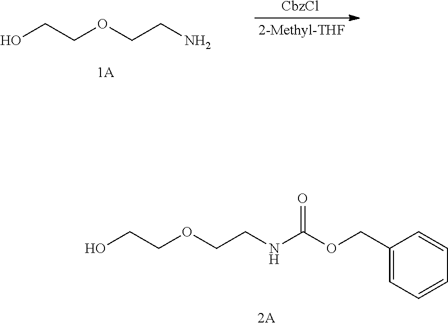 [SMILES: NCCOCCO ; "CbzCl" ; O=C(NCCOCCO)OCc1ccccc1]

To a solution of Compound 1A (500 g, 4.76 mol, 476 mL) in 2-Methly-THF (2.00 L) is added CbzCl (406 g, 2.38 mol, 338 mL) in 2-Methyl-THF (750 mL) dropwise at 0° C. The mixture is stirred at 25° C. for 2 hrs under N₂ atmosphere. TLC (DCM:MeOH=20:1, PMA) may indicate CbzCl is consumed completely and one new spot (R_{f}=0.43) formed. The reaction mixture is added HCl/EtOAc (1 N, 180 mL) and stirred for 30 mins, white solid is removed by filtration through celite, the filtrate is concentrated under vacuum to give Compound 2A (540 g, 2.26 mol, 47.5% yield) as a pale yellow oil and used into the next step without further purification. ¹H NMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

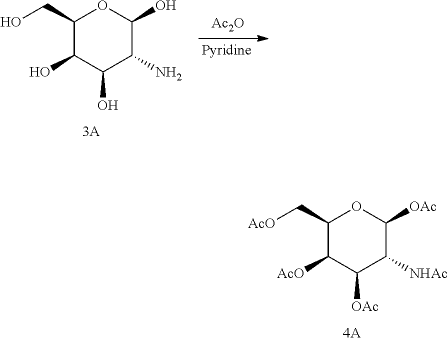 [SMILES: "Ac2O" ; N[C@@H]1[C@@H](O)[C@@H](O)[C@@H](CO)O[C@H]1O ; CC(=O)N[C@H]1[C@H](OC(C)=O)O[C@H](COC(C)=O)[C@H](OC(C)=O)[C@@H]1OC(C)=O]

To a solution of Compound 3A (1.00 kg, 4.64 mol, HCl) in pyridine (5.00 L) is added acetyl acetate (4.73 kg, 46.4 mol, 4.34 L) dropwise at 0° C. under N₂ atmosphere. The mixture is stirred at 25° C. for 16 hrs under N₂ atmosphere. TLC (DCM:MeOH=20:1, PMA) indicated Compound 3A is consumed completely and two new spots (R_{f}=0.35) formed. The reaction mixture is added to cold water (30.0 L) and stirred at 0° C. for 0.5 hr, white solid formed, filtered and dried to give Compound 4A (1.55 kg, 3.98 mol, 85.8% yield) as a white solid and used in the next step without further purification. ¹H NMR: δ 7.90 (d, J=9.29 Hz, 1H), 5.64 (d, J=8.78 Hz, 1H), 5.26 (d, J=3.01 Hz, 1H), 5.06 (dd, J=11.29, 3.26 Hz, 1H), 4.22 (t, J=6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5A

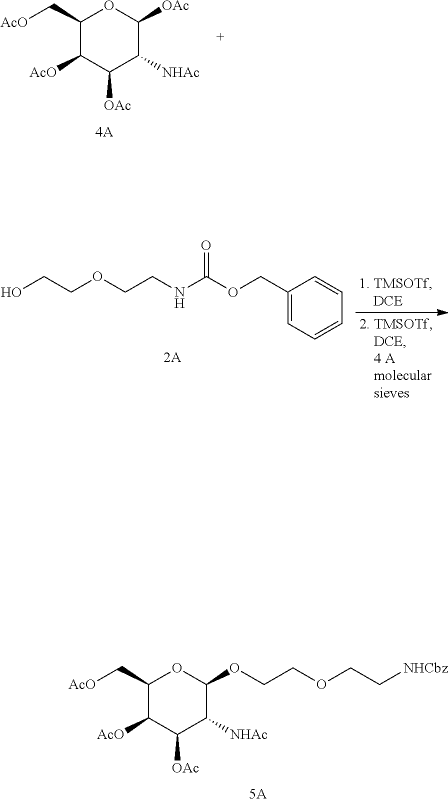

To a solution of Compound 4A (300 g, 771 mmol) in DCE (1.50 L) is added TMSOTf (257 g, 1.16 mol, 209 mL) and stirred for 2 hrs at 60° C., and then stirred for 1 hr at 25° C. Compound 2A (203 g, 848 mmol) is dissolved in DCE (1.50 L) and added 4 Å powder molecular sieves (150 g) stirring for 30 mins under N₂ atmosphere. Then the solution of Compound 4A in DCE is added dropwise to the mixture at 0° C. The mixture is stirred at 25° C. for 16 hrs under N₂ atmosphere. TLC (DCM:MeOH=25:1, PMA) indicated Compound 4A is consumed completely and new spot (R_{f}=0.24) formed. The reaction mixture is filtered and washed with sat. NaHCO₃ (2.00 L), water (2.00 L) and sat. brine (2.00 L). The organic layer is dried over anhydrous Na₂SO₄, filtered and concentrated under reduced pressure to give a residue. The residue is triturated with 2-Me-THE/heptane (5/3, v/v, 1.80 L) for 2 hrs, filtered and dried to give Compound 5A (225 g, 389 mmol, 50.3% yield, 98.4% purity) as a white solid. ¹H NMR: δ 7.81 (d, J 9.29 Hz, 1H), 7.20-7.42 (m, 6H), 5.21 (d, J=3.26 Hz, 1H), 4.92-5.05 (m, 3H), 4.55 (d, J=8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J=5.77 Hz, 2H), 2.10 (s, 3H), 1.99 (s, 3H), 1.89 (s, 3H), 1.77 (s, 3H).

### General Procedure for Preparation of NAcegal-Linker-Tosylate Salt

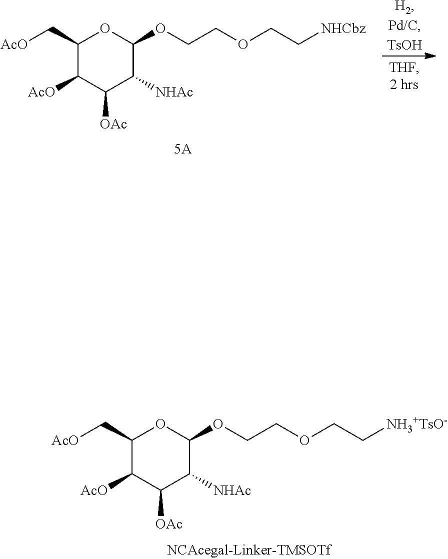

To a solution of Compound 5A (200 g, 352 mmol) in THF (1.0 L) is added dry Pd/C (15.0 g, 10% purity) and TsOH (60.6 g, 352 mmol) under N₂ atmosphere. The suspension is degassed under vacuum and purged with H₂ several times. The mixture is stirred at 25° C. for 3 hrs under H₂ (45 psi) atmosphere. TLC (DCM:MeOH=10:1, PMA) indicated Compound 5A is consumed completely and one new spot (R_{f}=0.04) is formed. The reaction mixture is filtered and concentrated (≤40° C.) under reduced pressure to give a residue. Diluted with anhydrous DCM (500 mL, dried overnight with 4 Å molecular sieves (dried at 300° C. for 12 hrs)) and concentrate to give a residue and run Karl Fisher (KF) to check for water content. This is repeated 3 times with anhydrous DCM (500 mL) dilutions and concentration to give NAcegal-Linker-TMSOTf (205 g, 95.8% yield, TsOH salt) as a foamy white solid. ¹H NMR: δ 7.91 (d, J 9.03 Hz, 1H), 7.53-7.86 (m, 2H), 7.49 (d, J=8.03 Hz, 2H), 7.13 (d, J=8.03 Hz, 2H), 5.22 (d, J 3.26 Hz, 1H), 4.98 (dd, J=11.29, 3.26 Hz, 1H), 4.57 (d, J=8.53 Hz, 1H), 3.99-4.05 (m, 3H), 3.87-3.94 (m, 1H), 3.79-3.85 (m, 1H), 3.51-3.62 (m, 5H), 2.96 (br t, J=5.14 Hz, 2H), 2.29 (s, 3H), 2.10 (s, 3H), 2.00 (s, 3H), 1.89 (s, 3H), 1.78 (s, 3H).

### Scheme for the Preparation of TRIS-PEG2-CBZ

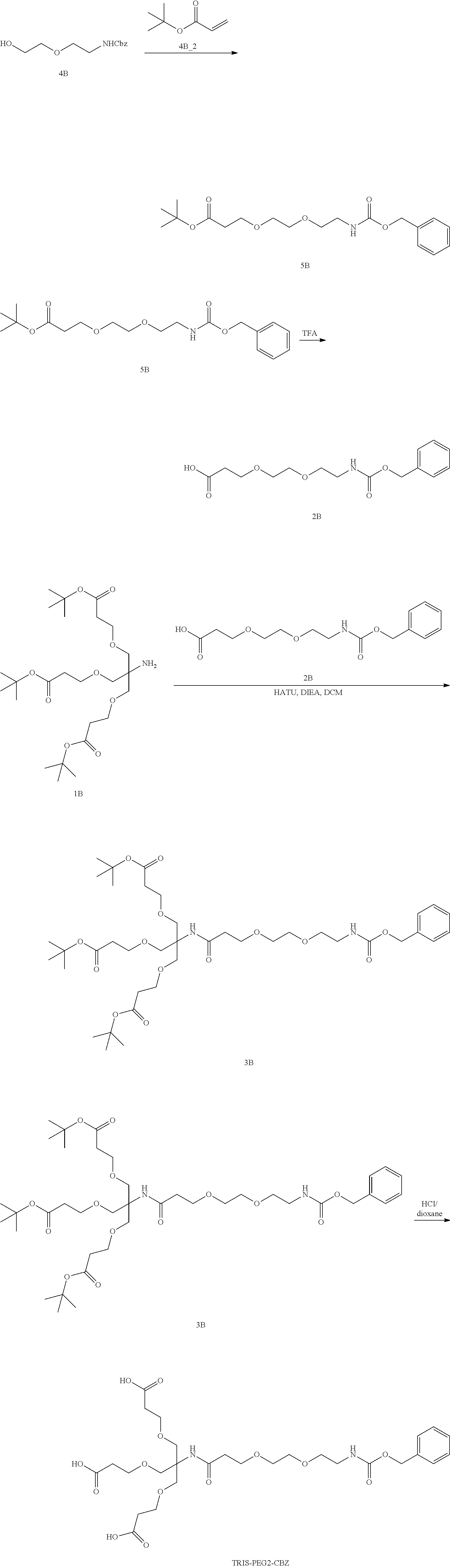

### General Procedure for Preparation of Compound 5B

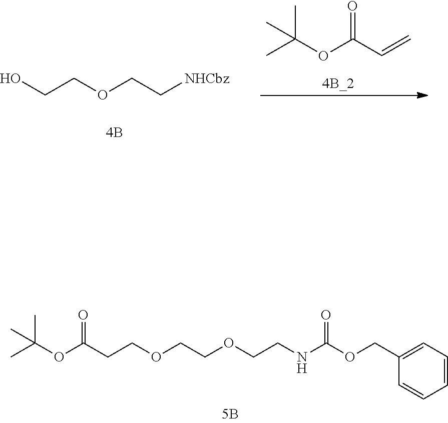

To a solution of Compound 4B (400 g, 1.67 mol, 1.00 eq) and NaOH (10 M, 16.7 mL, 0.10 eq) in THF (2.00 L) is added Compound 4B_2 (1.07 kg, 8.36 mol, 1.20 L, 5.00 eq), the mixture is stirred at 30° C. for 2 hrs. LCMS showed the desired MS is given. Five batches of solution are combined to one batch, then the mixture is diluted with water (6.00 L), extracted with ethyl acetate (3.00 L*3), the combined organic layer is washed with brine (3.00 L), dried over Na₂SO₄, filtered and concentrated under vacuum. The crude is purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

### General procedure for preparation of 3-oxo-1-phenyl-2,7,10-trioxa-4-azatridecan-13-oic acid (Compound 2B below)

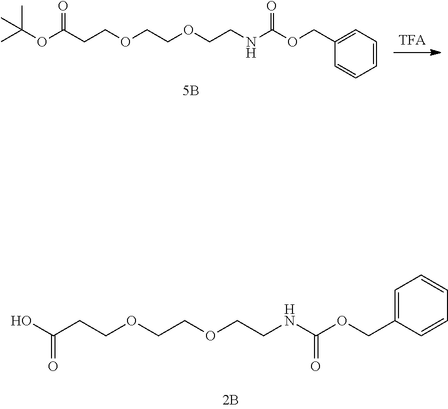 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) is added TFA (1.43 kg, 12.5 mol, 928 mL, 6.22 eq), the mixture is stirred at 25° C. for 3 hrs. LCMS showed the desired MS is given. The mixture is diluted with DCM (5.00 L), washed with water (3.00 L*3), brine (2.00 L), the combined organic layer is dried over Na₂SO₄, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

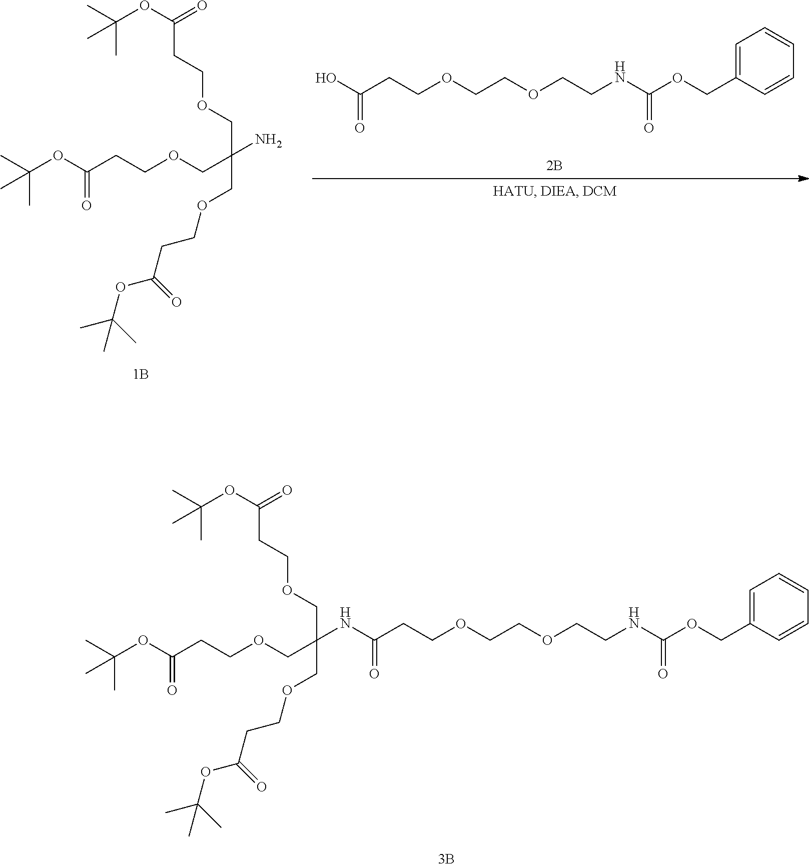 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) is added HATU (570 g, 1.50 mol, 1.50 eq) and DIEA (258 g, 2.00 mol, 348 mL, 2.00 eq) at 0° C., the mixture is stirred at 0° C. for 30 min, then Compound 1B (606 g, 1.20 mol, 1.20 eq) is added, the mixture is stirred at 25° C. for 1 hr. LCMS showed desired MS is given. The mixture is combined to one batch, then the mixture is diluted with DCM (5.00 L), washed with 1 N HCl aqueous solution (2.00 L*2), then the organic layer is washed with saturated Na₂CO₃ aqueous solution (2.00 L*2) and brine (2.00 L), the organic layer is dried over Na₂SO₄, filtered and concentrated under vacuum to give Compound 3B (3.88 kg, crude) as yellow oil.

### General Procedure for Preparation of TRIS-PEG2-CBZ.

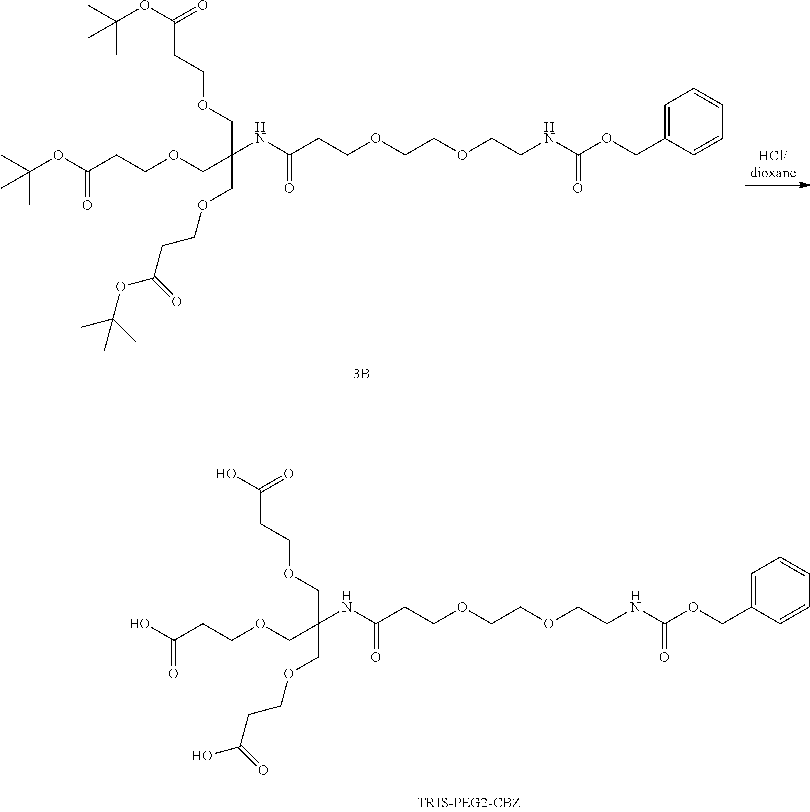 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) is stirred at 25° C. for 2 hrs. LCMS showed the desired MS is given. The mixture is concentrated under vacuum to give a residue. Then the combined residue is diluted with DCM (5.00 L), adjusted to pH=8 with 2.5 M NaOH aqueous solution, and separated. The aqueous phase is extracted with DCM (3.00 L) again, then the aqueous solution is adjusted to pH=3 with 1 N HCl aqueous solution, then extracted with DCM (5.00 L*2), the combined organic layer is washed with brine (3.00 L), dried over Na₂SO₄, filtered and concentrated under vacuum. The crude is purified by column chromatography (SiO₂, DCM:MeOH=0:1-12:1, 0.1% HOAc, R_{f}=0.4). The residue is diluted with DCM (5.00 L), adjusted to pH=8 with 2.5 M NaOH aqueous solution, separated, the aqueous solution is extracted with DCM (3.00 L) again, then the aqueous solution is adjusted to pH=3 with 6 N HCl aqueous solution, extracted with DCM:MeOH=10:1 (5.00 L*2), the combined organic layer is washed with brine (2.00 L), dried over Na₂SO₄, filtered and concentrated under vacuum to give a residue. Then the residue is diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHz, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

### Scheme for the Preparation of TriNGal-TRIS-Peg2-Phosph 8c

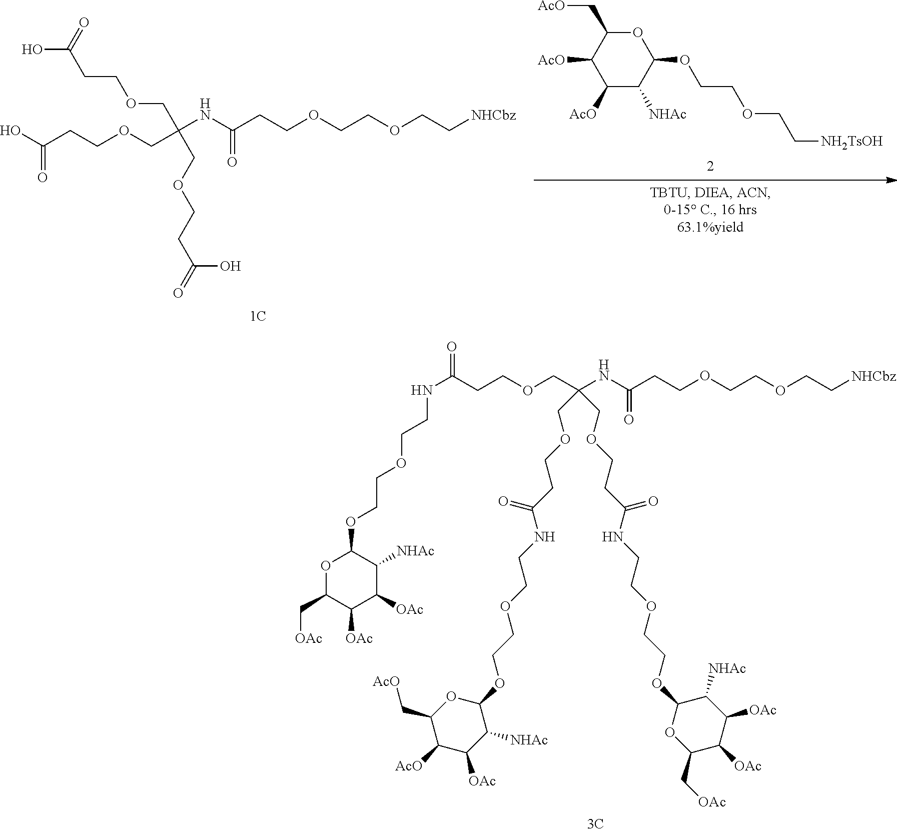

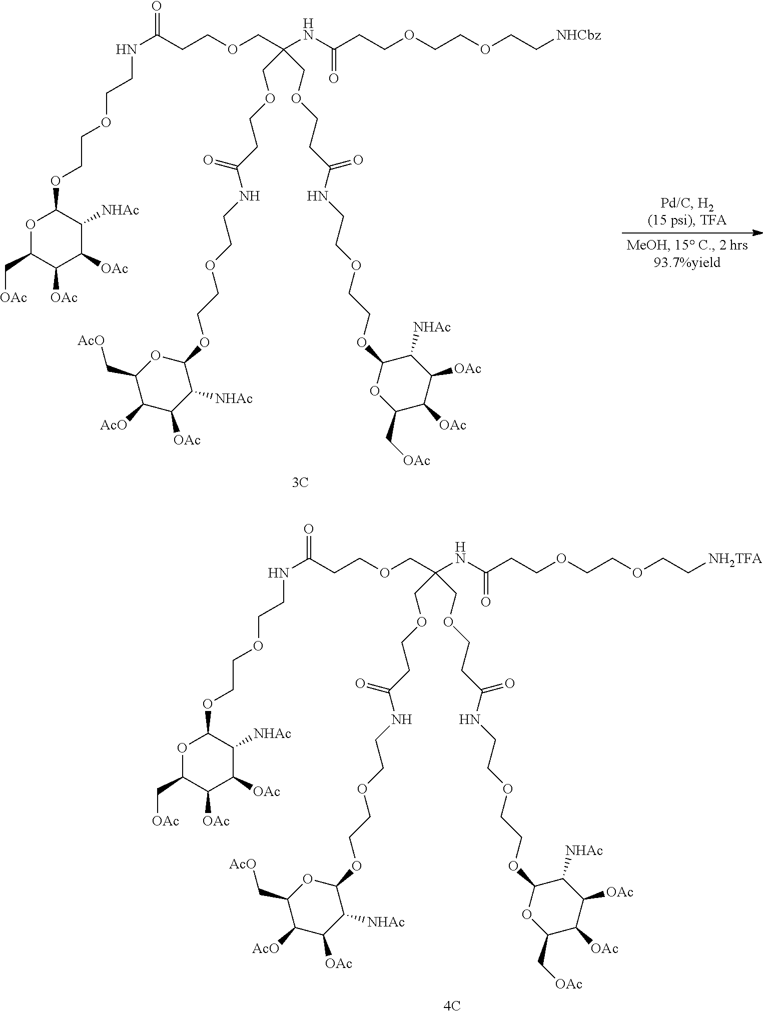

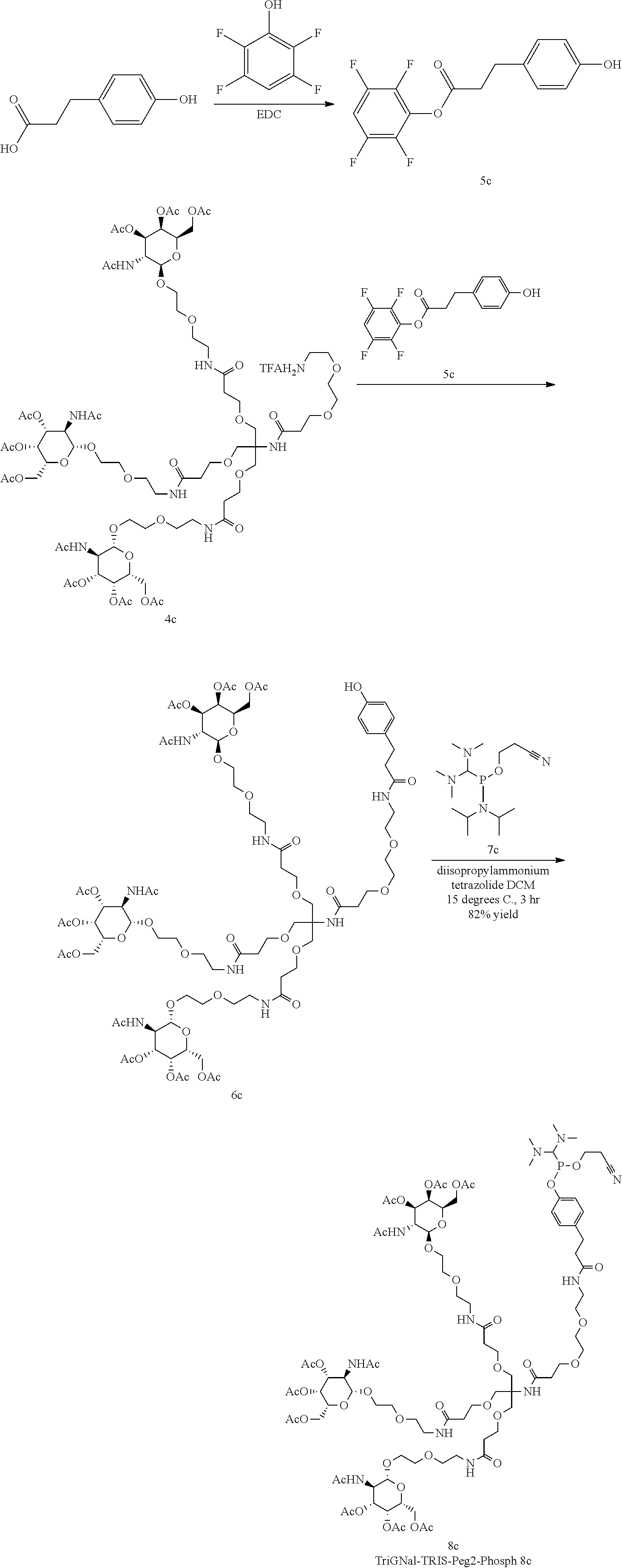

### General Procedure for Preparation of Compound 3C

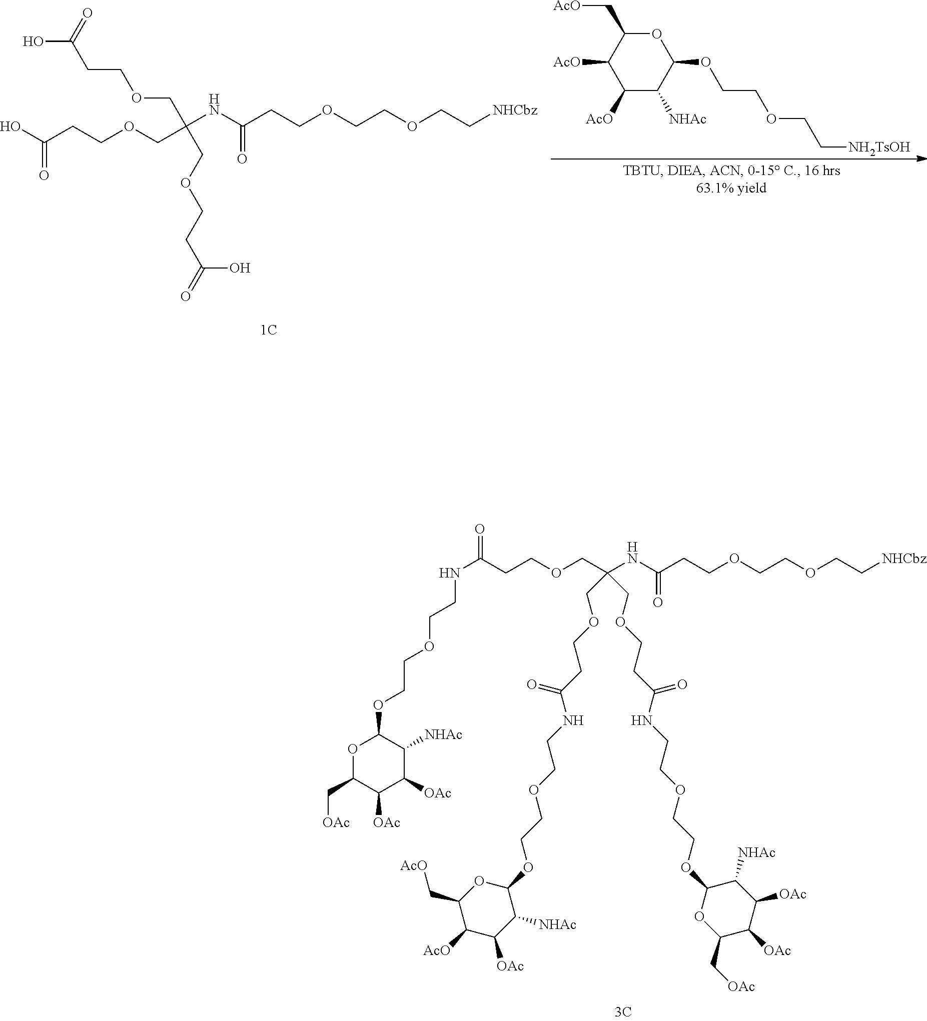

To a solution of Compound 1C (155 g, 245 mmol, 1.00 eq) in ACN (1500 mL) is added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture is stirred at 15° C. for 16 hrs. LCMS showed the desired MS is given. The mixture is concentrated under vacuum to give a residue, then the mixture is diluted with DCM (2000 mL), washed with 1 N HCl aqueous solution (700 mL*2), then saturated NaHCO₃ aqueous solution (700 mL*2) and concentrated under vacuum. The crude is purified by column chromatography to give Compound 3C (304 g, 155 mmol, 63.1% yield, 96.0% purity) as a yellow solid.

### General Procedure for Preparation of Compound 4C

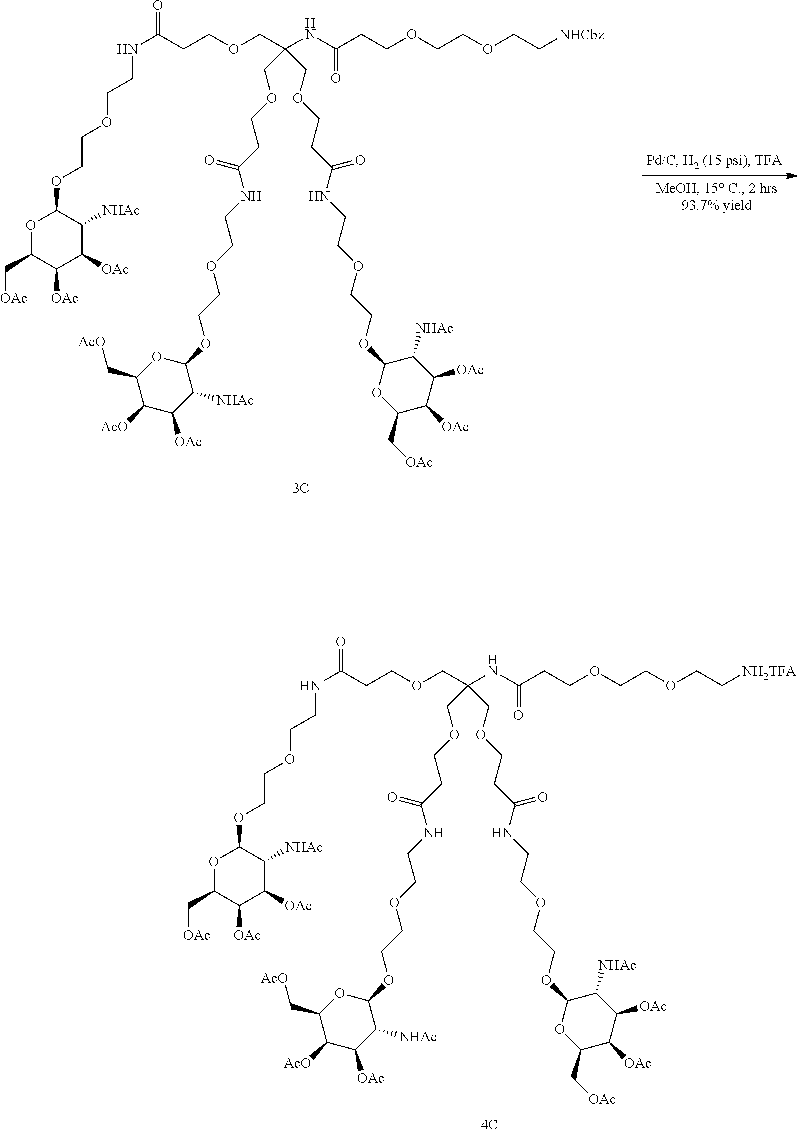

Two batches solution of Compound 3C (55.0 g, 29.2 mmol, 1.00 eq) in MeOH (1600 mL) is added Pd/C (6.60 g, 19.1 mmol, 10.0% purity) and TFA (3.34 g, 29.2 mmol, 2.17 mL, 1.00 eq), the mixture is degassed under vacuum and purged with H₂. The mixture is stirred under H₂ (15 psi) at 15° C. for 2 hours. LCMS showed the desired MS is given. The mixture is filtered and the filtrate is concentrated under vacuum to give Compound 4C (106 g, 54.8 mmol, 93.7% yield, 96.2% purity, TFA) as a white solid.

### General Procedure for Preparation of Compound 5C

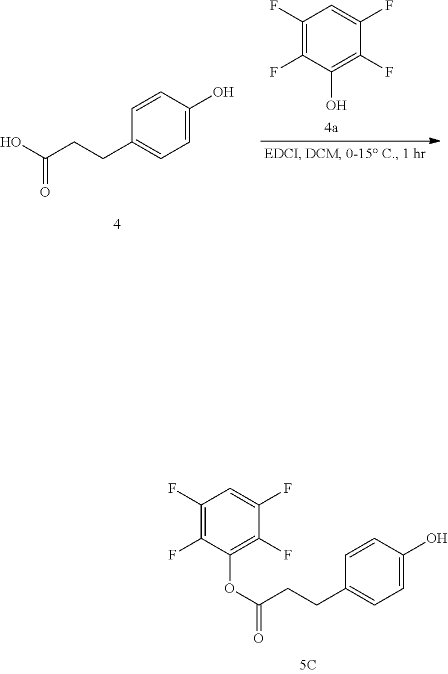 [SMILES: O=C(O)CCc1ccc(O)cc1 ; Oc1c(F)c(F)cc(F)c1F ; O=C(CCc1ccc(O)cc1)Oc1c(F)c(F)cc(F)c1F]

Two batches in parallel. To a solution of EDCI (28.8 g, 150 mmol, 1.00 eq) in DCM (125 mL) is added compound 4a (25.0 g, 150 mmol, 1.00 eq) dropwise at 0° C., then the mixture is added to compound 4 (25.0 g, 150 mmol, 1.00 eq) in DCM (125 mL) at 0° C., then the mixture is stirred at 25° C. for 1 hr. TLC (Petroleum ether:Ethyl acetate=3:1, R_{f}=0.45) showed the reactant is consumed and one new spot is formed. The reaction mixture is diluted with DCM (100 mL) then washed with aq·NaHCO₃ (250 mL*1) and brine (250 mL), dried over Na₂SO₄, filtered and concentrated under reduced pressure to give a residue. The residue is purified by column chromatography (SiO₂, Petroleum ether:Ethyl acetate=100:1 to 3:1), TLC (SiO₂, Petroleum ether:Ethyl acetate=3:1), R_{f}=0.45, then concentrated under reduced pressure to give a residue. Compound 5C (57.0 g, 176 mmol, 58.4% yield, 96.9% purity) is obtained as colorless oil and confirmed ¹HNMR: EW33072-2-P1A, 400 MHz, DMSO δ 9.21 (s, 1H), 7.07-7.09 (m, 2H), 6.67-6.70 (m, 2H), 3.02-3.04 (m, 2H), 2.86-2.90 (m, 2H)

### General Procedure for Preparation of Compound 6

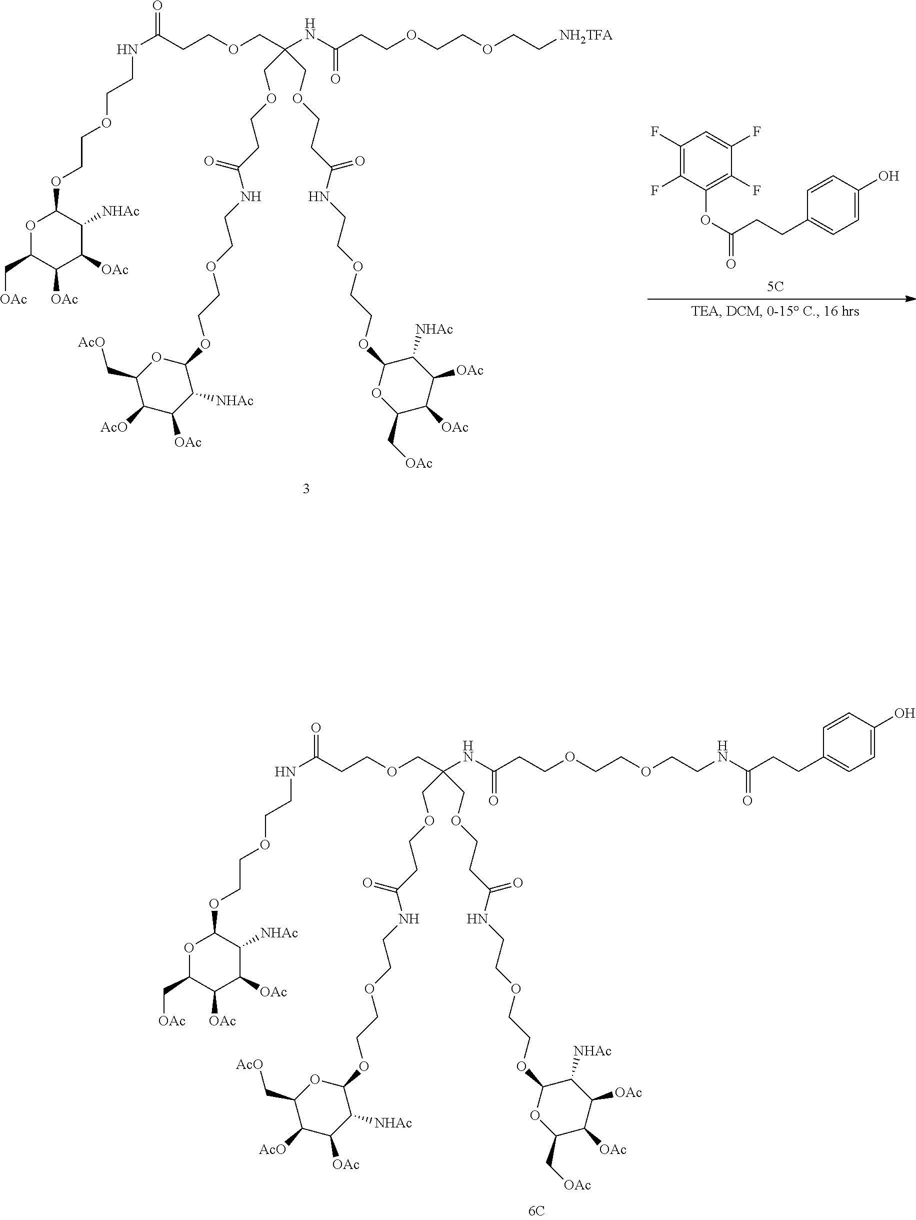

To a mixture of compound 3 (79.0 g, 41.0 mmol, 96.4% purity, 1.00 eq, TFA) and compound 6C (14.2 g, 43.8 mmol, 96.9% purity, 1.07 eq) in DCM (800 mL) is added TEA (16.6 g, 164 mmol, 22.8 mL, 4.00 eq) dropwise at 0° C., the mixture is stirred at 15° C. for 16 hrs. LCMS (EW33072-12-P1B, Rt=0.844 min) showed the desired mass is detected. The reaction mixture is diluted with DCM (400 mL) and washed with aq·NaHCO₃ (400 mL*1) and brine (400 mL*1), then the mixture is diluted with DCM (2.00 L) and washed with 0.7 M Na₂CO₃ (1000 mL*3) and brine (800 mL*3), dried over Na₂SO₄, filtered and concentrated under reduced pressure to give a residue. The residue is used to next step directly without purification. Compound 6 (80.0 g, crude) is obtained as white solid and confirmed via ¹HNMR: EW33072-12-P1A, 400 MHz, MeOD δ 7.02-7.04 (m, 2H), 6.68-6.70 (m, 2H), 5.34-5.35 (s, 3H), 5.07-5.08 (d, J=4.00 Hz, 3H), 4.62-4.64 (d, J=8.00 Hz, 3H), 3.71-4.16 (m, 16H), 3.31-3.70 (m, 44H), 2.80-2.83 (m, 2H), 2.68 (m, 2H), 2.46-2.47 (m, 10H), 2.14 (s, 9H), 2.03 (s, 9H), 1.94-1.95 (d, J=4.00 Hz, 18H).

### General Procedure for Preparation of TriGNal-TRIS-Peg2-Phosph 8c

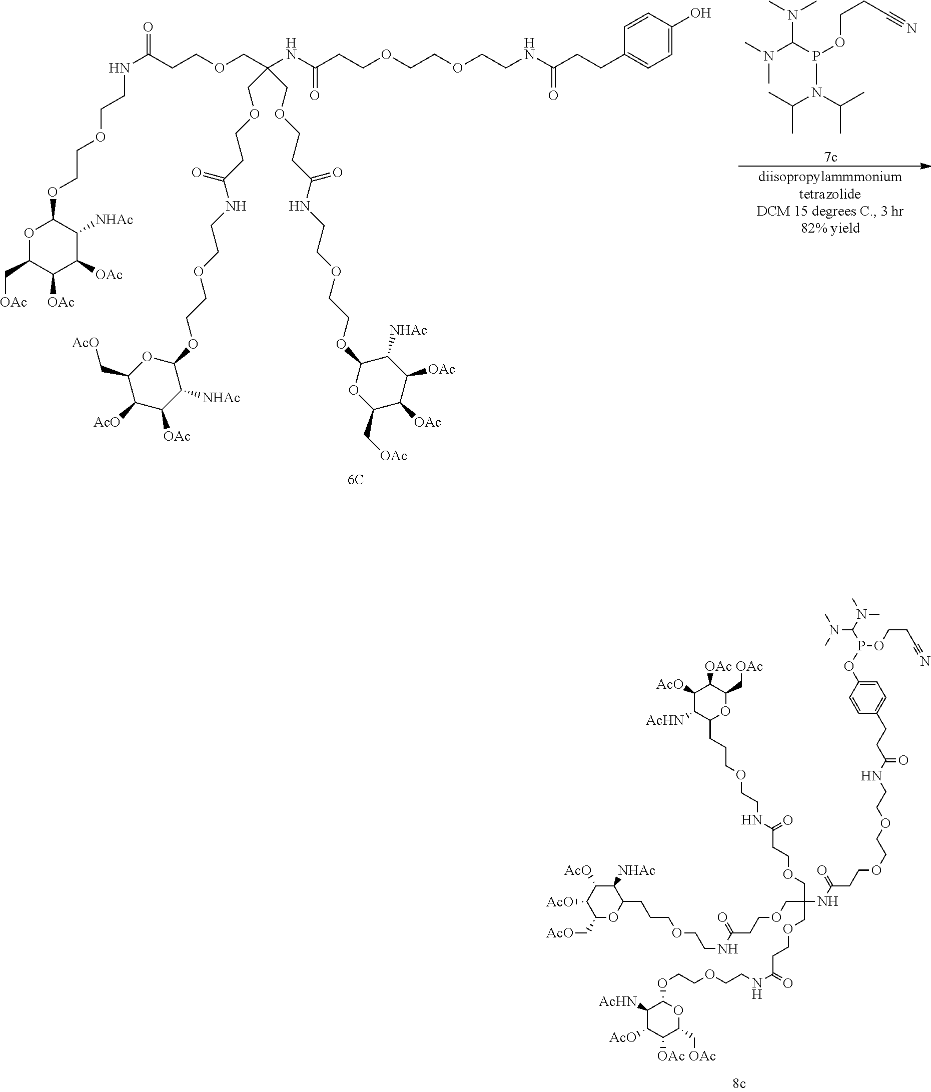

Two batches are synthesized in parallel. To a solution of compound 6C (40.0 g, 21.1 mmol, 1.00 eq in DCM (600 mL) is added diisopropylammonium tetrazolide (3.62 g, 21.1 mmol, 1.00 eq) and compound 7c (6.37 g, 21.1 mmol, 6.71 mL, 1.00 eq) in DCM (8.00 mL) drop-wise, the mixture is stirred at 30° C. for 1 hr, then added compound 7c (3.18 g, 10.6 mmol, 3.35 mL, 0.50 eq) in DCM (8.00 mL) drop-wise, the mixture is stirred at 30° C. for 30 mins, then added compound 7c (3.18 g, 10.6 mmol, 3.35 mL, 0.50 eq) in DCM (8.00 mL) drop-wise, the mixture is stirred at 30° C. for 1.5 hrs. LCMS (EW33072-17-P1C1, Rt=0.921 min) showed the desired MS+1 is detected. LCMS (EW33072-17-P1C2, Rt=0.919 min) showed the desired MS+1 is detected. Two batches are combined for work-up. The mixture is diluted with DCM (1.20 L), washed with saturated NaHCO₃ aqueous solution (1.60 L*2), 3% DMF in H₂O (1.60 L*2), H₂O (1.60 L*3), brine (1.60 L), dried over Na₂SO₄, filtered and concentrated under reduced pressure to give a residue. The residue is purified by column chromatography (SiO₂, DCM:MeOH:TEA=100:3:2) TLC (SiO₂, DCM:MeOH=10:1, R_{f}=0.45), then concentrated under reduced pressure to give a residue. Compound 8C (76.0 g, 34.8 mmol, 82.5% yield, 96.0% purity) is obtained as white solid and confirmed via ¹HNMR: EW33072-19-P1C, 400 MHz, MeOD δ 7.13-7.15 (d, J=8.50 Hz, 2H), 6.95-6.97 (dd, J=8.38, 1.13 Hz, 2H), 5.34 (d, J=2.88 Hz, 3H), 0.09 (dd, J=11.26, 3.38 Hz, 3H), 4.64 (d, J=8.50 Hz, 3H), 3.99-4.20 (m, 12H), 3.88-3.98 (m, 5H), 3.66-3.83 (m, 20H), 3.51-3.65 (m, 17H), 3.33-3.50 (m, 9H), 2.87 (t, J=7.63 Hz, 2H), 2.76 (t, J=5.94 Hz, 2H), 2.42-2.50 (m, 10H), 2.14 (s, 9H), 2.03 (s, 9H), 1.94-1.95 (d, J=6.13 Hz, 18H), 1.24-1.26 (d, J=6.75 Hz, 6H), 1.18-1.20 (d, J=6.75 Hz, 6H)

### Example 12: Modification Motif 1

An example MST1R siRNA includes a combination of the following modifications:
Position 9 (from 5′ to 3′) of the sense strand is 2′ F.
If position 9 is a pyrimidine then all purines in the Sense Strand are 2′OMe, and 1-5 pyrimidines between positions 5 and 11 are 2′ F provided that there are never three 2′F modifications in a row.
If position 9 is a purine then all pyrimidines in the Sense Strand are 2′OMe, and 1-5 purines between positions 5 and 11 are 2′ F provided that there are never three 2′F modifications in a row.
Antisense strand odd-numbered positions are 2′OMe and even-numbered positions are a mixture of 2′ F, 2′OMe and 2′ deoxy.

### Example 13: Modification Motif 2

An example MST1R siRNA includes a combination of the following modifications:
Position 9 (from 5′ to 3′) of the sense strand is 2′ deoxy.
Sense strand positions 5, 7 and 8 are 2′ F.
All pyrimidines in positions 10-21 are 2′OMe, and purines are a mixture of 2′OMe and 2′ F. Alternatively, all purines in positions 10-21 are 2′OMe and all pyrimidines in positions 10-21 are a mixture of 2′OMe and 2′ F.
Antisense strand odd-numbered positions are 2′OMe and even-numbered positions are a mixture of 2′F, 2′OMe and 2′ deoxy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

### IV. SEQUENCE INFORMATION

Some embodiments include one or more nucleic acid sequences as described in Table 13:

TABLE 13
Sequence Information

| SEQ ID | |
|---|---|
| NO: | Description |
| 1-4754 | MST1R siRNA sense strand sequences |
| 4755-9508 | MST1R siRNA antisense strand sequences |
| 9509-9611 | Base sequences of modified MST1R siRNA sense strand |
| | sequences |
| 9612-9714 | Base sequences of modified MST1R siRNA antisense |
| | strand sequences |
| 9715-9817 | Base sequences of alternatively modified MST1R siRNA |
| | sense strand sequences |
| 9818 | Full-length human MST1R mRNA sequence (Ensembl |
| | transcript ID: ENST00000296474.8) (human RNA) |
| 9819-9836 | Modification patterns 1S to 9S, 1AS to 8AS, and ASO1 |
| 9837-9839 | Examples of RGD peptide sequences |

1. A composition comprising an oligonucleotide that targets MST1R and when administered to a subject in an effective amount improves:
(a) a lung function measurement;
(b) a leukocyte measurement;
(c) a chronic obstructive pulomonary disease (COPD) symptom or exacerbation measurement; or
(d) an asthma symptom or exacerbation measurement.

2. The composition of claim 1, wherein the lung function measurement comprises a forced expiratory volume in 1 second (FEV1) measurement, a forced expiratory volume in 1 second percent predicted (FEV1pp) measurement, a forced vital capacity (FVC) measurement, a FEV1/FVC ratio measurement, a forced expiratory volume, or a peak expiratory flow measurement.

3-10. (canceled)

11. The composition of claim 1, wherein the oligonucleotide comprises a modified internucleoside linkage.

12. The composition of claim 11, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

13. (canceled)

14. The composition of claim 1, wherein the oligonucleotide comprises 1, 2, 3, 4, 5, 6, 7, 8, 9, 10, 11, 12, 13, 14, 15, 16, 17, 18, 19, or 20 modified internucleoside linkages.

15. The composition of claim 1, wherein the oligonucleotide comprises a modified nucleoside.

16. The composition of claim 15, wherein the modified nucleoside comprises a locked nucleic acid (LNA), hexitol nucleic acid (HNA), cyclohexene nucleic acid (CeNA), 2′-O-methoxyethyl, 2′-O-alkyl, 2′-O-allyl, 2′-fluoro, or 2′-deoxy, 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

17-21. (canceled)

22. The composition of claim 1, wherein the oligonucleotide comprises 1, 2, 3, 4, 5, 6, 7, 8, 9, 10, 11, 12, 13, 14, 15, 16, 17, 18, 19, 20, or 21 modified nucleosides.

23. The composition of claim 1, wherein the oligonucleotide comprises a lipid, a sugar moiety, or an integrin targeting ligand attached at a 3′ or 5′ terminus of the oligonucleotide.

24. The composition of claim 23, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

25. (canceled)

26. The composition of claim 23, wherein the integrin comprises integrin alpha-v-beta-6 or the integrin targeting ligand comprises an arginine-glycine-aspartic acid (RGD) peptide.

27. (canceled)

28. The composition of claim 1, wherein the oligonucleotide comprises a small interfering RNA (siRNA) comprising a sense strand and an antisense strand, and each strand is independently 12-30 nucleosides in length.

29. (canceled)

30. (canceled)

31. A composition comprising an oligonucleotide that inhibits the expression of MST1R, wherein the oligonucleotide comprises
(a) an siRNA comprising a sense strand and an antisense strand, each strand is independently 12-30 nucleosides in length, and at least one of the sense strand and the antisense strand comprises a nucleoside sequence comprising 12-30 contiguous nucleosides of SEQ ID NO: 9818; or
(b) antisense oligonucleotide (ASO) comprising 12-30 nucleosides in length and a nucleoside sequence complementary to about 12-30 contiguous nucleosides of SEQ ID NO: 9818.

32. The composition of claim 28, wherein any one of the following is true with regard to the sense strand:
all purines comprise 2′-fluoro modified purines, and all pyrimidines comprise a mixture of 2′-fluoro and 2′-O-methyl modified pyrimidines;
all purines comprise 2′-O-methyl modified purines, and all pyrimidines comprise a mixture of 2′-fluoro and 2′-O-methyl modified pyrimidines;
all purines comprise 2′-fluoro modified purines, and all pyrimidines comprise 2′-O-methyl modified pyrimidines;
all pyrimidines comprise 2′-fluoro modified pyrimidines, and all purines comprise a mixture of 2′-fluoro and 2′-O-methyl modified purines;
all pyrimidines comprise 2′-O-methyl modified pyrimidines, and all purines comprise a mixture of 2′-fluoro and 2′-O-methyl modified purines; or
all pyrimidines comprise 2′-fluoro modified pyrimidines, and all purines comprise 2′-Q-methyl modified purines.

33. The composition of claim 28, wherein any one of the following is true with regard to the antisense strand:
all purines comprise 2′-fluoro modified purines, and all pyrimidines comprise a mixture of 2′-fluoro and 2′-O-methyl modified pyrimidines;
all purines comprise 2′-O-methyl modified purines, and all pyrimidines comprise a mixture of 2′-fluoro and 2′-O-methyl modified pyrimidines;
all purines comprise 2′-O-methyl modified purines, and all pyrimidines comprise 2′-fluoro modified pyrimidines;
all pyrimidines comprise 2′-fluoro modified pyrimidines, and all purines comprise a mixture of 2′-fluoro and 2′-O-methyl modified purines;
all pyrimidines comprise 2′-O-methyl modified pyrimidines, and all purines comprise a mixture of 2′-fluoro and 2′-O-methyl modified purines; or
all pyrimidines comprise 2′-O-methyl modified pyrimidines, and all purines comprise 2′-fluoro modified purines.

34-36. (canceled)

37. The composition of claim 1, further comprising a pharmaceutically acceptable carrier.

38. A method of treating a subject having a lung disorder, comprising administering an effective amount of a composition comprising an oligonucleotide that modulates the expression of MST1R, wherein the oligonucleotide comprises
(a) an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 9818; or
(b) antisense oligonucleotide (ASO) comprising 12-30 nucleosides in length and a nucleoside sequence complementary to about 12-30 contiguous nucleosides of SEQ ID NO: 9818 to the subject.

39. The method of claim 38, wherein the lung disorder comprises COPD, acute exacerbation of COPD, emphysema, chronic bronchitis, asthma, status asthmaticus, asthma-COPD overlap syndrome (ACOS), bronchiectasis, cough, dyspnea, mucas hypersecretion, lung cancer, interstitial lung disease, or pulmonary fibrosis.

40. The composition of claim 1, wherein the leukocyte measurement comprises a neutrophil measurement, an eosinophil measurement, a basophil measurement, a monocyte measurement, a lymphocyte measurement, a macrophage measurement, a neutrophil lymphocyte ratio measurement, or a combination thereof.

41. The composition of claim 23, wherein the sugar comprises N-acetylgalactosamine (GalNAc), N-acetylglucosamine (GlcNAc), or mannose.
